(19) United States
(12) Patent Application Publication
(10) Pub. No.: US 2024/0317871 A1
(43) Pub. Date: Sep. 26, 2024

(54) Anti-HVEM Antibodies

(71) Applicant: Immunomic Therapeutics, Inc., Rockville, MD (US)

(72) Inventors: Teri Heiland, New Market, MD (US); Wenhai Liu, Germantown, MD (US)

(73) Assignee: Immunomic Therapeutics, Inc., Rockville, MD (US)

(21) Appl. No.: 18/270,036

(22) PCT Filed: Dec. 29, 2021
(86) PCT No.: PCT/US2021/065491

§ 371 Date: Jun. 28, 2023

Related U.S. Application Data
(60) Provisional application No. 63/131,829, filed on Dec. 30, 2020.

(51) Int. Cl. C07K 16/28; G01N 33/569; G01N 33/574

(52) U.S. Cl. CPC C07K 16/2878; G01N 33/56988; G01N 33/57484; C07K 2317/92; G01N 2333/16; G01N 2333/70578

(57) ABSTRACT

The present application provides specific antibodies that bind to HVEM and that were generated using LAMP technology, which allowed for the presentation of novel, three dimensional epitopes improving the production of anti-HVEM antibodies. In the past, therapeutically effective antibodies directed to HVEM were difficult to generate which the present invention has overcome. Also provided are uses of these antibodies, methods of making these antibodies and polynucleotides and host cells related to these antibodies.

### FIELD OF THE INVENTION

The invention relates to specifically disclosed antibodies that bind to the HVEM protein as well as methods and compositions for detecting, diagnosing, or prognosing a disease or disorder associated with aberrant HVEM expression or inappropriate function of HVEM protein using antibodies or fragments or variants thereof, or related molecules, that bind to HVEM.

### DISCUSSION OF THE RELATED ART

In the following discussion, certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Cancer is the second leading cause of death in the United States, exceeded only by heart disease. Despite recent advances in cancer diagnosis and treatment, surgery and radiotherapy may be curative if a cancer is found early, but current drug therapies for metastatic disease are mostly palliative and seldom offer a long-term cure. Even with new chemotherapies entering the market, the need continues for new drugs effective in monotherapy or in combination with existing agents as first line therapy, and as second and third line therapies in treatment of resistant tumors.

Recent efforts in treating cancer focus on targeted therapeutics or treatments that specifically inhibit vital signaling pathways. However, drug resistance and cancer progression invariably develop. Antibodies are increasingly being developed as anti-cancer therapies. However, the ability to generate antibodies, even fully human antibodies, even with the state-of-the-art tools, can still be difficult.

Herpesvirus entry mediator (HVEM), also known as tumor necrosis factor receptor superfamily member 14 (TNFRSF14) or CD270, is a human cell surface receptor of the TNF-receptor superfamily. In recent years, HVEM has been found highly expressed on hematopoietic cells and a variety of parenchymal cells, such as breast, melanoma, colorectal, and ovarian cancer cells, as well as gut epithelium. HVEM is a bidirectional protein, either inhibiting or stimulating T cells, through binding to BTLA or LIGHT (TNFSF14). However, effective therapeutic antibodies to HVEM have been historically difficult to obtain.

Therefore, a clear need continues to exist for efficient and cost-effective methods of producing antibodies, especially where there has been difficulty in obtaining such antibodies to a particular antigen in the past. Thus, there is a need to develop new and improved antibodies directed to HVEM to be used to treat cancer and HIV in patients, as well as to be used to diagnose and/or prognose irregularities in the HVEM protein.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present invention comprises the results of generating antibodies in a non-human vertebrate wherein the non-human vertebrate was injected with a LAMP Construct comprising a HVEM antigen. The HVEM antigen was then efficiently presented to the immune system with the help of LAMP in the non-human vertebrate to raise novel antibodies against the HVEM antigen.

Specifically, by combining presentation of the specifically selected HVEM antigens with LAMP, the HVEM antigens were effectively transported to the cytoplasmic endosomal/lysosomal compartments, where the HVEM antigens were processed and peptides from it presented on the cell surface in association with major histocompatibility (MHC) class II molecules. This novel presentation generated unexpectedly functional antibodies to an antigen that was known in the past to be particularly difficult to raise therapeutically effective antibodies. Attempts in the past to raise such anti-HVEM antibodies were either unsuccessful or lacked activity. In contrast, the novel antibodies described herein were unexpectedly activity. Thus, in some embodiments, an anti-HVEM antibody comprises: (a) an antibody selected from any one of the antibodies listed by either AntibodyID or Ab_Num_Id as described in Table 1; (b) an antibody comprising a heavy chain amino acid sequence selected from any one of the amino acid sequences of SEQ ID NO:1-201; (c) an antibody comprising a light chain amino acid sequence selected from any one of the amino acid sequences of SEQ ID NO:874-1032; (d) an antibody comprising a heavy chain amino acid sequence selected from any one of the amino acid sequences of SEQ ID NO:1-201 and a light chain amino acid sequence selected from any one of the amino acid sequences of SEQ ID NO:874-1032; (e) an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to any one of (a)-(d); (f) the amino acid sequence of (e), wherein CDRH1, CDRH2 and CDRH3 of SEQ ID NO:1-201 is maintained; (g) the amino acid sequence of (e), wherein CDRL1, CDRL2 and CDRL3 of SEQ ID NO:874-1032 is maintained; (h) the amino acid sequence of (e), wherein the CDRH1, CDRH2, and CDRH3 of SEQ ID NO:1-201, CDRL1, CDRL2 and CDRL3 of SEQ ID NO:874-1032 is maintained; (i) an antibody comprising a CDRH1, a CDRH2, and a CDRH3 selected from an amino acid sequence of any one of SEQ ID NO:1-201; (j) an antibody comprising a CDRL1, a CDRL2, and a CDRL3 selected from an amino acid sequence of any one of SEQ ID NO:874-1032; (k) an antibody comprising a CDRH1, a CDRH2, and a CDRH3 selected from an amino acid sequence of any one of SEQ ID NO:1-201 and a CDRL1, a CDRL2, and a CDRL3 selected from an amino acid sequence of any one of SEQ ID NO:874-1032; (I) an antibody comprising a CDRH1, a CDRH2, and a CDRH3 selected from an amino acid sequence of any one of SEQ ID NO:1-201 and a CDRL1, a CDRL2, and a CDRL3 selected from an amino acid sequence of any one of SEQ ID NO:874-1032, wherein said selection of CDRH1, CDRH2, CDRH3, CDRL1, CDRL2, and CDRL3 are selected from the same AntibodyId as described in Table 1; (m) an antibody comprising at least one of SEQ ID NO: 202-873 and/or at least one of SEQ ID NO: 1033-1449; (n) a single-chain variable fragment (“scFV”) comprising any one of (a)-(m); or (o) a variable domain comprising any one of (a)-(m); and wherein said antibody binds to HVEM. The amino acid sequences for each variable domain of a heavy (SEQ ID NO:1-201) and light chains (SEQ ID NO: 874-1032) are described in Table 3.

Thus, the present disclosure also encompasses, for example, an isolated antibody that binds to HVEM, comprising: (a) a heavy chain comprising VH CDR1, VH CDR2, and VH CDR3 comprising, respectively: SEQ ID Nos 285, 464, and 709 (consensus cluster 11); SEQ ID Nos 298, 470, and 720 (consensus cluster 20); SEQ ID Nos 304, 478, and 729 (consensus cluster 5); SEQ ID Nos 310, 481, and 733 (consensus cluster 23); SEQ ID Nos 321, 495, and 751 (consensus cluster 21); SEQ ID Nos 328, 504, and 753 (consensus cluster 10); SEQ ID Nos 336, 513, and 776 (consensus cluster 8); SEQ ID Nos 340, 514, and 783 (consensus cluser 15); SEQ ID Nos 347, 522, and 795 (consensus cluster 19); SEQ ID Nos 351, 525, and 801 (consensus cluster 14); SEQ ID Nos 355, 530, and 808 (consensus cluster 6); SEQ ID Nos 356, 531, and 811 (consensus cluster 12); SEQ ID Nos 358, 535, and 815 (consensus cluster 4); SEQ ID Nos 361, 538, and 816 (consensus cluster 9); SEQ ID Nos 364, 541, and 821 (consensus cluster 17); SEQ ID Nos 366, 544, and 826 (consensus cluster 7); SEQ ID Nos 367, 547, and 829 (consensus cluster 13); SEQ ID Nos 369, 550, and 833 (consensus cluster 18); SEQ ID Nos 371, 553, and 837 (consensus cluster 22); SEQ ID Nos 374, 557, and 841 (consensus cluster 16); SEQ ID Nos 338, 513, and 844 (consensus cluster 1); SEQ ID Nos 375, 559, and 845 (consensus cluster 2); or SEQ ID Nos 376, 560, and 846 (consensus cluster 3); and (b) a light chain comprising VL CDR1, VL CDR2, and VL CDR3 comprising, respectively: SEQ ID Nos 1099, 1230, and 1343 (consensus cluster 6); SEQ ID Nos 1129, 1246, and 1376 (consensus cluster 7); SEQ ID Nos 1136, 1249, and 1387 (consensus cluster 3); SEQ ID Nos 1142, 1251, and 1399 (consensus cluster 5); SEQ ID Nos 1152, 1248, and 1411 (consensus cluster 1); SEQ ID Nos 1155, 1256, and 1416 (consensus cluster 4); and SEQ ID Nos 1159, 1258, and 1422 (consensus cluster 2). the heavy chain further comprises an FR1, FR2, FR3, and FR4 corresponding to the consensus cluster of the VH CDR1, VH CDR2, and VH CDR3, and/or wherein the light chain further comprises an FR1, FR2, FR3, and FR4 corresponding to the consensus cluster of the VL CDR1, VL CDR2, and VL CDR3.

The disclosure also encompasses, for example, an anti-HVEM antibody that comprises a heavy chain comprising VH CDR1, VH CDR2, and VH CDR3 and the VL CDR1, VL CDR2, and VL CDR3 of any one of Ab_001, Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087. In some cases, the heavy chain comprises a heavy chain variable region (VH) with an amino acid sequence that is at least 90%, at least 95%, or at least 97% identical to that of the VH of Ab_001 Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087, and/or the light chain comprises a light chain variable region (VL) with an amino acid sequence that is at least 90%, at least 95%, or at least 97% identical to that of the VL of Ab_001, Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087. In some cases, the heavy chain comprises a VH with an amino acid sequence comprising the amino acid sequence of the VH of Ab_001, Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087, and/or the light chain comprises a VL with an amino acid sequence comprising the amino acid sequence of the VL of Ab_001, Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087.

In further embodiments, the antibody comprises: (a) a heavy chain constant domain selected from (1) a human IgM constant domain; (2) a human IgGI constant domain; (3) a human IgG2 constant domain; (4) a human IgG3 constant domain; (5) a human IgG4 constant domain; or (6) a human IgA constant domain; (b) a light chain constant domain selected from (1) a Ig kappa constant domain or (2) a human Ig lambda constant domain; or any combination of (a) or (b). In other embodiments, the antibody is a fully human antibody, a humanized antibody, a chimeric antibody, a whole antibody, a single chain (scFv) antibody, a monoclonal antibody, Fab fragment, a Fab′ fragment, a F(ab′)2, a Fv, a disulfide linked F, and/or a bispecific antibody. Thus, in some cases, the antibody comprises a full length heavy chain constant region and/or a full length light chain constant region. In other cases, the antibody is a Fab fragment, a Fab′ fragment, a F(ab′)2 fragment, a Fv fragment, a disulfide linked F fragment, or a scFv fragment.

In some cases, the antibody: (a) blocks the binding of human BTLA to human HVEM with an IC50 of 10 nM or less, 3 nM or less, or 2 nM or less; (b) blocks the binding of human LIGHT to human HVEM with an IC50 of 30 nM or less, 20 nM or less, or 10 nM or less; (c) blocks the binding of human BTLA to human HVEM with an IC50 of 10 nM or less, 3 nM or less, or 2 nM or less, and also blocks the binding of human LIGHT to human HVEM; or (d) blocks the binding of human LIGHT to human HVEM with an IC50 of 30 nM or less, 20 nM or less, or 10 nM or less, and also blocks the binding of human BTLA to human HVEM. In some cases, the antibody binds to human HVEM with a K_{D} of 50 nM or less, or 10 nM or less. In some cases, the antibody binds to cynomolgus monkey HVEM with a K_{D} of 50 nM or less, or 10 nM or less.

In some cases, the antibody is bispecific or multispecific. For example, in some embodiments, a bispecific antibody is selected from: a bispecific T-cell engager (BiTE) antibody, a dual-affinity retargeting molecule (DART), a CrossMAb antibody, a DutaMab™ antibody, a DuoBody antibody; a Triomab, a TandAb, a bispecific NanoBody, Tandem scFv, a diabody, a single chain diabody, a HSA body, a (scFv)2 HSA Antibody, an scFv-IgG antibody, a Dock and Lock bispecific antibody, a DVD-IgG antibody, a TBTI DVD-IgG, an IgG-fynomer, a Tetravalent bispecific tandem IgG antibody, a dual-targeting domain antibody, a chemically linked bispecific (Fab′)2 molecule, a crosslinked mAb, a Dual-action Fab IgG (DAF-IgG), an orthoFab-IgG, a bispecific CovX-Body, a bispecific hexavalent trimerbody, 2 scFv linked to diphtheria toxin, and an ART-Ig.

In further embodiments, the bispecific antibody comprises (a) an anti-CXCL12 antibody; (b) an anti-CXCR4 antibody; (c) an anti-CD47 antibody; (d) a checkpoint inhibitor antibody, preferably an anti-PD-1 antibody, an anti-PD-L1 antibody, an anti-CTLA-4 antibody, an anti-TIM-3 antibody, and/or an anti-LAG3 antibody, (e) an anti-T-cell co-receptor antibody (e.g., an anti-4-1BB (CD137) antibody or an anti-ICOS (CD278) antibody); and/or (f) an anti-neoantigen antibody.

In some embodiments, the neoantigen is selected from: MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-1, SSX-I, SSX-2(HOM-MEL-40), SSX-3, SSX-4, SSX-5, SCP-1 and XAGE, melanocyte differentiation antigens, p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAA0205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-1), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding proteincyclophilin C-associated protein), TAAL6, TAG72, TLP, TPS, tyrosinase related proteins, TRP-1, TRP-2, or mesothelin.

In other embodiments, the antibody further comprises: (a) a detectable label, preferably wherein said detectable label is a radiolabel, an enzyme, a fluorescent label, a luminescent label, or a bioluminescent label; or (b) a conjugated therapeutic or cytotoxic agent.

In some embodiments, the detectable label is selected from ¹²⁵I, ¹³¹I, In, ⁹⁰Y, ⁹⁹Tc, ¹⁷⁷Lu, ¹⁶⁶Ho, or ¹⁵³Sm, or a biotinylated molecule. In other embodiments, the conjugated therapeutic or cytotoxic agent is selected from (a) an anti-metabolite; (b) an alkylating agent; (c) an antibiotic; (d) a growth factor; (e) a cytokine; (f) an anti-angiogenic agent; (g) an anti-mitotic agent; (h) an anthracycline; (i) toxin; and/or (j) an apoptotic agent.

Also provided are pharmaceutical compositions comprising antibodies herein and a pharmaceutically acceptable carrier and/or excipient, as well as kits comprising antibodies herein and/or nucleic acids encoding the anti-HVEM antibodies as described herein. Additionally, vectors and host cells comprising such nucleic acid molecules are also provided.

Uses of the anti-HVEM antibodies are also provided, including uses selected from (a) a method of detecting aberrant expression of the HVEM protein; (b) a method for diagnosing a disease or disorder associated with aberrant HVEM protein expression or activity; (c) a method of inhibiting HVEM activity; (d) a method of increasing HVEM activity; (e) a method of inhibiting HVEM binding to BTLA and/or LIGHT and/or (f) a method of treating a disease or disorder associated with aberrant HVEM expression or activity.

In some embodiments, uses of the anti-HVEM antibodies can be used to treat HIV infection; cancer, preferably, wherein the cancer is an adenocarcinoma, sarcoma, skin cancer, melanoma, bladder cancer, brain cancer, breast cancer, uterus cancer, ovarian cancer, prostate cancer, lung cancer, colorectal cancer, cervical cancer, liver cancer, head and neck cancer, esophageal cancer, pancreas cancer, pancreatic ductal adenocarcinoma (PDA), renal cancer, stomach cancer, multiple myeloma or cerebral cancer. In treating cancer, the use further comprises co-administering other anti-cancer therapies, such as a chemotherapeutic agent, radiation therapy, a cancer therapy, an immunotherapy, or a cancer vaccine, a cytokine, a toxin, a pro-apoptotic protein or a chemotherapeutic agent.

In some embodiments, the cancer vaccine recognizes one or more tumor antigens expressed on cancer cells, preferably, wherein the tumor antigen is selected from MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-1, SSX-I, SSX-2(HOM-MEL-40), SSX-3, SSX-4, SSX-5, SCP-I and XAGE, melanocyte differentiation antigens, p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAA0205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-1), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, TPS, tyrosinase related proteins, TRP-1, TRP-2, or mesothelin.

In other embodiments, the anti-cancer therapy is selected from: aspirin, sulindac, curcumin, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); thylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics, such as actimomycin D, daunomycin (rubidomycin), doxorubicin, mitoxantrone, idarubicin, bleomycins, plicamycin (mithramycin), mitomycinC, and actinomycin; enzymes such as L-asparaginase, cytokines such as interferon (IFN)-gamma, tumor necrosis factor (TNF)-alpha, TNF-beta and GM-CSF, anti-angiogenic factors, such as angiostatin and endostatin, inhibitors of FGF or VEGF such as soluble forms of receptors for angiogenic factors, including soluble VGFNEGF receptors, platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Glivac) and erlotinib (an EGF receptor inhibitor) now marketed as Tarveca; and anti-virals such as oseltamivir phosphate, Amphotericin B, and palivizumab.

In other embodiments, the anti-HVEM antibody is co-administered with a molecule selected from (a) an anti-CXCL12 antibody; (b) an anti-CXCR4 antibody; (c) an anti-CD47 antibody; (d) a checkpoint inhibitor antibody, preferably an anti-PD-1 antibody, an anti-PD-L1 antibody, an anti-CTLA-4 antibody, an anti-TIM-3 antibody, and/or an anti-LAG3 antibody, (e) an anti-T-cell co-receptor antibody (e.g., an anti-4-1BB (CD137) antibody or an anti-ICOS (CD278) antibody); (f) an anti-neoantigen antibody.

In such embodiments, the neoantigen is preferably selected from MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-1, LAGE-1, SSX-I, SSX-2(HOM-MEL-40), SSX-3, SSX-4, SSX-5, SCP-1 and XAGE, melanocyte differentiation antigens, p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAA0205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-1), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, TPS, tyrosinase related proteins, TRP-1, TRP-2, or mesothelin.

In some embodiments, co-administration can occur simultaneously, separately, or sequentially with the antibody.

The disclosure herein also encompasses methods of detecting HVEM in vitro in a sample, comprising contacting the sample with the antibody.

These and other aspects, objects and features are described in more detail below.

### BRIEF DESCRIPTION OF THE FIGURES

The objects and features of the invention can be better understood with reference to the following detailed description and accompanying drawings.

FIG. 1 illustrates the antibody discovery and lead confirmation workflow used to generate the anti-HVEM antibodies as described herein.

FIG. 2 summarizes the screening results obtained after following the work-flow described in FIG. 1.

FIGS. 3a and 3b show intensities from ELISA screens for binding of anti-HVEM antibodies to HVEM, as further described in the Examples.

### DETAILED DESCRIPTION

The invention is directed to specific anti-HVEM antibodies, related compositions, and their use.

### Definitions

The following definitions are provided for specific terms which are used in the following written description.

As used in the specification and claims, the singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules.

As used herein, the term “comprising” is intended to mean that the HVEM antibodies and methods include the recited elements, but do not exclude other elements. “Consisting essentially of”, when used to define HVEM antibodies and methods, shall mean excluding other elements of any essential significance to the combination. Thus, an anti-HVEM antibody consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the HVEM antibody of this invention. Embodiments defined by each of these transition terms are within the scope of this invention.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5 fold, and more preferably within 2 fold, of a value. Unless otherwise stated, the term ‘about’ means within an acceptable error range for the particular value, such as ±1-20%, preferably ±1-10% and more preferably ±1-5%.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

As used herein, the terms “polynucleotide” and “nucleic acid molecule” are used interchangeably to refer to polymeric forms of nucleotides of any length. The polynucleotides may contain deoxyribonucleotides, ribonucleotides, and/or their analogs. Nucleotides may have any three-dimensional structure, and may perform any function, known or unknown. The term “polynucleotide” includes, for example, single-double-stranded and triple helical molecules, a gene or gene fragment, exons, introns, mRNA, tRNA, rRNA, ribozymes, antisense molecules, cDNA, recombinant polynucleotides, branched polynucleotides, aptamers, plasmids, vectors, isolated DNA of any sequence, isolated RNA of any sequence, nucleic acid probes, and primers. A nucleic acid molecule may also comprise modified nucleic acid molecules (e.g., comprising modified bases, sugars, and/or internucleotide linkers).

As used herein, the term “peptide” refers to a compound of two or more subunit amino acids, amino acid analogs, or peptidomimetics. The subunits may be linked by peptide bonds or by other bonds (e.g., as esters, ethers, and the like).

As used herein, the term “amino acid” refers to either natural and/or unnatural or synthetic amino acids, including glycine and both D or L optical isomers, and amino acid analogs and peptidomimetics. A peptide of three or more amino acids is commonly called an oligopeptide if the peptide chain is short. If the peptide chain is long (e.g., greater than about 10 amino acids), the peptide is commonly called a polypeptide or a protein. While the term “protein” encompasses the term “polypeptide”, a “polypeptide” may be a less than full-length protein.

As used herein a “LAMP polypeptide” or “LAMP” refers to the mammalian lysosomal associated membrane proteins human LAMP-1, human LAMP-2, human LAMP-3, human LIMP-2, human Endolyn, human LIMBIC, human LAMP-5, or human Macrosailin as described herein, as well as orthologs, and allelic variants.

As used herein, a “LAMP Construct” is defined as those constructs described in U.S. Ser. No. 16/607,082 filed on Oct. 21, 2019 and is hereby incorporated by reference in its entirety. In preferred embodiments, the LAMP Construct used to generate the anti-HVEM antibodies is ILC-4 as described in this document.

The HVEM, BTLA, and LIGHT proteins referenced herein refer to the human proteins unless specifically noted otherwise herein (e.g., cynomolgus monkey HVEM and the like).

As used herein, “expression” refers to the process by which polynucleotides are transcribed into mRNA and/or translated into peptides, polypeptides, or proteins. If the polynucleotide is derived from genomic DNA, expression may include splicing of the mRNA transcribed from the genomic DNA.

As used herein, “under transcriptional control” or “operably linked” refers to expression (e.g., transcription or translation) of a polynucleotide sequence which is controlled by an appropriate juxtaposition of an expression control element and a coding sequence. In one aspect, a DNA sequence is “operatively linked” to an expression control sequence when the expression control sequence controls and regulates the transcription of that DNA sequence.

As used herein, “coding sequence” is a sequence which is transcribed and translated into a polypeptide when placed under the control of appropriate expression control sequences. The boundaries of a coding sequence are determined by a start codon at the 5′ (amino) terminus and a translation stop codon at the 3′ (carboxyl) terminus. A coding sequence can include, but is not limited to, a prokaryotic sequence, cDNA from eukaryotic mRNA, a genomic DNA sequence from eukaryotic (e.g., mammalian) DNA, and even synthetic DNA sequences. A polyadenylation signal and transcription termination sequence will usually be located 3′ to the coding sequence.

As used herein, two coding sequences “correspond” to each other if the sequences or their complementary sequences encode the same amino acid sequences.

As used herein, “signal sequence” denotes the endoplasmic reticulum translocation sequence. This sequence encodes a signal peptide that communicates to a cell to direct a polypeptide to which it is linked (e.g., via a chemical bond) to an endoplasmic reticulum vesicular compartment, to enter an exocytic/endocytic organelle, to be delivered either to a cellular vesicular compartment, the cell surface or to secrete the polypeptide. This signal sequence is sometimes clipped off by the cell in the maturation of a protein. Signal sequences can be found associated with a variety of proteins native to prokaryotes and eukaryotes.

As used herein, the phrase “prime boost” describes an immunization scheme where an animal is exposed to an antigen and then reexposed to the same or different antigen in order to “boost” the immune system. For example, the use of a LAMP Construct comprising a HVEM antigen could be used to prime a T-cell response followed by the use of a second LAMP Construct comprising a second HVEM antigen, or a DNA vaccine comprising a HVEM antigen or a recombinant HVEM antigen to boost the response. These heterologous prime-boost immunizations elicit immune responses of greater height and breadth than can be achieved by priming and boosting with the same antigen. The priming with a LAMP Construct comprising a HVEM antigen initiates memory cells; the boost step expands the memory response. Preferably, use of the two different agents do not raise responses against each other and thus do not interfere with each other's activity. Mixtures of HVEM antigens are specifically contemplated in the prime and/or boost step. Boosting can occur one or multiple times.

As used herein, “hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

As used herein, a polynucleotide or polynucleotide region (or a polypeptide or polypeptide region) which has a certain percentage (for example, at least about 50%, at least about 60%, at least about 70%, at least about 80%, at least about 85%, at least about 90%, at least about 95%, at least about 99%) of “sequence identity” to another sequence means that, when maximally aligned, using software programs routine in the art, that percentage of bases (or amino acids) are the same in comparing the two sequences.

Two sequences are “substantially homologous” or “substantially similar” when at least about 50%, at least about 60%, at least about 70%, at least about 75%, and preferably at least about 80%, and most preferably at least about 90 or 95% of the nucleotides match over the defined length of the DNA sequences. Similarly, two polypeptide sequences are “substantially homologous” or “substantially similar” when at least about 50%, at least about 60%, at least about 66%, at least about 70%, at least about 75%, and preferably at least about 80%, and most preferably at least about 90 or 95% of the amino acid residues of the polypeptide match over a defined length of the polypeptide sequence. Sequences that are substantially homologous can be identified by comparing the sequences using standard software available in sequence data banks. Substantially homologous nucleic acid sequences also can be identified in a Southern hybridization experiment under, for example, stringent conditions as defined for that particular system. Defining appropriate hybridization conditions is within the skill of the art. For example, stringent conditions can be: hybridization at 5×SSC and 50% formamide at 42° C., and washing at 0.1×SSC and 0.1% sodium dodecyl sulfate at 60° C. Further examples of stringent hybridization conditions include: incubation temperatures of about 25 degrees C. to about 37 degrees C.; hybridization buffer concentrations of about 6×SSC to about 10×SSC; formamide concentrations of about 0% to about 25%; and wash solutions of about 6×SSC. Examples of moderate hybridization conditions include: incubation temperatures of about 40 degrees C. to about 50 degrees C.; buffer concentrations of about 9×SSC to about 2×SSC; formamide concentrations of about 30% to about 50%; and wash solutions of about 5×SSC to about 2×SSC. Examples of high stringency conditions include: incubation temperatures of about 55 degrees C. to about 68 degrees C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed. Similarity can be verified by sequencing, but preferably, is also or alternatively, verified by function (e.g., ability to traffic to an endosomal compartment, and the like), using assays suitable for the particular domain in question.

The terms “percent (%) sequence similarity”, “percent (%) sequence identity”, and the like, generally refer to the degree of identity or correspondence between different nucleotide sequences of nucleic acid molecules or amino acid sequences of polypeptides that may or may not share a common evolutionary origin (see Reeck et al., supra). Sequence identity can be determined using any of a number of publicly available sequence comparison algorithms, such as BLAST, FASTA, DNA Strider, GCG (Genetics Computer Group, Program Manual for the GCG Package, Version 7, Madison, Wisconsin), etc.

To determine the percent identity between two amino acid sequences or two nucleic acid molecules, the sequences are aligned for optimal comparison purposes. The percent identity between the two sequences is a function of the number of identical positions shared by the sequences (i.e., percent identity=number of identical positions/total number of positions (e.g., overlapping positions)×100). In one embodiment, the two sequences are, or are about, of the same length. The percent identity between two sequences can be determined using techniques similar to those described below, with or without allowing gaps. In calculating percent sequence identity, typically exact matches are counted.

The determination of percent identity between two sequences can be accomplished using a mathematical algorithm. A non-limiting example of a mathematical algorithm utilized for the comparison of two sequences is the algorithm of Karlin and Altschul, Proc. Natl. Acad. Sci. USA 1990, 87:2264, modified as in Karlin and Altschul, Proc. Natl. Acad. Sci. USA 1993, 90:5873-5877. Such an algorithm is incorporated into the NBLAST and XBLAST programs of Altschul et al, J. Mol. Biol. 1990; 215: 403. BLAST nucleotide searches can be performed with the NBLAST program, score=100, wordlength=12, to obtain nucleotide sequences homologous to sequences of the invention. BLAST protein searches can be performed with the XBLAST program, score=50, wordlength=3, to obtain amino acid sequences homologous to protein sequences of the invention. To obtain gapped alignments for comparison purposes, Gapped BLAST can be utilized as described in Altschul et al, Nucleic Acids Res. 1997, 25:3389. Alternatively, PSI-Blast can be used to perform an iterated search that detects distant relationship between molecules. See Altschul et al. (1997) supra. When utilizing BLAST, Gapped BLAST, and PSI-Blast programs, the default parameters of the respective programs (e.g., XBLAST and NBLAST) can be used. See ncbi.nlm.nih.gov/BLAST/on the WorldWideWeb.

Another non-limiting example of a mathematical algorithm utilized for the comparison of sequences is the algorithm of Myers and Miller, CABIOS 1988; 4: 11-17. Such an algorithm is incorporated into the ALIGN program (version 2.0), which is part of the GCG sequence alignment software package. When utilizing the ALIGN program for comparing amino acid sequences, a PAM120 weight residue table, a gap length penalty of 12, and a gap penalty of 4 can be used.

In a preferred embodiment, the percent identity between two amino acid sequences is determined using the algorithm of Needleman and Wunsch (J. Mol. Biol. 1970, 48:444-453), which has been incorporated into the GAP program in the GCG software package (Accelrys, Burlington, MA; available at accelrys.com on the WorldWideWeb), using either a Blossum 62 matrix or a PAM250 matrix, a gap weight of 16, 14, 12, 10, 8, 6, or 4, and a length weight of 1, 2, 3, 4, 5, or 6. In yet another preferred embodiment, the percent identity between two nucleotide sequences is determined using the GAP program in the GCG software package using a NWSgapdna.CMP matrix, a gap weight of 40, 50, 60, 70, or 80, and a length weight of 1, 2, 3, 4, 5, or 6. A particularly preferred set of parameters (and the one that can be used if the practitioner is uncertain about what parameters should be applied to determine if a molecule is a sequence identity or homology limitation of the invention) is using a Blossum 62 scoring matrix with a gap open penalty of 12, a gap extend penalty of 4, and a frameshift gap penalty of 5.

Another non-limiting example of how percent identity can be determined is by using software programs such as those described in Current Protocols In Molecular Biology (F. M. Ausubel et al., eds., 1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/cgi-bin/BLAST.

Statistical analysis of the properties described herein may be carried out by standard tests, for example, t-tests, ANOVA, or Chi squared tests. Typically, statistical significance will be measured to a level of p=0.05 (5%), more preferably p=0.01, p=0.001, p=0.0001, p=0.000001

“Conservatively modified variants” of domain sequences also can be provided. With respect to particular nucleic acid sequences, conservatively modified variants refer to those nucleic acids which encode identical or essentially identical amino acid sequences, or where the nucleic acid does not encode an amino acid sequence, to essentially identical sequences. Specifically, degenerate codon substitutions can be achieved by generating sequences in which the third position of one or more selected (or all) codons is substituted with mixed-base and/or deoxyinosine residues (Batzer, et al., 1991, Nucleic Acid Res. 19: 5081; Ohtsuka, et al., 1985, J. Biol. Chem. 260: 2605-2608; Rossolini et al., 1994, Mol. Cell. Probes 8: 91-98).

The term “variant” as used herein refers to a polypeptide that possesses a similar or identical function as an anti-HVEM antibody, but does not necessarily comprise a similar or identical amino acid sequence of an anti-HVEM antibody or possess a similar or identical structure of an anti-HVEM antibody. A variant having a similar amino acid refers to a polypeptide that satisfies at least one of the following: (a) a polypeptide comprising, or alternatively consisting of, an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of an anti-HVEM antibody (including a VH domain, CDRH, VL domain, or CDRL) having an amino acid sequence of any one of those referred to in Tables 1-3); (b) a polypeptide encoded by a nucleotide sequence, the complementary sequence of which hybridizes under stringent conditions to a nucleotide sequence encoding an anti-HVEM antibody (including a VH domain, CDRH, VL domain, or CDRL) having an amino acid sequence of any one of those referred to in Tables 1-3); and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99%, identical to the nucleotide sequence encoding anti-HVEM antibody (including a VH domain, CDRH, VL domain, or CDRL) having an amino acid sequence of any one of those referred to in Tables 1-3) A polypeptide with similar structure to an anti-HVEM antibody or antibody fragment thereof, described herein refers to a polypeptide that has a similar secondary, tertiary or quarternary structure of an anti-HVEM antibody or antibody fragment thereof as described herein. The structure of a polypeptide can be determined by methods known to those skilled in the art, including but not limited to, X-ray crystallography, nuclear magnetic resonance, and crystallographic electron microscopy.

The term “biologically active fragment”, “biologically active form”, “biologically active equivalent” of and “functional derivative” of a wild-type protein, possesses a biological activity that is at least substantially equal (e.g., not significantly different from) the biological activity of the wild type protein as measured using an assay suitable for detecting the activity.

As used herein, the term “isolated” or “purified” means separated (or substantially free) from constituents, cellular and otherwise, in which the polynucleotide, peptide, polypeptide, protein, antibody, or fragments thereof, are normally associated with in nature. For example, isolated polynucleotide is one that is separated from the 5′ and 3′ sequences with which it is normally associated in the chromosome. As is apparent to those of skill in the art, a non-naturally occurring polynucleotide, peptide, polypeptide, protein, antibody, or fragments thereof, does not require “isolation” to distinguish it from its naturally occurring counterpart. By substantially free or substantially purified, it is meant at least 50% of the population, preferably at least 70%, more preferably at least 80%, and even more preferably at least 90%, are free of the components with which they are associated in nature.

As used herein, a “target cell” or “recipient cell” refers to an individual cell or cell which is desired to be, or has been, a recipient of the polynucleotide described herein. The term is also intended to include progeny of a single cell, and the progeny may not necessarily be completely identical (in morphology or in genomic or total DNA complement) to the original parent cell due to natural, accidental, or deliberate mutation. A target cell may be in contact with other cells (e.g., as in a tissue) or may be found circulating within the body of an organism.

As used herein, a “non-human vertebrate” is any vertebrate that can be used to generate antibodies. Examples include, but are not limited to, a rat, a mouse, a rabbit, a llama, camels, a cow, a guinea pig, a hamster, a dog, a cat, a horse, a non-human primate, a simian (e.g. a monkey, ape, marmoset, baboon, rhesus macaque), or an ape (e.g. gorilla, chimpanzee, orangutan, gibbon), a chicken. Other classes of non-human vertebrates include murines, simians, farm animals, sport animals, and pets.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. Compositions comprising the anti-HVEM antibodies described herein also can include stabilizers and preservatives. For examples of carriers, stabilizers and adjuvants, see Martin Remington's Pharm. Sci., 15th Ed. (Mack Publ. Co., Easton (1975)).

A cell has been “transformed”, “transduced”, or “transfected” by the polynucleotide when such nucleic acids have been introduced inside the cell.

Transforming DNA may or may not be integrated (covalently linked) with chromosomal DNA making up the genome of the cell. In prokaryotes, yeast, and mammalian cells for example, the polynucleotide may be maintained on an episomal element, such as a plasmid. In a eukaryotic cell, a stably transformed cell is one in which the polynucleotides have become integrated into a chromosome so that it is inherited by daughter cells through chromosome replication. This stability is demonstrated by the ability of the eukaryotic cell to establish cell lines or clones comprised of a population of daughter cells containing the polynucleotides. A “clone” is a population of cells derived from a single cell or common ancestor by mitosis. A “cell line” is a clone of a primary cell that is capable of stable growth in vitro for many generations (e.g., at least about 10).

As used herein, an “effective amount” is an amount sufficient to affect beneficial or desired results, e.g., such as an effective amount of an anti-HVEM antibody or expression of an anti-HVEM antibody to attain a desired therapeutic endpoint. An effective amount can be administered in one or more administrations, applications or dosages. In one aspect, an effective amount of an anti-HVEM antibody is an amount sufficient to treat and/or ameliorate a tumor when injected into a non-human vertebrate.

The term “treat” or “treatment” other like, as used herein, refers broadly to an improvement or amelioration of a disease or disorder in a subject, such as the improvement or amelioration of at least one symptom or marker associated with the disease or disorder, such as, in the case of a tumor, for example, reduction in the size of the tumor, or a change in biochemical markers associated with the tumor, or reduction in disease symptoms. Treat or treatment also refers to prevention of the onset or slowing of the onset of a disease or disorder, for example.

An “antigen” refers to the target of an antibody, i.e., the molecule to which the antibody specifically binds. The term “epitope” denotes the site on an antigen, either proteinaceous or non-proteinaceous, to which an antibody binds. Epitopes on a protein can be formed both from contiguous amino acid stretches (linear epitope) or comprise non-contiguous amino acids (conformational epitope), e.g., coming in spatial proximity due to the folding of the antigen, i.e., by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents.

The term “antibody” herein refers to an immunoglobulin molecule comprising at least complementarity-determining region (CDR) 1, CDR2, and CDR3 of a heavy chain and at least CDR1, CDR2, and CDR3 of a light chain, wherein the molecule is capable of binding to antigen. An “anti-HVEM antibody” or an “HVEM-antibody” or an “antibody that specifically binds to HVEM” or an “antibody that binds to HVEM” and similar phrases refer to an anti-HVEM antibody as described herein.

The term is used in the broadest sense and encompasses various antibody structures, including but not limited to monoclonal antibodies, polyclonal antibodies, multispecific antibodies (e.g., bispecific antibodies, diabodies, etc.), full length antibodies, single-chain antibodies, antibody conjugates, and antibody fragments, so long as they exhibit the desired HVEM-specific binding activity.

An “anti-HVEM antibody” is an “antibody” that specifically binds a HVEM antigen and, includes antibodies comprising one or more of the sequences described herein in Tables 1-3. An anti-HVEM antibody specifically excludes antibodies known in the art that are capable of binding HVEM. The term encompasses polyclonal, monoclonal, and chimeric antibodies, including bispecific antibodies. An “antibody combining site” is that structural portion of an antibody molecule comprised of heavy and light chain variable and hypervariable regions that specifically binds a HVEM antigen. Exemplary anti-HVEM antibody molecules are intact immunoglobulin molecules, substantially intact immunoglobulin molecules, and those portions of an immunoglobulin molecule that contains the paratope, including Fab, Fab′, F(ab′)2 and F(v) portions, which portions are preferred for use in the therapeutic methods described herein.

Thus, the term an anti-HVEM antibody encompasses not only whole antibody molecules, but also antibody fragments as well as variants (including derivatives such as fusion proteins) of anti-HVEM antibodies and antibody fragments. Examples of molecules which are described by the term “anti-HVEM antibody” in this application include, but are not limited to: single chain Fvs (scFvs), Fab fragments, Fab′ fragments, F(ab′)2, disulfide linked Fvs (sdFvs), Fvs, and fragments comprising or alternatively consisting of, either a VL or a VH domain(s). The term “single chain Fv” or “scFv” as used herein refers to a polypeptide comprising a VL domain of an anti-HVEM antibody described in Table 3 linked to a VH domain of an anti-HVEM antibody described in Table 3. Preferred scFV anti-HVEM antibodies comprise the VL and VH domains of the same antibody selected from antibodies identified in column 1 (“AntibodyID”) in Table 1. See Carter (2006) Nature Rev. Immunol. 6:243. It is understood that linkages can vary, so long as the VL and VH domains are linked in a way maintain functionality of the anti-HVEM antibodies.

Additionally, anti-HVEM antibodies of the invention include, but are not limited to, monoclonal, multi-specific, bi-specific, human, humanized, mouse, or chimeric antibodies, single chain antibodies, camelid antibodies, Fab fragments, F(ab′) fragments, anti-idiotypic (anti-Id) antibodies (including, e.g., anti-Id antibodies to antibodies of the invention), domain antibodies and epitope-binding fragments of any of the above. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

Most preferably, the anti-HVEM antibodies are human antibodies comprising the sequences described in any one of the Tables 2-3. As used herein, “human” antibodies include antibodies having the amino acid sequence of a human immunoglobulin and include antibodies isolated from human immunoglobulin libraries and xenomice or other organisms that have been genetically engineered to produce human antibodies.

The term “heavy chain” or “HC” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “light chain” or “LC” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence.

The term “complementarity determining regions” (“CDRs”) as used herein refers to each of the regions of an antibody variable region which are hypervariable in sequence and which determine antigen binding specificity. Generally, antibodies comprise six CDRs: three in the VH (CDR-H1 or heavy chain CDR1, CDR-H2, CDR-H3), and three in the VL (CDR-L1, CDR-L2, CDR-L3). Unless otherwise indicated, exemplary CDRs are shown in Tables 1-4 herein.

“Framework” or “FR” refers to the residues of the variable region residues that are not part of the complementary determining regions (CDRs). The FR of a variable region generally consists of four FRs: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4. Exemplary FRs are shown in Tables 1-4 herein.

The term “variable region” or “variable domain” interchangeably refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). See, e.g., Kindt et al. Kuby Immunology, 6th ed., W.H. Freeman and Co., page 91 (2007). A variable domain may comprise heavy chain (HC) CDR1-FR2-CDR2-FR3-CDR3 with or without all or a portion of FR1 and/or FR4; and light chain (LC) CDR1-FR2-CDR2-FR3-CDR3 with or without all or a portion of FR1 and/or FR4. That is, a variable domain may lack a portion of FR1 and/or FR4 so long as it retains antigen-binding activity. A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352: 624-628 (1991).

An “antibody fragment” or “antigen binding fragment” refers to a molecule other than an intact antibody that comprises a portion of an intact antibody that binds the antigen (i.e., HVEM) to which the intact antibody binds. Examples of antibody fragments include but are not limited to Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The terms “full length antibody”, “intact antibody”, and “whole antibody” are used herein interchangeably to refer to an antibody having a structure substantially similar to a native antibody structure or, in the case of an IgG antibody, having heavy chains that contain an Fc region as defined herein above.

The light chain and heavy chain “constant regions” of an antibody refer to additional sequence portions outside of the FRs and CDRs and variable regions. Certain antibody fragments may lack all or some of the constant regions. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “Fc region” or “Fc domain” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain at Gly446 and Lys447 (EU numbering). Antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine and lysine, respectively. Therefore, the C-terminal lysine, or the C-terminal glycine and lysine, of the Fc region may or may not be present. Thus, a “full-length heavy chain constant region” or a “full length antibody” for example, which is a human IgG1 antibody, includes an IgG1 with both a C-terminal glycine and lysine, without the C-terminal lysine, or without both the C-terminal glycine and lysine. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. In certain aspects, the antibody is of the IgG1 isotype. In certain aspects, the antibody is of the IgG1 isotype with the P329G, L234A and L235A mutation to reduce Fc-region effector function. In other aspects, the antibody is of the IgG2 isotype. In certain aspects, the antibody is of the IgG4 isotype with the S228P mutation in the hinge region to improve stability of IgG4 antibody. In some aspects, the antibody may have a non-human IgG constant region, and may be, for example, a murine IgG2a antibody such as a murine IgG2a LALAPG antibody. The light chain of an antibody may be assigned to one of two types, called kappa (κ) and lambda (λ), based on the amino acid sequence of its constant domain.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method.

The term “chimeric” antibody refers to an antibody in which a portion of the heavy and/or light chain is derived from a particular source or species, while the remainder of the heavy and/or light chain is derived from a different source or species.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

“Humanized” or chimeric anti-HVEM monoclonal antibodies as described in Tables 1-3 can be produced using techniques described herein or otherwise known in the art. For example, standard methods for producing chimeric antibodies are known in the art. See, for review the following references: Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

The anti-HVEM antibodies provided herein may be monovalent, bivalent, trivalent or multivalent. For example, monovalent scFvs can be multimerized either chemically or by association with another protein or substance. A scFv that is fused to a hexahistidine tag or a Flag tag can be multimerized using Ni-NTA agarose (Qiagen) or using anti-Flag antibodies (Stratagene, Inc.). Additionally, monospecific, bispecific, trispecific or of greater multispecificity for HVEM antigen(s) can also be generated. See, e.g., PCT publications WO 93/17715; WO 92/08802; WO 91/00360; WO 92/05793; Tutt, et al., J. Immunol. 147:60-69 (1991); U.S. Pat. Nos. 4,474,893; 4,714,681; 4,925,648; 5,573,920; 5,601,819; Kostelny et. al., J. Immunol. 148:1547-1553 (1992).

A “multispecific” antibody is one that binds specifically to more than one target antigen, while a “bispecific” antibody is one that binds specifically to two antigens. An “antibody conjugate” is an antibody conjugated to one or more heterologous molecule(s), including but not limited to a therapeutic agent or a label.

As used herein, “bispecific anti-HVEM antibodies” are recombinant monoclonal antibodies and antibody-like molecules that combine the specificities of two distinct antibodies in one molecule. Thus, they can therefore simultaneously target two distinct antigens. As provided herein, one of the antigens targeted by the anti-HVEM bispecific antibody is a HVEM antigen and comprises any of the amino acid sequences shown in Tables 2-3.

Preferred examples of bispecific anti-HVEM antibodies include, but are not limited to, bispecific T-cell engager (BiTE) antibodies, dual-affinity retargeting molecules (DARTs), CrossMAb antibodies, DutaMab™ antibodies, DuoBody antibodies; Triomabs, TandAbs, bispecific NanoBodies, T-cells preloaded with bispecific antibodies, polyclonally-activated T-cells preloaded with bispecific antibodies, Tandem scFvs, diabodies, single chain diabodies, HSA bodies, (scFv)2 HSA antibodies, scFv-igG antibodies, Dock and Lock bispecific antibodies, DVD-IgG antibodies, TBTI DVD IgG antibodies, IgG-fynomers, Tetravalent bispecific tandem IgG antibodies, dual-targeting domain antibodies, chemically linked bispecific (Fab′)2 molecules, crosslinked mAbs, dual-action Fab IgG antibodies (DAF-IgGs), orthoFab-IgG antibodies, bispecific CovX-Bodies, bispecific hexavalent trimerbodies, 2 scFv linked to diphtheria toxin antibodies, and ART-Igs.

As used herein, Dual-Affinity Retargeting (DART) platform technology is a type of bispecific antibody developed by MacroGenics. The platform is capable of targeting multiple different epitopes with a single recombinant molecule and is specifically engineered to accommodate various region sequences in a “plug-and-play” fashion. In this technology, a proprietary covalent linkage is developed and thus, the molecule possesses exceptional stability, optimal heavy and light chain pairing, and predictable antigen recognition. The DART platform is believed to reduce the probability for immunogenicity.

As used herein, Cross monoclonal antibodies (CrossMAbs) are a type of bispecific antibody invented by Roche. The purpose of this technology is to create a bispecific antibody that closely resembles a natural IgG mAb as a tetramer consisting of two light chain-heavy chain pairs, and to solve the problem of light chain mispairing. This technology is believed to prevent unspecific binding of the light chain to its heavy counterpart thereby prevent unwanted side products. In addition, this method leaves the antigen-binding regions of the parental antibodies intact and thus can convert any antibodies into a bispecific IgG.

As used herein, a DutaMab is a type of bispecific antibody invented by Dutalys (acquired by Roche). This platform differs by developing fully human bispecific antibodies that show high affinity in each arm and simultaneously bind both targets. DutaMabs are also believed to possess excellent stability and good manufacturing properties.

Duobody antibodies are a type of bispecific antibodies created by Genmab. This platform generates stable bispecific human IgG1 antibodies and is able to fully retain IgG1 structure and function. Two parental IgG1 monoclonal antibodies are first separately produced, each containing single matched mutations in the third constant domain. Subsequently, these IgG1 antibodies are purified according to standard processes for recovery and purification. After production and purification (post-production), the two antibodies are recombined under tailored laboratory conditions resulting in a bispecific antibody product with a very high yield (typically >95%) (Labrijn et al, PNAS 2013; 110(13):5145-5150). The Duobody platform is believed to have minimal immunogenicity and can combine any antigen binding sequence derived from any antibody-generating platform to generate a bispecific product.

Additionally, the anti-HVEM antibodies described herein could be fused to a heterologous molecule, substance, or agent that possesses anti-cancer capabilities. This approach leverages the anti-HVEM antibody's ability to target tumor cells, thereby delivering the heterologous molecule, substance, or agent directly to the tumor site. For example, cytotoxic agents, when fused to the anti-HVEM antibody, can be delivered to a tumor cell. In some embodiments, the fused anti-HVEM antibody may have potent anti-cancer effects (e.g., synergism) as compared to administering the monoclonal antibody and the heterologous molecule, substance, or agent separately. Observed anti-tumor effects that can be improved, include but are not limited to, reduced cell proliferation, enhanced immunomodulatory functions, site-specific delivery, improved safety, and increased tolerability (i.e., decreased toxicity).

For example, the anti-HVEM antibody can be fused with antitumor cytokines, including but not limited to IL-2, IL-6, IL-7, IL-10, IL-12, IL-15, IL-17, IL-21, GM-CSF, TNF, IFN-α, IFN-3, IFN-γ, and FasL. Additionally, the anti-HVEM antibody can also be fused with 2 different cytokines simultaneously such as GM-CSF/IL-2, IL/12/IL-2, IL-12/GM-CSF, IL-and 12/TNF-α and therefore, form a “di-cytokine fusion protein.”

In a further preferred embodiment, the anti-HVEM antibody can be fused with a radionuclide, including but not limited to ¹³¹Iodine, ⁹⁰γYttrium, ¹⁷⁷Lutetium, ¹⁸⁸Rhenium, ⁶⁷Copper, ²¹¹Astatine, ²¹³Bismuth, ¹²⁵Iodine, and ¹¹¹Indium to form a radioconjugate.

In another preferred embodiment, the anti-HVEM antibody can be fused with a toxin to produce an immunotoxin. Examples of such toxins include, but are not limited to Pseudomonas exotoxin, staphylococcal enterotoxin A, ricin A-chain, and plant ribosome-inactivating protein saporin.

In another preferred embodiment, the anti-HVEM antibody can be fused with a pro-apoptotic protein. Examples of such proteins include, but are not limited to, caspase-3, FOXP3, and death ligand TNF-related apoptosis-inducing ligand (TRAIL).

In another preferred embodiment, the anti-HVEM antibody can be fused to an enzyme that is capable of converting a prodrug to a potent cytotoxic drug, resulting in an antibody-enzyme conjugate that can be used in antibody-directed enzyme prodrug therapy (ADEPT). Examples of such enzymes include, but are not limited to, carboxypeptidase G2, carboxypeptidase A, alkaline phosphatase, penicillin amidase, β-glucuronidase, β-lactamase, cytosine deaminase, aminopeptidase, and glycosidase.

In yet another preferred embodiment, the anti-HVEM antibody is fused with an anti-cancer drug (Kermer et al., Mol Cancer Ther, 11(6); 1279-88, 2012, Sharkey et al., CA Cancer J Clin; 56:226-243, 2006; Ortiz-Sanchez et al., Expert Opin Biol Ther, 8(5): 609-632, 2008; Kosobokova et al., CTM; 5(4): 102-110, List et al., Clinical Pharmacology: Advances and Applications; 5 (Suppl I): 29-45, 2013; Tse et al., PNAS; 97(22): 12266-12271, 2000, Heinze et al., International Journal of Oncology; 35: 167-173, 2009, EI-Mesery et al., Cell Death and Disease; 4, e916, 2013, Wiersma et al., British Journal of Haematology; 164, 296-310, 2013, Dohlsten et al., Proc. Natl. Acad. Sci; 91: 8945-8949, 1994, Melton et al., J Natl Cancer Inst; 88: 153-65, 1996, Cristina et al., Microbial Cell Factories; 14: 19, 2015, Weidle et al., Cancer Genomics and Proteomics; 9: 357-372, 2012, Helguera et al., Methods Mol Med; 109:347-74, 2005, and Young et al., Semin Oncol; 41(5):623-36, 2014).

As used herein, CD47, also known as Integrin Associated Protein, is a transmembrane receptor that belongs to the immunoglobulin superfamily and is ubiquitously expressed on the surface of normal and solid tumor cells. CD47 is also involved in numerous normal and pathological processes including immunity, apoptosis, proliferation, migration, and inflammation. Previous studies have demonstrated the expression of CD47 on various cancer cells and revealed its role in promoting cancer progression. By binding with signal regulatory protein (SIRPα), the primary ligand of CD47 expressed on phagocytic cells (dendritic cells, macrophages, and neutrophils), CD47 prohibits phagocytosis and thus allows tumor cells to evade immune surveillance. Thus, CD47 appears as an important therapeutic target for cancer treatments. Anti-CD47 monoclonal antibodies for clinical uses are currently being developed by Stanford University (phase I, cancer treatment), by the Ukraine Antitumor Center (phase I, cancer treatment), and by Vasculox, Inc. (Preclinical, organ transplantation).

As used herein, “anti-CD47 antibody” is defined as a monoclonal antibody that exclusively recognizes and binds to the antigen, CD47. Binding prevents the interaction between CD47 and SIRPα, a protein on phagocytes, thereby reversing the inhibition of phagocytosis normally caused by the CD47/SIRPα interaction. When co-administered with an anti-HVEM antibody (for example as separate antibodies or as a bi-specific antibody), the anti-CD47 antibody eliminates the “don't eat me signal” and allows the cancer antigen-specific antibody to more efficiently induce a tumor antigen-specific immune response.

As used herein, “antibody-dependent cell-mediated cytotoxicity” is a mechanism of cell-mediated immune defense whereby an effector cell of the immune system actively lyses a target cell, whose membrane-surface antigens have been bound by specific antibodies.

An “epitope” is a structure, usually made up of a short peptide sequence or oligosaccharide, that is specifically recognized or specifically bound by a component of the immune system. T-cell epitopes have generally been shown to be linear oligopeptides. Two epitopes correspond to each other if they can be specifically bound by the same antibody. Two epitopes correspond to each other if both are capable of binding to the same B cell receptor or to the same T cell receptor, and binding of one antibody to its epitope substantially prevents binding by the other epitope (e.g., less than about 30%, preferably, less than about 20%, and more preferably, less than about 10%, 5%, 1%, or about 0.1% of the other epitope binds). In the present invention, multiple epitopes can make up a HVEM antigen.

The term “HVEM antigen” as used herein covers the polypeptide sequence encoded by a polynucleotide sequence cloned into the LAMP Construct which was used to elicit an innate or adaptive immune response in a non-human vertebrate. A “HVEM antigen” encompasses both a single HVEM antigen as well as multiple HVEM antigenic sequences (derived from the same or different proteins) cloned into the LAMP construct.

The term “anti-HVEM antibody presenting cell” as used herein includes any cell which presents on its surface an anti-HVEM antibody as described herein in association with a major histocompatibility complex molecule, or portion thereof, or, alternatively, one or more non-classical MHC molecules, or a portion thereof. Examples of suitable APCs are discussed in detail below and include, but are not limited to, whole cells such as macrophages, dendritic cells, B cells, hybrid APCs, and foster HVEM antigen presenting cells.

As used herein, “partially human” refers to a nucleic acid having sequences from both a human and a non-human vertebrate. In the context of partially human sequences, the partially human nucleic acids have sequences of human immunoglobulin coding regions and sequences based on the non-coding sequences of the endogenous immunoglobulin region of the non-human vertebrate. The term “based on” when used with reference to endogenous non-coding sequences from a non-human vertebrate refers to sequences that correspond to the non-coding sequence and share a relatively high degree of homology with the non-coding sequences of the endogenous loci of the host vertebrate, e.g., the non-human vertebrate from which the ES cell is derived. Preferably, the non-coding sequences share at least an 80%, more preferably 90% homology with the corresponding non-coding sequences found in the endogenous loci of the non-human vertebrate host cell into which a partially human molecule comprising the non-coding sequences has been introduced.

The term “immunoglobulin variable region” as used herein refers to a nucleotide sequence that encodes all or a portion of a variable region of an anti-HVEM antibody as described in Tables 2-3. Immunoglobulin regions for heavy chains may include but are not limited to all or a portion of the V, D, J, and switch regions, including introns. Immunoglobulin region for light chains may include but are not limited to the V and J regions, their upstream flanking sequences, introns, associated with or adjacent to the light chain constant region gene.

By “transgenic animal” is meant a non-human animal, usually a mammal, having an exogenous nucleic acid sequence present as an extrachromosomal element in a portion of its cells or stably integrated into its germ line DNA (i.e., in the genomic sequence of most or all of its cells). In generating a transgenic animal comprising human sequences, a partially human nucleic acid is introduced into the germ line of such transgenic animals by genetic manipulation of, for example, embryos or embryonic stem cells of the host animal according to methods well known in the art.

A “vector” includes plasmids and viruses and any DNA or RNA molecule, whether self-replicating or not, which can be used to transform or transfect a cell.

As used herein, a “genetic modification” refers to any addition, deletion or disruption to a cell's normal nucleotides. Art recognized methods include viral mediated gene transfer, liposome mediated transfer, transformation, transfection and transduction, e.g., viral-mediated gene transfer using adenovirus, adeno-associated virus and herpes virus, as well as retroviral based vectors.

In the present invention, a “PD-1 signaling inhibitor” is an exogenous factor, such as a pharmaceutical compound or molecule that inhibits or prevents the activation of PD-1 by its ligand PD-L1 and thereby blocks or inhibits PD-1 signaling in cells within the cancerous tumor. A PD-1 signaling inhibitor is defined broadly as any molecule that prevents the negatively regulation by PD-1 of T-cell activation. Preferred examples of a PD-1 signaling inhibitor includes, but is not limited to, a PD-1 antagonist and/or a PD-L1 antagonist.

In the present invention, a “PD-1 antagonist” is defined as a molecule that inhibits PD-1 signaling by binding to or interacting with PD-1 to prevent or inhibit the binding and/or activation of PD-1 by PD-L1, thereby inhibiting PD-1 signaling and/or enhancing T-cell activation. Preferred examples of a PD-1 antagonist, include, but are not limited to an anti-PD-1 antibody which are well known in the art. See, Topalian, et al. NEJM 2012.

In the present invention, a “PD-L1 antagonist” is defined as a molecule that inhibits PD-1 signaling by binding to or inhibiting PD-L1 from binding and/or activating PD-1, thereby inhibiting PD-1 signaling and/or enhancing T-cell activation. Preferred examples of a PD-L1 antagonist, include, but are not limited to an anti-PD-L1 antibody which are well known in the art. See, Brahmer, et al. NEJM 2012.

In the present invention, a “CTLA-4 antagonist” is defined as a molecule that inhibits CTLA-4 signaling by binding to or inhibiting CTLA-4 from binding and/or activating to B7 molecules, known in the art to be present on antigen-presenting cells, thereby preventing interactions of B7 molecules with the co-stimulatory molecule CD28, and inhibiting T-cell function. Preferred embodiments of a CTLA-4 antagonist, include, but are not limited to anti-CTLA-4 antibodies.

In the present invention, a “LAG3 antagonist” is defined as a molecule that inhibits LAG3 signaling by binding to or inhibiting LAG3 from binding and/or activating MHC molecules and any other molecule, known in the art to be present on antigen-presenting cells, thereby preventing LAG3 interactions and promoting T-cell function. Preferred embodiments of a LAG3 antagonist, include, but are not limited to anti-LAG3 antibodies.

In the present invention, a “TIM-3 antagonist” is defined as a molecule that inhibits the CD8+ and CD4+Th1-specific cell surface protein, TIM-3, which, when ligated by galectin-9, for example, causes T-cell death. Preferred embodiments of a TIM-3 antagonist, include, but are not limited to anti-TIM-3 antibodies that block interaction with its ligands.

In the present invention, a PD-1 antagonist, a CTLA-4 antagonist, a TIM-3 antagonist, and a LAG3 antagonist are considered as “check-point inhibitors” or “check-point antagonists” or “T-cell checkpoint antagonists”. Other examples of checkpoint antagonists are well known in the art. These molecules can all be administered in combination with an anti-HVEM antibody or can be included in a bi-specific anti-HVEM antibody described herein.

As used herein, “anti-CXCL12 antibody” or a “CXC12 antagonist” is defined as a monoclonal antibody or small molecule that exclusively recognizes the antigen, CXCL12, and thereby elicits immune responses, such as Fc receptor-mediated phagocytosis and antibody-dependent cell-mediated cytotoxicity. Preferred examples of anti-CXCL12 antibodies include, but are not limited to, MAB310 (R&D Systems) and hu30D8. It has been reported in the literature that anti-CXCL12 antibodies can coat tumor cells and therefore are particularly useful in co-administration and/or in making bi-specific antibodies with the anti-HVEM antibodies as described herein.

Similarly, as used herein, an “anti-CXCR4 antibody” or a “CXCR antagonist” is defined as a monoclonal antibody or small molecule that exclusively recognizes the CXCR4 receptor on T cells and thereby elicits immune responses, such as Fc receptor-mediated phagocytosis and antibody-dependent cell-mediated cytotoxicity. Examples of anti-CXCR4 inhibitors include AMD3100, BMS-936564/MDX-1338, AMD11070, or LY2510924. Co-administration and/or in making bi-specific antibodies with an anti-CXCR4 antibody and the anti-HVEM antibodies are preferred embodiments.

As used herein, CAR T-cells, also known as chimeric antigen receptor T-cells, are produced by using adoptive cell transfer technique. T-cells are first collected from patients' blood and recombinant receptors are introduced into these T-cells using genetic engineering methods such as retroviruses. CAR T-cells are then infused into the patient, the tumor-associated antigen is recognized by the CAR T-cell, and is destroyed. Thus, CAR T-cells enhance tumor specific immunosurveillance. The structure of CAR most commonly incorporates a single-chain variable fragment (scFv) derived from a monoclonal antibody that links to intracellular signaling domains and forms a single chimeric protein. In the present invention, the CAR T-cell is developed using scFV, variable regions or CDRs as described herein.

Thus, in preferred embodiments, the HVEM-targeted immune response agent of the present invention, whether it be an anti-HVEM antibody (e.g., a bispecific anti-HVEM antibody), a CAR T-cell engineered to express a chimeric antigen receptor comprising the anti-HVEM antibody sequences described herein, or a T-cell preloaded with anti-HVEM antibodies sequences, has synergistic activity with a second molecule co-administered with the anti-HVEM targeted agent.

In the present invention, a “T-cell co-receptor” is a cell surface receptor that binds to ligands on antigen-presenting cells that are distinct from the peptide-MHC complex that engages the T-cell receptor. Ligation of T-cell co-receptors enhance the antigen-specific activation of the T-cell by recruiting intracellular signaling proteins (e.g., NFkappaB and PI3-kinase) inside the cell involved in the signaling of the activated T lymphocyte. Preferred embodiments of a T-cell co-receptor antagonist, include, but are not limited to anti-T-cell co-receptor antibodies, such as, for example, antibodies directed to 4-1 BB(CD137) and ICOS (CD278).

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Additionally, the present invention employs, unless otherwise indicated, conventional molecular biology, microbiology, and recombinant DNA techniques within the skill of the art. Such techniques are explained fully in the literature. See, e.g., Maniatis, Fritsch & Sambrook, In Molecular Cloning: A Laboratory Manual (1982); DNA Cloning: A Practical Approach, Volumes I and II (D. N. Glover, ed., 1985); Oligonucleotide Synthesis (M. J. Gait, ed., 1984); Nucleic Acid Hybridization (B. D. Hames & S. J. Higgins, eds., 1985); Transcription and Translation (B. D. Hames & S. I. Higgins, eds., 1984); Animal Cell Culture (R. I. Freshney, ed., 1986); Immobilized Cells and Enzymes (IRL Press, 1986); B. Perbal, A Practical Guide to Molecular Cloning (1984

### Anti-HVEM Antibodies

The present invention encompasses the anti-HVEM antibody amino acid sequences described in Tables 1-3. These antibodies were obtained by using Immunomic Therapeutics Universal Intracellular Targeted Expression (UNITE™) platform technology as described in U.S. Ser. No. 16/607,082 filed on Oct. 21, 2019 (published as US Published Appl. No. 2020/0377570), which is hereby incorporated by reference in its entirety.

It is known that the generation of antibodies to HVEM is particularly difficult. In the past, the number and repertoire of obtained antibodies to HVEM has been minimal, lacked variation and failed to produce desired therapeutic efficacy. Applicants used their proprietary ILC-4 LAMP Construct as described in U.S. Ser. No. 16/607,082 with carefully selected HVEM antigens to unexpectedly obtain the new antibodies described herein, and specifically in Tables 1-3.

Tables 1-3 describe different anti-HVEM antibodies. Specifically, Table 1 provides the names of each heavy chain (“Heavy_chain_id”) and light chain (“Light_chain_id”) variable domains making up each antibody identified by “AntibodyId” or “Ab_Num_id”. Table 1 also provides binding data information of selected antibodies tested, based on bio-layer interferometry assays described in the Examples herein, and IC50 results from BTLA and LIGHT competition a says also described in the Examples. “NA” in the BTLA or LIGHT competition assay columns in Table 1 indicates that the antibody showed some degree of competition with either BTLA or LIGHT for HVEM binding, but that an IC50 was not measurable. “NA*” in Table 1 indicates that the antibody did not detectably compete with BTLA or LIGHT for HVEM binding in the assay.

Table 2 provides the amino acid sequence of the variable domain (“VH_Full_lenght_AA”) of the heavy chain (“Heavy_chain_id”) making up the different HVEM antibodies described in Table 1. Table 2 also provides the amino acid sequences making up each of the three complementarity-determining regions (“CDRs”) for each heavy chain (the CDRs identified in Table 2 as “CDRH1,” “CDRH2”, and “CDRH3” and the full variable domain of the heavy chains are shown in Table 3 as SEQ ID NO: 1-201) and and each light chain (the CDRs identified in Table 2 as “CDRL1,” “CDRL2”, and “CDRL3” and the full variable domain of the light chains are shown Table 3 as SEQ ID NO: 874-1032). Importantly Table 2 also groups the obtained antibodies heavy and light chain sequences into “clusters” or “clades” based on the overall similarity of the full length sequences. From these clusters, consensus sequences for each domain (FR1, CDR1, FR2, CDR2, FR3, CDR3, and FR4) for both he heavy and light chains) are created and shown. In preferred embodiments, antibodies comprising the consensus domains are specifically contemplated;

Table 3 provides the amino acid sequence of the variable domain (“VL_Full_lenght_AA”) of the light chain (“Light_chain_id”) making up the different HVEM antibodies described in Table 1;

Table 4 provides the SEQ ID Nos: of each domain, including the consensus sequences of each domain within a particular cluster. In preferred embodiments, an antibody described herein comprises at least one of the domains of SEQ ID NO: 202-873 and/or at least one of SEQ ID NO:1033-1449. In further preferred embodiment, the antibody comprises at least one of the consensus domains identified in Table 2.

TABLE 1

| | | | | | | | BTLA | Light |
|---|---|---|---|---|---|---|---|---|
| | Ab_{—} | Heavy_{—} | Light_{—} | K_{D} | kₒₙ | K_{off} | IC50 | IC50 |
| AntibodyId | Num_Id | chain_id | chain_id | (M) | (1/Ms) | (1/s) | nM | nM |
| H5S14-1A1A | Ab_001 | H5S14-1AH | H5S14-1AL | 5.50E−07 | 2.80E+05 | 1.54E−01 | 2.2 | 714.7 |
| H5S14-2A2A | Ab_002 | H5S14-2AH | H5S14-2AL | | | | | |
| H5S14-3A3A | Ab_003 | H5S14-3AH | H5S14-3AL | | | | | |
| H5S14-24A4A | Ab_004 | H5S14-24AH | H5S14-4AL | 5.40E+12 | 7.69E+02 | 4.15E+15 | | |
| H5S14-4A4A | Ab_005 | H5S14-4AH | H5S14-4AL | 1.24E+11 | 2.24E+03 | 2.77E+14 | | |
| H5S14-5A5A | Ab_006 | H5S14-5AH | H5S14-5AL | 1.96E−08 | 3.53E+05 | 6.92E−03 | NA* | 24.6 |
| H5S14-6A6A | Ab_007 | H5S14-6AH | H5S14-6AL | | | | | |
| H5S14-11A10A | Ab_008 | H5S14-11AH | H5S14-10AL | 4.80E−09 | 4.82E+05 | 2.31E−03 | 1.9 | NA |
| H5S14-8A7A | Ab_009 | H5S14-8AH | H5S14-7AL | 3.51E−08 | 4.97E+05 | 1.75E−02 | 2.1 | NA |
| H5S14-10A9A | Ab_010 | H5S14-10AH | H5S14-9AL | 7.21E−06 | 1.59E+05 | 1.15E+00 | 11.3 | NA |
| H5S14-12A11A | Ab_011 | H5S14-12AH | H5S14-11AL | 2.78E−09 | 3.85E+05 | 1.07E−03 | NA* | 20.5 |
| H5S14-13A11A | Ab_012 | H5S14-13AH | H5S14-11AL | 3.22E−09 | 3.75E+05 | 1.21E−03 | NA* | 20.9 |
| H5S14-14A11A | Ab_013 | H5S14-14AH | H5S14-11AL | 4.39E−09 | 3.40E+05 | 1.49E−03 | NA* | 28.1 |
| H5S14-15A12A | Ab_014 | H5S14-15AH | H5S14-12AL | | | | | |
| H5S14-16A13A | Ab_015 | H5S14-16AH | H5S14-13AL | | | | | |
| H5S14-17A15A | Ab_016 | H5S14-17AH | H5S14-15AL | | | | | |
| H5S14-18A16A | Ab_017 | H5S14-18AH | H5S14-16AL | | | | | |
| H5S14-21A19A | Ab_018 and Ab_019 | H5S14-21AH | H5S14-19AL | 2.65E−08 | 2.45E+05 | 6.47E−03 | NA* | NA |
| H5S14-22A20A | Ab_020 | H5S14-22AH | H5S14-20AL | | | | | |
| H5S14-29A20A | Ab_021 | H5S14-29AH | H5S14-20AL | | | | | |
| H5S14-23A21A | Ab_022 | H5S14-23AH | H5S14-21AL | | | | | |
| H5S14-27A23A | Ab_023 | H5S14-27AH | H5S14-23AL | | | | | |
| H5S15-1A1A | Ab_024 | H5S15-1AH | H5S15-1AL | | | | | |
| H5S15-2A2A | Ab_025 | H5S15-2AH | H5S15-2AL | 1.16E−08 | 5.15E+05 | 5.96E−03 | 10 | NA |
| H5S15-11A3B | Ab_026 | H5S15-11AH | H5S15-3BL | 1.11E−08 | 7.76E+05 | 8.64E−03 | 2.4 | NA |
| H5S15-17A16A | Ab_027 | H5S15-17AH | H5S15-16AL | 2.68E−09 | 6.39E+05 | 1.71E−03 | 2.2 | NA |
| H5S15-33A29A | Ab_028 | H5S15-33AH | H5S15-29AL | 6.90E−09 | 3.86E+05 | 2.66E−03 | 1.4 | NA |
| H5S15-14A14B | Ab_029 | H5S15-14AH | H5S15-14BL | 4.70E−08 | 8.58E+05 | 4.03E−02 | 1.3 | NA |
| H5S15-18A17A | Ab_030 | H5S15-18AH | H5S15-17AL | 1.03E−08 | 4.05E+05 | 4.19E−03 | NA* | 21.9 |
| H5S15-8A17A | Ab_031 | H5S15-8AH | H5S15-17AL | 1.10E−08 | 2.91E+05 | 3.21E−03 | NA* | 22 |
| H5S15-7A8A | Ab_032 | H5S15-7AH | H5S15-8AL | | | | | |
| H5S15-7B8A | Ab_033 | H5S15-7BH | H5S15-8AL | | | | | |
| H5S15-16A11B | Ab_034 | H5S15-16AH | H5S15-11BL | 8.67E−09 | 2.70E+05 | 2.35E−03 | 1.3 | NA |
| H5S15-16A11E | | H5S15-16AH | H5S15-11EL | | | | | |
| H5S15-9A10A | Ab_035 | H5S15-9AH | H5S15-10AL | 6.58E−09 | 3.57E+05 | 2.35E−03 | 1.4 | NA |
| H5S15-12A13A | Ab_036 | H5S15-12AH | H5S15-13AL | 1.87E−08 | 5.71E+05 | 1.07E−02 | 0.8 | 18.7 |
| H5S15-13A15A | Ab_037 | H5S15-13AH | H5S15-15AL | | | | | |
| H5S15-19A18A | Ab_038 | H5S15-19AH | H5S15-18AL | | | | | |
| H5S15-21A19A | Ab_039 | H5S15-21AH | H5S15-19AL | | | | | |
| H5S15-23A21A | Ab_040 | H5S15-23AH | H5S15-21AL | | | | | |
| H5S15-27A22A | Ab_041 | H5S15-27AH | H5S15-22AL | | | | | |
| H5S15-28A23A | Ab_042 | H5S15-28AH | H5S15-23AL | | | | | |
| H5S15-29A24A | Ab_043 | H5S15-29AH | H5S15-24AL | 3.92E−09 | 3.78E+05 | 1.48E−03 | 1.1 | 15.3 |
| H5S15-30A26A | Ab_044 | H5S15-30AH | H5S15-26AL | 1.97E−09 | 3.08E+06 | 6.05E−03 | NA | NA |
| H5S15-31A12C | Ab_045 | H5S15-31AH | H5S15-12CL | 7.32E−09 | 3.62E+05 | 2.65E−03 | NA* | 31 |
| H5S15-35A31A | Ab_046 | H5S15-35AH | H5S15-31AL | 4.59E−09 | 3.68E+05 | 1.69E−03 | NA* | 27.2 |
| H5S15-36A32A | Ab_047 | H5S15-36AH | H5S15-32AL | | | | | |
| H5S19-24A22A | Ab_048 | H5S19-24AH | H5S19-22AL | | | | | |
| H5S19-25A24A | Ab_049 | H5S19-25AH | H5S19-24AL | | | | | |
| H5S19-9A11A | Ab_050 | H5S19-9AH | H5S19-11AL | 4.22E−08 | 1.14E+05 | 4.81E−03 | 3 | 116.6 |
| H5S19-4A6A | Ab_051 | H5S19-4AH | H5S19-6AL | 2.88E−08 | 4.96E+05 | 1.43E−02 | 1.3 | 18.6 |
| H5S19-18A17B | Ab_052 | H5S19-18AH | H5S19-17BL | 9.09E−08 | 8.07E+04 | 7.34E−03 | NA | NA |
| H5S19-20A20A | Ab_053 | H5S19-20AH | H5S19-20AL | 1.03E−07 | 4.27E+04 | 4.39E−03 | NA | NA |
| H5S19-21A17C | Ab_054 | H5S19-21AH | H5S19-17CL | 2.04E−08 | 1.43E+05 | 2.91E−03 | NA | 368.9 |
| H5S19-1A1A | Ab_055 | H5S19-1AH | H5S19-1AL | 5.44E−07 | 3.02E+04 | 1.64E−02 | NA* | NA |
| H5S19-11A9B | Ab_056 | H5S19-11AH | H5S19-9BL | | | | | |
| H5S19-5A7A | Ab_057 | H5S19-5AH | H5S19-7AL | | | | | |
| H5S19-6A8A | Ab_058 | H5S19-6AH | H5S19-8AL | 2.67E−09 | 4.69E+05 | 1.25E−03 | 1.3 | NA |
| H5S19-3B10A | | H5S19-3BH | H5S19-10AL | | | | | |
| H5S19-3B10B | Ab_059 | H5S19-3BH | H5S19-10BL | | | | | |
| H5S19-8A16A | Ab_060 | H5S19-8AH | H5S19-16AL | | | | | |
| H5S19-13A16B | Ab_061 | H5S19-13AH | H5S19-16BL | | | | | |
| H5S19-16A18A | Ab_062 | H5S19-16AH | H5S19-18AL | | | | | |
| H5S20-4A33A | | H5S20-4AH | H5S20-33AL | | | | | |
| H5S20-4A4A | Ab_063 and Ab_159 | H5S20-4AH | H5S20-4AL | 4.09E−09 | 5.28E+05 | 2.16E−03 | 2.2 | 5.7 |
| H5S20-6A6A | Ab_064 | H5S20-6AH | H5S20-6AL | 1.07E−08 | 3.33E+05 | 3.55E−03 | 1.5 | 8.7 |
| H5S20-39A29A | Ab_065 | H5S20-39AH | H5S20-29AL | 1.49E−08 | 3.30E+05 | 4.90E−03 | 1.7 | 8 |
| H5S20-1A1A | Ab_066 | H5S20-1AH | H5S20-1AL | 9.59E−09 | 2.58E+05 | 2.47E−03 | 1.5 | 10.4 |
| H5S20-7B7B | Ab_067 | H5S20-7BH | H5S20-7BL | 2.65E−09 | 5.05E+05 | 1.34E−03 | NA* | 13.6 |
| H5S20-24A19A | Ab_068 | H5S20-24AH | H5S20-19AL | 5.99E−09 | 3.20E+05 | 1.92E−03 | NA* | 20.5 |
| H5S20-43A19B | Ab_069 and Ab_155 | H5S20-43AH | H5S20-19BL | 2.97E−09 | 2.71E+05 | 8.03E−04 | NA* | 22.3 |
| H5S20-7A7A | Ab_070 | H5S20-7AH | H5S20-7AL | 2.33E−09 | 4.38E+05 | 1.02E−03 | NA* | 17.8 |
| H5S20-3B7A | Ab_071 and Ab_149 | H5S20-3BH | H5S20-7AL | 2.20E−09 | 4.23E+05 | 9.30E−04 | NA* | 20.4 |
| H5S20-15A13A | Ab_072 | H5S20-15AH | H5S20-13AL | 1.17E−08 | 1.69E+06 | 1.97E−02 | 1.6 | 252.3 |
| H5S20-9A9A | Ab_073 | H5S20-9AH | H5S20-9AL | 3.70E−07 | 2.94E+05 | 1.09E−01 | 3.2 | NA |
| H5S20-52A21A | Ab_074 | H5S20-52AH | H5S20-21AL | 4.82E−08 | 5.65E+05 | 2.72E−02 | 1.6 | NA |
| H5S20-12A10A | Ab_075 | H5S20-12AH | H5S20-10AL | | | | | |
| H5S20-26A22A | Ab_076 | H5S20-26AH | H5S20-22AL | | | | | |
| H5S20-31A16B | Ab_077 | H5S20-31AH | H5S20-16BL | 1.24E−05 | 8.66E+04 | 1.08E+00 | NA | NA |
| H5S20-45A34A | Ab_078 | H5S20-45AH | H5S20-34AL | 4.95E−09 | 4.66E+05 | 2.30E−03 | 1.5 | 14.5 |
| H5S20-22A18A | Ab_079 | H5S20-22AH | H5S20-18AL | 1.39E−08 | 1.56E+06 | 2.16E−02 | 11 | NA |
| H5S20-27A23A | Ab_080 | H5S20-27AH | H5S20-23AL | 7.09E−09 | 1.62E+05 | 1.15E−03 | 1.8 | 37.3 |
| H5S20-44A32A | Ab_081 | H5S20-44AH | H5S20-32AL | | | | | |
| H5S20-55A36A | Ab_082 | H5S20-55AH | H5S20-36AL | | | | | |
| H5S20-49A39A | Ab_083 and Ab_153 | H5S20-49AH | H5S20-39AL | 6.58E−08 | 2.54E+05 | 1.67E−02 | 1.9 | NA |
| H5S20-51A40A | Ab_084 | H5S20-51AH | H5S20-40AL | | | | | |
| H5S20-53A41A | Ab_085 | H5S20-53AH | H5S20-41AL | | | | | |
| H5S20-56A25A | Ab_086 | H5S20-56AH | H5S20-25AL | | | | | |
| H5S20-59A43A | Ab_087 | H5S20-59AH | H5S20-43AL | 1.57E−06 | 2.06E+05 | 3.23E−01 | 5.2 | NA |
| H5S14-19A17A | Ab_088 | H5S14-19AH | H5S14-17AL | | | | | |
| H5S14-30A25A | Ab_089 | H5S14-30AH | H5S14-25AL | | | | | |
| H5S14-7A4A | Ab_090 | H5S14-7AH | H5S14-4AL | | | | | |
| H5S14-7A4B | Ab_090 | H5S14-7AH | H5S14-4BL | | | | | |
| H5S15-10A11A | Ab_091 | H5S15-10AH | H5S15-11AL | | | | | |
| H5S15-15A8A | Ab_092 | H5S15-15AH | H5S15-8AL | | | | | |
| H5S15-16B11C | Ab_093 | H5S15-16BH | H5S15-11CL | | | | | |
| H5S15-20A11D | Ab_094 | H5S15-20AH | H5S15-11DL | | | | | |
| H5S15-24A3A | Ab_095 | H5S15-24AH | H5S15-3AL | | | | | |
| H5S15-25A14C | Ab_096 | H5S15-25AH | H5S15-14CL | | | | | |
| H5S15-26A17A | Ab_097 | H5S15-26AH | H5S15-17AL | | | | | |
| H5S15-32A28A | Ab_098 | H5S15-32AH | H5S15-28AL | | | | | |
| H5S15-37A21A | Ab_099 | H5S15-37AH | H5S15-21AL | | | | | |
| H5S15-38A4A | Ab_100 | H5S15-38AH | H5S15-4AL | | | | | |
| H5S15-39A22A | Ab_101 | H5S15-39AH | H5S15-22AL | | | | | |
| H5S15-3A3A | Ab_102 | H5S15-3AH | H5S15-3AL | | | | | |
| H5S15-40A18A | Ab_103 | H5S15-40AH | H5S15-18AL | | | | | |
| H5S15-5A14A | Ab_104 | H5S15-5AH | H5S15-14AL | | | | | |
| H5S15-6A6A | Ab_105 | H5S15-6AH | H5S15-6AL | | | | | |
| H5S19-12B17A | Ab_106 | H5S19-12BH | H5S19-17AL | | | | | |
| H5S19-12C17A | Ab_107 | H5S19-12CH | H5S19-17AL | | | | | |
| H5S19-14A17A | Ab_108 | H5S19-14AH | H5S19-17AL | | | | | |
| H5S19-14B23A | Ab_109 | H5S19-14BH | H5S19-23AL | | | | | |
| H5S19-17A17A | Ab_110 | H5S19-17AH | H5S19-17AL | | | | | |
| H5S19-19A19A | Ab_111 | H5S19-19AH | H5S19-19AL | | | | | |
| H5S19-20B26A | Ab_112 | H5S19-20BH | H5S19-26AL | | | | | |
| H5S19-20C28A | Ab_113 | H5S19-20CH | H5S19-28AL | | | | | |
| H5S19-22A17D | Ab_114 | H5S19-22AH | H5S19-17DL | | | | | |
| H5S19-26A17E | Ab_115 | H5S19-26AH | H5S19-17EL | | | | | |
| H5S19-27A17F | Ab_116 | H5S19-27AH | H5S19-17FL | | | | | |
| H5S19-3A5A | Ab_117 | H5S19-3AH | H5S19-5AL | | | | | |
| H5S19-3C10B | Ab_118 | H5S19-3CH | H5S19-10BL | | | | | |
| H5S20-10A9B | Ab_119 | H5S20-10AH | H5S20-9BL | | | | | |
| H5S20-10B9A | Ab_120 | H5S20-10BH | H5S20-9AL | | | | | |
| H5S20-10B9D | Ab_120 | H5S20-10BH | H5S20-9DL | | | | | |
| H5S20-10B9E | Ab_120 | H5S20-10BH | H5S20-9EL | | | | | |
| H5S20-11A1A | Ab_121 | H5S20-11AH | H5S20-1AL | | | | | |
| H5S20-11B1B | Ab_122 | H5S20-11BH | H5S20-1BL | | | | | |
| H5S20-11C28A | Ab_123 | H5S20-11CH | H5S20-28AL | | | | | |
| H5S20-14A12A | Ab_124 | H5S20-14AH | H5S20-12AL | | | | | |
| H5S20-14A1A | Ab_124 | H5S20-14AH | H5S20-1AL | | | | | |
| H5S20-14B12A | Ab_125 | H5S20-14BH | H5S20-12AL | | | | | |
| H5S20-14C12A | Ab_126 | H5S20-14CH | H5S20-12AL | | | | | |
| H5S20-14D12B | Ab_127 | H5S20-14DH | H5S20-12BL | | | | | |
| H5S20-17A21A | Ab_128 | H5S20-17AH | H5S20-21AL | | | | | |
| H5S20-18A15A | Ab_129 | H5S20-18AH | H5S20-15AL | | | | | |
| H5S20-20A16A | Ab_130 | H5S20-20AH | H5S20-16AL | | | | | |
| H5S20-20B16B | Ab_131 | H5S20-20BH | H5S20-16BL | | | | | |
| H5S20-21A17A | Ab_132 | H5S20-21AH | H5S20-17AL | | | | | |
| H5S20-23A3B | Ab_133 | H5S20-23AH | H5S20-3BL | | | | | |
| H5S20-25A20A | Ab_134 | H5S20-25AH | H5S20-20AL | | | | | |
| H5S20-28A24A | Ab_135 | H5S20-28AH | H5S20-24AL | | | | | |
| H5S20-28B24A | Ab_136 | H5S20-28BH | H5S20-24AL | | | | | |
| H5S20-29A1A | Ab_137 | H5S20-29AH | H5S20-1AL | | | | | |
| H5S20-30A24A | Ab_138 | H5S20-30AH | H5S20-24AL | | | | | |
| H5S20-31B16B | Ab_139 | H5S20-31BH | H5S20-16BL | | | | | |
| H5S20-32A7C | Ab_140 | H5S20-32AH | H5S20-7CL | | | | | |
| H5S20-32B31A | Ab_141 | H5S20-32BH | H5S20-31AL | | | | | |
| H5S20-32B7A | Ab_141 | H5S20-32BH | H5S20-7AL | | | | | |
| H5S20-32C7A | Ab_142 | H5S20-32CH | H5S20-7AL | | | | | |
| H5S20-33A7D | Ab_143 | H5S20-33AH | H5S20-7DL | | | | | |
| H5S20-34A8B | Ab_144 | H5S20-34AH | H5S20-8BL | | | | | |
| H5S20-35A25A | Ab_145 | H5S20-35AH | H5S20-25AL | | | | | |
| H5S20-36A26A | Ab_146 | H5S20-36AH | H5S20-26AL | | | | | |
| H5S20-37A27A | Ab_147 | H5S20-37AH | H5S20-27AL | | | | | |
| H5S20-38A7D | Ab_148 | H5S20-38AH | H5S20-7DL | | | | | |
| H5S20-3C7A | Ab_150 | H5S20-3CH | H5S20-7AL | | | | | |
| H5S20-40A30A | Ab_151 | H5S20-40AH | H5S20-30AL | | | | | |
| H5S20-41A25A | Ab_152 | H5S20-41AH | H5S20-25AL | | | | | |
| H5S20-41B25A | Ab_153 | H5S20-41BH | H5S20-25AL | | | | | |
| H5S20-42A8A | Ab_154 | H5S20-42AH | H5S20-8AL | | | | | |
| H5S20-46A36A | Ab_156 | H5S20-46AH | H5S20-36AL | | | | | |
| H5S20-48A7D | Ab_157 | H5S20-48AH | H5S20-7DL | | | | | |
| H5S20-4B12A | Ab_160 | H5S20-4BH | H5S20-12AL | | | | | |
| H5S20-4B33A | Ab_160 | H5S20-4BH | H5S20-33AL | | | | | |
| H5S20-4B42A | Ab_160 | H5S20-4BH | H5S20-42AL | | | | | |
| H5S20-50A7D | Ab_161 | H5S20-50AH | H5S20-7DL | | | | | |
| H5S20-54A24A | Ab_162 | H5S20-54AH | H5S20-24AL | | | | | |
| H5S20-57A25A | Ab_163 | H5S20-57AH | H5S20-25AL | | | | | |
| H5S20-58A21A | Ab_164 | H5S20-58AH | H5S20-21AL | | | | | |
| H5S20-60A7E | Ab_165 | H5S20-60AH | H5S20-7EL | | | | | |
| H5S20-61A29A | Ab_166 | H5S20-61AH | H5S20-29AL | | | | | |
| H5S20-62A7A | Ab_167 | H5S20-62AH | H5S20-7AL | | | | | |
| H5S20-8A8A | Ab_168 | H5S20-8AH | H5S20-8AL | | | | | |
| H5S14-17A16A | | H5S14-17AH | H5S14-16AL | | | | | |
| H5S14-20A18A | | H5S14-20AH | H5S14-18AL | | | | | |
| H5S14-25A16A | | H5S14-25AH | H5S14-16AL | | | | | |
| H5S14-26A22A | | H5S14-26AH | H5S14-22AL | | | | | |
| H5S14-28A24A | | H5S14-28AH | H5S14-24AL | | | | | |
| H5S19-11A15A | | H5S19-11AH | H5S19-15AL | | | | | |
| H5S19-19A18A | | H5S19-19AH | H5S19-18AL | | | | | |
| H5S19-3A3A | | H5S19-3AH | H5S19-3AL | | | | | |
| H5S19-3B14A | | H5S19-3BH | H5S19-14AL | | | | | |
| H5S19-3B3A | | H5S19-3BH | H5S19-3AL | | | | | |
| H5S19-3B4A | | H5S19-3BH | H5S19-4AL | | | | | |
| H5S19-8A10A | | H5S19-8AH | H5S19-10AL | | | | | |
| ITI_021 | | ITI_21H | ITI_21L | | | | | |
| ITI_023 | | ITI_23H | ITI_23L | | | | | |
| ITI_027 | | | ITI_27L | | | | | |
| ITI_028 | | | ITI_28L | | | | | |
| ITI_029 | | | ITI_29L | | | | | |
| ITI_030 | | | ITI_30L | | | | | |
| ITI_032 | | ITI_32H | | | | | | |
| ITI_033 | | | ITI_33L | | | | | |
| ITI_035 | | ITI_35H | | | | | | |
| ITI_038 | | | ITI_38L | | | | | |
| ITI_040 | | ITI_40H | ITI_40L | | | | | |
| ITI_045 | | ITI_45H | ITI_45L | | | | | |
| ITI_046 | | ITI_46H | | | | | | |
| ITI_047 | | ITI_47H | ITI_47L | | | | | |
| ITI_053 | | ITI_53H | | | | | | |
| ITI_082 | | ITI_82H | ITI_82L | | | | | |
| ITI_083 | | ITI_83H | ITI_83L | | | | | |
| ITI_091 | | ITI_91H | ITI_91L | | | | | |
| ITI_100 | | ITI_100H | | | | | | |
| ITI_101 | | ITI_101H | | | | | | |
| ITI_122 | | ITI_122H | ITI_122L | | | | | |
| ITI_127 | | ITI_127H | ITI_127L | | | | | |
| ITI_128 | | ITI_128H | ITI_128L | | | | | |
| ITI_130 | | ITI_130H | | | | | | |
| ITI_131 | | ITI_131H | ITI_131L | | | | | |
| ITI_144 | | ITI_144H | ITI_144L | | | | | |
| ITI_145 | | ITI_145H | ITI_145L | | | | | |
| ITI_146 | | ITI_146H | ITI_146L | | | | | |
| ITI_162 | | ITI_162H | ITI_162L | | | | | |
| ITI_164 | | ITI_164H | | | | | | |
| ITI_165 | | ITI_165H | | | | | | |
| ITI_166 | | | ITI_166L | | | | | |
| ITI_168 | | ITI_168H | | | | | | |
| ITI_169 | | | ITI_169L | | | | | |
| ITI_173 | | ITI_173H | ITI_173L | | | | | |
| ITI_180 | | ITI_180H | | | | | | |
| ITI_200 | | ITI_200H | ITI_200L | | | | | |
| ITI_203 | | ITI_203H | ITI_203L | | | | | |
| ITI_236 | | ITI_236H | ITI_236L | | | | | |
| ITI_237 | | ITI_237H | ITI_237L | | | | | |
| ITI_238 | | ITI_238H | | | | | | |
| ITI_240 | | ITI_240H | ITI_240L | | | | | |

TABLE 2

| | | | | | | | | | | | | | | |
|---|---|---|---|---|---|---|---|---|---|---|---|---|---|---|
| Sequence | | Seq. | | Seq. | | Seq. | | Seq. | | Seq. | | Seq. | | Seq. |
| name | FR1 | ID | CDR1 | ID | FR2 | ID | CDR2 | ID | FR3 | ID | CDR3 | ID | FR4 ID | ID |

Consensus Cluster #11

| | | | | | | | | | [K/N/I/ | | | | | |
|---|---|---|---|---|---|---|---|---|---|---|---|---|---|---|
| | | | | | | | | | M]YNE[N | | | | | |
| | | | | | | | | | /K]F[K/ | | | | | |
| | | | | | | | | | R]GKA[T | | | | | |
| | | | | | | | | | /I]L[T/ | | | | | |
| | | | | | | | | | S][A/V/ | | | | | |
| | | | | | | | | | I]D[T/S | | | | | |
| | | | | | | | | | ]S[S/A] | | | | | |
| | | | | | | | | | [S/N/T] | | | | | |
| | QVQLQQS | | | | | | | | TAY[M/I | | | | | |
| | G[A/T]E | | G[Y/F/N | | | | | | ]QL[S/N | | | | | |
| | [L/V][V | | ][T/R/A | | LGW[V/I | | | | /G][R/S | | [V/A/I] | | | |
| | /I]RPGT | | /I/S][L | | ][K/R][ | | | | /G]LTS[ | | GA[V/M/ | | | |
| | SV[R/K/ | | /F][T/I | | Q/L][R/ | | [I/V]YP | | E/G]DS[ | | L][A/D] | | WGQGT[S | |
| | Q/M][I/ | | ]N[S/Y] | | W]PGHGL | | GGGY[T/ | | G/A]VYF | | [Y/N/H/ | | /A]V[T/ | |
| | M]SCKAS | 202 | W | 285 | EWIGD | 377 | N/I/A] | 464 | C | 561 | S/K] | 709 | I]VSS | 847 |
| H5S14- | QVQLQQS | 203 | GNIFTNS | 286 | LGWIKQR | 378 | VYPGGGY | 465 | KYNEKFK | 562 | AGAMDY | 710 | WGQGTSV | 848 |
| 12AH | GAELVRP | | W | | PGHGLEW | | N | | GKATLTI | | | | TVSS | |
| | GTSVKIS | | | | IGD | | | | DTSASTA | | | | | |
| | CKAS | | | | | | | | YMQLSRL | | | | | |
| | | | | | | | | | TSEDSGV | | | | | |
| | | | | | | | | | YFC | | | | | |
| H5S14- | QVQLQQS | 203 | GYIFTNS | 287 | LGWIKQR | 378 | VYPGGGY | 465 | KYNEKFK | 563 | AGAMDY | 710 | WGQGTSV | 848 |
| 13AH | GAELVRP | | W | | PGHGLEW | | N | | GKATLTI | | | | TVSS | |
| | GTSVKIS | | | | IGD | | | | DTSASTA | | | | | |
| | CKAS | | | | | | | | YMQLSRL | | | | | |
| | | | | | | | | | TSEDSAV | | | | | |
| | | | | | | | | | YFC | | | | | |
| H5S14- | QVQLQQS | 203 | GYIFTNY | 288 | LGWIKQR | 378 | VYPGGGY | 465 | KYNEKFK | 564 | AGAMDY | 710 | WGQGTSV | 848 |
| 14AH | GAELVRP | | W | | PGHGLEW | | N | | GKATLTV | | | | TVSS | |
| | GTSVKIS | | | | IGD | | | | DTSASTA | | | | | |
| | CKAS | | | | | | | | YMQLSRL | | | | | |
| | | | | | | | | | TSEDSAV | | | | | |
| | | | | | | | | | YFC | | | | | |
| H5S14- | QVQLQQS | 203 | GYTFTNS | 289 | LGWVKQR | 379 | IYPGGGY | 466 | KYNEKFK | 565 | AGAMDS | 711 | WGQGTSV | 848 |
| 19AH | GAELVRP | | W | | PGHGLEW | | N | | GKATLTA | | | | TVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S15- | QVQLQQS | 203 | GYAFTNS | 290 | LGWVKQR | 379 | IYPGGGY | 466 | KYNEKFK | 565 | AGALDY | 712 | WGQGTSV | 848 |
| 31AH | GAELVRP | | W | | PGHGLEW | | N | | GKATLTA | | | | TVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S15- | QVQLQQS | 203 | GYTFTNS | 289 | LGWVKQR | 379 | IYPGGGY | 466 | KYNEKFK | 566 | AGAMDY | 710 | WGQGTSV | 848 |
| 35AH | GAELVRP | | W | | PGHGLEW | | N | | GKATLTA | | | | TVSS | |
| | GTSVKIS | | | | IGD | | | | DTSSSTA | | | | | |
| | CKAS | | | | | | | | YMQLGSL | | | | | |
| | | | | | | | | | TSEDSAV | | | | | |
| | | | | | | | | | YFC | | | | | |
| H5S20- | QVQLQQS | 203 | GYTLINS | 291 | LGWVKQR | 379 | IYPGGGY | 466 | KYNEKFK | 567 | AGAMDY | 710 | WGQGTSV | 848 |
| 23AH | GAELVRP | | W | | PGHGLEW | | N | | GKAILTA | | | | TVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 203 | GYTFTNY | 292 | LGWVKQR | 379 | IYPGGGY | 467 | IYNEKFK | 568 | AGAMDH | 713 | WGQGTSV | 848 |
| 24AH | GAELVRP | | W | | PGHGLEW | | I | | GKATLTV | | | | TVSS | |
| | GTSVKIS | | | | IGD | | | | DSSATTA | | | | | |
| | CKAS | | | | | | | | YIQLNSL | | | | | |
| | | | | | | | | | TSEDSAV | | | | | |
| | | | | | | | | | YFC | | | | | |
| H5S20- | QVQLQQS | 204 | GFTLTNY | 293 | LGWVKQR | 379 | IYPGGGY | 468 | NYNEKFK | 569 | AGAMDK | 714 | WGQGTSV | 848 |
| 28AH | GAEVVRP | | W | | PGHGLEW | | T | | GKATLTA | | | | TVSS | |
| | GTSVQIS | | | | IGD | | | | DTSSNTA | | | | | |
| | CKAS | | | | | | | | YMQLSGL | | | | | |
| | | | | | | | | | TSEDSAV | | | | | |
| | | | | | | | | | YFC | | | | | |
| H5S20- | QVQLQQS | 205 | GFTLTNY | 293 | LGWVKQR | 379 | IYPGGGY | 468 | NYNEKFK | 569 | AGAMDK | 714 | WGQGTSV | 848 |
| 28BH | GAEVVRP | | W | | PGHGLEW | | T | | GKATLTA | | | | TVSS | |
| | GTSVKIS | | | | IGD | | | | DTSSNTA | | | | | |
| | CKAS | | | | | | | | YMQLSGL | | | | | |
| | | | | | | | | | TSEDSAV | | | | | |
| | | | | | | | | | YFC | | | | | |
| H5S20- | QVQLQQS | 206 | GFTLTNY | 293 | LGWVKQR | 379 | IYPGGGY | 469 | NYNEKFK | 569 | AGAMDK | 714 | WGQGTSV | 848 |
| 30AH | GTEVVRP | | W | | PGHGLEW | | A | | GKATLTA | | | | TVSS | |
| | GTSVKIS | | | | IGD | | | | DTSSNTA | | | | | |
| | CKAS | | | | | | | | YMQLSGL | | | | | |
| | | | | | | | | | TSEDSAV | | | | | |
| | | | | | | | | | YFC | | | | | |
| H5S20- | QVQLQQS | 203 | GYTFTNY | 292 | LGWVKQW | 380 | IYPGGGY | 466 | IYNEKFK | 570 | AGAMDY | 710 | WGQGTSV | 848 |
| 32AH | GAELVRP | | W | | PGHGLEW | | N | | GKATLTV | | | | TVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 203 | GYTFTNY | 292 | LGWVKQR | 379 | IYPGGGY | 466 | IYNEKFK | 570 | AGAMDY | 710 | WGQGTSV | 848 |
| 32BH | GAELVRP | | W | | PGHGLEW | | N | | GKATLTV | | | | TVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 203 | GYTFTNY | 292 | LGWVKQR | 379 | IYPGGGY | 466 | IYNEKFK | 571 | AGAMDY | 710 | WGQGTSV | 848 |
| 32CH | GAELVRP | | W | | PGHGLEW | | N | | GKATLTI | | | | TVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 207 | GYTLTNS | 294 | LGWVKQR | 379 | IYPGGGY | 468 | KYNENFK | 572 | VGAVAY | 715 | WGQGTSV | 848 |
| 33AH | GAELVRP | | W | | PGHGLEW | | T | | GKATLTA | | | | TVSS | |
| | GTSVRIS | | | | IGD | | | | DTSSSTA | | | | | |
| | CKAS | | | | | | | | YMQLSRL | | | | | |
| | | | | | | | | | TSEDSGV | | | | | |
| | | | | | | | | | YFC | | | | | |
| H5S20- | QVQLQQS | 203 | GYTFTNY | 292 | LGWVKQR | 379 | IYPGGGY | 466 | KYNEKFK | 565 | AGAMDY | 710 | WGQGTSV | 848 |
| 35AH | GAELVRP | | W | | PGHGLEW | | N | | GKATLTA | | | | TVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 208 | GYTFTNS | 289 | LGWVKQR | 379 | IYPGGGY | 467 | KYNEKFK | 565 | AGAMDY | 710 | WGQGTSV | 848 |
| 36AH | GAELVRP | | W | | PGHGLEW | | I | | GKATLTA | | | | TVSS | |
| | GTSVKMS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 207 | GYTLTNS | 294 | LGWVKQR | 379 | IYPGGGY | 468 | KYNEKFK | 565 | AGAVAY | 716 | WGQGTSV | 848 |
| 38AH | GAELVRP | | W | | PGHGLEW | | T | | GKATLTA | | | | TVSS | |
| | GTSVRIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 203 | GYTFTNS | 289 | LGWVKQR | 379 | IYPGGGY | 466 | KYNEKFR | 573 | AGAMDY | 710 | WGQGTSV | 848 |
| 3BH | GAELVRP | | W | | PGHGLEW | | N | | GKATLTA | | | | TVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 203 | GYTFTNS | 289 | LGWVKQR | 379 | IYPGGGY | 466 | KYNEKFK | 565 | AGAMDY | 710 | WGQGTSV | 848 |
| 3CH | GAELVRP | | W | | PGHGLEW | | N | | GKATLTA | | | | TVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 203 | GYRFTNY | 295 | LGWVKQR | 379 | IYPGGGY | 466 | KYNEKFK | 565 | AGAMDY | 710 | WGQGTSV | 848 |
| 41AH | GAELVRP | | W | | PGHGLEW | | N | | GKATLTA | | | | TVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 203 | GYRFTNY | 295 | LGWIKQR | 378 | IYPGGGY | 466 | KYNEKFK | 565 | AGAMDY | 710 | WGQGTSV | 849 |
| 41BH | GAELVRP | | W | | PGHGLEW | | N | | GKATLTA | | | | IVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 209 | GYTFTNY | 292 | LGWVKQR | 379 | IYPGGGY | 467 | IYNEKFK | 574 | AGAMDY | 710 | WGQGTSV | 848 |
| 43AH | GAELVRP | | W | | PGHGLEW | | I | | GKATLTV | | | | TVSS | |
| | GTSVMIS | | | | IGD | | | | DSSATTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 207 | GYTLTNS | 294 | LGWVKQR | 379 | IYPGGGY | 466 | KYNEKFK | 565 | AGAVAY | 716 | WGQGTSV | 848 |
| 48AH | GAELVRP | | W | | PGHGLEW | | N | | GKATLTA | | | | TVSS | |
| | GTSVRIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 203 | GYRFTNS | 296 | LGWVKQR | 379 | IYPGGGY | 466 | KYNEKFK | 575 | AGALDY | 712 | WGQGTSV | 848 |
| 50AH | GAELVRP | | W | | PGHGLEW | | N | | GKATLTA | | | | TVSS | |
| | GTSVKIS | | | | IGD | | | | DTSSNTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 205 | GFTLTNY | 293 | LGWVKQR | 379 | IYPGGGY | 468 | NYNEKFK | 569 | AGAMDN | 717 | WGQGTSV | 848 |
| 54AH | GAEVVRP | | W | | PGHGLEW | | T | | GKATLTA | | | | TVSS | |
| | GTSVKIS | | | | IGD | | | | DTSSNTA | | | | | |
| | CKAS | | | | | | | | YMQLSGL | | | | | |
| | | | | | | | | | TSEDSAV | | | | | |
| | | | | | | | | | YFC | | | | | |
| H5S20- | QVQLQQS | 203 | GYTFTNS | 289 | LGWVKQR | 379 | IYPGGGY | 466 | KYNEKFK | 565 | IGAMDY | 718 | WGQGTSV | 848 |
| 57AH | GAELVRP | | W | | PGHGLEW | | N | | GKATLTA | | | | TVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 207 | GYTLTNS | 294 | LGWVKQR | 379 | IYPGGGY | 468 | KYNENFK | 576 | AGAMAY | 719 | WGQGTSV | 848 |
| 60AH | GAELVRP | | W | | PGHGLEW | | T | | GKATLSA | | | | TVSS | |
| | GTSVRIS | | | | IGD | | | | DTSSSTA | | | | | |
| | CKAS | | | | | | | | YMQLSSL | | | | | |
| | | | | | | | | | TSGDSAV | | | | | |
| | | | | | | | | | YFC | | | | | |
| H5S20- | QVQLQQS | 203 | GYSFTNY | 297 | LGWVKQR | 379 | IYPGGGY | 466 | MYNEKFK | 577 | AGAMDY | 710 | WGQGTSV | 848 |
| 62AH | GAELVRP | | W | | PGHGLEW | | N | | GKATLTV | | | | TVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 203 | GYRFTNS | 296 | LGWIKQR | 378 | IYPGGGY | 466 | KYNEKFR | 573 | AGAMDS | 711 | WGQGTSV | 848 |
| 7AH | GAELVRP | | W | | PGHGLEW | | N | | GKATLTA | | | | TVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| H5S20- | QVQLQQS | 210 | GYRFTNS | 296 | LGWIKLR | 381 | IYPGGGY | 466 | KYNEKFR | 573 | AGAMDS | 711 | WGQGTSV | 848 |
| 7BH | GAELIRP | | W | | PGHGLEW | | N | | GKATLTA | | | | TVSS | |
| | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| ITI_ | QVQLQQS | 203 | GYTFTNS | 289 | LGWVKQR | 379 | IYPGGGY | 466 | KYNEKFK | 578 | AGAMDY | 710 | WGQGTAV | 850 |
| 04 | GAELVRP | | W | | PGHGLEW | | N | | GKATLTA | | | | TVSS | |
| 7H | GTSVKIS | | | | IGD | | | | DTSSSTA | | | | | |
| | CKAS | | | | | | | | YMQLSSL | | | | | |
| | | | | | | | | | TSEDSGV | | | | | |
| | | | | | | | | | YFC | | | | | |
| ITI_ | QVQLQQS | 203 | GYAFTNS | 290 | LGWVRQR | 382 | IYPGGGY | 466 | KYNEKFK | 565 | AGALDY | 712 | WGQGTSV | 848 |
| 05 | GAELVRP | | W | | PGHGLEW | | N | | GKATLTA | | | | TVSS | |
| 3H | GTSVKIS | | | | IGD | | | | DTSSSTA | | | | | |
| | CKAS | | | | | | | | YMQLSSL | | | | | |
| | | | | | | | | | TSEDSAV | | | | | |
| | | | | | | | | | YFC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #20

| | | | | | | | | | [F/Y][Y | | | | | |
|---|---|---|---|---|---|---|---|---|---|---|---|---|---|---|
| | | | | | | | | | /S/F][R | | | | | |
| | | | | | | | | | /P/S/A] | | | | | |
| | | | | | | | | | D[S/N/T | | | | | |
| | | | | | | | | | ][V/L]K | | | | | |
| | | | | | | | | | [A/G]RF | | | | | |
| | | | | | | | | | TISRD[D | | | | | |
| | | | | | | | | | /N][V/A | | [T/A]RG | | | |
| | | | | | | | | | /P][R/G | | [G/A][Y | | | |
| | [E/D]V[ | | | | | | | | /K][D/N | | /G]G[-/ | | | |
| | K/Q]LVE | | | | | | I[S/T][ | | ][I/T][ | | N][-/Y/ | | | |
| | SGG[G/D | | | | M[S/H]W | | S/T]G[- | | L/V][Y/ | | F][S/F/ | | | |
| | ]L[V/M] | | | | [V/I]RQ | | /G/S][G | | F]LQM[S | | A][S/W] | | WGQGT[T | |
| | [K/M/Q] | | GFTFS[S | | [T/A]PE | | /T/S][D | | /T]SL[R | | [F/H/Y] | | /L][L/V | |
| | PGGS[L/ | | /N/D][Y | | [T/K][R | | /N/S/K/ | | /K]SEDT | | [V/T/A/ | | ]T[V/I] | |
| | R][K/R] | | /F][A/G | | /G]LEWV | | T][T/S/ | | AMY[F/Y | | P][I/Y/ | | S[S/A/T | |
| | LSCAAS | 211 | ] | 298 | A[S/H] | 383 | I] | 470 | /A]C | 579 | F] | 720 | ] | 851 |
| H5S14- | EVKLVES | 212 | GFTFSSF | 299 | MSWIRQT | 384 | ITTG-GS | 471 | YSPDSLK | 580 | ARGGGGN | 721 | WGQGTLV | 852 |
| 11AH | GGGLVKP | | A | | PEKGLEW | | S | | GRFTISR | | YFWFAY | | TVSA | |
| | GGSLRLS | | | | VAS | | | | DNVRNIV | | | | | |
| | CAAS | | | | | | | | YLQMSSL | | | | | |
| | | | | | | | | | RSEDTAM | | | | | |
| | | | | | | | | | YAC | | | | | |
| H5S14- | EVKLVES | 213 | GFTFSNY | 300 | MSWVRQT | 385 | ISTGGTT | 472 | YYSDSVK | 581 | ARGGGGN | 722 | WGQGTLV | 853 |
| 8AH | GGDLVKP | | A | | PEKRLEW | | S | | GRFTISR | | YFWFTY | | TISA | |
| | GGSLKLS | | | | VAS | | | | DNARNIL | | | | | |
| | CAAS | | | | | | | | YLQMSSL | | | | | |
| | | | | | | | | | RSEDTAM | | | | | |
| | | | | | | | | | YYC | | | | | |
| H5S15- | DVQLVES | 214 | GFTFSSF | 301 | MHWIRQA | 386 | ISSGSST | 473 | YFADTVK | 582 | ARGAYGN | 723 | WGQGTLV | 852 |
| 10AH | GGGLVQP | | G | | PEKGLEW | | I | | GRFTISR | | FAWFPY | | TVSA | |
| | GGSRKLS | | | | VAS | | | | DNPKNTL | | | | | |
| | CAAS | | | | | | | | FLQMTSL | | | | | |
| | | | | | | | | | RSEDTAM | | | | | |
| | | | | | | | | | YYC | | | | | |
| H5S15- | DVQLVES | 214 | GFTFSSF | 301 | MHWVRQA | 387 | ISSGSST | 473 | YYADTVK | 583 | ARGAYGN | 723 | WGQGTLV | 852 |
| 16AH | GGGLVQP | | G | | PEKGLEW | | I | | GRFTISR | | FAWFPY | | TVSA | |
| | GGSRKLS | | | | VAH | | | | DNPKNTL | | | | | |
| | CAAS | | | | | | | | FLQMTSL | | | | | |
| | | | | | | | | | RSEDTAM | | | | | |
| | | | | | | | | | YYC | | | | | |
| H5S15- | DVQLVES | 214 | GFTFSSF | 301 | MHWVRQA | 387 | ISSGSST | 473 | YYADTVK | 583 | ARGAYGN | 723 | WGQGTLV | 854 |
| 16BH | GGGLVQP | | G | | PEKGLEW | | I | | GRFTISR | | FAWFPY | | TVST | |
| | GGSRKLS | | | | VAH | | | | DNPKNTL | | | | | |
| | CAAS | | | | | | | | FLQMTSL | | | | | |
| | | | | | | | | | RSEDTAM | | | | | |
| | | | | | | | | | YYC | | | | | |
| H5S15- | DVQLVES | 214 | GFTFSSF | 301 | MHWVRQA | 388 | ISSGSST | 473 | YYADTVK | 583 | ARGAYGN | 724 | WGQGTLV | 852 |
| 20AH | GGGLVQP | | G | | PEKGLEW | | I | | GRFTISR | | FAWFAF | | TVSA | |
| | GGSRKLS | | | | VAS | | | | DNPKNTL | | | | | |
| | CAAS | | | | | | | | FLQMTSL | | | | | |
| | | | | | | | | | RSEDTAM | | | | | |
| | | | | | | | | | YYC | | | | | |
| H5S15- | DVQLVES | 214 | GFTFSDF | 302 | MHWVRQA | 387 | ISSGSST | 473 | YYADTVK | 583 | ARGAYGN | 723 | WGQGTLV | 852 |
| 9AH | GGGLVQP | | G | | PEKGLEW | | I | | GRFTISR | | FAWFPY | | TVSA | |
| | GGSRKLS | | | | VAH | | | | DNPKNTL | | | | | |
| | CAAS | | | | | | | | FLQMTSL | | | | | |
| | | | | | | | | | RSEDTAM | | | | | |
| | | | | | | | | | YYC | | | | | |
| H5S20- | EVKLVES | 215 | GFTFSSY | 303 | MSWVRQT | 389 | ISSG-GN | 474 | FYPDSVK | 584 | TRGGYG- | 725 | WGQGTTV | 855 |
| 11AH | GGGLVKP | | A | | PETRLEW | | T | | GRFTISR | | -SSYVI | | TVSS | |
| | GGSLKLS | | | | VAS | | | | DNVRDIL | | | | | |
| | CAAS | | | | | | | | YLQMSSL | | | | | |
| | | | | | | | | | RSEDTAM | | | | | |
| | | | | | | | | | YFC | | | | | |
| H5S20- | EVKLVES | 215 | GFTFSSY | 303 | MSWVRQT | 389 | ISSG-GN | 474 | FYPDSVK | 584 | TRGGYG- | 725 | WGQGTTL | 856 |
| 11BH | GGGLVKP | | A | | PETRLEW | | T | | GRFTISR | | -SSYVI | | TVSS | |
| | GGSLKLS | | | | VAS | | | | DNVRDIL | | | | | |
| | CAAS | | | | | | | | YLQMSSL | | | | | |
| | | | | | | | | | RSEDTAM | | | | | |
| | | | | | | | | | YFC | | | | | |
| H5S20- | EVKLVES | 215 | GFTFSSY | 303 | MSWVRQT | 389 | ISSG-GN | 474 | FYPDSVK | 585 | TRGGYG- | 725 | WGQGTTL | 856 |
| 11CH | GGGLVKP | | A | | PETRLEW | | T | | GRFTISR | | -SSYVI | | TVSS | |
| | GGSLKLS | | | | VAS | | | | DDVRDIL | | | | | |
| | CAAS | | | | | | | | YLQMSSL | | | | | |
| | | | | | | | | | RSEDTAM | | | | | |
| | | | | | | | | | YFC | | | | | |
| H5S20- | EVKLVES | 215 | GFTFSSY | 303 | MSWVRQT | 389 | ISSG-GN | 474 | YYPDSVK | 586 | TRGGYG- | 725 | WGQGTTL | 856 |
| 14AH | GGGLVKP | | A | | PETRLEW | | T | | GRFTISR | | -SSYVI | | TVSS | |
| | GGSLKLS | | | | VAS | | | | DNVRNIL | | | | | |
| | CAAS | | | | | | | | YLQMSSL | | | | | |
| | | | | | | | | | RSEDTAM | | | | | |
| | | | | | | | | | YFC | | | | | |
| H5S20- | EVKLVES | 215 | GFTFSSY | 303 | MSWVRQT | 389 | ISSG-GN | 474 | YYPDSVK | 587 | TRGGYG- | 725 | WGQGTTL | 856 |
| 14BH | GGGLVKP | | A | | PETRLEW | | T | | GRFTISR | | -SSYVI | | TVSS | |
| | GGSLKLS | | | | VAS | | | | DDVRNIL | | | | | |
| | CAAS | | | | | | | | YLQMSSL | | | | | |
| | | | | | | | | | RSEDTAM | | | | | |
| | | | | | | | | | YFC | | | | | |
| H5S20- | EVKLVES | 215 | GFTFSSY | 303 | MSWVRQT | 389 | ISSG-GN | 474 | YYPDSVK | 588 | TRGGYG- | 725 | WGQGTTL | 856 |
| 14CH | GGGLVKP | | A | | PETRLEW | | T | | GRFTISR | | -SSYVI | | TVSS | |
| | GGSLKLS | | | | VAS | | | | DNVRDIL | | | | | |
| | CAAS | | | | | | | | YLQMSSL | | | | | |
| | | | | | | | | | KSEDTAM | | | | | |
| | | | | | | | | | YFC | | | | | |
| H5S20- | EVKLVES | 216 | GFTFSSY | 303 | MSWVRQT | 389 | ISSG-GN | 474 | YYPDSVK | 586 | TRGGYG- | 725 | WGQGTTL | 856 |
| 14DH | GGGLVMP | | A | | PETRLEW | | T | | GRFTISR | | -SSYVI | | TVSS | |
| | GGSLKLS | | | | VAS | | | | DNVRNIL | | | | | |
| | CAAS | | | | | | | | YLQMSSL | | | | | |
| | | | | | | | | | RSEDTAM | | | | | |
| | | | | | | | | | YFC | | | | | |
| H5S20- | EVKLVES | 215 | GFTFSSY | 303 | MSWVRQT | 389 | ISSG-GD | 475 | FYRDSVK | 589 | TRGGYG- | 726 | WGQGTTL | 856 |
| 1AH | GGGLVKP | | A | | PETRLEW | | T | | ARFTISR | | -SSFVI | | TVSS | |
| | GGSLKLS | | | | VAS | | | | DDVRDIL | | | | | |
| | CAAS | | | | | | | | YLQMSSL | | | | | |
| | | | | | | | | | RSEDTAM | | | | | |
| | | | | | | | | | YFC | | | | | |
| H5S20- | EVKLVES | 215 | GFTFSSY | 303 | MSWVRQT | 389 | ISSG-GN | 474 | FYPDSVK | 585 | TRGGYG- | 726 | WGQGTTL | 856 |
| 29AH | GGGLVKP | | A | | PETRLEW | | T | | GRFTISR | | -SSFVI | | TVSS | |
| | GGSLKLS | | | | VAS | | | | DDVRDIL | | | | | |
| | CAAS | | | | | | | | YLQMSSL | | | | | |
| | | | | | | | | | RSEDTAM | | | | | |
| | | | | | | | | | YFC | | | | | |
| H5S20- | EVKLVES | 215 | GFTFSSY | 303 | MSWVRQT | 389 | ISSG-GK | 476 | FYPDSVK | 584 | TRGGYG- | 725 | WGQGTTL | 856 |
| 37AH | GGGLVKP | | A | | PETRLEW | | T | | GRFTISR | | -SSYVI | | TVSS | |
| | GGSLKLS | | | | VAS | | | | DNVRDIL | | | | | |
| | CAAS | | | | | | | | YLQMSSL | | | | | |
| | | | | | | | | | RSEDTAM | | | | | |
| | | | | | | | | | YFC | | | | | |
| H5S20- | EVKLVES | 215 | GFTFSSY | 303 | MSWVRQT | 389 | ISSG-GN | 474 | YYPDSVK | 586 | TRGGYG- | 727 | WGQGTTL | 856 |
| 39AH | GGGLVKP | | A | | PETRLEW | | T | | GRFTISR | | -SSHVI | | TVSS | |
| | GGSLKLS | | | | VAS | | | | DNVRNIL | | | | | |
| | CAAS | | | | | | | | YLQMSSL | | | | | |
| | | | | | | | | | RSEDTAM | | | | | |
| | | | | | | | | | YFC | | | | | |
| H5S20- | EVKLVES | 215 | GFTFSNY | 300 | MSWVRQT | 389 | ISSG-GN | 474 | YYPDSVK | 586 | TRGGYG- | 725 | WGQGTTL | 856 |
| 40AH | GGGLVKP | | A | | PETRLEW | | T | | GRFTISR | | -SSYVI | | TVSS | |
| | GGSLKLS | | | | VAS | | | | DNVRNIL | | | | | |
| | CAAS | | | | | | | | YLQMSSL | | | | | |
| | | | | | | | | | RSEDTAM | | | | | |
| | | | | | | | | | YFC | | | | | |
| H5S20- | EVKLVES | 217 | GFTFSSY | 303 | MSWVRQT | 389 | ISSG-GS | 477 | YYPDSVK | 586 | TRGGYG- | 725 | WGQGTTL | 856 |
| 4AH | GGGLMKP | | A | | PETRLEW | | T | | GRFTISR | | -SSYVI | | TVSS | |
| | GGSLKLS | | | | VAS | | | | DNVRNIL | | | | | |
| | CAAS | | | | | | | | YLQMSSL | | | | | |
| | | | | | | | | | RSEDTAM | | | | | |
| | | | | | | | | | YFC | | | | | |
| H5S20- | EVKLVES | 215 | GFTFSSY | 303 | MSWVRQT | 389 | ISSG-GS | 477 | YYPDSVK | 586 | TRGGYG- | 725 | WGQGTTL | 856 |
| 4BH | GGGLVKP | | A | | PETRLEW | | T | | GRFTISR | | -SSYVI | | TVSS | |
| | GGSLKLS | | | | VAS | | | | DNVRNIL | | | | | |
| | CAAS | | | | | | | | YLQMSSL | | | | | |
| | | | | | | | | | RSEDTAM | | | | | |
| | | | | | | | | | YFC | | | | | |
| H5S20- | EVKLVES | 215 | GFTFSSY | 303 | MSWVRQT | 389 | ISSG-GS | 477 | YYPDSVK | 586 | TRGGYG- | 727 | WGQGTTL | 856 |
| 61AH | GGGLVKP | | A | | PETRLEW | | T | | GRFTISR | | -SSHVI | | TVSS | |
| | GGSLKLS | | | | VAS | | | | DNVRNIL | | | | | |
| | CAAS | | | | | | | | YLQMSSL | | | | | |
| | | | | | | | | | RSEDTAM | | | | | |
| | | | | | | | | | YFC | | | | | |
| H5S20- | EVKLVES | 215 | GFTFSSF | 299 | MSWVRQT | 385 | ISSG-GN | 474 | YYPDNVK | 590 | ARGGYG- | 728 | WGQGTTL | 856 |
| 6AH | GGGLVKP | | A | | PEKRLEW | | T | | GRFTISR | | -SSYVI | | TVSS | |
| | GGSLKLS | | | | VAS | | | | DNAGNIL | | | | | |
| | CAAS | | | | | | | | YLQMSSL | | | | | |
| | | | | | | | | | RSEDTAM | | | | | |
| | | | | | | | | | YYC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #5

| | | | | | | | | | [N/S]YN | | | | | |
|---|---|---|---|---|---|---|---|---|---|---|---|---|---|---|
| | | | | | | | | | SALMSRL | | | | | |
| | | | | | | | | | [S/N/T] | | | | | |
| | [Q/G][V | | | | | | | | I[S/N/T | | | | | |
| | /A][Q/A | | | | | | | | /I][K/N | | | | | |
| | ][L/E][ | | | | | | | | /Q/I/T/ | | | | | |
| | K/G][E/ | | | | | | | | S]DN[S/ | | | | | |
| | V][S/R] | | | | | | | | F][K/R] | | | | | |
| | [G/R]P[ | | | | V[H/Y]W | | | | SQVFLKM | | | | | |
| | G/V]LVA | | | | [V/L]RQ | | | | NSLQ[S/ | | | | | |
| | PS[Q/R] | | GFSL[T/ | | P[P/A]G | | | | T][D/G] | | ARDWERD | | | |
| | [S/N]LS | | S][I/T/ | | KGLEWLG | | IWAGGI[ | | DTA[M/I | | SSGPF[A | | WGQGTLV | |
| | ITCTVS | 218 | S/N]YG | 304 | V | 390 | I/T] | 478 | ]YYC | 591 | /V/P]Y | 729 | TVSA | 852 |
| H5S19- | QVQLKES | 219 | GFSLTTY | 305 | VHWVRQP | 391 | IWAGGIT | 479 | NYNSALM | 592 | ARDWERD | 730 | WGQGTLV | 852 |
| 12BH | GPGLVAP | | G | | PGKGLEW | | | | SRLTISN | | SSGPFPY | | TVSA | |
| | SRSLSIT | | | | LGV | | | | DNSRSQV | | | | | |
| | CTVS | | | | | | | | FLKMNSL | | | | | |
| | | | | | | | | | QTDDTAM | | | | | |
| | | | | | | | | | YYC | | | | | |
| H5S19- | QVQLKES | 219 | GFSLTTY | 305 | VHWVRQP | 391 | IWAGGIT | 479 | NYNSALM | 593 | ARDWERD | 730 | WGQGTLV | 852 |
| 12CH | GPGLVAP | | G | | PGKGLEW | | | | SRLSISN | | SSGPFPY | | TVSA | |
| | SRSLSIT | | | | LGV | | | | DNSRSQV | | | | | |
| | CTVS | | | | | | | | FLKMNSL | | | | | |
| | | | | | | | | | QTDDTAM | | | | | |
| | | | | | | | | | YYC | | | | | |
| H5S19- | QVQLKES | 220 | GFSLTSY | 306 | VHWVRQP | 391 | IWAGGIT | 479 | NYNSALM | 593 | ARDWERD | 731 | WGQGTLV | 852 |
| 14AH | GPGLVAP | | G | | PGKGLEW | | | | SRLSISN | | SSGPFAY | | TVSA | |
| | SQSLSIT | | | | LGV | | | | DNSRSQV | | | | | |
| | CTVS | | | | | | | | FLKMNSL | | | | | |
| | | | | | | | | | QTDDTAM | | | | | |
| | | | | | | | | | YYC | | | | | |
| H5S19- | QVQLKES | 220 | GFSLTSY | 306 | VHWVRQP | 391 | IWAGGIT | 479 | NYNSALM | 594 | ARDWERD | 731 | WGQGTLV | 852 |
| 14BH | GPGLVAP | | G | | PGKGLEW | | | | SRLSINN | | SSGPFAY | | TVSA | |
| | SQSLSIT | | | | LGV | | | | DNSRSQV | | | | | |
| | CTVS | | | | | | | | FLKMNSL | | | | | |
| | | | | | | | | | QTDDTAM | | | | | |
| | | | | | | | | | YYC | | | | | |
| H5S19- | QVQLKES | 220 | GFSLTTY | 305 | VHWVRQP | 391 | IWAGGIT | 479 | NYNSALM | 595 | ARDWERD | 731 | WGQGTLV | 852 |
| 17AH | GPGLVAP | | G | | PGKGLEW | | | | SRLSISK | | SSGPFAY | | TVSA | |
| | SQSLSIT | | | | LGV | | | | DNSKSQV | | | | | |
| | CTVS | | | | | | | | FLKMNSL | | | | | |
| | | | | | | | | | QTGDTAM | | | | | |
| | | | | | | | | | YYC | | | | | |
| H5S19- | QVQLKES | 220 | GFSLTIY | 307 | VHWVRQP | 391 | IWAGGII | 480 | NYNSALM | 596 | ARDWERD | 731 | WGQGTLV | 852 |
| 18AH | GPGLVAP | | G | | PGKGLEW | | | | SRLSISK | | SSGPFAY | | TVSA | |
| | SQSLSIT | | | | LGV | | | | DNSKSQV | | | | | |
| | CTVS | | | | | | | | FLKMNSL | | | | | |
| | | | | | | | | | QSDDTAM | | | | | |
| | | | | | | | | | YYC | | | | | |
| H5S19- | QVQLKES | 220 | GFSLTSY | 306 | VHWVRQP | 391 | IWAGGIT | 479 | NYNSALM | 597 | ARDWERD | 732 | WGQGTLV | 852 |
| 20AH | GPGLVAP | | G | | PGKGLEW | | | | SRLSISK | | SSGPFVY | | TVSA | |
| | SQSLSIT | | | | LGV | | | | DNSKSQV | | | | | |
| | CTVS | | | | | | | | FLKMNSL | | | | | |
| | | | | | | | | | QTDDTAM | | | | | |
| | | | | | | | | | YYC | | | | | |
| H5S19- | QVQLKES | 220 | GFSLTSY | 306 | VHWVRQP | 392 | IWAGGIT | 479 | NYNSALM | 598 | ARDWERD | 732 | WGQGTLV | 852 |
| 20BH | GPGLVAP | | G | | AGKGLEW | | | | SRLSIIQ | | SSGPFVY | | TVSA | |
| | SQSLSIT | | | | LGV | | | | DNSKSQV | | | | | |
| | CTVS | | | | | | | | FLKMNSL | | | | | |
| | | | | | | | | | QTDDTAM | | | | | |
| | | | | | | | | | YYC | | | | | |
| H5S19- | QVQLKES | 221 | GFSLTSY | 306 | VHWVRQP | 391 | IWAGGIT | 479 | NYNSALM | 597 | ARDWERD | 732 | WGQGTLV | 852 |
| 20CH | GPVLVAP | | G | | PGKGLEW | | | | SRLSISK | | SSGPFVY | | TVSA | |
| | SQSLSIT | | | | LGV | | | | DNSKSQV | | | | | |
| | CTVS | | | | | | | | FLKMNSL | | | | | |
| | | | | | | | | | QTDDTAM | | | | | |
| | | | | | | | | | YYC | | | | | |
| H5S19- | QVQLKES | 220 | GFSLTSY | 306 | VHWVRQP | 391 | IWAGGIT | 479 | NYNSALM | 599 | ARDWERD | 730 | WGQGTLV | 852 |
| 21AH | GPGLVAP | | G | | PGKGLEW | | | | SRLSIST | | SSGPFPY | | TVSA | |
| | SQSLSIT | | | | LGV | | | | DNSRSQV | | | | | |
| | CTVS | | | | | | | | FLKMNSL | | | | | |
| | | | | | | | | | QTDDTAM | | | | | |
| | | | | | | | | | YYC | | | | | |
| H5S19- | QVQLKES | 220 | GFSLTNY | 308 | VHWLRQP | 393 | IWAGGIT | 479 | NYNSALM | 593 | ARDWERD | 731 | WGQGTLV | 852 |
| 22AH | GPGLVAP | | G | | PGKGLEW | | | | SRLSISN | | SSGPFAY | | TVSA | |
| | SQSLSIT | | | | LGV | | | | DNSRSQV | | | | | |
| | CTVS | | | | | | | | FLKMNSL | | | | | |
| | | | | | | | | | QTDDTAM | | | | | |
| | | | | | | | | | YYC | | | | | |
| H5S19- | QVQLKES | 220 | GFSLTTY | 305 | VYWVRQP | 394 | IWAGGIT | 479 | NYNSALM | 595 | ARDWERD | 731 | WGQGTLV | 852 |
| 26AH | GPGLVAP | | G | | PGKGLEW | | | | SRLSISK | | SSGPFAY | | TVSA | |
| | SQSLSIT | | | | LGV | | | | DNSKSQV | | | | | |
| | CTVS | | | | | | | | FLKMNSL | | | | | |
| | | | | | | | | | QTGDTAM | | | | | |
| | | | | | | | | | YYC | | | | | |
| H5S19- | QVQLKES | 220 | GFSLTSY | 306 | VHWVRQP | 391 | IWAGGIT | 479 | SYNSALM | 600 | ARDWERD | 730 | WGQGTLV | 852 |
| 27AH | GPGLVAP | | G | | PGKGLEW | | | | SRLSISS | | SSGPFPY | | TVSA | |
| | SQSLSIT | | | | LGV | | | | DNSRSQV | | | | | |
| | CTVS | | | | | | | | FLKMNSL | | | | | |
| | | | | | | | | | QTDDTAM | | | | | |
| | | | | | | | | | YYC | | | | | |
| ITI_ | QVQLKES | 222 | GFSLTTY | 305 | VHWVRQP | 391 | IWAGGIT | 479 | NYNSALM | 601 | ARDWERD | 730 | WGQGTLV | 852 |
| 14 | GPGLVAP | | G | | PGKGLEW | | | | SRLSISK | | SSGPFPY | | TVSA | |
| 4H | SQNLSIT | | | | LGV | | | | DNFKSQV | | | | | |
| | CTVS | | | | | | | | FLKMNSL | | | | | |
| | | | | | | | | | QTDDTAI | | | | | |
| | | | | | | | | | YYC | | | | | |
| ITI_ | QVQLKES | 222 | GFSLSTY | 309 | VHWVRQP | 391 | IWAGGIT | 479 | NYNSALM | 601 | ARDWERD | 730 | WGQGTLV | 852 |
| 14 | GPGLVAP | | G | | PGKGLEW | | | | SRLSISK | | SSGPFPY | | TVSA | |
| 5H | SQNLSIT | | | | LGV | | | | DNFKSQV | | | | | |
| | CTVS | | | | | | | | FLKMNSL | | | | | |
| | | | | | | | | | QTDDTAI | | | | | |
| | | | | | | | | | YYC | | | | | |
| ITI_ | QVQLKES | 220 | GFSLTTY | 305 | VHWVRQP | 391 | IWAGGIT | 479 | NYNSALM | 602 | ARDWERD | 731 | WGQGTLV | 852 |
| 14 | GPGLVAP | | G | | PGKGLEW | | | | SRLNISK | | SSGPFAY | | TVSA | |
| 6H | SQSLSIT | | | | LGV | | | | DNSKSQV | | | | | |
| | CTVS | | | | | | | | FLKMNSL | | | | | |
| | | | | | | | | | QSDDTAM | | | | | |
| | | | | | | | | | YYC | | | | | |
| ITI_ | QVQLKES | 220 | GFSLTSY | 306 | VHWVRQP | 391 | IWAGGIT | 479 | NYNSALM | 603 | ARDWERD | 730 | WGQGTLV | 852 |
| 16 | GPGLVAP | | G | | PGKGLEW | | | | SRLSINI | | SSGPFPY | | TVSA | |
| 2H | SQSLSIT | | | | LGV | | | | DNSKSQV | | | | | |
| | CTVS | | | | | | | | FLKMNSL | | | | | |
| | | | | | | | | | QTDDTAM | | | | | |
| | | | | | | | | | YYC | | | | | |
| ITI_ | GAAEGVR | 223 | GFSLTTY | 305 | VHWVRQP | 391 | IWAGGIT | 479 | NYNSALM | 604 | ARDWERD | 731 | WGQGTLV | 852 |
| 16 | RPGLVAP | | G | | PGKGLEW | | | | SRLNINK | | SSGPFAY | | TVSA | |
| 4H | SQSLSIT | | | | LGV | | | | DNSKSQV | | | | | |
| | CTVS | | | | | | | | FLKMNSL | | | | | |
| | | | | | | | | | QTGDTAM | | | | | |
| | | | | | | | | | YYC | | | | | |
| ITI_ | QVQLKES | 220 | GFSLTSY | 306 | VHWVRQP | 391 | IWAGGIT | 479 | NYNSALM | 605 | ARDWERD | 731 | WGQGTLV | 852 |
| 16 | GPGLVAP | | G | | PGKGLEW | | | | SRLSITK | | SSGPFAY | | TVSA | |
| 5H | SQSLSIT | | | | LGV | | | | DNSKSQV | | | | | |
| | CTVS | | | | | | | | FLKMNSL | | | | | |
| | | | | | | | | | QTDDTAM | | | | | |
| | | | | | | | | | YYC | | | | | |
| ITI_ | QVQLKES | 220 | GFSLTTY | 305 | VHWVRQP | 391 | IWAGGIT | 479 | NYNSALM | 604 | ARDWERD | 731 | WGQGTLV | 852 |
| 16 | GPGLVAP | | G | | PGKGLEW | | | | SRLNINK | | SSGPFAY | | TVSA | |
| 8H | SQSLSIT | | | | LGV | | | | DNSKSQV | | | | | |
| | CTVS | | | | | | | | FLKMNSL | | | | | |
| | | | | | | | | | QTGDTAM | | | | | |
| | | | | | | | | | YYC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #23

| | | | | | | | | | [K/E/G/ | | | | | |
|---|---|---|---|---|---|---|---|---|---|---|---|---|---|---|
| | | | | | | | | | Y/N/D/H | | | | | |
| | | | | | | | | | /S][Y/F | | | | | |
| | | | | | | | | | ][D/N/S | | | | | |
| | | | | | | | | | /P][P/E | | | | | |
| | | | | | | | | | /V/D/A/ | | | | | |
| | | | | | | | | | Q][K/S/ | | | | | |
| | | | | | | | | | T/A][F/ | | | | | |
| | | | | | | | | | V/L][Q/ | | | | | |
| | | | | | | | | | K/T/I][ | | | | | |
| | | | | | | | | | G/S/C/D | | | | | |
| | | | | | | | | | ][K/R][ | | | | | |
| | | | | | | | | | A/F/I/L | | | | | |
| | | | | | | | | | ][T/S/K | | | | | |
| | | | | | | | | | ][I/L/F | | | | | |
| | | | | | | | | | ][M/T/S | | | | | |
| | | | | | | | | | ][A/S/R | | | | | |
| | | | | | | | | | /K/V][D | | | | | |
| | [E/D/Q] | | | | | | | | /E][T/K | | | | | |
| | V[Q/K]L | | | | | | | | /N/S][S | | | | | |
| | [Q/V/K] | | | | [I/M/W/ | | | | /A][S/Q | | | | | |
| | [Q/E][S | | | | V][H/S/ | | | | /K][N/S | | A[Q/S/R | | | |
| | /P]G[A/ | | | | N/G/E]W | | | | ][T/I/Q | | /I/T/G] | | | |
| | P/G][E/ | | | | [V/I/M] | | [I/F][D | | ][A/L/F | | [G/S/R/ | | | |
| | G]L[V/M | | | | [K/R]Q[ | | /N/R/S/ | | /V][Y/F | | M/V/W/N | | | |
| | ][K/Q/N | | | | R/K/T/F | | Y/L/W/H | | ][L/M/F | | /Y/E/C] | | | |
| | /R]P[G/ | | | | /S/N][P | | ][P/N/Y | | /I][Q/E | | [G/-/P/ | | | |
| | S][A/G/ | | G[F/Y][ | | /H][E/G | | /S][A/Y | | /K][L/M | | L/Y][G/ | | | |
| | Q/T][S/ | | N/T/S][ | | ][Q/K/N | | /Q/S/G] | | ][S/N][ | | /L/S/N/ | | | |
| | P][V/L] | | I/F/L][ | | /M/H/R] | | [-/A][- | | S/T/R][ | | R/Y][-/ | | | |
| | [K/R/S] | | -/T][K/ | | [G/A/K/ | | /N][N/A | | L/V][T/ | | W/G][G/ | | | |
| | [L/M/I] | | T/S/N][ | | R/S]LEW | | /-/G/S/ | | R/Q][S/ | | s/-/N][ | | | |
| | [S/T]C[ | | D/S/I/N | | [I/L/M/ | | D][G/D/ | | V/A/T][ | | A/S/-/R | | | |
| | T/K/A][ | | /T][T/Y | | V][G/A] | | Y/S][N/ | | E/D]D[T | | /L/K/W/ | | | |
| | A/T/V][ | | ][Y/V/A | | [R/Y/F/ | | G/T/H/Y | | /S]A[V/ | | H][M/F/ | | WGQGT[S | |
| | S/T/A/F | | /T/W/G/ | | E/D/V/N | | /S/E/D] | | T/I/M]Y | | Y/P/I/L | | /T][V/L | |
| | ] | 224 | P] | 310 | ] | 395 | [I/T] | 481 | YC | 606 | /V/W]DY | 733 | ]TVSS | 857 |
| H5S14- | QVQLKQS | 225 | GFSL-TS | 311 | VHWVRQS | 396 | IWSG--- | 482 | DYNAAFI | 607 | ARNLGGS | 734 | WGQGTSV | 848 |
| 15AH | GPGLVQP | | YG | | PGKGLEW | | GST | | CRLSISK | | WVDY | | TVSS | |
| | SQSLSIT | | | | LGV | | | | DSSKSQV | | | | | |
| | CTVS | | | | | | | | FFKMNSL | | | | | |
| | | | | | | | | | QADDTAM | | | | | |
| | | | | | | | | | YYC | | | | | |
| H5S14- | QVQLQQS | 226 | GYTF-TN | 312 | IGWVKQR | 397 | IYPG--G | 483 | NYNEKFK | 608 | ARNGN-- | 735 | WGQGTTL | 856 |
| 17AH | GAELVRP | | YW | | PGHGLEW | | GYT | | GKATLTA | | SLDY | | TVSS | |
| | GTSVKMS | | | | IGD | | | | DTSSSTA | | | | | |
| | CKAA | | | | | | | | YMQLSSL | | | | | |
| | | | | | | | | | TSEDSAI | | | | | |
| | | | | | | | | | YYC | | | | | |
| H5S14- | QVQLQQS | 227 | GYTF-TT | 313 | IEWMKQN | 398 | FHPY--N | 484 | KYNEKFK | 609 | ARRLYGG | 736 | WGQGTSV | 848 |
| 25AH | GAELVKP | | YP | | HGKSLEW | | DDT | | GKAKLTV | | AMDY | | TVSS | |
| | GASVKMS | | | | IGN | | | | EKSSSTV | | | | | |
| | CKAF | | | | | | | | YLELSRL | | | | | |
| | | | | | | | | | TSDDSAV | | | | | |
| | | | | | | | | | YYC | | | | | |
| H5S14- | EVKLVES | 228 | GFTF-TD | 314 | MSWVRQT | 399 | IRNQANA | 485 | EYSVSVK | 610 | ARV---- | 737 | WGQGTTL | 856 |
| 6AH | GGGLVQP | | YY | | PGKALEW | | YTT | | GRFTISR | | -PDY | | TVSS | |
| | GGSLRLS | | | | LGF | | | | DNSQSIL | | | | | |
| | CATS | | | | | | | | YLQMNTL | | | | | |
| | | | | | | | | | RVEDSAT | | | | | |
| | | | | | | | | | YYC | | | | | |
| H5S15- | EVQLQQS | 229 | GFNI-KD | 315 | MHWVKQR | 400 | IDPA--N | 486 | KFDPKFQ | 611 | ASRGG-S | 738 | WGQGTTL | 856 |
| 12AH | GAELVKP | | TY | | PEQGLEW | | GNT | | GKATITA | | SFDY | | TVSS | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S15- | QVQLQQP | 230 | GYTF-TS | 316 | MNWVKQR | 401 | IDPS--D | 487 | HYNQKFK | 612 | AREYYGN | 739 | WGQGTTL | 856 |
| 30AH | GAELVKP | | YW | | PGRGLEW | | SET | | DKATLTV | | HFDY | | TVSS | |
| | GAPVKLS | | | | IGR | | | | DKSSSTA | | | | | |
| | CKAS | | | | | | | | YIQLSSL | | | | | |
| | | | | | | | | | TSEDSAV | | | | | |
| | | | | | | | | | YYC | | | | | |
| H5S15- | EVQLQQS | 231 | GYTF-TS | 317 | MHWVKQK | 402 | INPY--N | 488 | KYNEKFK | 613 | ARM---- | 740 | WGQGTTL | 856 |
| 38AH | GPELVKP | | YV | | PGQGLEW | | DGT | | GKATLTS | | -YDY | | TVSS | |
| | GASVKMS | | | | IGY | | | | DKSSSTA | | | | | |
| | CKAS | | | | | | | | YMELSSL | | | | | |
| | | | | | | | | | TSEDSAV | | | | | |
| | | | | | | | | | YYC | | | | | |
| H5S19- | DVQLQES | 232 | GYSITSD | 318 | WNWIRQF | 403 | ISYS--- | 489 | GYNPSLK | 614 | ARWGL-- | 741 | WGQGTTL | 856 |
| 11AH | GPGLVNP | | YA | | PGNKLEW | | GNT | | SRISITR | | RIDY | | TVSS | |
| | SQSLSLT | | | | MGY | | | | DTSKNQF | | | | | |
| | CTVT | | | | | | | | FLQLNSV | | | | | |
| | | | | | | | | | TSEDTAT | | | | | |
| | | | | | | | | | YYC | | | | | |
| H5S19- | QVQLQQS | 233 | GYTF-SS | 319 | IEWVKQR | 404 | ILPG--S | 490 | NYNEKFK | 615 | ARYPRWG | 742 | WGQGTTL | 856 |
| 25AH | GAELMKP | | YW | | PGHGLEW | | GST | | GKATFTA | | KIDY | | TVSS | |
| | GASVKIS | | | | IGE | | | | DTSSNTA | | | | | |
| | CKAT | | | | | | | | YMQLSSL | | | | | |
| | | | | | | | | | TSEDSAV | | | | | |
| | | | | | | | | | YYC | | | | | |
| H5S19- | EVQLVES | 234 | GFTF-NI | 320 | MSWVRQS | 405 | ISSG--G | 491 | YYPDTVT | 616 | ARGGS-- | 743 | WGQGTTL | 856 |
| 6AH | GGGLVKP | | YT | | PEMRLEW | | SHT | | GRFTISR | | LFDY | | TVSS | |
| | GGSLKLS | | | | VAE | | | | DNAKNTL | | | | | |
| | CAAS | | | | | | | | YLEMSSL | | | | | |
| | | | | | | | | | RSEDTAI | | | | | |
| | | | | | | | | | YYC | | | | | |
| H5S20- | EVQLQQS | 229 | GFNI-KD | 315 | MHWVKQR | 400 | IDPA--N | 486 | KYDPKFQ | 617 | ATSGG-S | 744 | WGQGTTL | 856 |
| 15AH | GAELVKP | | TY | | PEQGLEW | | GNT | | GKATITA | | SYDY | | TVSS | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S20- | EVQLQQS | 229 | GFNI-KD | 315 | MHWVKQR | 400 | IDPA--N | 486 | KYDPKFQ | 617 | ARSGG-S | 745 | WGQGTTL | 856 |
| 25AH | GAELVKP | | TY | | PEQGLEW | | GNT | | GKATITA | | SYDY | | TVSS | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S20- | EVQLQQS | 229 | GFNI-KD | 315 | MHWVKQR | 400 | IDPA--N | 486 | KYDPKFQ | 617 | ASSGG-S | 746 | WGQGTTL | 856 |
| 34AH | GAELVKP | | TY | | PEQGLEW | | GNT | | GKATITA | | SFDY | | TVSS | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S20- | EVQLQQS | 229 | GFNI-KD | 315 | MHWVKQR | 400 | IDPA--N | 486 | KYDPKFQ | 617 | AGSGG-S | 747 | WGQGTTL | 856 |
| 42AH | GAELVKP | | TY | | PEQGLEW | | GNT | | GKATITA | | SYDY | | TVSS | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S20- | EVQLQQS | 229 | GFNI-KD | 315 | MHWVKQR | 400 | IDPA--N | 486 | KYDPKFQ | 617 | AISGG-S | 748 | WGQGTTL | 856 |
| 8AH | GAELVKP | | TY | | PEQGLEW | | GNT | | GKATITA | | SYDY | | TVSS | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| ITI_ | DVQLQES | 235 | GYSITSD | 318 | WNWVRQF | 406 | ISYS--- | 492 | SYNPSLK | 618 | ARCYYGG | 749 | WGQGTTL | 856 |
| 02 | GPGLVKP | | YA | | PGNKLEW | | GST | | SRISITR | | RWDY | | TVSS | |
| 1H | SQSLSLT | | | | VGY | | | | DTSKNQF | | | | | |
| | CTVT | | | | | | | | FLQLNSV | | | | | |
| | | | | | | | | | TTEDTAT | | | | | |
| | | | | | | | | | YYC | | | | | |
| ITI_ | DVQLQES | 232 | GYSITSD | 318 | WNWIRQF | 403 | INYS--- | 493 | GYNPSLK | 614 | ARWGL-- | 741 | WGQGTTL | 856 |
| 17 | GPGLVNP | | YA | | PGNKLEW | | GNT | | SRISITR | | RIDY | | TVSS | |
| 3H | SQSLSLT | | | | MGY | | | | DTSKNQF | | | | | |
| | CTVT | | | | | | | | FLQLNSV | | | | | |
| | | | | | | | | | TSEDTAT | | | | | |
| | | | | | | | | | YYC | | | | | |
| ITI_ | EVQLQQS | 229 | GFNI-KD | 315 | IHWVKQR | 407 | IDPA--N | 494 | KYDPKFQ | 619 | AQGGG-G | 750 | WGQGTSV | 848 |
| 20 | GAELVKP | | TY | | PEQGLEW | | GNI | | GKATIMA | | AMDY | | TVSS | |
| 0H | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #21

| | | | | | | | | | [H/N/K] | | | | | |
|---|---|---|---|---|---|---|---|---|---|---|---|---|---|---|
| | | | | | | | | | [Y/F][N | | | | | |
| | | | | | | | | | /D/A][Q | | | | | |
| | | | | | | | | | /E/P]KF | | | | | |
| | | | | | | | | | [K/R/Q] | | | | | |
| | | | | | | | | | [D/G/V] | | [A/V][I | | | |
| | | | | | | | | | [K/R]AT | | /R/P/A/ | | | |
| | | | | | | | | | [L/I]T[ | | G/T/N][ | | | |
| | | | | | | | | | V/A/T]D | | Y/R/P][ | | | |
| | | | | | | | | | [K/T/S] | | Y/G/F][ | | | |
| | [Q/E]VQ | | | | | | | | SS[S/N] | | S/H/G/N | | | |
| | LQQ[P/S | | | | [M/I/V] | | | | T[A/V]Y | | /A][N/Y | | | |
| | ]GA[E/D | | | | [N/E/H] | | | | [I/M/L] | | /S/R][- | | | |
| | ]L[V/L] | | G[Y/F][ | | W[V/L][ | | I[D/N/A | | [Q/H]LS | | /Y/T/S] | | | |
| | [K/R]PG | | T/A/N/Y | | K/N][Q/ | | ]P[S/G/ | | [S/R]LT | | [-/G/P/ | | | |
| | [A/T][P | | ][F/I][ | | E]RP[G/ | | A][D/S/ | | S[E/D]D | | Y/N][P/ | | | |
| | /S]V[K/ | | S/T/K/R | | E][R/Q] | | N][S/G/ | | [S/T/N/ | | Y/A/D][ | | WGQGT[T | |
| | R/M][L/ | | ][T/N/D | | GL[E/A] | | D][E/G/ | | A]AV[Y/ | | V/W/G]F | | /L][L/V | |
| | V]SC[K/ | | ][Y/T][ | | WIG[R/V | | N/K/Y/R | | F][Y/F] | | [D/A/V] | | ]TVS[S/ | |
| | T]AS | 236 | W/L/Y] | 321 | ] | 408 | ]T | 495 | C | 620 | [Y/F] | 751 | A] | 858 |
| H5S15- | EVQLQQS | 237 | GFNIKDT | 322 | MHWVKQR | 400 | IAPANGR | 496 | KYDPKFQ | 617 | ANYYASS | 752 | WGQGTLV | 852 |
| 11AH | GADLVKP | | Y | | PEQGLEW | | T | | GKATITA | | YDWFAY | | TVSA | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S15- | EVQLQQS | 229 | GFNIKDT | 322 | MHWVKQR | 400 | IAPANGK | 497 | KFDPKFQ | 611 | ANYYGRS | 753 | WGQGTLV | 852 |
| 17AH | GAELVKP | | Y | | PEQGLEW | | T | | GKATITA | | NDWFVY | | TVSA | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S15- | EVQLQQS | 229 | GFNIKDT | 322 | MHWVKQR | 409 | IAPANGY | 498 | KYDPKFQ | 621 | ANYFGNT | 754 | WGQGTLV | 852 |
| 1AH | GAELVKP | | Y | | PEQGLAW | | T | | GKATITT | | YDWFAF | | TVSA | |
| | GASVKLS | | | | IGR | | | | DTSSNTA | | | | | |
| | CTAS | | | | | | | | YLHLSRL | | | | | |
| | | | | | | | | | TSEDTAV | | | | | |
| | | | | | | | | | YYC | | | | | |
| H5S15- | EVQLQQS | 229 | GFNIKDT | 322 | MHWVKQR | 400 | IAPANGR | 496 | KYDPKFQ | 617 | ANYYASS | 755 | WGQGTLV | 852 |
| 24AH | GAELVKP | | Y | | PEQGLEW | | T | | GKATITA | | YDWFVY | | TVSA | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S15- | EVQLQQS | 229 | GFNIKDT | 322 | MHWVKQR | 400 | IDPANGN | 499 | KYDPKFQ | 617 | ARPYGN- | 756 | WGQGTLV | 852 |
| 36AH | GAELVKP | | Y | | PEQGLEW | | T | | GKATITA | | -YGFAY | | TVSA | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S15- | QVQLQQP | 230 | GYTFSTY | 323 | MNWVKQR | 401 | IDPSDSE | 500 | HYNQKFK | 612 | AIYYSN- | 757 | WGQGTTL | 856 |
| 37AH | GAELVKP | | W | | PGRGLEW | | T | | DKATLTV | | -PVFDY | | TVSS | |
| | GAPVKLS | | | | IGR | | | | DKSSSTA | | | | | |
| | CKAS | | | | | | | | YIQLSSL | | | | | |
| | | | | | | | | | TSEDSAV | | | | | |
| | | | | | | | | | YYC | | | | | |
| H5S15- | EVQLQQS | 229 | GFNIKDT | 322 | MHWVKQR | 409 | IAPANGY | 498 | KYDPKFQ | 622 | VTYFGNT | 758 | WGQGTLV | 852 |
| 3AH | GAELVKP | | Y | | PEQGLAW | | T | | GKATITT | | YDWFAY | | TVSA | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S20- | EVQLQQS | 229 | GFNIKDT | 322 | MHWVNQR | 410 | IDPANGN | 499 | KYAPKFQ | 623 | AGYGNS- | 759 | WGQGTLV | 852 |
| 10AH | GAELVKP | | Y | | PEQGLEW | | T | | GKATITA | | -PWFAY | | TVSA | |
| | GASVKLS | | | | IGR | | | | DTSSNTV | | | | | |
| | CTAS | | | | | | | | YLQLSSL | | | | | |
| | | | | | | | | | TSEDTAV | | | | | |
| | | | | | | | | | YYC | | | | | |
| H5S20- | EVQLQQS | 229 | GFNIKDT | 322 | MHWVNQR | 410 | IDPANGN | 499 | KYAPKFQ | 624 | AGYGNS- | 759 | WGQGTLV | 852 |
| 10BH | GAELVKP | | Y | | PEQGLEW | | T | | GKATITA | | -PWFAY | | TVSA | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S20- | EVQLQQS | 229 | GFNIKDT | 322 | MHWVNQR | 410 | IDPANGK | 501 | KYAPKFQ | 625 | AGYGNS- | 759 | WGQGTLV | 852 |
| 17AH | GAELVKP | | Y | | PEQGLEW | | T | | DKATITA | | -PWFAY | | TVSA | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S20- | EVQLQQS | 229 | GFNIKDT | 322 | VHWVKER | 411 | IDPANDN | 502 | KYAPKFQ | 626 | APYGNY- | 760 | WGQGTLV | 852 |
| 49AH | GAELVKP | | Y | | PEQGLEW | | T | | VKATITA | | PAWFAY | | TVSA | |
| | GASVKLS | | | | IGR | | | | DTSSNTA | | | | | |
| | CTAS | | | | | | | | YLQLSSL | | | | | |
| | | | | | | | | | TSEDNAV | | | | | |
| | | | | | | | | | YYC | | | | | |
| H5S20- | EVQLQQS | 229 | GFNIKDT | 322 | IHWLNQR | 412 | IDPANGK | 501 | KFAPKFQ | 627 | AGYGNS- | 759 | WGQGTLV | 852 |
| 52AH | GAELVKP | | Y | | PEQGLEW | | T | | DKATITA | | -PWFAY | | TVSA | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S20- | EVQLQQS | 238 | GFYIKDT | 324 | MHWVNQR | 410 | IDPANGK | 501 | KYAPKFQ | 628 | AGYGNS- | 759 | WGQGTLV | 852 |
| 58AH | GAELVKP | | Y | | PEQGLEW | | T | | DKATITA | | -PWFAY | | TVSA | |
| | GASVMLS | | | | IGR | | | | DSSSNTA | | | | | |
| | CTAS | | | | | | | | YLQLSSL | | | | | |
| | | | | | | | | | TSEDTAV | | | | | |
| | | | | | | | | | YYQ | | | | | |
| H5S20- | EVQLQQS | 237 | GFNIRDT | 325 | MHWVNQR | 410 | IDPANGN | 499 | KYAPKFQ | 629 | AGYGNS- | 759 | WGQGTLV | 852 |
| 9AH | GADLVKP | | Y | | PEQGLEW | | T | | GRATITA | | -PWFAY | | TVSA | |
| | GASVKLS | | | | IGR | | | | DTSSNTA | | | | | |
| | CTAS | | | | | | | | YLHLSSL | | | | | |
| | | | | | | | | | TSEDTAV | | | | | |
| | | | | | | | | | YYC | | | | | |
| ITI_ | EVQLQQS | 239 | GFNFKDT | 326 | MHWVNQR | 410 | IDPANGK | 501 | KYAPKFQ | 630 | AAYGNS- | 761 | WGQGTLV | 852 |
| 09 | GAELLKP | | Y | | PEQGLEW | | T | | GKATITA | | -PWFAY | | TVSA | |
| 1H | GASVRLS | | | | IGR | | | | DTSSNTA | | | | | |
| | CTAS | | | | | | | | YLQLSSL | | | | | |
| | | | | | | | | | TSEDAAV | | | | | |
| | | | | | | | | | FYC | | | | | |
| ITI_ | QVQLQQS | 240 | GYAFTNY | 327 | IEWVKQR | 413 | INPGSGG | 503 | NYNEKFR | 631 | ARRGHNY | 762 | WGQGTLV | 852 |
| 12 | GAELVRP | | L | | PGQGLEW | | T | | GKATLTA | | GPWFAY | | TVSA | |
| 2H | GTSVKVS | | | | IGV | | | | DKSSSTA | | | | | |
| | CKAS | | | | | | | | YMQLSSL | | | | | |
| | | | | | | | | | TSDDSAV | | | | | |
| | | | | | | | | | YFC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #10

| | | | | | | | | | [K/N/Y/ | | | | | |
|---|---|---|---|---|---|---|---|---|---|---|---|---|---|---|
| | | | | | | | | | R/D]Y[N | | | | | |
| | | | | | | | | | /P][E/Q | | | | | |
| | | | | | | | | | /D/P/A] | | | | | |
| | | | | | | | | | [K/S/T/ | | | | | |
| | | | | | | | | | A/P][F/ | | | | | |
| | | | | | | | | | V/L][K/ | | | | | |
| | | | | | | | | | R/I][G/ | | | | | |
| | | | | | | | | | D/S][K/ | | | | | |
| | | | | | | | | | R][A/F/ | | | | | |
| | | | | | | | | | L][T/S] | | | | | |
| | | | | | | | | | [L/I][T | | | | | |
| | | | | | | | | | /S][S/V | | | | | |
| | | | | | | | | | /A/R/K] | | | | | |
| | | | | | | | | | D[K/T/N | | | | | |
| | | | | | | | | | ][S/A][ | | [A/S/T] | | | |
| | [E/Q]V[ | | | | | | | | S/K/R][ | | R[G/R/L | | | |
| | Q/T]L[Q | | | | | | | | S/N][T/ | | /D/S]I/ | | | |
| | /V/K][Q | | | | | | | | N/Q][A/ | | E/H/V/L | | | |
| | /E][S/P | | | | | | | | L/V][Y/ | | /G/M/Y] | | | |
| | ]G[P/A/ | | | | [M/I/L/ | | | | F][M/L/ | | [I/R/Y/ | | | |
| | G][E/G] | | | | V][H/N/ | | | | F][E/R/ | | D/N/G][ | | | |
| | [L/I][V | | | | S/Y/E]W | | | | Q/K][L/ | | T/R/G/- | | | |
| | /L][K/R | | | | [V/I][K | | | | M/I][S/ | | /Y/S/N] | | | |
| | /Q]P[G/ | | | | /R]Q[K/ | | I[N/D/Y | | T/N][S/ | | [T/G/N/ | | | |
| | S][A/T/ | | G[Y/F][ | | R/T/P/S | | /S/W][P | | T][L/P/ | | -/S/K][ | | | |
| | G/Q][S/ | | T/A/S][ | | ][P/S][ | | /S/D/W] | | V][T/K/ | | V/-/Y/P | | | |
| | T][V/L] | | F/L][-/ | | G/E][Q/ | | [Y/S/G/ | | D/Q][S/ | | ][V/I/- | | | |
| | [K/M/S] | | S][-/T] | | H/K][G/ | | D][N/D/ | | T/A][E/ | | ][E/-][ | | | |
| | [M/L/I/ | | [T/S][S | | R]LEW[I | | G/-/S][ | | A/N/D/S | | P/-][I/ | | | |
| | V][S/T] | | /N/D/G/ | | /V/L][G | | D/S/G][ | | ]D[S/T] | | -/D/Y][ | | | |
| | C[K/A/S | | T][Y/M] | | /A][Y/V | | G/F/Y/S | | A[V/M/T | | L/I/P/- | | WGQG[T/ | |
| | /T][A/F | | [V/W/D/ | | /N/D/T/ | | /D/N][T | | /I][Y/C | | /N/Y/G] | | S]S[V/F | |
| | /V]S | 241 | Y/G/L] | 328 | H] | 414 | /K] | 504 | ][Y/F]C | 632 | YAMDY | 763 | ]TVSS | 859 |
| H5S14- | QVTLKES | 242 | GFSLSTS | 329 | VSWIRQP | 415 | IYWD-DD | 505 | RYNPSLK | 633 | ARSMYGN | 764 | WGQGTSV | 848 |
| 10AH | GPGILQP | | GMG | | SGKGLEW | | K | | SRLTISK | | -YNYAMD | | TVSS | |
| | SQTLSLT | | | | LAH | | | | DTSRNQV | | Y | | | |
| | CSFS | | | | | | | | FLKITSV | | | | | |
| | | | | | | | | | DTADTAT | | | | | |
| | | | | | | | | | YYC | | | | | |
| H5S14- | EVQLVES | 234 | GFTF--S | 330 | MYWVRQT | 416 | ISDGGSY | 506 | YYPDSVK | 634 | ARDGNY- | 765 | WGQGTSV | 848 |
| 16AH | GGGLVKP | | DYY | | PEKRLEW | | T | | GRFTISR | | ------Y | | TVSS | |
| | GGSLKLS | | | | VAT | | | | DNAKNNL | | AMDY | | | |
| | CAAS | | | | | | | | YLQMSSL | | | | | |
| | | | | | | | | | KSEDTAM | | | | | |
| | | | | | | | | | YYC | | | | | |
| H5S14- | QVQLQQP | 243 | GYTF--T | 331 | INWVKQR | 417 | IYPSDSY | 507 | NYNQKFK | 635 | TRGHYGN | 766 | WGQGTSV | 848 |
| 20AH | GAELVRP | | NYW | | PGQGLEW | | T | | DKATLTV | | YDPYAMD | | TVSS | |
| | GASVKLS | | | | IGN | | | | DKSSSTA | | Y | | | |
| | CKAS | | | | | | | | YMQLSSP | | | | | |
| | | | | | | | | | TSEDSAV | | | | | |
| | | | | | | | | | YYC | | | | | |
| H5S14- | EVQLQQS | 231 | GYTF--T | 332 | MHWVKQK | 402 | INPYNDG | 508 | KYNEKFK | 613 | ARGIITT | 767 | WGQGTSV | 848 |
| 22AH | GPELVKP | | SYV | | PGQGLEW | | T | | GKATLTS | | VIEPILY | | TVSS | |
| | GASVKMS | | | | IGY | | | | DKSSSTA | | AMDY | | | |
| | CKAS | | | | | | | | YMELSSL | | | | | |
| | | | | | | | | | TSEDSAV | | | | | |
| | | | | | | | | | YYC | | | | | |
| H5S14- | QVQLQQS | 240 | GYAF--T | 333 | IEWVKQR | 413 | INPGSGG | 503 | KYNEKFK | 636 | ARDYGSS | 768 | WGQGTSV | 848 |
| 26AH | GAELVRP | | NYL | | PGQGLEW | | T | | GKATLTA | | --YGYAM | | TVSS | |
| | GTSVKVS | | | | IGV | | | | DKSSSTA | | DY | | | |
| | CKAS | | | | | | | | YMQLSSL | | | | | |
| | | | | | | | | | TSDDSAV | | | | | |
| | | | | | | | | | YFC | | | | | |
| H5S14- | QVQLQQS | 203 | GYTF--T | 331 | LSWVKQR | 418 | IYPGGGY | 468 | NYNEKFK | 637 | ARRVD-- | 769 | WGQGTSV | 848 |
| 28AH | GAELVRP | | NYW | | PGHGLEW | | T | | GKATLTA | | ----YAM | | TVSS | |
| | GTSVKIS | | | | IGD | | | | DTSSSTA | | DY | | | |
| | CKAS | | | | | | | | YMQLSSL | | | | | |
| | | | | | | | | | TSEDSAV | | | | | |
| | | | | | | | | | CFC | | | | | |
| H5S14- | EVQLQQS | 231 | GYTF--T | 332 | MHWVKQK | 402 | INPYNDG | 508 | KYNEKFK | 613 | ARGIITT | 770 | WGQGTSV | 848 |
| 29AH | GPELVKP | | SYV | | PGQGLEW | | T | | GKATLTS | | VVEPILY | | TVSS | |
| | GASVKMS | | | | IGY | | | | DKSSSTA | | AMDY | | | |
| | CKAS | | | | | | | | YMELSSL | | | | | |
| | | | | | | | | | TSEDSAV | | | | | |
| | | | | | | | | | YYC | | | | | |
| H5S15- | QVQLKQS | 225 | GFSL--T | 334 | VHWVRQS | 396 | IWSG-GN | 509 | DYNPAFI | 638 | ARRGYNK | 771 | WGQGTSF | 860 |
| 33AH | GPGLVQP | | TYG | | PGKGLEW | | T | | SRLSISK | | -----GY | | TVSS | |
| | SQSLSIT | | | | LGV | | | | DNSKSQV | | AMDY | | | |
| | CTVS | | | | | | | | FFKMNTL | | | | | |
| | | | | | | | | | QASDTAI | | | | | |
| | | | | | | | | | YYC | | | | | |
| H5S15- | EVQLVES | 234 | GFAF--S | 335 | MSWVRQT | 419 | ISSGGGS | 510 | YYPDTVK | 639 | ARLLRY- | 772 | WGQGTSV | 848 |
| 40AH | GGGLVKP | | SYD | | PEKRLEW | | T | | GRFTISR | | ---YAMD | | TVSS | |
| | GGSLKLS | | | | VAY | | | | DNAKNTL | | Y | | | |
| | CAAS | | | | | | | | YLQMSSL | | | | | |
| | | | | | | | | | KSEDTAM | | | | | |
| | | | | | | | | | YYC | | | | | |
| H5S19- | QVQLKQS | 225 | GFSL--T | 334 | VHWVRQS | 396 | IWSG-GS | 511 | DYNAAFI | 640 | ARRGYGS | 773 | WGQGTSV | 848 |
| 9AH | GPGLVQP | | TYG | | PGKGLEW | | T | | SRLSISK | | P---YYY | | TVSS | |
| | SQSLSIT | | | | LGV | | | | DNSKSQV | | AMDY | | | |
| | CTVS | | | | | | | | FFKMNSL | | | | | |
| | | | | | | | | | QANDTAI | | | | | |
| | | | | | | | | | YYC | | | | | |
| H5S20- | QVQLQQP | 244 | GYTF--T | 331 | MHWVRQR | 420 | IDPSDSF | 512 | NYNQSFR | 641 | SRGERRG | 774 | WGQGSSV | 861 |
| 21AH | GAELVKP | | NYW | | PGQGLEW | | T | | GKATLTV | | -----IY | | TVSS | |
| | GASVMMS | | | | IGV | | | | DTSSSTA | | AMDY | | | |
| | CKAS | | | | | | | | YMRLSSL | | | | | |
| | | | | | | | | | TSEDSAV | | | | | |
| | | | | | | | | | YFC | | | | | |
| ITI_ | QVQLKQS | 225 | GFSL--T | 334 | VHWVRQS | 396 | IWSG-GS | 511 | DYNAAFI | 640 | ARRGYGK | 775 | WGQGTSV | 848 |
| 13 | GPGLVQP | | TYG | | PGKGLEW | | T | | SRLSISK | | -----GY | | TVSS | |
| 0H | SQSLSIT | | | | LGV | | | | DNSKSQV | | AMDY | | | |
| | CTVS | | | | | | | | FFKMNSL | | | | | |
| | | | | | | | | | QANDTAI | | | | | |
| | | | | | | | | | YYC | | | | | |
| ITI_ | QVQLKQS | 225 | GFSL--T | 334 | VHWVRQS | 396 | IWSG-GS | 511 | DYNAPFI | 642 | ARRGYNK | 771 | WGQGTSV | 848 |
| 13 | GPGLVQP | | TYG | | PGKGLEW | | T | | SRLSISK | | -----GY | | TVSS | |
| 1H | SQSLSIT | | | | LGV | | | | DNSKSQV | | AMDY | | | |
| | CTVS | | | | | | | | FFKMNSL | | | | | |
| | | | | | | | | | QANDTAI | | | | | |
| | | | | | | | | | YYC | | | | | |
| ITI_ | QVQLKQS | 225 | GFSL--T | 334 | IHWVRQS | 421 | IWSG-GS | 511 | DYNAAFI | 643 | ARRGYGS | 773 | WGQGTSV | 848 |
| 18 | GPGLVQP | | TYG | | PGKGLEW | | T | | SRLSITK | | P---YYY | | TVSS | |
| 0H | SQSLSIT | | | | LGV | | | | DKSKSQV | | AMDY | | | |
| | CTVS | | | | | | | | FFKMNSL | | | | | |
| | | | | | | | | | QANDTAI | | | | | |
| | | | | | | | | | YYC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #8

| | | | | | | | | | TY[D/A] | | | | | |
|---|---|---|---|---|---|---|---|---|---|---|---|---|---|---|
| | | | | | | | | | [D/E]DF | | | | | |
| | | | | | | | | | KGRFAFS | | ARS[F/Y | | | |
| | | | | | | | | | LETSAST | | ][T/Y][ | | | |
| | QIQLVQS | | | | [M/V]NW | | | | AYL[Q/R | | T/A/G/K | | WGQ[G/S | |
| | GPE[L/V | | | | VKQAPGK | | | | ]INNLKN | | ][A/N/S | | ]T[T/L/ | |
| | ]KKPGET | | GYT[F/L | | [G/D]LK | | | | ED[T/M/ | | ][T/N/Y | | I][L/V] | |
| | VKISCKA | | ]TN[Y/F | | W[M/V]G | | INTYTGE | | S]A[T/S | | /E][C/A | | TVS[S/A | |
| | S | 245 | ]G | 336 | W | 422 | P | 513 | ]YFC | 644 | ][Y/F] | 776 | ] | 862 |
| H5S15- | QIQLVQS | 246 | GYTLTNY | 337 | MNWVKQA | 423 | INTYTGE | 513 | TYADDFK | 645 | ARSFYGS | 777 | WGQGTLV | 852 |
| 15AH | GPEVKKP | | G | | PGKGLKW | | P | | GRFAFSL | | EAY | | TVSA | |
| | GETVKIS | | | | VGW | | | | ETSASTA | | | | | |
| | CKAS | | | | | | | | YLQINNL | | | | | |
| | | | | | | | | | KNEDMAT | | | | | |
| | | | | | | | | | YFC | | | | | |
| H5S15- | QIQLVQS | 247 | GYTFTNY | 338 | MNWVKQA | 423 | INTYTGE | 513 | TYAEDFK | 646 | ARSFYGS | 777 | WGQGTLV | 852 |
| 7AH | GPELKKP | | G | | PGKGLKW | | P | | GRFAFSL | | EAY | | TVSA | |
| | GETVKIS | | | | VGW | | | | ETSASTA | | | | | |
| | CKAS | | | | | | | | YLQINNL | | | | | |
| | | | | | | | | | KNEDMAT | | | | | |
| | | | | | | | | | YFC | | | | | |
| H5S15- | QIQLVQS | 247 | GYTFTNY | 338 | MNWVKQA | 423 | INTYTGE | 513 | TYADDFK | 645 | ARSFYGS | 777 | WGQGTLV | 852 |
| 7BH | GPELKKP | | G | | PGKGLKW | | P | | GRFAFSL | | EAY | | TVSA | |
| | GETVKIS | | | | VGW | | | | ETSASTA | | | | | |
| | CKAS | | | | | | | | YLQINNL | | | | | |
| | | | | | | | | | KNEDMAT | | | | | |
| | | | | | | | | | YFC | | | | | |
| H5S19- | QIQLVQS | 247 | GYTFTNF | 339 | VNWVKQA | 424 | INTYTGE | 513 | TYADDFK | 647 | ARSYYAN | 778 | WGQGTTL | 856 |
| 13AH | GPELKKP | | G | | PGKGLKW | | P | | GRFAFSL | | YAY | | TVSS | |
| | GETVKIS | | | | MGW | | | | ETSASTA | | | | | |
| | CKAS | | | | | | | | YLRINNL | | | | | |
| | | | | | | | | | KNEDTAT | | | | | |
| | | | | | | | | | YFC | | | | | |
| H5S19- | QIQLVQS | 247 | GYTFTNF | 339 | VNWVKQA | 424 | INTYTGE | 513 | TYADDFK | 648 | ARSFYKN | 779 | WGQGTIL | 863 |
| 3AH | GPELKKP | | G | | PGKGLKW | | P | | GRFAFSL | | YAF | | TVSS | |
| | GETVKIS | | | | MGW | | | | ETSASTA | | | | | |
| | CKAS | | | | | | | | YLRINNL | | | | | |
| | | | | | | | | | KNEDSAT | | | | | |
| | | | | | | | | | YFC | | | | | |
| H5S19- | QIQLVQS | 247 | GYTFTNF | 339 | VNWVKQA | 424 | INTYTGE | 513 | TYADDFK | 647 | ARSFYKN | 779 | WGQGTIL | 863 |
| 3BH | GPELKKP | | G | | PGKGLKW | | P | | GRFAFSL | | YAF | | TVSS | |
| | GETVKIS | | | | MGW | | | | ETSASTA | | | | | |
| | CKAS | | | | | | | | YLRINNL | | | | | |
| | | | | | | | | | KNEDTAT | | | | | |
| | | | | | | | | | YFC | | | | | |
| H5S19- | QIQLVQS | 247 | GYTFTNF | 339 | VNWVKQA | 425 | INTYTGE | 513 | TYADDFK | 647 | ARSFYKN | 779 | WGQGTIL | 863 |
| 3CH | GPELKKP | | G | | PGKDLKW | | P | | GRFAFSL | | YAF | | TVSS | |
| | GETVKIS | | | | MGW | | | | ETSASTA | | | | | |
| | CKAS | | | | | | | | YLRINNL | | | | | |
| | | | | | | | | | KNEDTAT | | | | | |
| | | | | | | | | | YFC | | | | | |
| H5S19- | QIQLVQS | 247 | GYTFTNF | 339 | VNWVKQA | 424 | INTYTGE | 513 | TYADDFK | 649 | ARSYYAN | 780 | WGQGTTL | 856 |
| 8AH | GPELKKP | | G | | PGKGLKW | | P | | GRFAFSL | | NAY | | TVSS | |
| | GETVKIS | | | | MGW | | | | ETSASTA | | | | | |
| | CKAS | | | | | | | | YLRINNL | | | | | |
| | | | | | | | | | KNEDTAS | | | | | |
| | | | | | | | | | YFC | | | | | |
| ITI_ | QIQLVQS | 247 | GYTFTNF | 339 | VNWVKQA | 424 | INTYTGE | 513 | TYADDFK | 647 | ARSFYKN | 779 | WGQSTIL | 864 |
| 03 | GPELKKP | | G | | PGKGLKW | | P | | GRFAFSL | | YAF | | TVSS | |
| 2H | GETVKIS | | | | MGW | | | | ETSASTA | | | | | |
| | CKAS | | | | | | | | YLRINNL | | | | | |
| | | | | | | | | | KNEDTAT | | | | | |
| | | | | | | | | | YFC | | | | | |
| ITI_ | QIQLVQS | 247 | GYTFTNF | 339 | VNWVKQA | 424 | INTYTGE | 513 | TYADDFK | 647 | ARSYYGN | 781 | WGQGTTL | 856 |
| 03 | GPELKKP | | G | | PGKGLKW | | P | | GRFAFSL | | YAY | | TVSS | |
| 5H | GETVKIS | | | | MGW | | | | ETSASTA | | | | | |
| | CKAS | | | | | | | | YLRINNL | | | | | |
| | | | | | | | | | KNEDTAT | | | | | |
| | | | | | | | | | YFC | | | | | |
| ITI_ | QIQLVQS | 247 | GYTFTNY | 338 | MNWVKQA | 426 | INTYTGE | 513 | TYADDFK | 650 | ARSFTTA | 782 | WGQGTTL | 856 |
| 08 | GPELKKP | | G | | PGKGLKW | | P | | GRFAFSL | | TCY | | TVSS | |
| 2H | GETVKIS | | | | MGW | | | | ETSASTA | | | | | |
| | CKAS | | | | | | | | YLQINNL | | | | | |
| | | | | | | | | | KNEDTAT | | | | | |
| | | | | | | | | | YFC | | | | | |
| ITI_ | QIQLVQS | 247 | GYTFTNY | 338 | MNWVKQA | 426 | INTYTGE | 513 | TYDDDFK | 651 | ARSFTTA | 782 | WGQGTTL | 856 |
| 08 | GPELKKP | | G | | PGKGLKW | | P | | GRFAFSL | | TCY | | TVSS | |
| 3H | GETVKIS | | | | MGW | | | | ETSASTA | | | | | |
| | CKAS | | | | | | | | YLQINNL | | | | | |
| | | | | | | | | | KNEDTAT | | | | | |
| | | | | | | | | | YFC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #15

| | | | | | | | | | [Y/S/N/ | | | | | |
|---|---|---|---|---|---|---|---|---|---|---|---|---|---|---|
| | | | | | | | | | D][Y/C] | | | | | |
| | | | | | | | | | [P/N/A] | | | | | |
| | | | | | | | | | [D/Q/E/ | | | | | |
| | | | | | | | | | A][S/K/ | | | | | |
| | | | | | | | | | T/A][V/ | | | | | |
| | | | | | | | | | F][K/M/ | | | | | |
| | | | | | | | | | T][G/S/ | | | | | |
| | | | | | | | | | D][R/K] | | | | | |
| | | | | | | | | | [F/A/L] | | | | | |
| | | | | | | | | | [T/S][I | | | | | |
| | | | | | | | | | /F/L][S | | | | | |
| | | | | | | | | | /T][R/V | | | | | |
| | [E/-/Q] | | | | | | | | /S/K]D[ | | | | | |
| | [V/-][Q | | | | | | | | N/T/K/D | | | | | |
| | /-/K][L | | | | | | | | ][A/S][ | | [A/V][R | | | |
| | /-][V/- | | | | | | | | K/S/R/Q | | /K][Q/T | | | |
| | /Q/K][E | | | | | | | | ][N/S][ | | /H/A/G/ | | | |
| | /-/Q][S | | | | | | | | T/I/Q/M | | N/E][G/ | | | |
| | /-/T][G | | | | [M/V][S | | | | ][L/A/V | | Y/R][G/ | | | |
| | /-][G/- | | | | /H/N]WV | | | | ][Y/F][ | | Y/L][H/ | | | |
| | /P][D/- | | | | [R/K]Q[ | | | | L/M/F][ | | Y/R][G/ | | | |
| | /G/E]LV | | | | T/S/K/A | | I[S/N/W | | Q/E/D/K | | R][-/S/ | | | |
| | [K/Q][P | | | | ][P/H][ | | /R][S/C | | ][M/F/L | | N/G/E][ | | | |
| | /T][G/S | | | | D/G/E][ | | /N/P/R] | | ][S/N][ | | -/S/T/N | | | |
| | /K][G/A | | G[F/Y][ | | K/Q][R/ | | [G/Y/K] | | S/R/N]L | | ][N/S/Y | | | |
| | /Q]S[L/ | | T/S/K][ | | S/G][L/ | | [-/S][- | | [K/T/R/ | | /F/G/D] | | | |
| | V][K/S] | | F/L][S/ | | P]EW[V/ | | /N][G/N | | Q][S/A/ | | [Y/N/V/ | | | |
| | [L/I/M] | | T/N][S/ | | I/L][A/ | | /-][S/G | | T][E/D] | | L][G/Y/ | | | |
| | [S/T]C[ | | G/T][Y/ | | G][T/Y/ | | /D/Y][Y | | D[T/S]A | | P][A/S/ | | WGQGT[S | |
| | A/K/T][ | | N][G/Y/ | | D/S/V/E | | /A/S/D] | | [M/V/I] | | Y][M/L/ | | /T][V/L | |
| | A/V]S | 248 | T/V/A] | 340 | /R] | 427 | T | 514 | YYC | 652 | F]DY | 783 | ]TVSS | 857 |
| H5S14- | EVQLVET | 249 | GFTFNTN | 341 | MNWVRQA | 428 | IRSKSNN | 515 | YYADSVK | 653 | VREGGYG | 784 | WGQGTTL | 856 |
| 18AH | GGGLVQP | | A | | PGKGLEW | | YAT | | DRFTISR | | --NYPYF | | TVSS | |
| | KGSLKLS | | | | VAR | | | | DDSQSML | | DY | | | |
| | CAAS | | | | | | | | YLQMNNL | | | | | |
| | | | | | | | | | KTEDTAM | | | | | |
| | | | | | | | | | YYC | | | | | |
| H5S14- | ------- | 250 | GYSFTGY | 342 | MHWVKQS | 429 | ISCY--N | 516 | SYNQKFK | 654 | ARTYYYG | 785 | WGQGTSV | 848 |
| 23AH | --- | | Y | | HGKSLEW | | GAT | | GKATFTV | | S-SYGAM | | TVSS | |
| | LVKTGAS | | | | IGY | | | | DTSSSTA | | DY | | | |
| | VKISCKA | | | | | | | | YMQFNSL | | | | | |
| | S | | | | | | | | TSEDSAV | | | | | |
| | | | | | | | | | YYC | | | | | |
| H5S14- | EVQLVES | 251 | GFTFSSY | 343 | MSWVRQT | 430 | ISSG--G | 517 | YYPDSVK | 655 | ARQGGHG | 786 | WGQGTSV | 848 |
| 30AH | GGDLVKP | | G | | PDKRLEW | | SYT | | GRFTISR | | NYGAMDY | | TVSS | |
| | GGSLKLS | | | | VAT | | | | DNAKNTL | | | | | |
| | CAAS | | | | | | | | YLQMSSL | | | | | |
| | | | | | | | | | KSEDTAM | | | | | |
| | | | | | | | | | YYC | | | | | |
| H5S15- | EVQLQQS | 231 | GYTFTSY | 344 | MHWVKQK | 402 | INPY--N | 518 | NCNEKFK | 656 | AKARGYG | 787 | WGQGTSV | 848 |
| 14AH | GPELVKP | | V | | PGQGLEW | | DDT | | GKATLTS | | STFYYSM | | TVSS | |
| | GASVKMS | | | | IGY | | | | DKSSSTA | | DY | | | |
| | CKAS | | | | | | | | YMELSRL | | | | | |
| | | | | | | | | | TSEDSAV | | | | | |
| | | | | | | | | | YYC | | | | | |
| H5S15- | EVQLQQS | 231 | GYKFNSY | 345 | MHWVKQK | 431 | INPY--N | 518 | NCNEKFK | 657 | AKARGYG | 788 | WGQGTSV | 848 |
| 25AH | GPELVKP | | V | | PGQGPEW | | DDT | | GKATLTS | | GNFYYSM | | TVSS | |
| | GASVKMS | | | | IGY | | | | DKSSSTA | | DY | | | |
| | CKAS | | | | | | | | YMELSSL | | | | | |
| | | | | | | | | | TSEDSAV | | | | | |
| | | | | | | | | | YYC | | | | | |
| H5S15- | EVQLQQS | 231 | GYTFTSY | 344 | MHWVKQK | 402 | INPY--N | 518 | NCNEKFK | 658 | AKARGYG | 789 | WGQGTSV | 848 |
| 5AH | GPELVKP | | V | | PGQGLEW | | DDT | | GKATLTS | | GSFYYSM | | TVSS | |
| | GASVKMS | | | | IGY | | | | DKSSSTA | | DY | | | |
| | CKAS | | | | | | | | YMDLSSL | | | | | |
| | | | | | | | | | TSEDSAV | | | | | |
| | | | | | | | | | YYC | | | | | |
| H5S20- | EVKLVES | 252 | GFTFSSY | 346 | MSWVRQT | 419 | ISNG--G | 519 | YYPDTVK | 639 | ARHRGYG | 790 | WGQGTSV | 848 |
| 44AH | GGGLVQP | | T | | PEKRLEW | | GST | | GRFTISR | | SSYNYAM | | TVSS | |
| | GGSLKLS | | | | VAY | | | | DNAKNTL | | DY | | | |
| | CAAS | | | | | | | | YLQMSSL | | | | | |
| | | | | | | | | | KSEDTAM | | | | | |
| | | | | | | | | | YYC | | | | | |
| H5S20- | EVKLVES | 252 | GFTFSSY | 346 | MSWVRQT | 419 | ISNG--G | 519 | YYPDTVK | 639 | ARHRGYG | 791 | WGQGTSV | 848 |
| 46AH | GGGLVQP | | T | | PEKRLEW | | GST | | GRFTISR | | N-YVYAM | | TVSS | |
| | GGSLKLS | | | | VAY | | | | DNAKNTL | | DY | | | |
| | CAAS | | | | | | | | YLQMSSL | | | | | |
| | | | | | | | | | KSEDTAM | | | | | |
| | | | | | | | | | YYC | | | | | |
| H5S20- | QVQLKQS | 225 | GFSLTSY | 306 | VHWVRQS | 396 | IWRG--- | 520 | DYNAAFM | 659 | AKNRGYG | 792 | WGQGTSV | 848 |
| 51AH | GPGLVQP | | G | | PGKGLEW | | GST | | SRLSITK | | E-GYYAM | | TVSS | |
| | SQSLSIT | | | | LGV | | | | DNSKSQV | | DY | | | |
| | CTVS | | | | | | | | FFKMNSL | | | | | |
| | | | | | | | | | QADDTAI | | | | | |
| | | | | | | | | | YYC | | | | | |
| H5S20- | EVQLVES | 234 | GFTFSSY | 303 | MSWVRQS | 432 | ISSG--G | 517 | YYPDTVT | 660 | AREGLRR | 793 | WGQGTSV | 848 |
| 53AH | GGGLVKP | | A | | PEKRLEW | | SYT | | GRFTISR | | --DYYAL | | TVSS | |
| | GGSLKLS | | | | VAE | | | | DNAKNTL | | DY | | | |
| | CAAS | | | | | | | | YLEMSSL | | | | | |
| | | | | | | | | | RSEDTAM | | | | | |
| | | | | | | | | | YYC | | | | | |
| H5S20- | EVKLVES | 252 | GFTFSSY | 346 | MSWVRQT | 433 | ISNG--G | 519 | YYPDTVK | 639 | ARHRGYG | 791 | WGQGTSV | 848 |
| 55AH | GGGLVQP | | T | | PEKRLEW | | GST | | GRFTISR | | N-YVYAM | | TVSS | |
| | GGSLKLS | | | | VAD | | | | DNAKNTL | | DY | | | |
| | CAAS | | | | | | | | YLQMSSL | | | | | |
| | | | | | | | | | KSEDTAM | | | | | |
| | | | | | | | | | YYC | | | | | |
| H5S20- | EVKLVES | 215 | GFTFSSY | 303 | MSWVRQT | 385 | ISSG--- | 521 | YYPDSVK | 661 | AKGRGYG | 794 | WGQGTSV | 848 |
| 59AH | GGGLVKP | | A | | PEKRLEW | | SST | | GRFTISR | | N-YLYAM | | TVSS | |
| | GGSLKLS | | | | VAS | | | | DNARNIL | | DY | | | |
| | CAAS | | | | | | | | YLQMSSL | | | | | |
| | | | | | | | | | RSEDTAM | | | | | |
| | | | | | | | | | YYC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #19

| | | | | | | | | | [D/T]Y[ | | | | | |
|---|---|---|---|---|---|---|---|---|---|---|---|---|---|---|
| | | | | | | | | | N/A][S/ | | | | | |
| | | | | | | | | | D/G][A/ | | | | | |
| | | | | | | | | | D][L/F] | | | | | |
| | | | | | | | | | K[S/G]R | | | | | |
| | | | | | | | | | [L/F][S | | | | | |
| | | | | | | | | | /A][I/F | | | | | |
| | | | | | | | | | ]S[K/L] | | | | | |
| | | | | | | | | | [D/E][N | | | | | |
| | Q[V/I]Q | | | | | | | | /T]S[K/ | | | | | |
| | L[K/V][ | | | | | | | | A]S[Q/T | | | | | |
| | E/Q]SGP | | | | | | | | ][V/I/A | | | | | |
| | [G/E]L[ | | | | | | | | ][F/Y]L | | [A/V]R[ | | | |
| | V/K][A/ | | | | [V/M]NW | | | | [K/Q][M | | D/S][N/ | | | |
| | K]P[S/G | | | | [V/M][R | | | | /I]N[S/ | | S/Y/L/F | | | |
| | ][Q/E][ | | | | /K]Q[P/ | | | | N][L/V] | | ]Y[H/R/ | | | |
| | S/T][L/ | | G[F/Y][ | | S/A]PGK | | I[W/N][ | | [Q/K][T | | -]IT/-] | | | |
| | V][S/K] | | S/T/I][ | | [G/D]L[ | | G/T][D/ | | /N][D/E | | [V/M/-] | | | |
| | I[T/S]C | | L/F]T[G | | E/K]W[L | | Y][-/T] | | ]D[T/M] | | [V/T/G] | | WGQGT[S | |
| | [T/K][V | | /N][Y/F | | /M]G[M/ | | G[G/S/E | | A[R/T]Y | | N[G/S/R | | /T][V/L | |
| | /A]S | 253 | ]G | 347 | W] | 434 | ][T/P] | 522 | [Y/F]C | 662 | ][D/G]Y | 795 | ]TVSS | 857 |
| H5S15- | QVQLKES | 220 | GFSLTGY | 348 | VNWVRQP | 435 | IWGD-GG | 523 | DYNSALK | 663 | ARDNYHT | 796 | WGQGTSV | 848 |
| 19AH | GPGLVAP | | G | | PGKGLEW | | T | | SRLSISK | | VVNGDY | | TVSS | |
| | SQSLSIT | | | | LGM | | | | DNSKSQV | | | | | |
| | CTVS | | | | | | | | FLKMNSL | | | | | |
| | | | | | | | | | QTDDTAR | | | | | |
| | | | | | | | | | YYC | | | | | |
| H5S15- | QVQLKES | 220 | GFSLTGY | 348 | VNWVRQS | 436 | IWGD-GS | 524 | DYNSALK | 664 | ARDSYRT | 797 | WGQGTSV | 848 |
| 32AH | GPGLVAP | | G | | PGKGLEW | | T | | SRLSISK | | MTNGDY | | TVSS | |
| | SQSLSIT | | | | LGM | | | | DNSKSQI | | | | | |
| | CTVS | | | | | | | | FLKMNSV | | | | | |
| | | | | | | | | | QTEDTAR | | | | | |
| | | | | | | | | | YYC | | | | | |
| H5S19- | QIQLVQS | 247 | GYTFTNF | 339 | VNWVKQA | 424 | INTYTGE | 513 | TYADDFK | 650 | VRSYY-- | 798 | WGQGTTL | 856 |
| 16AH | GPELKKP | | G | | PGKGLKW | | P | | GRFAFSL | | -GNSGY | | TVSS | |
| | GETVKIS | | | | MGW | | | | ETSASTA | | | | | |
| | CKAS | | | | | | | | YLQINNL | | | | | |
| | | | | | | | | | KNEDTAT | | | | | |
| | | | | | | | | | YFC | | | | | |
| H5S19- | QIQLVQS | 247 | GYTFTNY | 338 | MNWVKQA | 426 | INTYTGE | 513 | TYADDFK | 650 | VRSYY-- | 798 | WGQGTTL | 856 |
| 19AH | GPELKKP | | G | | PGKGLKW | | P | | GRFAFSL | | -GNSGY | | TVSS | |
| | GETVKIS | | | | MGW | | | | ETSASTA | | | | | |
| | CKAS | | | | | | | | YLQINNL | | | | | |
| | | | | | | | | | KNEDTAT | | | | | |
| | | | | | | | | | YFC | | | | | |
| H5S20- | QIQLVQS | 247 | GYTFTNF | 339 | MNWVRQA | 437 | INTYTGE | 513 | TYADDFK | 665 | ARSLY-- | 799 | WGQGTTL | 856 |
| 12AH | GPELKKP | | G | | PGKGLKW | | P | | GRFAFSL | | -GNRDY | | TVSS | |
| | GETVKIS | | | | MGW | | | | ETSASTA | | | | | |
| | CKAS | | | | | | | | YLQINNL | | | | | |
| | | | | | | | | | KNDDMAT | | | | | |
| | | | | | | | | | YFC | | | | | |
| H5S20- | QIQLVQS | 247 | GYIFTNY | 349 | MNWVRQA | 437 | INTYTGE | 513 | TYADDFK | 645 | ARSFY-- | 800 | WGQGTTL | 856 |
| 18AH | GPELKKP | | G | | PGKGLKW | | P | | GRFAFSL | | -GNRDY | | TVSS | |
| | GETVKIS | | | | MGW | | | | ETSASTA | | | | | |
| | CKAS | | | | | | | | YLQINNL | | | | | |
| | | | | | | | | | KNEDMAT | | | | | |
| | | | | | | | | | YFC | | | | | |
| ITI_ | QIQLVQS | 247 | GYTLTNF | 350 | MNWMKQA | 438 | INTYTGE | 513 | TYAGDFK | 666 | VRSYY-- | 798 | WGQGTTL | 856 |
| 10 | GPELKKP | | G | | PGKGLKW | | P | | GRFAFSL | | -GNSGY | | TVSS | |
| 0H | GETVKIS | | | | MGW | | | | ETSASTA | | | | | |
| | CKAS | | | | | | | | YLQINNL | | | | | |
| | | | | | | | | | KNEDTAT | | | | | |
| | | | | | | | | | YFC | | | | | |
| ITI_ | QIQLVQS | 247 | GYTLTNF | 350 | MNWMKQA | 439 | INTYTGE | 513 | TYAGDFK | 666 | VRSYY-- | 798 | WGQGTTL | 856 |
| 10 | GPELKKP | | G | | PGKDLKW | | P | | GRFAFSL | | -GNSGY | | TVSS | |
| 1H | GETVKIS | | | | MGW | | | | ETSASTA | | | | | |
| | CKAS | | | | | | | | YLQINNL | | | | | |
| | | | | | | | | | KNEDTAT | | | | | |
| | | | | | | | | | YFC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #14

| | | | | | | | | | [T/K/D/ | | | | | |
|---|---|---|---|---|---|---|---|---|---|---|---|---|---|---|
| | | | | | | | | | S]Y[A/N | | | | | |
| | | | | | | | | | ][D/E/A | | | | | |
| | | | | | | | | | /Q][D/K | | | | | |
| | | | | | | | | | /A]F[K/ | | | | | |
| | | | | | | | | | M][G/S] | | | | | |
| | | | | | | | | | [R/K][L | | | | | |
| | | | | | | | | | /A/F][A | | | | | |
| | | | | | | | | | /K/S/T] | | | | | |
| | | | | | | | | | [F/L/I] | | | | | |
| | | | | | | | | | [S/T][L | | | | | |
| | | | | | | | | | /V/K/S] | | | | | |
| | | | | | | | | | [E/D][T | | | | | |
| | [Q/E][I | | | | | | | | /K/N/E] | | | | | |
| | /V]QL[V | | | | | | | | S[A/S/K | | | | | |
| | /Q/K]Q[ | | | | | | | | ]S[T/Q] | | | | | |
| | S/P]G[P | | | | [M/I/V] | | | | [A/V][F | | | | | |
| | /A][E/G | | | | [H/E/N] | | | | /Y][L/F | | | | | |
| | ]L[K/V] | | | | W[V/M][ | | | | /M][Q/E | | A[N/R/K | | | |
| | [K/Q/M] | | | | K/R]Q[A | | [I/F][N | | /K][I/L | | ][W/G/T | | | |
| | P[G/S][ | | | | /N/S/K/ | | /H/W/D] | | /M][N/S | | /E/S][A | | | |
| | E/A/Q][ | | | | R][P/H] | | [T/P/R] | | ][N/R/S | | /G/R/L] | | | |
| | T/S][V/ | | G[Y/F][ | | G[K/Q][ | | [E/Y/G/ | | ]L[K/T/ | | [-/T/L/ | | | |
| | L][K/S] | | T/S][F/ | | G/S]L[K | | S][T/N/ | | Q][N/S/ | | R][-/R/ | | | |
| | [I/M][S | | L]T[D/T | | /E]W[M/ | | -/D][G/ | | A][E/D] | | A][-/S] | | | |
| | /T]C[K/ | | /S/N]Y[ | | I/L]G[W | | D/S][E/ | | D[T/S]A | | [-/G/A] | | | |
| | T][A/V] | | S/P/G/V | | /N/V/Y/ | | D/S/G/Y | | [T/V/I] | | [G/-/P/ | | WGQGTLV | |
| | [S/F] | 254 | /W] | 351 | A] | 440 | ][P/T | 525 | Y[F/Y]C | 667 | W]FAY | 801 | TVSA | 852 |
| H5S15- | QVQLKQS | 225 | GFSLTSY | 306 | VHWVRQS | 396 | IWRG-GS | 526 | DYNAAFM | 659 | AKTG--- | 802 | WGQGTLV | 852 |
| 13AH | GPGLVQP | | G | | PGKGLEW | | T | | SRLSITK | | FAY | | TVSA | |
| | SQSLSIT | | | | LGV | | | | DNSKSQV | | | | | |
| | CTVS | | | | | | | | FFKMNSL | | | | | |
| | | | | | | | | | QADDTAI | | | | | |
| | | | | | | | | | YYC | | | | | |
| H5S15- | QVQLQQS | 227 | GYTFTTY | 352 | IEWMKQN | 398 | FHPYNDD | 527 | KYNEKFK | 609 | ARGG--- | 803 | WGQGTLV | 852 |
| 28AH | GAELVKP | | P | | HGKSLEW | | T | | GKAKLTV | | FAY | | TVSA | |
| | GASVKMS | | | | IGN | | | | EKSSSTV | | | | | |
| | CKAF | | | | | | | | YLELSRL | | | | | |
| | | | | | | | | | TSDDSAV | | | | | |
| | | | | | | | | | YYC | | | | | |
| H5S15- | QIQLVQS | 247 | GYTFTDY | 353 | MHWVKQA | 441 | INTETGE | 528 | TYADDFK | 668 | ANWA--- | 804 | WGQGTLV | 852 |
| 39AH | GPELKKP | | S | | PGKGLKW | | P | | GRLAFSL | | -GFAY | | TVSA | |
| | GETVKIS | | | | MGW | | | | ETSASTA | | | | | |
| | CKAS | | | | | | | | FLQINNL | | | | | |
| | | | | | | | | | KNEDTAT | | | | | |
| | | | | | | | | | YFC | | | | | |
| ITI_ | EVQLQQS | 231 | GYTFTSY | 344 | MHWVKQK | 402 | INPYNDG | 508 | KYNEKFK | 613 | ARERT-- | 805 | WGQGTLV | 852 |
| 23 | GPELVKP | | V | | PGQGLEW | | T | | GKATLTS | | GPFAY | | TVSA | |
| 6H | GASVKMS | | | | IGY | | | | DKSSSTA | | | | | |
| | CKAS | | | | | | | | YMELSSL | | | | | |
| | | | | | | | | | TSEDSAV | | | | | |
| | | | | | | | | | YYC | | | | | |
| ITI_ | QVQLQQP | 255 | GYTFTDY | 354 | MHWVKQR | 442 | IDTSDSY | 529 | SYNQKFK | 669 | ARSARA- | 806 | WGQGTLV | 852 |
| 23 | GAELVMP | | W | | PGQGLEW | | T | | GKATLTV | | AWFAY | | TVSA | |
| 8H | GASVKMS | | | | IGA | | | | DESSSTA | | | | | |
| | CKAS | | | | | | | | YMQLSSL | | | | | |
| | | | | | | | | | TSEDSAV | | | | | |
| | | | | | | | | | YYC | | | | | |
| ITI_ | QIQLVQS | 247 | GYTFTNY | 338 | MNWVKQA | 426 | INTYTGE | 513 | TYADDFK | 670 | ARELLRS | 807 | WGQGTLV | 852 |
| 24 | GPELKKP | | G | | PGKGLKW | | P | | GRFAFSL | | AWFAY | | TVSA | |
| 0H | GETVKIS | | | | MGW | | | | ETSASTA | | | | | |
| | CKAS | | | | | | | | YLQINNL | | | | | |
| | | | | | | | | | KNDDTAT | | | | | |
| | | | | | | | | | YFC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #6

| | | | | | | | | | E[F/Y]A | | | | | |
|---|---|---|---|---|---|---|---|---|---|---|---|---|---|---|
| | | | | | | | | | PKFQGKA | | | | | |
| | | | | | | | | | T[L/M]T | | | | | |
| | EVQLQQS | | | | | | | | ADT[S/C | | | | | |
| | GAELVRS | | | | | | | | ]SN[T/A | | | | | |
| | GASVKLS | | | | MHWVKQR | | | | ]AYLQLS | | | | | |
| | C[A/T][ | | GFNIKDY | | PEQGLEW | | IDPENGD | | SLTSEDT | | N[T/S]R | | WGQGTLV | |
| | A/T]S | 256 | Y | 355 | IGW | 443 | T | 530 | AVYYC | 671 | TLGY | 808 | TVSA | 852 |
| H5S20- | EVQLQQS | 257 | GFNIKDY | 355 | MHWVKQR | 443 | IDPENGD | 530 | EYAPKFQ | 672 | NSRTLGY | 809 | WGQGTLV | 852 |
| 20AH | GAELVRS | | Y | | PEQGLEW | | T | | GKATMTA | | | | TVSA | |
| | GASVKLS | | | | IGW | | | | DTSSNAA | | | | | |
| | CTAS | | | | | | | | YLQLSSL | | | | | |
| | | | | | | | | | TSEDTAV | | | | | |
| | | | | | | | | | YYC | | | | | |
| H5S20- | EVQLQQS | 257 | GFNIKDY | 355 | MHWVKQR | 443 | IDPENGD | 530 | EYAPKFQ | 673 | NSRTLGY | 809 | WGQGTLV | 852 |
| 20BH | GAELVRS | | Y | | PEQGLEW | | T | | GKATMTA | | | | TVSA | |
| | GASVKLS | | | | IGW | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S20- | EVQLQQS | 258 | GFNIKDY | 355 | MHWVKQR | 443 | IDPENGD | 530 | EYAPKFQ | 673 | NSRTLGY | 809 | WGQGTLV | 852 |
| 31AH | GAELVRS | | Y | | PEQGLEW | | T | | GKATMTA | | | | TVSA | |
| | GASVKLS | | | | IGW | | | | DTSSNTA | | | | | |
| | CTTS | | | | | | | | YLQLSSL | | | | | |
| | | | | | | | | | TSEDTAV | | | | | |
| | | | | | | | | | YYC | | | | | |
| H5S20- | EVQLQQS | 258 | GFNIKDY | 355 | MHWVKQR | 443 | IDPENGD | 530 | EYAPKFQ | 674 | NSRTLGY | 809 | WGQGTLV | 852 |
| 31BH | GAELVRS | | Y | | PEQGLEW | | T | | GKATMTA | | | | TVSA | |
| | GASVKLS | | | | IGW | | | | DTCSNTA | | | | | |
| | CTTS | | | | | | | | YLQLSSL | | | | | |
| | | | | | | | | | TSEDTAV | | | | | |
| | | | | | | | | | YYC | | | | | |
| H5S20- | EVQLQQS | 259 | GFNIKDY | 355 | MHWVKQR | 443 | IDPENGD | 530 | EFAPKFQ | 675 | NTRTLGY | 810 | WGQGTLV | 852 |
| 45AH | GAELVRS | | Y | | PEQGLEW | | T | | GKATLTA | | | | TVSA | |
| | GASVKLS | | | | IGW | | | | DTSSNTA | | | | | |
| | CAAS | | | | | | | | YLQLSSL | | | | | |
| | | | | | | | | | TSEDTAV | | | | | |
| | | | | | | | | | YYC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #12

| | | | | | | | | | [F/D/N] | | | | | |
|---|---|---|---|---|---|---|---|---|---|---|---|---|---|---|
| | | | | | | | | | Y[P/N/Y | | | | | |
| | | | | | | | | | ][D/S/E | | | | | |
| | | | | | | | | | ][S/A/K | | | | | |
| | | | | | | | | | ][V/L/F | | | | | |
| | | | | | | | | | ]K[G/S/ | | | | | |
| | | | | | | | | | V][R/K] | | | | | |
| | | | | | | | | | [F/L/A] | | | | | |
| | | | | | | | | | [T/S][I | | | | | |
| | | | | | | | | | /L][S/T | | | | | |
| | | | | | | | | | ][R/K/A | | | | | |
| | | | | | | | | | ]D[N/T] | | | | | |
| | [E/Q]V[ | | | | | | | | [A/S][K | | | | | |
| | K/Q]L[V | | | | | | | | /S][N/S | | | | | |
| | /K/Q][E | | | | | | | | ][N/Q/T | | | | | |
| | /Q]SG[G | | | | | | | | ][L/V/A | | | | | |
| | /P/A][G | | | | | | | | ][Y/F][ | | | | | |
| | /E]LV[K | | | | [M/V/I] | | | | L/M][Q/ | | | | | |
| | /A/R]P[ | | | | [S/N/G] | | | | K][V/M/ | | | | | |
| | G/S][G/ | | | | WV[R/K] | | | | L][S/N] | | | | | |
| | Q/T]S[L | | | | Q[T/P/R | | | | [S | | | | | |
| | /V][K/S | | G[F/Y][ | | ]P[E/G] | | I[S/W/Y | | /R]L[R/ | | | | | |
| | ][L/I/M | | T/S][F/ | | [K/H][R | | ][G/P][ | | Q/T][S/ | | [I/A][Y | | | |
| | ][S/T]C | | L][S/T] | | /G]LEW[ | | G/D][G/ | | T][E/D] | | /R/S][D | | WGQGT[L | |
| | [A/T/K] | | [S/G/D/ | | V/L/I][ | | -][S/G] | | D[T/S]A | | /S][-/Y | | /T][V/L | |
| | [A/V][S | | N][Y]S] | | A/G][T/ | | [Y/N][T | | [L/R/I] | | ]G[S/A] | | ]TVS[A/ | |
| | /A] | 260 | [G/W] | 356 | M/D] | 444 | /S] | 531 | YYC | 676 | Y | 811 | S] | 865 |
| H5S15- | QVQLQQS | 226 | GYTFTNS | 289 | IGWVKQR | 397 | IYPGGGY | 468 | NYYEKFK | 677 | ASS-GAY | 812 | WGQGTTL | 856 |
| 18AH | GAELVRP | | W | | PGHGLEW | | T | | VKATLTA | | | | TVSS | |
| | GTSVKMS | | | | IGD | | | | DTSSSTA | | | | | |
| | CKAA | | | | | | | | YMQLSSL | | | | | |
| | | | | | | | | | TSEDSAI | | | | | |
| | | | | | | | | | YYC | | | | | |
| H5S15- | QVQLQQS | 226 | GYTFTNS | 289 | MGWVKQR | 445 | IYPGGGY | 532 | NYNEKFK | 608 | ASS-GAY | 812 | WGQGTTL | 856 |
| 26AH | GAELVRP | | W | | PGHGLEW | | S | | GKATLTA | | | | TVSS | |
| | GTSVKMS | | | | IAD | | | | DTSSSTA | | | | | |
| | CKAA | | | | | | | | YMQLSSL | | | | | |
| | | | | | | | | | TSEDSAI | | | | | |
| | | | | | | | | | YYC | | | | | |
| H5S15- | QVQLQQS | 226 | GYTFTDS | 357 | IGWVKQR | 397 | IYPGGGY | 468 | NYNEKFK | 678 | ASS-GAY | 812 | WGQGTTL | 856 |
| 8AH | GAELVRP | | W | | PGHGLEW | | T | | GKATLTA | | | | TVSS | |
| | GTSVKMS | | | | IGD | | | | DTSSSTA | | | | | |
| | CKAA | | | | | | | | YMQLSRL | | | | | |
| | | | | | | | | | TSEDSAI | | | | | |
| | | | | | | | | | YYC | | | | | |
| H5S19- | QVQLKES | 220 | GFSLTGY | 348 | VNWVRQP | 435 | IWGD-GN | 533 | DYNSALK | 663 | ARSYGSY | 813 | WGQGTLV | 852 |
| 4AH | GPGLVAP | | G | | PGKGLEW | | T | | SRLSISK | | | | TVSA | |
| | SQSLSIT | | | | LGM | | | | DNSKSQV | | | | | |
| | CTVS | | | | | | | | FLKMNSL | | | | | |
| | | | | | | | | | QTDDTAR | | | | | |
| | | | | | | | | | YYC | | | | | |
| H5S20- | EVKLVES | 215 | GFTFSSY | 343 | MSWVRQT | 446 | ISGGGSY | 534 | FYPDSVK | 679 | IYD-GSY | 814 | WGQGTLV | 852 |
| 56AH | GGGLVKP | | G | | PEKRLEW | | T | | GRFTISR | | | | TVSA | |
| | GGSLKLS | | | | VAT | | | | DNAKNNL | | | | | |
| | CAAS | | | | | | | | YLQVSSL | | | | | |
| | | | | | | | | | RSEDTAL | | | | | |
| | | | | | | | | | YYC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #4

| | | | | | | | | | INNQKFK | | | | | |
|---|---|---|---|---|---|---|---|---|---|---|---|---|---|---|
| | EVQLQQS | | | | | | | | [G/D]KA | | | | | |
| | GPELVKP | | | | | | | | TLTVDMS | | | | | |
| | G[A/T]S | | | | MHWVKQS | | | | SSTAYME | | | | | |
| | VKISCKT | | GYT[F/I | | HGKSLEW | | INP[Y/N | | LRSLTSE | | AGSVVDR | | WGAGTTV | |
| | S | 261 | ]TEYT | 358 | IGG | 447 | ]NGGT | 535 | DSAVYYC | 680 | YWYFDV | 815 | TVSS | 866 |
| H5S14- | EVQLQQS | 262 | GYTITEY | 359 | MHWVKQS | 447 | INPNNGG | 536 | INNQKFK | 681 | AGSVVDR | 815 | WGAGTTV | 866 |
| 24AH | GPELVKP | | T | | HGKSLEW | | T | | DKATLTV | | YWYFDV | | TVSS | |
| | GTSVKIS | | | | IGG | | | | DMSSSTA | | | | | |
| | CKTS | | | | | | | | YMELRSL | | | | | |
| | | | | | | | | | TSEDSAV | | | | | |
| | | | | | | | | | YYC | | | | | |
| H5S14- | EVQLQQS | 263 | GYTFTEY | 360 | MHWVKQS | 447 | INPYNGG | 537 | INNQKFK | 682 | AGSVVDR | 815 | WGAGTTV | 866 |
| 4AH | GPELVKP | | T | | HGKSLEW | | T | | GKATLTV | | YWYFDV | | TVSS | |
| | GASVKIS | | | | IGG | | | | DMSSSTA | | | | | |
| | CKTS | | | | | | | | YMELRSL | | | | | |
| | | | | | | | | | TSEDSAV | | | | | |
| | | | | | | | | | YYC | | | | | |
| H5S14- | EVQLQQS | 263 | GYTFTEY | 360 | MHWVKQS | 447 | INPNNGG | 536 | INNQKFK | 682 | AGSVVDR | 815 | WGAGTTV | 866 |
| 7AH | GPELVKP | | T | | HGKSLEW | | T | | GKATLTV | | YWYFDV | | TVSS | |
| | GASVKIS | | | | IGG | | | | DMSSSTA | | | | | |
| | CKTS | | | | | | | | YMELRSL | | | | | |
| | | | | | | | | | TSEDSAV | | | | | |
| | | | | | | | | | YYC | | | | | |
| ITI_ | EVQLQQS | 262 | GYTFTEY | 360 | MHWVKQS | 447 | INPNNGG | 536 | INNQKFK | 682 | AGSVVDR | 815 | WGAGTTV | 866 |
| 04 | GPELVKP | | T | | HGKSLEW | | T | | GKATLTV | | YWYFDV | | TVSS | |
| 0H | GTSVKIS | | | | IGG | | | | DMSSSTA | | | | | |
| | CKTS | | | | | | | | YMELRSL | | | | | |
| | | | | | | | | | TSEDSAV | | | | | |
| | | | | | | | | | YYC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #9

| | | | | | | | | | [K/R/N] | | | | | |
|---|---|---|---|---|---|---|---|---|---|---|---|---|---|---|
| | | | | | | | | | Y[D/A/N | | | | | |
| | | | | | | | | | ][P/E]K | | | | | |
| | | | | | | | | | F[Q/K][ | | | | | |
| | | | | | | | | | G/D/S]K | | | | | |
| | | | | | | | | | AT[I/L] | | | | | |
| | | | | | | | | | T[A/V]D | | | | | |
| | [E/-/Q] | | | | | | | | [T/K]SS | | AR[S/H] | | | |
| | [V/-][Q | | | | | | | | [N/T/S] | | [R/F/E/ | | | |
| | /-]LQQ[ | | | | | | | | T[A/V]Y | | G][R/G/ | | | |
| | S/P]GA[ | | G[F/Y][ | | [M/I][H | | [I/F][D | | [L/M][Q | | D/Y][-/ | | | |
| | E/G]LVK | | N/T][I/ | | /Y]WVKQ | | /Y]P[A/ | | /D] | | Y][-/G] | | | |
| | PG[A/T] | | F][K/T] | | R[P/S][ | | G][N/S] | | LS[S/R] | | [- | | | |
| | SVK[L/M | | [D/E/S] | | E/G]QGL | | [G/D][N | | LTSED[T | | /N/G/S] | | WG[A/Q] | |
| | ]SC[T/K | | [T/Y][Y | | EWIG[R/ | | /S][T/I | | /S]AVY[ | | Y[F/L]D | | GTT[V/L | |
| | ]AS | 264 | /I/W] | 361 | W/D] | 448 | ] | 538 | Y/F]C | 683 | [V/Y] | 816 | ]TVSS | 867 |
| H5S14- | QVQLQQP | 265 | GYTFTSY | 362 | MHWVKQR | 449 | IYPGSDS | 539 | NYNEKFK | 684 | ARSGYYG | 817 | WGQGTTL | 856 |
| 2AH | GAELVKP | | W | | PGQGLEW | | T | | SKATLTV | | SYLDY | | TVSS | |
| | GTSVKMS | | | | IGD | | | | DTSSSTA | | | | | |
| | CKAS | | | | | | | | YMQLSSL | | | | | |
| | | | | | | | | | TSEDSAV | | | | | |
| | | | | | | | | | YYC | | | | | |
| H5S15- | EVQLQQS | 229 | GFNIKDT | 322 | MHWVKQR | 400 | IDPANGN | 499 | KYDPKFQ | 617 | ARSRR-- | 818 | WGAGTTV | 866 |
| 27AH | GAELVKP | | Y | | PEQGLEW | | T | | GKATITA | | -YFDV | | TVSS | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S20- | EVQLQQS | 229 | GFNIKDT | 322 | MHWVKQR | 400 | IDPANGN | 499 | KYAPKFQ | 624 | ARSFG-- | 819 | WGQGTTL | 856 |
| 22AH | GAELVKP | | Y | | PEQGLEW | | T | | GKATITA | | NYFDY | | TVSS | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| ITI_ | LQQSGAG | 266 | GYTFTEY | 363 | IYWVKQR | 450 | FYPGSGS | 540 | RYNEKFK | 685 | ARHED-- | 820 | WGQGTTL | 856 |
| 23 | LVKPGAS | | I | | SGQGLEW | | I | | DKATLTA | | GYLDY | | TVSS | |
| 7H | VKLSCKA | | | | IGW | | | | DKSSTTV | | | | | |
| | S | | | | | | | | YMDLSRL | | | | | |
| | | | | | | | | | TSEDSAV | | | | | |
| | | | | | | | | | YFC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #17

| | | | | | | | | | TYADDFK | | | | | |
|---|---|---|---|---|---|---|---|---|---|---|---|---|---|---|
| | | | | | | | | | GRFAFSL | | ARS[I/L | | | |
| | QIQLVQS | | | | | | | | ETSAS[T | | ][N/Y]Y | | | |
| | GP[D/E] | | | | | | | | /S]AYLQ | | [-/G/V] | | | |
| | [L/V]KK | | | | | | | | I[N/S]N | | [-/N/D] | | | |
| | [P/H]GE | | | | [M/I]NW | | | | L[K/T][ | | [D/N][S | | | |
| | TV[K/R] | | GY[T/I] | | VKQAPGK | | INTYT[G | | N/T][D/ | | /Y][D/E | | WGQGT[S | |
| | ISC[K/R | | FT[N/D] | | [D/G]LK | | /R][E/K | | E]D[T/M | | ][E/A][ | | /L]VTVS | |
| | ]AS | 267 | YG | 364 | WMGW | 451 | ]P | 541 | ]ATYFC | 686 | K/C/Y] | 821 | [S/-/A] | 868 |
| H5S15- | QIQLVQS | 268 | GYTFTNY | 338 | MNWVKQA | 452 | INTYTGE | 513 | TYADDFK | 670 | ARSINY- | 822 | WGQGTSV | 848 |
| 21AH | GPDLKKP | | G | | PGKDLKW | | P | | GRFAFSL | | -DSDEK | | TVSS | |
| | GETVKIS | | | | MGW | | | | ETSASTA | | | | | |
| | CKAS | | | | | | | | YLQINNL | | | | | |
| | | | | | | | | | KNDDTAT | | | | | |
| | | | | | | | | | YFC | | | | | |
| H5S20- | QIQLVQS | 269 | GYTFTNY | 338 | MNWVKQA | 426 | INTYTGE | 513 | TYADDFK | 687 | ARSLYYG | 823 | WGQGTLV | 852 |
| 26AH | GPELKKP | | G | | PGKGLKW | | P | | GRFAFSL | | DNYEAY | | TVSA | |
| | GETVKIS | | | | MGW | | | | ETSASSA | | | | | |
| | CRAS | | | | | | | | YLQINNL | | | | | |
| | | | | | | | | | KNEDMAT | | | | | |
| | | | | | | | | | YFC | | | | | |
| ITI_ | QIQLVQS | 270 | GYIFTNY | 349 | INWVKQA | 453 | INTYTRE | 542 | TYADDFK | 688 | ARSLYYV | 824 | WGQGTLV | 852 |
| 12 | GPEVKKH | | G | | PGKGLKW | | P | | GRFAFSL | | NNYEAY | | TVSA | |
| 7H | GETVKIS | | | | MGW | | | | ETSASSA | | | | | |
| | CKAS | | | | | | | | YLQISNL | | | | | |
| | | | | | | | | | TTEDMAT | | | | | |
| | | | | | | | | | YFC | | | | | |
| ITI_ | QIQLVQS | 271 | GYIFTDY | 365 | INWVKQA | 453 | INTYTGK | 543 | TYADDFK | 689 | ARSLYYG | 825 | WGQGTLV | 869 |
| 12 | GPELKKH | | G | | PGKGLKW | | P | | GRFAFSL | | NNYEAC | | TVS- | |
| 8H | GETVRIS | | | | MGW | | | | ETSASTA | | | | | |
| | CKAS | | | | | | | | YLQINNL | | | | | |
| | | | | | | | | | KTEDMAT | | | | | |
| | | | | | | | | | YFC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #7

| | | | | | | | | | [K/D]YN | | | | | |
|---|---|---|---|---|---|---|---|---|---|---|---|---|---|---|
| | | | | | | | | | [E/A][K | | | | | |
| | QVQL[Q/ | | | | | | | | /A]F[K] | | | | | |
| | K]QSG[A | | | | | | | | [L/I][T | | | | | |
| | /P][E/G | | | | | | | | /S][A/K | | | | | |
| | ]LV[R/Q | | | | | | | | ]D[T/N] | | | | | |
| | ]P[G/S] | | | | | | | | S[S/K]S | | | | | |
| | [T/Q][S | | | | [I/V][G | | | | [T/Q][A | | | | | |
| | [N][V/L | | | | /H]W[V/ | | | | /V][Y/F | | | | | |
| | ][K/S][ | | G[Y/F][ | | I][K/R] | | | | ] | | | | | |
| | M/I] | | T/S][F/ | | Q[R/S]P | | I[Y/W][ | | SL[T/Q] | | | | WGQGT[T | |
| | [S/T]C[ | | L]T[N/S | | G[H/K]G | | P/S]G[G | | [S/A][E | | AS[G/L] | | /L][L/V | |
| | K/T][A/ | | ][S/Y][ | | LEW[I/L | | /-][A/G | | /D]D[S/ | | [R/-][D | | ]TVS[S/ | |
| | V][A/S] | 272 | W/G] | 366 | ]G[D/V] | 454 | ][Y/G]T | 544 | T]AIYYC | 690 | /Y]Y | 826 | A] | 858 |
| H5S15- | QVQLQQS | 226 | GYTFTNS | 289 | IGWVKQR | 397 | IYPGGAY | 545 | KYNEKFK | 691 | ASGRDY | 827 | WGQGTTL | 856 |
| 6AH | GAELVRP | | W | | PGHGLEW | | T | | GKATLTA | | | | TVSS | |
| | GTSVKMS | | | | IGD | | | | DTSSSTA | | | | | |
| | CKAA | | | | | | | | YMQLSSL | | | | | |
| | | | | | | | | | TSEDSAI | | | | | |
| | | | | | | | | | YYC | | | | | |
| ITI_ | QVQLKQS | 273 | GFSLTSY | 306 | VHWIRQS | 455 | IWSG-GG | 546 | DYNAAFI | 692 | ASL-YY | 828 | WGQGTLV | 852 |
| 04 | GPGLVQP | | G | | PGKGLEW | | T | | SRLSISK | | | | TVSA | |
| 5H | SQNLSIT | | | | LGV | | | | DNSKSQV | | | | | |
| | CTVS | | | | | | | | FFKMNSL | | | | | |
| | | | | | | | | | QADDTAI | | | | | |
| | | | | | | | | | YYC | | | | | |
| ITI_ | QVQLKQS | 273 | GFSLTSY | 306 | VHWIRQS | 455 | IWSG-GG | 546 | DYNAAFI | 693 | ASL-YY | 828 | WGQGTLV | 852 |
| 04 | GPGLVQP | | G | | PGKGLEW | | T | | SRLTISK | | | | TVSA | |
| 6H | SQNLSIT | | | | LGV | | | | DNSKSQV | | | | | |
| | CTVS | | | | | | | | FFKMNSL | | | | | |
| | | | | | | | | | QADDTAI | | | | | |
| | | | | | | | | | YYC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #13

| | | | | | | | | | [I/K/Y] | | | | | |
|---|---|---|---|---|---|---|---|---|---|---|---|---|---|---|
| | | | | | | | | | Y[D/K][ | | | | | |
| | | | | | | | | | P/E]KF[ | | | | | |
| | | | | | | | | | Q/K][G/ | | | | | |
| | | | | | | | | | D]KA[S/ | | | | | |
| | [E/Q]VQ | | | | | | | | T/I][I/ | | | | | |
| | LQQSG[A | | | | | | | | L]TAD[T | | | | | |
| | /G]EL[V | | | | | | | | /K]SS[N | | | | | |
| | /L][R/K | | G[F/Y][ | | | | | | /S]TAY[ | | AR[G/V] | | | |
| | ]PG[A/T | | N/A][I/ | | [M/I][H | | I[D/N]P | | L/M]QLS | | [Y/D][S | | | |
| | ][L/S]V | | F][K/T] | | /E]WVKQ | | [E/A/G] | | SLTS[E/ | | /Y/-][S | | | |
| | K[L/V]S | | [D/N][D | | RP[E/G] | | [N/S][G | | D]D[T/S | | /A][S/M | | WGQGT[L | |
| | C[K/T]A | | /T/Y][Y | | QGLEWIG | | /D][N/G | | ]AVY[Y/ | | ][P/D][ | | /S]VTVS | |
| | S | 274 | /L] | 367 | [W/R/V] | 456 | ][T/I] | 547 | F]C | 694 | Y/F1 | 829 | [A/S] | 870 |
| H5S15- | EVQLQQS | 275 | GFNIKDD | 368 | MHWVKQR | 443 | IDPENGN | 548 | IYDPKFQ | 695 | ARGYSSS | 830 | WGQGTLV | 852 |
| 29AH | GAELVRP | | Y | | PEQGLEW | | T | | GKASITA | | PY | | TVSA | |
| | GALVKLS | | | | IGW | | | | DTSSNTA | | | | | |
| | CKAS | | | | | | | | YLQLSSL | | | | | |
| | | | | | | | | | TSEDTAV | | | | | |
| | | | | | | | | | YYC | | | | | |
| H5S15- | EVQLQQS | 229 | GFNIKDT | 322 | MHWVKQR | 400 | IDPANDN | 502 | KYDPKFQ | 617 | ARVYYAM | 831 | WGQGTSV | 848 |
| 2AH | GAELVKP | | Y | | PEQGLEW | | T | | GKATITA | | DY | | TVSS | |
| | GASVKLS | | | | IGR | | | | DTSSNTA | | | | | |
| | CTAS | | | | | | | | YLQLSSL | | | | | |
| | | | | | | | | | TSEDTAV | | | | | |
| | | | | | | | | | YYC | | | | | |
| H5S19- | QVQLQQS | 276 | GYAFTNY | 327 | IEWVKQR | 413 | INPGSGG | 549 | YYKEKFK | 696 | ARGD-AM | 832 | WGQGTSV | 848 |
| 24AH | GGELLRP | | L | | PGQGLEW | | I | | DKAILTA | | DF | | TVSS | |
| | GTSVKVS | | | | IGV | | | | DKSSSTA | | | | | |
| | CKAS | | | | | | | | YMQLSSL | | | | | |
| | | | | | | | | | TSDDSAV | | | | | |
| | | | | | | | | | YFC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #18

| | | | | | | | | | | | TR[H/G] | | | |
|---|---|---|---|---|---|---|---|---|---|---|---|---|---|---|
| | | | | | | | | | YYPDSVK | | [E/D/G] | | | |
| | [E/D]V[ | | | | | | | | GRFTISR | | [L/D][G | | | |
| | Q/K]LVE | | | | | | | | DNAKNTL | | /-][N/- | | | |
| | SGG[D/G | | | | | | | | YLQM[N/ | | ][R/-][ | | WGQGT[L | |
| | ]LVKPGG | | [G/V]FT | | MSWVRQT | | | | S]SLKSE | | S/Y/G][ | | /T/A][V | |
| | SLKLSCA | | FS[S/R] | | P[D/E]K | | ISSGGS[ | | DTA[M/I | | R/G/L]F | | /L]TVS[ | |
| | AS | 277 | Y[G/T] | 369 | RLEWVAT | 457 | S/Y]T | 550 | ]YYC | 697 | [P/D]Y | 833 | A/S] | 871 |
| H5S14- | DVKLVES | 278 | VFTFSRY | 370 | MSWVRQT | 446 | ISSGGSY | 551 | YYPDSVK | 698 | TRGGD-- | 834 | WGQGTAL | 872 |
| 1AH | GGGLVKP | | T | | PEKRLEW | | T | | GRFTISR | | -GLFDY | | TVSS | |
| | GGSLKLS | | | | VAT | | | | DNAKNTL | | | | | |
| | CAAS | | | | | | | | YLQMSSL | | | | | |
| | | | | | | | | | KSEDTAI | | | | | |
| | | | | | | | | | YYC | | | | | |
| H5S19- | DVKLVES | 278 | GFTFSSY | 346 | MSWVRQT | 446 | ISSGGSY | 551 | YYPDSVK | 655 | TRGDD-- | 835 | WGQGTTL | 856 |
| 5AH | GGGLVKP | | T | | PEKRLEW | | T | | GRFTISR | | -YGFDY | | TVSS | |
| | GGSLKLS | | | | VAT | | | | DNAKNTL | | | | | |
| | CAAS | | | | | | | | YLQMSSL | | | | | |
| | | | | | | | | | KSEDTAM | | | | | |
| | | | | | | | | | YYC | | | | | |
| ITI_ | EVQLVES | 251 | GFTFSSY | 343 | MSWVRQT | 430 | ISSGGSS | 552 | YYPDSVK | 699 | TRHELGN | 836 | WGQGTLV | 852 |
| 20 | GGDLVKP | | G | | PDKRLEW | | T | | GRFTISR | | RSRFPY | | TVSA | |
| 3H | GGSLKLS | | | | VAT | | | | DNAKNTL | | | | | |
| | CAAS | | | | | | | | YLQMNSL | | | | | |
| | | | | | | | | | KSEDTAM | | | | | |
| | | | | | | | | | YYC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #22

| | | | | | | | | | [N/S/Y] | | | | | |
|---|---|---|---|---|---|---|---|---|---|---|---|---|---|---|
| | | | | | | | | | Y[D/N/S | | | | | |
| | | | | | | | | | ][E/P/D | | | | | |
| | | | | | | | | | ][K/S][ | | | | | |
| | | | | | | | | | F/L/V]K | | | | | |
| | | | | | | | | | [S/G][K | | | | | |
| | | | | | | | | | /R][A/I | | | | | |
| | | | | | | | | | /F][T/S | | | | | |
| | | | | | | | | | ][L/I][ | | | | | |
| | [Q/D/E] | | | | | | | | T/S][V/ | | | | | |
| | V[Q/K]L | | | | | | | | R]D[T/D | | | | | |
| | [Q/D][Q | | | | | | | | ]S[S/K] | | | | | |
| | /E][P/S | | | | | | | | [S/N][T | | | | | |
| | /T]G[S/ | | | | | | | | /Q/S][A | | | | | |
| | P/G][E/ | | | | | | | | /F/V][Y | | | | | |
| | G]LV[R/ | | | | [M/W][H | | | | /F][M/L | | | | | |
| | K/Q]P[G | | | | /N]W[V/ | | | | ]Q[L/M] | | | | | |
| | /S][A/Q | | | | I][K/R] | | I[Y/V/R | | [S/N][S | | | | | |
| | /R][S/P | | | | Q[R/F/S | | ][P/Y/N | | /N][L/V | | | | | |
| | ][V/L/M | | G[Y/F][ | | ]P[G/E] | | ][G/S/K | | ][T/R][ | | | | | |
| | ][K/S]L | | T/S][F/ | | [Q/N/K] | | ][-/P][ | | S/A]ED[ | | T[I/R/W | | | |
| | [S/T][K | | I][-/T] | | [G/K]LE | | -/Y][S/ | | S/T/M][ | | ][Y/G][ | | WGQGT[L | |
| | /T/V][A | | [T/S][S | | W[I/M/V | | -/N][G/ | | A/G][V/ | | D/-][G/ | | /S/T][V | |
| | /V][S/T | | /D][Y/F | | ][G/A][ | | Y][S/E] | | T/I]Y[Y | | M/-][Y/ | | /L]TVS[ | |
| | ] | 279 | ][W/A] | 371 | N/Y/Q] | 458 | T | 553 | /F]C | 700 | D/N]Y | 837 | A/S] | 873 |
| H5S14- | QVQLQQP | 280 | GYTF-TS | 316 | MHWVKQR | 459 | IYPG--S | 554 | NYDEKFK | 701 | TIYDGYY | 838 | WGQGTLV | 852 |
| 21AH | GSELVRP | | YW | | PGQGLEW | | GST | | SKATLTV | | | | TVSA | |
| | GASVKLS | | | | IGN | | | | DTSSSTA | | | | | |
| | CKAS | | | | | | | | YMQLSSL | | | | | |
| | | | | | | | | | TSEDSAV | | | | | |
| | | | | | | | | | YYC | | | | | |
| H5S14- | EVKLDET | 281 | GFTF-SD | 372 | MNWVRQS | 460 | IRNKPYN | 555 | YYSDSVK | 702 | TWG--NY | 839 | WGQGTTL | 856 |
| 27AH | GGGLVQP | | YW | | PEKGLEW | | YET | | GRFTISR | | | | TVSS | |
| | GRPMKLS | | | | VAQ | | | | DDSKSSV | | | | | |
| | CVAS | | | | | | | | YLQMNNL | | | | | |
| | | | | | | | | | RAEDMGI | | | | | |
| | | | | | | | | | YYC | | | | | |
| H5S14- | DVQLQES | 235 | GYSITSD | 373 | WNWIRQF | 403 | IVYS--- | 556 | SYNPSLK | 703 | TRG-MDY | 840 | WGQGTSV | 848 |
| 5AH | GPGLVKP | | FA | | PGNKLEW | | -GST | | SRISITR | | | | TVSS | |
| | SQSLSLT | | | | MGY | | | | DTSKNQF | | | | | |
| | CTVT | | | | | | | | FLQLNSV | | | | | |
| | | | | | | | | | TAEDTAT | | | | | |
| | | | | | | | | | YFC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #16

| | | | | | | | | | [S/K]Y[ | 704 | | | | |
|---|---|---|---|---|---|---|---|---|---|---|---|---|---|---|
| | | | | | | | | | S/D]P[S | | | | | |
| | | | | | | | | | /K][L/F | | | | | |
| | | | | | | | | | ][K/Q][ | | | | | |
| | | | | | | | | | S/D][R/ | | | | | |
| | | | | | | | | | K][I/A] | | | | | |
| | [D/E]VQ | | | | | | | | [S/T]IT | | | | | |
| | LQ[E/Q] | | | | | | | | [R/T]D[ | | | | | |
| | SG[P/A] | | | | [W/I][N | | | | T/A]S[K | | | | | |
| | [G/E]L[ | | | | /H]W[I/ | | | | /S]N[Q/ | | | | | |
| | V/M][K/ | | | | V][R/K] | | | | T][F/A] | | [V/T][Y | | | |
| | S]P[S/G | | | | Q[F/R]P | | | | [F/Y]LQ | | /K][F/S | | | |
| | ][Q/A]S | | G[Y/F][ | | [G/E][N | | | | L[N/S]S | | ][K/L][ | | | |
| | [L/V][S | | S/N]I[T | | /Q][K/G | | [A/D][Y | | [V/L]T[ | | Y/L][G/ | | | |
| | /N]L[T/ | | /-][S/K | | ]LEW[M/ | | /P][S/A | | T/S]EDT | | W][-/S] | | | |
| | S]CT[V/ | | ]D[Y/T] | | I]G[Y/K | | ][-/N]G | | A[T/V]Y | | [-/L]G[ | | WGQGTLV | |
| | A][T/S] | 282 | [A/Y] | 374 | ] | 461 | [G/N]T | 557 | YC | | A/G]FAY | 841 | TVSA | 852 |
| H5S19- | DVQLQES | 235 | GYSITSD | 318 | WNWIRQF | 403 | IAYS-GG | 558 | SYSPSLK | 705 | VYFKYG- | 842 | WGQGTLV | 852 |
| 1AH | GPGLVKP | | YA | | PGNKLEW | | T | | SRISITR | | -GAFAY | | TVSA | |
| | SQSLSLT | | | | MGY | | | | DTSKNQF | | | | | |
| | CTVT | | | | | | | | FLQLNSV | | | | | |
| | | | | | | | | | TTEDTAT | | | | | |
| | | | | | | | | | YYC | | | | | |
| ITI_ | EVQLQQS | 283 | GFNI-KD | 315 | IHWVKQR | 462 | IDPANGN | 499 | KYDPKFQ | 706 | TKSLLWS | 843 | WGQGTLV | 852 |
| 02 | GAELMSP | | TY | | PEQGLEW | | T | | DKATITT | | LGGFAY | | TVSA | |
| 3H | GASVNLS | | | | IGK | | | | DASSNTA | | | | | |
| | CTAS | | | | | | | | YLQLSSL | | | | | |
| | | | | | | | | | TSEDTAV | | | | | |
| | | | | | | | | | YYC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #1

| | | | | | | | | | TYADDFK | | | | | |
|---|---|---|---|---|---|---|---|---|---|---|---|---|---|---|
| | | | | | | | | | GRFAFSL | | | | | |
| | QIQLVQS | | | | | | | | ETSASTA | | | | | |
| | GPELKKP | | | | VNWVKQA | | | | YLQINNL | | | | | |
| | GETVKIS | | GYTFTNY | | PGKDLKW | | INTYTGE | | KNEDMAT | | | | WGQGTLV | |
| | CKAS | 247 | G | 338 | MGW | 425 | P | 513 | YFC | 645 | TSRSWVL | 844 | TVSA | 852 |
| H5S14- | QIQLVQS | 247 | GYTFTNY | 338 | VNWVKQA | 425 | INTYTGE | 513 | TYADDFK | 645 | TSRSWVL | 844 | WGQGTLV | 852 |
| 3AH | GPELKKP | | G | | PGKDLKW | | P | | GRFAFSL | | | | TVSA | |
| | GETVKIS | | | | MGW | | | | ETSASTA | | | | | |
| | CKAS | | | | | | | | YLQINNL | | | | | |
| | | | | | | | | | KNEDMAT | | | | | |
| | | | | | | | | | YFC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #2

| | | | | | | | | | VFNQKFK | | | | | |
|---|---|---|---|---|---|---|---|---|---|---|---|---|---|---|
| | | | | | | | | | GKAKLTA | | | | | |
| | EVQLQQS | | | | | | | | VTSATTA | | | | | |
| | GTVLARP | | | | MHWLKQR | | | | YMELSSL | | | | | |
| | GASVKMS | | GYSFTSY | | PGQGLEW | | IYPGNSD | | TNEDSAV | | TKEPRTI | | WGQGTLV | |
| | CKAS | 284 | W | 375 | IGA | 463 | T | 559 | YYC | 707 | EGAWFTY | 845 | TVSA | 852 |
| H5S20- | EVQLQQS | 284 | GYSFTSY | 375 | MHWLKQR | 463 | IYPGNSD | 559 | VFNQKFK | 707 | TKEPRTI | 845 | WGQGTLV | 852 |
| 27AH | GTVLARP | | W | | PGQGLEW | | T | | GKAKLTA | | EGAWFTY | | TVSA | |
| | GASVKMS | | | | IGA | | | | VTSATTA | | | | | |
| | CKAS | | | | | | | | YMELSSL | | | | | |
| | | | | | | | | | TNEDSAV | | | | | |
| | | | | | | | | | YYC | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |
| | | | | | | | | | TYNQKFT | 708 | | | | |
| | | | | | | | | | GKAKLTA | | | | | |
| | EVQLQQS | | | | | | | | VTSTSTA | | | | | |
| Consensus | GTVLARP | | | | MHWVKQR | | | | YMELSSL | | | | | |
| Cluster | GASVKMS | | GYTFTSF | | PGQGLEW | | ISPGNSE | | TNEDSAV | | TKIYYDY | | WGQGTTL | |
| #3 | CKAS | 284 | W | 376 | IGA | 442 | T | 560 | YYC | | DDGY | 846 | TVSS | 856 |
| H5S15- | EVQLQQS | 284 | GYTFTSF | 376 | MHWVKQR | 442 | ISPGNSE | 560 | TYNQKFT | 708 | TKIYYDY | 846 | WGQGTTL | 856 |
| 23AH | GTVLARP | | W | | PGQGLEW | | T | | GKAKLTA | | DDGY | | TVSS | |
| | GASVKMS | | | | IGA | | | | VTSTSTA | | | | | |
| | CKAS | | | | | | | | YMELSSL | | | | | |
| | | | | | | | | | TNEDSAV | | | | | |
| | | | | | | | | | YYC | | | | | |
| | | Seq. | | Seq. | | Seq. | | Seq. | | Seq. | | Seq. | | Seq. |
| | FR1 | ID | CDR1 | ID | FR2 | ID | | ID | FR3 | ID | CDR3 | ID | FR4 ID | ID |

Consensus Cluster #6

| | | | | | | | | | [N/Q/T/ | | | | | |
|---|---|---|---|---|---|---|---|---|---|---|---|---|---|---|
| | | | | | | | | | Y/R/S/K | | | | | |
| | | | | | | | | | ][L/S/R | | | | | |
| | | | | | | | | | ]E/D/I/ | | | | | |
| | | | | | | | | | Y/T/A/Q | | | | | |
| | | | | | | | | | /H][S/T | | | | | |
| | | | | | | | | | /P/D]G[ | | | | | |
| | | | | | | | | | I/V/A]P | | | | | |
| | [D/N/S/ | | | | | | | | [A/S/D/ | | | | | |
| | E/Q][I/ | | | | | | | | V/P/I]R | | | | | |
| | N/V][V/ | | | | | | | | F[S/R/T | | | | | |
| | Q][L/M/ | | | | | | | | ]GSG[S/ | | | | | |
| | I]TQ[S/ | | | | | | | | Y][G/R] | | | | | |
| | T][P/Q/ | | | | | | | | [T/S/N/ | | | | | |
| | T/S][A/ | | | | | | | | K][D/S/ | | | | | |
| | S/K/T][ | | [Q/E/R/ | | | | | | K][F/Y] | | | | | |
| | S/T/F/I | | S/K/D][ | | | | | | [T/S][L | | | | | |
| | /Y/L][L | | S/T/N/D | | | | | | /F][N/T | | | | | |
| | /M/Q][A | | /G/H][V | | | | | | /S/K][I | | | | | |
| | /S/T/L] | | /I/L][D | | | | | | /F][H/D | | Q[Q/H/N | | | |
| | [V/A/T] | | /-/L/S/ | | | | | | /S/N/G/ | | /S][S/N | | | |
| | [S/T/P] | | N/R][Y/ | | | | | | T][P/S/ | | /C/G/D/ | | | |
| | [L/P/A/ | | S/-/N][ | | | | | | T/N/R][ | | F/H/W/Y | | | |
| | V]G[Q/D | | -/S][-/ | | | | | | V/M/L][ | | /L][N/Y | | | |
| | /E/G][R | | E][D/Y/ | | | | | | E/Q/D] | | /H/T/F/ | | | |
| | /K/T/S] | | - | | | | | | [E/A/P/ | | S/W][E/ | | | |
| | [A/V/I] | | /N][G/- | | | | | | G/S/Q/T | | A/S/T/K | | | |
| | [T/S] | | /Q][D/N | | | | | | ][E/D]D | | /G][D/F | | | |
| | [I/ | | /E/S/K/ | | | | | | [A/V/L/ | | /Y/S/L/ | | FG[G/A/ | |
| | L/M/V][ | | -/G/T/R | | | | [A/L/Y/ | | F/I][A/ | | I/T/N][ | | S]G[T/A | |
| | S/T/N]C | | ][S/D/N | | | | W/S/G/R | | G/V][T/ | | P/T] | | ][K/NIL | |
| | [K/R/S/ | | /K/R/T] | | | | /D][A/E | | S/M/V/E | | [W/L | | [E/A][I | |
| | T/L][A/ | | [Y/F/L/ | | | | /T/G][S | | /D]Y[Y/ | | /F/Y/P/ | | /L][K/- | |
| | S/T/V]S | 1033 | D/N/W] | 1099 | CDR2 | 1163 | /T] | 1230 | F/H]C | 1262 | R]T | 1343 | /R] | 1426 |
| H5S14- | SIVMTQT | 1034 | QSV---- | 1100 | VAWYQQK | 1164 | YES | 1231 | NRYTGVP | 1263 | QQDYSSP | 1344 | FGGGTKL | 1427 |
| 15AL | PKFLLVS | | ----SND | | PGQSPKL | | | | DRFTGSG | | WT | | EIK | |
| | AGDRVTI | | | | LIY | | | | YGTDFTF | | | | | |
| | TCKAS | | | | | | | | TISTVQA | | | | | |
| | | | | | | | | | EDLAVYF | | | | | |
| | | | | | | | | | C | | | | | |
| H5S14- | SIVMTQT | 1034 | QSV---- | 1100 | VAWYQQK | 1164 | YAS | 1232 | NRYTGVP | 1263 | QQDYSSP | 1344 | FGGGTKL | 1427 |
| 16AL | PKFLLVS | | ----SND | | PGQSPKL | | | | DRFTGSG | | WT | | EIK | |
| | AGDRVTI | | | | LIY | | | | YGTDFTF | | | | | |
| | TCKAS | | | | | | | | TISTVQA | | | | | |
| | | | | | | | | | EDLAVYF | | | | | |
| | | | | | | | | | C | | | | | |
| H5S14- | QIVLTQS | 1035 | SSV---- | 1101 | MHWYQQK | 1165 | DTS | 1233 | KLASGVP | 1264 | QQWSSNP | 1345 | FGSGTKL | 1428 |
| 18AL | PAIMSAS | | -----SY | | SGTSPKR | | | | ARFSGSG | | FT | | EIK | |
| | PGEKVTM | | | | WIY | | | | SGTSYSL | | | | | |
| | TCSAS | | | | | | | | TISSMEA | | | | | |
| | | | | | | | | | EDAATYY | | | | | |
| | | | | | | | | | C | | | | | |
| H5S14- | DVQITQS | 1036 | KSI---- | 1102 | LAWYQEK | 1166 | SGS | 1234 | TLQSGIP | 1265 | QQHNEYP | 1346 | FGGGTKL | 1427 |
| 1AL | PSYLAAS | | ----SKY | | PGKTNQL | | | | SRFSGSG | | WT | | EIK | |
| | PGETITI | | | | LIY | | | | SGTDFTL | | | | | |
| | _ | | | | | | | | TISSLEP | | | | | |
| | NCRAS | | | | | | | | EDFAMYY | | | | | |
| | | | | | | | | | C | | | | | |
| H5S14- | NIVLTQS | 1037 | ESVDS-- | 1103 | MHWYQQK | 1167 | LAS | 1235 | NLESGVP | 1266 | QQNNEDP | 1347 | FGGGTKL | 1427 |
| 20AL | PASLAVS | | --YGNSF | | PGQPPKL | | | | ARFSGSG | | YT | | EIK | |
| | LGQRATI | | | | LIY | | | | SRTDFTL | | | | | |
| | SCRAS | | | | | | | | TIDPVEA | | | | | |
| | | | | | | | | | DDAATYY | | | | | |
| | | | | | | | | | C | | | | | |
| H5S14- | SIVMTQT | 1034 | QSV---- | 1100 | VAWYQQK | 1164 | YAS | 1232 | NRYTGVP | 1263 | QQDYSSP | 1348 | FGAGTKL | 1429 |
| 24AL | PKFLLVS | | ----SND | | PGQSPKL | | | | DRFTGSG | | LT | | ELK | |
| | AGDRVTI | | | | LIY | | | | YGTDFTF | | | | | |
| | TCKAS | | | | | | | | TISTVQA | | | | | |
| | | | | | | | | | EDLAVYF | | | | | |
| | | | | | | | | | C | | | | | |
| H5S14- | DIQMTQS | 1038 | DHI---- | 1104 | LAWYQQK | 1168 | GAT | 1236 | SLETGVP | 1267 | QQYWSTP | 1349 | FGGGTKL | 1427 |
| 25AL | SSYLSVS | | ----NNW | | PGNAPRL | | | | PRFSGSG | | YT | | EIK | |
| | LGGRVTI | | | | LIS | | | | SGKDYTL | | | | | |
| | TCKAS | | | | | | | | SITSLQT | | | | | |
| | | | | | | | | | EDVATYY | | | | | |
| | | | | | | | | | C | | | | | |
| H5S14- | QIVLTQS | 1039 | SSV---- | 1101 | MYWYQQK | 1169 | RTS | 1237 | NLASGVP | 1268 | QQYHSYP | 1350 | FGAGTKL | 1429 |
| 4AL | PAIMSAS | | -----SY | | PGSSPKP | | | | ARFSGSG | | LT | | ELK | |
| | PGEKVTI | | | | WIY | | | | SGTSYSL | | | | | |
| | SCSAS | | | | | | | | TISSMEA | | | | | |
| | | | | | | | | | EDAATYY | | | | | |
| | | | | | | | | | C | | | | | |
| H5S14- | QIVLTQS | 1039 | SSV---- | 1101 | MYWYQQK | 1169 | RTS | 1237 | NLASGVP | 1269 | QQYHSYP | 1350 | FGAGTKL | 1429 |
| 4BL | PAIMSAS | | -----SY | | PGSSPKP | | | | ARFSGSG | | LT | | ELK | |
| | PGEKVTI | | | | WIY | | | | SGSSYSL | | | | | |
| | SCSAS | | | | | | | | TISSMEA | | | | | |
| | | | | | | | | | EDAATYY | | | | | |
| | | | | | | | | | C | | | | | |
| H5S14- | DIQMTQS | 1040 | QTI---- | 1105 | LAWYQQK | 1170 | AAT | 1238 | SLADGVP | 1270 | QQLHSTP | 1351 | FGGGTKL | 1427 |
| 6AL | PASQSAS | | ----GTW | | PGKSPQL | | | | SRFSGSG | | YT | | EIK | |
| | LGESVTI | | | | LIY | | | | SGTKFSF | | | | | |
| | TCLAS | | | | | | | | KISSLQA | | | | | |
| | | | | | | | | | EDFVSYY | | | | | |
| | | | | | | | | | C | | | | | |
| H5S15- | DIVLTQS | 1041 | QSVDY-- | 1106 | MNWYQQK | 1171 | AAS | 1239 | NLDSGIP | 1271 | QQSNEDP | 1352 | FGAGTKL | 1429 |
| 13AL | PASLAVS | | --DGESY | | PGQPPKL | | | | ARFSGSG | | LT | | ELK | |
| | LGQRATI | | | | LIY | | | | SGTDFTL | | | | | |
| | SCKAS | | | | | | | | NIHPVEE | | | | | |
| | | | | | | | | | EDAATYY | | | | | |
| | | | | | | | | | C | | | | | |
| H5S15- | DIVMTQS | 1042 | QNV---- | 1107 | VAWYQQK | 1172 | SAS | 1240 | YRYSGVP | 1272 | QQYNSYP | 1353 | FGGGTNL | 1430 |
| 14AL | QKFMSTS | | ----GTN | | PGQSPKA | | | | DRFTGSG | | YT | | EIK | |
| | VGDRVSV | | | | LIY | | | | SGTDFTL | | | | | |
| | TCKAS | | | | | | | | TISNVQS | | | | | |
| | | | | | | | | | EDLAEYF | | | | | |
| | | | | | | | | | C | | | | | |
| H5S15- | DIVMTQS | 1042 | QNV---- | 1107 | VAWYQQR | 1173 | SAS | 1240 | YRYSGVP | 1273 | QQYNSYP | 1353 | FGGGAKL | 1431 |
| 14BL | QKFMSTS | | --GTN | | PGQSPKS | | | | DRFTGSG | | YT | | EIK | |
| | VGDRVSV | | | | LIY | | | | SGTDFTL | | | | | |
| | TCKAS | | | | | | | | TISNVQS | | | | | |
| | | | | | | | | | EDLADYF | | | | | |
| | | | | | | | | | C | | | | | |
| H5S15- | DIVMTQS | 1042 | QNV---- | 1107 | VAWYQQK | 1172 | SAS | 1240 | YRYSGVP | 1272 | QQYNSYP | 1353 | FGGGTKL | 1432 |
| 14CL | QKFMSTS | | --GTN | | PGQSPKA | | | | DRFTGSG | | YT | | EI- | |
| | VGDRVSV | | | | LIY | | | | SGTDFTL | | | | | |
| | TCKAS | | | | | | | | TISNVQS | | | | | |
| | | | | | | | | | EDLAEYF | | | | | |
| | | | | | | | | | C | | | | | |
| H5S15- | QIVLTQS | 1035 | SSV---- | 1101 | MHWYQQK | 1165 | DTS | 1233 | KLASGVP | 1264 | QQWSSNP | 1354 | FGAGTKL | 1429 |
| 15AL | PAIMSAS | | ---SY | | SGTSPKR | | | | ARFSGSG | | LT | | ELK | |
| | PGEKVTM | | | | WIY | | | | SGTSYSL | | | | | |
| | TCSAS | | | | | | | | TISSMEA | | | | | |
| | | | | | | | | | EDAATYY | | | | | |
| | | | | | | | | | C | | | | | |
| H5S15- | ENVLTQS | 1043 | SSVS--- | 1108 | LHWYQQK | 1174 | STS | 1241 | NLASGVP | 1274 | QQYSGYP | 1355 | FGGGTKL | 1427 |
| 18AL | PAIMSAS | | --SSY | | SGASPKL | | | | ARFSGSG | | LT | | EIK | |
| | PGEKVTM | | | | WIY | | | | SGTSYSL | | | | | |
| | TCRAS | | | | | | | | TISSVEA | | | | | |
| | | | | | | | | | EDAATYY | | | | | |
| | | | | | | | | | C | | | | | |
| H5S15- | DIVLTQS | 1041 | QSVDY-- | 1109 | MNWYQQK | 1175 | AAS | 1239 | NLESGIP | 1275 | QQSNEDP | 1356 | FGSGTKL | 1428 |
| 24AL | PASLAVS | | DGNSY | | PRQPPKL | | | | ARFRGSG | | FT | | EIK | |
| | LGQRATI | | | | LIY | | | | SGTDFTL | | | | | |
| | SCKAS | | | | | | | | NIHPVEE | | | | | |
| | | | | | | | | | EDAATYY | | | | | |
| | | | | | | | | | C | | | | | |
| H5S15- | DIQMTQT | 1044 | QDI---- | 1110 | LNWYQQK | 1176 | YTS | 1242 | RLHSGVP | 1276 | QQGNSLP | 1357 | FGGGTKL | 1427 |
| 28AL | TSSLSVS | | --SNY | | PDGTVKL | | | | SRFSGSG | | WT | | EIK | |
| | LGDRVTI | | | | LIY | | | | SGSDYSL | | | | | |
| | SCRAS | | | | | | | | TISNLEQ | | | | | |
| | | | | | | | | | EDIATYF | | | | | |
| | | | | | | | | | C | | | | | |
| H5S15- | EIVLTQS | 1045 | STIS--- | 1111 | LHWYQQK | 1177 | GTS | 1243 | NLASGVP | 1277 | QQWNSYP | 1358 | FGSGTKL | 1428 |
| 29AL | PALMTAS | | --SRN | | SEASPKP | | | | VRFSGSG | | LT | | EIK | |
| | PGEKVTI | | | | WIY | | | | SGTSYSL | | | | | |
| | TCSVS | | | | | | | | TISSMEA | | | | | |
| | | | | | | | | | EDAATYY | | | | | |
| | | | | | | | | | C | | | | | |
| H5S15- | EIVLTQS | 1046 | SGIR--- | 1112 | LHWYQQK | 1178 | GTS | 1243 | NLASGVP | 1278 | QQWSSYP | 1359 | FGSGTKL | 1428 |
| 4AL | PALMAAS | | --SSN | | SETSPKP | | | | IRFSGSG | | LT | | EIK | |
| | PGEKVTI | | | | WIY | | | | SGTSYSL | | | | | |
| | TCSVS | | | | | | | | TISSMEA | | | | | |
| | | | | | | | | | EDAATYY | | | | | |
| | | | | | | | | | C | | | | | |
| H5S19- | EIVLTQS | 1046 | SSIS--- | 1113 | LHWYQQK | 1178 | GTS | 1243 | NLASGVP | 1277 | QQWSSYP | 1359 | FGAGTKL | 1429 |
| 11AL | PALMAAS | | --SSN | | SETSPKP | | | | VRFSGSG | | LT | | ELK | |
| | PGEKVTI | | | | WIY | | | | SGTSYSL | | | | | |
| | TCSVS | | | | | | | | TISSMEA | | | | | |
| | | | | | | | | | EDAATYY | | | | | |
| | | | | | | | | | C | | | | | |
| H5S19- | DIQMTQT | 1047 | QGI---- | 1114 | LNWYQQK | 1176 | YTS | 1242 | SLHSGVP | 1279 | QQYSKLP | 1360 | FGGGTKL | 1427 |
| 17AL | TSSLSAS | | --SNY | | PDGTVKL | | | | SRFSGSG | | WT | | EIK | |
| | LGDRVTI | | | | LIY | | | | SGTDYSL | | | | | |
| | SCSAS | | | | | | | | TISNLEP | | | | | |
| | | | | | | | | | EDIATYY | | | | | |
| | | | | | | | | | C | | | | | |
| H5S19- | DIQMTQT | 1047 | QGI---- | 1114 | LNWYQQK | 1176 | YTS | 1242 | SLHSGVP | 1280 | QQYSKLP | 1360 | FGGGTKL | 1427 |
| 17BL | TSSLSAS | | --SNY | | PDGTVKL | | | | SRFRGSG | | WT | | EIK | |
| | LGDRVTI | | | | LIY | | | | SGTDYSL | | | | | |
| | SCSAS | | | | | | | | TISNLEP | | | | | |
| | | | | | | | | | EDIATYY | | | | | |
| | | | | | | | | | C | | | | | |
| H5S19- | DIQMTQT | 1047 | QGI---- | 1114 | LNWYQQK | 1176 | YTS | 1242 | SLHSGVP | 1281 | QQYSKLP | 1360 | FGGGTKL | 1427 |
| 17CL | TSSLSAS | | --SNY | | PDGTVKL | | | | SRFSGSG | | WT | | EIK | |
| | LGDRVTI | | | | LIY | | | | SGTDYSL | | | | | |
| | SCSAS | | | | | | | | TISNLEP | | | | | |
| | | | | | | | | | EDIATYF | | | | | |
| | | | | | | | | | C | | | | | |
| H5S19- | DIQMTQT | 1047 | QGI---- | 1114 | LNWYQQK | 1176 | YTS | 1242 | SLHSGVP | 1282 | QQYSKLP | 1360 | FGGGTKL | 1427 |
| 17DL | TSSLSAS | | -SNY | | PDGTVKL | | | | SRFSGSG | | WT | | EIK | |
| | LGDRVTI | | | | LIY | | | | SGTDYSL | | | | | |
| | SCSAS | | | | | | | | SISNLEP | | | | | |
| | | | | | | | | | EDIATYY | | | | | |
| | | | | | | | | | C | | | | | |
| H5S19- | DIQMTQT | 1047 | QGI---- | 1114 | LNWYQQK | 1176 | YTS | 1242 | SLHSGVP | 1283 | QQYSKLP | 1360 | FGGGTKL | 1427 |
| 17EL | TSSLSAS | | --SNY | | PDGTVKL | | | | SRFSGSG | | WT | | EIK | |
| | LGDRVTI | | | | LIY | | | | SGTDYSL | | | | | |
| | SCSAS | | | | | | | | TFSNLEP | | | | | |
| | | | | | | | | | EDIATYY | | | | | |
| | | | | | | | | | C | | | | | |
| H5S19- | DIQMTQT | 1047 | QGI---- | 1114 | LNWYQQK | 1176 | YTS | 1242 | SLHSGVP | 1279 | QQYSKLP | 1360 | FGGGTKL | 1432 |
| 17FL | TSSLSAS | | --SNY | | PDGTVKL | | | | SRFSGSG | | WT | | EI- | |
| | LGDRVTI | | | | LIY | | | | SGTDYSL | | | | | |
| | SCSAS | | | | | | | | TISNLEP | | | | | |
| | | | | | | | | | EDIATYY | | | | | |
| | | | | | | | | | C | | | | | |
| H5S19- | NIVLTQS | 1037 | ESVDS-- | 1115 | MHWYQQK | 1167 | LAS | 1235 | NLESGVP | 1266 | QQNNEDP | 1361 | FGGGTKL | 1432 |
| 1AL | PASLAVS | | YGNSL | | PGQPPKL | | | | ARFSGSG | | PT | | EI- | |
| | LGQRATI | | | | LIY | | | | SRTDFTL | | | | | |
| | SCRAS | | | | | | | | TIDPVEA | | | | | |
| | | | | | | | | | DDAATYY | | | | | |
| | | | | | | | | | C | | | | | |
| H5S19- | DIQMTQT | 1048 | QGI---- | 1114 | LNWYQQK | 1176 | YTS | 1242 | TLHSGVP | 1284 | QQYFKLP | 1362 | FGGGTKL | 1427 |
| 20AL | TSSLSAS | | --SNY | | PDGTVKL | | | | SRFSGSG | | WT | | EIK | |
| | LGDRVTI | | | | LIY | | | | SGTDYSL | | | | | |
| | SCTAS | | | | | | | | TISNLEP | | | | | |
| | | | | | | | | | EDIATYY | | | | | |
| | | | | | | | | | C | | | | | |
| H5S19- | DIQMTQT | 1047 | QGI---- | 1116 | LNWYQQK | 1176 | YTS | 1242 | SLHSGVP | 1279 | QQYSKLP | 1360 | FGGGTKL | 1427 |
| 23AL | TSSLSAS | | --NNY | | PDGTVKL | | | | SRFSGSG | | WT | | EIK | |
| | LGDRVTI | | | | LIY | | | | SGTDYSL | | | | | |
| | SCSAS | | | | | | | | TISNLEP | | | | | |
| | | | | | | | | | EDIATYY | | | | | |
| | | | | | | | | | C | | | | | |
| H5S19- | DIQMTQT | 1048 | QGI---- | 1116 | LNWYQQK | 1176 | YTS | 1242 | TLHSGVP | 1284 | QQYFKLP | 1362 | FGGGTKL | 1427 |
| 26AL | TSSLSAS | | --NNY | | PDGTVKL | | | | SRFSGSG | | WT | | EIK | |
| | LGDRVTI | | | | LIY | | | | SGTDYSL | | | | | |
| | SCTAS | | | | | | | | TISNLEP | | | | | |
| | | | | | | | | | EDIATYY | | | | | |
| | | | | | | | | | C | | | | | |
| H5S19- | DIQMTQT | 1048 | QGI---- | 1114 | LNWYQQK | 1176 | YTS | 1242 | TLHSGVP | 1284 | QQYSKLP | 1360 | FGGGTKL | 1433 |
| 28AL | TSSLSAS | | --SNY | | PDGTVKL | | | | SRFSGSG | | WT | | AIK | |
| | LGDRVTI | | | | LIY | | | | SGTDYSL | | | | | |
| | SCTAS | | | | | | | | TISNLEP | | | | | |
| | | | | | | | | | EDIATYY | | | | | |
| | | | | | | | | | C | | | | | |
| H5S19- | QIVLTQS | 1035 | SSV---- | 1101 | MYWYQQK | 1179 | DTS | 1233 | NLASGVP | 1285 | QQWSSYP | 1363 | FGSGTKL | 1428 |
| 6AL | PAIMSAS | | ---SY | | PGSSPRL | | | | VRFSGSG | | FT | | EIK | |
| | PGEKVTM | | | | LIY | | | | SGTSYSL | | | | | |
| | TCSAS | | | | | | | | TISRMEA | | | | | |
| | | | | | | | | | EDAATYY | | | | | |
| | | | | | | | | | C | | | | | |
| H5S19- | DVQITQS | 1049 | KNI---- | 1117 | LAWYQEK | 1180 | SGS | 1234 | TLQSGIP | 1286 | QQHNEYP | 1346 | FGGGTKL | 1427 |
| 8AL | PSYLAAS | | --SKY | | PGKTNKL | | | | SRFSGSG | | WT | | EIK | |
| | PGETITI | | | | LIY | | | | SGTDFTL | | | | | |
| | _ | | | | | | | | TISSLEP | | | | | |
| | NCRTS | | | | | | | | EDFVMYH | | | | | |
| | | | | | | | | | C | | | | | |
| H5S20- | DIVLTQS | 1041 | QSVDY-- | 1109 | MNWYQQK | 1171 | AAS | 1239 | NLESGIP | 1287 | QQSNEDP | 1364 | FGGGTKL | 1427 |
| 13AL | PASLAVS | | DGNSY | | PGQPPKL | | | | ARFSGSG | | WT | | EIK | |
| | LGQRATI | | | | LIY | | | | SGTDFTL | | | | | |
| | SCKAS | | | | | | | | NIHPVEE | | | | | |
| | | | | | | | | | EDAATYY | | | | | |
| | | | | | | | | | C | | | | | |
| H5S20- | QIVLTQS | 1050 | SSV---- | 1118 | IYWYQQK | 1181 | LTS | 1244 | NLASGVP | 1268 | QQWSSNP | 1354 | FGAGTKL | 1434 |
| 16AL | PALMSAS | | ---SF | | PRSSPKP | | | | ARFSGSG | | LT | | EL- | |
| | PGEKVTM | | | | WIS | | | | SGTSYSL | | | | | |
| | TCSAS | | | | | | | | TISSMEA | | | | | |
| | | | | | | | | | EDAATYY | | | | | |
| | | | | | | | | | C | | | | | |
| H5S20- | QIVLTQS | 1050 | SSV---- | 1118 | IYWYQQK | 1182 | LTS | 1244 | NLASGVP | 1268 | QQWSSNP | 1354 | FGAGTKL | 1434 |
| 16BL | PALMSAS | | ---SF | | PRSSPKP | | | | ARFSGSG | | LT | | EL- | |
| | PGEKVTM | | | | WIY | | | | SGTSYSL | | | | | |
| | TCSAS | | | | | | | | TISSMEA | | | | | |
| | | | | | | | | | EDAATYY | | | | | |
| | | | | | | | | | C | | | | | |
| H5S20- | DIVMTQS | 1051 | RTI---- | 1119 | LHWYQQK | 1183 | YAS | 1232 | QSISGIP | 1288 | QNGHSFP | 1365 | FGAGTKL | 1429 |
| 17AL | PATLSVT | | --SDY | | SHESPRL | | | | SRFSGSG | | LT | | ELK | |
| | PGDRVSL | | | | LIK | | | | SGSDFTL | | | | | |
| | SCRAS | | | | | | | | SISSVEP | | | | | |
| | | | | | | | | | EDVGMYY | | | | | |
| | | | | | | | | | C | | | | | |
| H5S20- | NIVLTQS | 1037 | ESVDS-- | 1103 | MHWYQQK | 1167 | LAS | 1235 | NLESGVP | 1266 | QQNYADP | 1366 | FGGGTKL | 1427 |
| 18AL | PASLAVS | | YGNSF | | PGQPPKL | | | | ARFSGSG | | WT | | EIK | |
| | LGQRATI | | | | LIY | | | | SRTDFTL | | | | | |
| | SCRAS | | | | | | | | TIDPVEA | | | | | |
| | | | | | | | | | DDAATYY | | | | | |
| | | | | | | | | | C | | | | | |
| H5S20- | DIVLTQS | 1041 | QSVDY-- | 1120 | MNWYQQK | 1171 | AAS | 1239 | NLESGIP | 1287 | QQSNEDP | 1364 | FGGGTKL | 1427 |
| 20AL | PASLAVS | | DGDSF | | PGQPPKL | | | | ARFSGSG | | WT | | EIK | |
| | LGQRATI | | | | LIY | | | | SGTDFTL | | | | | |
| | SCKAS | | | | | | | | NIHPVEE | | | | | |
| | | | | | | | | | EDAATYY | | | | | |
| | | | | | | | | | C | | | | | |
| H5S20- | DIVMTQS | 1052 | QSLLNSE | 1121 | LTWYQQK | 1184 | WAS | 1245 | TRESGVP | 1289 | QSDYSYP | 1367 | FGAGTKL | 1429 |
| 23AL | PSSLTVP | | NQKNY | | PGQPPKL | | | | DRFTGSG | | LT | | ELK | |
| | AGEKVTM | | | | LIY | | | | SGTDFTL | | | | | |
| | SCKSS | | | | | | | | TISSVQA | | | | | |
| | | | | | | | | | EDLAVYY | | | | | |
| | | | | | | | | | C | | | | | |
| H5S20- | DIVMTQS | 1042 | QNV---- | 1107 | VAWYQQK | 1172 | SAS | 1240 | YRYSGVP | 1272 | QQYNSYP | 1368 | FGSGTKL | 1428 |
| 32AL | QKFMSTS | | --GTN | | PGQSPKA | | | | DRFTGSG | | FT | | EIK | |
| | VGDRVSV | | | | LIY | | | | SGTDFTL | | | | | |
| | TCKAS | | | | | | | | TISNVQS | | | | | |
| | | | | | | | | | EDLAEYF | | | | | |
| | | | | | | | | | C | | | | | |
| H5S20- | QIVLTQS | 1053 | SSV---- | 1101 | MYWYQQK | 1185 | LTS | 1244 | NLASGVP | 1268 | QQWSSNP | 1354 | FGAGTKL | 1435 |
| 34AL | PALMSAS | | ---SY | | PRSSPKP | | | | ARFSGSG | | LT | | ELR | |
| | PGEKVTM | | | | WIY | | | | SGTSYSL | | | | | |
| | TCTAS | | | | | | | | TISSMEA | | | | | |
| | | | | | | | | | EDAATYY | | | | | |
| | | | | | | | | | C | | | | | |
| H5S20- | DIVMTQS | 1042 | QNV---- | 1107 | VAWYQQK | 1172 | SAS | 1240 | YRYSGVP | 1272 | QQYNSYP | 1369 | FGGGTKL | 1427 |
| 36ALand | QKFMSTS | | --GTN | | PGQSPKA | | | | DRFTGSG | | LT | | EIK | |
| H5S20- | VGDRVSV | | | | LIY | | | | SGTDFTL | | | | | |
| 43AL | TCKAS | | | | | | | | TISNVQS | | | | | |
| | | | | | | | | | EDLAEYF | | | | | |
| | | | | | | | | | C | | | | | |
| H5S20- | DIVMTQS | 1042 | QNV---- | 1107 | VAWYQQK | 1172 | SAS | 1240 | YRYSGVP | 1272 | QQYNSYP | 1369 | FGAGTKL | 1429 |
| 40AL | QKFMSTS | | --GTN | | PGQSPKA | | | | DRFTGSG | | LT | | ELK | |
| | VGDRVSV | | | | LIY | | | | SGTDFTL | | | | | |
| | TCKAS | | | | | | | | TISNVQS | | | | | |
| | | | | | | | | | EDLAEYF | | | | | |
| | | | | | | | | | C | | | | | |
| H5S20- | DIVLTQS | 1041 | QSVDY-- | 1122 | MNWYQQK | 1171 | AAS | 1239 | NLESGIP | 1287 | QQSNEDP | 1364 | FGGGTKL | 1427 |
| 8AL | PASLAVS | | DGDSY | | PGQPPKL | | | | ARFSGSG | | WT | | EIK | |
| | LGQRATI | | | | LIY | | | | SGTDFTL | | | | | |
| | SCKAS | | | | | | | | NIHPVEE | | | | | |
| | | | | | | | | | EDAATYY | | | | | |
| | | | | | | | | | C | | | | | |
| H5S20- | DIVLTQS | 1041 | QSVDY-- | 1122 | MNWFQQK | 1186 | AAS | 1239 | NLESGIP | 1287 | QQSNEDP | 1364 | FGGGTKL | 1427 |
| 8BL | PASLAVS | | DGDSY | | PGQPPKL | | | | ARFSGSG | | WT | | EIK | |
| | LGQRATI | | | | LIY | | | | SGTDFTL | | | | | |
| | SCKAS | | | | | | | | NIHPVEE | | | | | |
| | | | | | | | | | EDAATYY | | | | | |
| | | | | | | | | | C | | | | | |
| ITI_ | NIVLTQS | 1037 | ESVDS-- | 1103 | MHWYQQK | 1167 | LAS | 1235 | NLESGVP | 1266 | QQNNEDP | 1370 | FGGGTKL | 1427 |
| 02 | PASLAVS | | YGNSF | | PGQPPKL | | | | ARFSGSG | | LT | | EIK | |
| 1L | LGQRATI | | | | LIY | | | | SRTDFTL | | | | | |
| | SCRAS | | | | | | | | TIDPVEA | | | | | |
| | | | | | | | | | DDAATYY | | | | | |
| | | | | | | | | | C | | | | | |
| ITI_ | QIVLTQS | 1039 | SSI---- | 1123 | MYWYQQK | 1169 | RTS | 1237 | NLASGVP | 1268 | QQYHSYP | 1350 | FGAGTKL | 1429 |
| 04 | PAIMSAS | | ---SY | | PGSSPKP | | | | ARFSGSG | | LT | | ELK | |
| 0L | PGEKVTI | | | | WIY | | | | SGTSYSL | | | | | |
| | SCSAS | | | | | | | | TISSMEA | | | | | |
| | | | | | | | | | EDAATYY | | | | | |
| | | | | | | | | | C | | | | | |
| ITI_ | EIVLTQS | 1054 | SSIN--- | 1124 | LHWYQQK | 1187 | RTS | 1237 | NLASGVP | 1290 | QQGSTIP | 1371 | FGGGAKL | 1431 |
| 04 | PTTMAAS | | --SNY | | PGFSPKL | | | | PRFSGSG | | YT | | EIK | |
| 5L | PGEKITI | | | | LIY | | | | SGTSYSL | | | | | |
| | _ | | | | | | | | TIGTMEA | | | | | |
| | TCSAS | | | | | | | | EDVATYY | | | | | |
| | | | | | | | | | C | | | | | |
| ITI_ | ENVLTQS | 1055 | SSV---- | 1125 | MYWYQQK | 1188 | YTS | 1242 | NLAPGVP | 1291 | QQFTSST | 1372 | FGGGTKL | 1427 |
| 08 | PAIMSAS | | ---NY | | SDASPKL | | | | ARFSGSG | | WT | | EIK | |
| 2L | LGEKVTM | | | | WIY | | | | SGNSYSL | | | | | |
| | SCRAS | | | | | | | | TISSMEG | | | | | |
| | | | | | | | | | EDAATYY | | | | | |
| | | | | | | | | | C | | | | | |
| ITI_ | ENVLTQS | 1055 | SSV---- | 1125 | MFWYQQK | 1189 | STS | 1241 | NLTPGVP | 1292 | QQFTSST | 1372 | FGGGTKL | 1427 |
| 08 | PAIMSAS | | ---NY | | SDASPKL | | | | ARFSGSG | | WT | | EIK | |
| 3L | LGEKVTM | | | | WIY | | | | SGNSYSL | | | | | |
| | SCRAS | | | | | | | | TISSMEA | | | | | |
| | | | | | | | | | EDAATYY | | | | | |
| | | | | | | | | | C | | | | | |
| ITI_ | EIVLTQS | 1046 | SSIS--- | 1113 | LHWYQQK | 1178 | GTS | 1243 | NLASGVP | 1277 | QQWSSYP | 1359 | FGSGTKL | 1428 |
| 13 | PALMAAS | | --SSN | | SETSPKP | | | | VRFSGSG | | LT | | EIK | |
| 1L | PGEKVTI | | | | WIY | | | | SGTSYSL | | | | | |
| | TCSVS | | | | | | | | TISSMEA | | | | | |
| | | | | | | | | | EDAATYY | | | | | |
| | | | | | | | | | C | | | | | |
| ITI_ | DIQMTQT | 1056 | QDI---- | 1126 | LNWYQQK | 1176 | YTS | 1242 | SLHSGAP | 1293 | QQYSKLP | 1360 | FGGGTKL | 1427 |
| 14 | PSSLSAS | | --NNY | | PDGTVKL | | | | SRFSGSG | | WT | | EIK | |
| 4L | LGDRVTI | | | | LIY | | | | SGTDYSL | | | | | |
| | SCSAS | | | | | | | | TISNLDP | | | | | |
| | | | | | | | | | EDIATYY | | | | | |
| | | | | | | | | | C | | | | | |
| ITI_ | DIQMTQT | 1056 | QDI-NNY | 1126 | LNWYQQK | 1190 | YTS | 1242 | SLHSGAP | 1293 | QQYSKLP | 1360 | FGGGTKL | 1427 |
| 14 | PSSLSAS | | | | PDGTVKL | | | | SRFSGSG | | WT | | EIK | |
| 5L | LGDRVTI | | | | LIF | | | | SGTDYSL | | | | | |
| | SCSAS | | | | | | | | TISNLDP | | | | | |
| | | | | | | | | | EDIATYY | | | | | |
| | | | | | | | | | C | | | | | |
| ITI_ | DIQMTQT | 1047 | QGI-RNY | 1127 | LNWYQQK | 1176 | YTS | 1242 | SLHSGVP | 1279 | QQYSKLP | 1360 | FGGGTKL | 1427 |
| 14 | TSSLSAS | | | | PDGTVKL | | | | SRFSGSG | | WT | | EIK | |
| 6L | LGDRVTI | | | | LIY | | | | SGTDYSL | | | | | |
| | SCSAS | | | | | | | | TISNLEP | | | | | |
| | | | | | | | | | EDIATYY | | | | | |
| | | | | | | | | | C | | | | | |
| ITI_ | DIQMTQT | 1048 | QGI-SNY | 1114 | LNWYQQK | 1176 | YTS | 1242 | TLHSGVP | 1294 | QQYSKLP | 1360 | FGGGTKL | 1427 |
| 16 | TSSLSAS | | | | PDGTVKL | | | | SRFSGSG | | WT | | EIK | |
| 2L | LGDRVTI | | | | LIY | | | | SGTDYSL | | | | | |
| | SCTAS | | | | | | | | TINNLEP | | | | | |
| | | | | | | | | | EDIATYY | | | | | |
| | | | | | | | | | C | | | | | |
| ITI_ | DIQMTQT | 1047 | QDI---- | 1110 | LNWYQQK | 1176 | YTS | 1242 | SLHSGVP | 1279 | QQYSKLP | 1360 | FGGGTKL | 1427 |
| 16 | TSSLSAS | | --SNY | | PDGTVKL | | | | SRFSGSG | | WT | | EIK | |
| 6L | LGDRVTI | | | | LIY | | | | SGTDYSL | | | | | |
| | SCSAS | | | | | | | | TISNLEP | | | | | |
| | | | | | | | | | EDIATYY | | | | | |
| | | | | | | | | | C | | | | | |
| ITI_ | DIQMTQT | 1047 | QGI---- | 1128 | LNWYQQK | 1176 | YTS | 1242 | SLHSGVP | 1279 | QQYSKLP | 1360 | FGGGTKL | 1427 |
| 16 | TSSLSAS | | --TKY | | PDGTVKL | | | | SRFSGSG | | WT | | EIK | |
| 9L | LGDRVTI | | | | LIY | | | | SGTDYSL | | | | | |
| | SCSAS | | | | | | | | TISNLEP | | | | | |
| | | | | | | | | | EDIATYY | | | | | |
| | | | | | | | | | C | | | | | |
| ITI_ | DIVLTQS | 1041 | QSVDY-- | 1106 | INWYQQR | 1191 | AAS | 1239 | NLESGIP | 1295 | QHCYEDP | 1373 | FGGGTKL | 1427 |
| 20 | PASLAVS | | DGESY | | PGQPAKL | | | | ARFSGSG | | WT | | EIK | |
| 0L | LGQRATI | | | | LIF | | | | SGTDFTL | | | | | |
| | SCKAS | | | | | | | | NIHPVEE | | | | | |
| | | | | | | | | | EDAASYY | | | | | |
| | | | | | | | | | C | | | | | |
| ITI_ | DIQMTQT | 1057 | QDI-SNY | 1110 | LNWYQQK | 1176 | YTS | 1242 | RLHSGVP | 1296 | QQGNTLP | 1374 | FGGGTKL | 1427 |
| 23 | TSSLSAS | | | | PDGTVKL | | | | SRFSGSG | | YT | | EIK | |
| 6L | LGDRVTI | | | | LIY | | | | SGTDYSL | | | | | |
| | SCRAS | | | | | | | | TISNLEQ | | | | | |
| | | | | | | | | | EDIATYF | | | | | |
| | | | | | | | | | C | | | | | |
| ITI_ | DIQMTQT | 1047 | QGI-SNY | 1114 | LNWYQQK | 1176 | YTS | 1242 | SLHSGVP | 1279 | QQYSKLP | 1375 | FGGGTKL | 1427 |
| 23 | TSSLSAS | | | | PDGTVKL | | | | SRFSGSG | | RT | | EIK | |
| 7L | LGDRVTI | | | | LIY | | | | SGTDYSL | | | | | |
| | SCSAS | | | | | | | | TISNLEP | | | | | |
| | | | | | | | | | EDIATYY | | | | | |
| | | | | | | | | | C | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #7

| | [D/E][V | | | | | | | | | | | | | |
|---|---|---|---|---|---|---|---|---|---|---|---|---|---|---|
| | /N][V/L | | | | | | | | | | | | | |
| | ][M/L]T | | | | | | | | [K/N][L | | | | | |
| | Q[T/S]P | | | | | | | | /R/V][D | | | | | |
| | [L/A][T | | | | | | | | /P/F/V] | | | | | |
| | /I/S][L | | | | | | | | SGVP[D/ | | | | | |
| | /M][S/P | | | | | | | | G]RF[T/ | | | | | |
| | /T][V/A | | | | | | | | S]G[S/N | | | | | |
| | /I][T/S | | | | | | | | ]GSG[T/ | | | | | |
| | /N][I/P | | [Q/S][S | | [L/M][N | | | | N][D/S/ | | | | | |
| | /L/V]G[ | | /N][L/V | | /H/E/Y/ | | | | Y/E][F/ | | [V/F/W] | | | |
| | Q/E/D/H | | /I][L/- | | S]W[L/Y | | | | Y][T/S] | | Q[G/A/N | | | |
| | ][P/K/Q | | /V][Y/- | | ][L/Q]Q | | | | L[K/T/N | | /L/V][T | | | |
| | /T][A/V | | /H/D][S | | [R/K][P | | | | ]IS[R/S | | /S][H/G | | | |
| | ][S/T][ | | /-][N/- | | /S][G/S | | | | ][V/M]E | | ][F/-][ | | [F/L]G[ | |
| | I/M][S/ | | /D][G/- | | ][Q/T]S | | | | AED[L/V | | P/F/V][ | | G/S/A/T | |
| | T]C[K/S | | ][K/-/N | | P[K/N/E | | | | ][G/A][ | | M/P][Y/ | | ]GT[K/N | |
| | /R][S/A | | /I][T/S | | ][R/L][ | | [L/D/K] | | V/T/I]Y | | L/W/H/Q | | /E]LE[I | |
| | ][S/G] | 1058 | ]Y | 1129 | L/W]IY | 1192 | [V/T]S | 1246 | YC | 1297 | ][T/A] | 1376 | /L]K | 1436 |
| H5S14- | DVVMTQT | 1059 | QSLLDSD | 1130 | LNWLLQR | 1193 | LVS | 1247 | KLDSGVP | 1298 | WQLTH-F | 1377 | FGGGTKL | 1427 |
| 11AL | PLTLSVT | | GKTY | | PGQSPKR | | | | DRFTGSG | | PQT | | EIK | |
| | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S14- | DVLMTQT | 1060 | QSIVHSN | 1131 | LEWYLĄK | 1194 | KVS | 1248 | NRFSGVP | 1299 | FQGSH-V | 1378 | FGGGTKL | 1427 |
| 13AL | PLSLPVS | | GNTY | | PGQSPKL | | | | DRFSGSG | | PWT | | EIK | |
| An | LGDQASI | | | | LIY | | | | SGTDFTL | | | | | |
| d | SCRSS | | | | | | | | KISRVEA | | | | | |
| H5S19 | | | | | | | | | EDLGVYY | | | | | |
| - | | | | | | | | | C | | | | | |
| 7AL | | | | | | | | | | | | | | |
| H5S14- | DVVMTQT | 1059 | QSLLDSD | 1130 | LNWLLQR | 1193 | LVS | 1247 | KLDSGVP | 1298 | WQVTH-F | 1379 | FGGGTKL | 1427 |
| 17AL | PLTLSVT | | GKTY | | PGQSPKR | | | | DRFTGSG | | PQT | | EIK | |
| | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S14- | DVVMTQT | 1059 | QSLLDSD | 1130 | LNWLLQR | 1193 | LVS | 1247 | KLDSGVP | 1298 | WQGTH-F | 1380 | FGGGTKL | 1427 |
| 2AL | PLTLSVT | | GKTY | | PGQSPKR | | | | DRFTGSG | | PHA | | EIK | |
| | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S15- | DVVMTQT | 1059 | QSLLDSD | 1130 | LNWLLQR | 1193 | LVS | 1247 | KLDSGVP | 1300 | WQGTH-F | 1381 | FGGGTKL | 1427 |
| 12CL | PLTLSVT | | GKTY | | PGQSPKR | | | | DRFTGSG | | PQT | | EIK | |
| | IGQPASI | | | | LIY | | | | SGTDFTL | | | | | |
| | SCKSS | | | | | | | | KISRVEA | | | | | |
| | | | | | | | | | EDLGIYY | | | | | |
| | | | | | | | | | C | | | | | |
| H5S15- | DVLMTQT | 1060 | QSIVHSN | 1132 | LEWYLQK | 1195 | KVS | 1248 | NRFSGVP | 1299 | FQGSH-V | 1382 | FGAGTKL | 1429 |
| 16AL | PLSLPVS | | GITY | | PGQSPEL | | | | DRFSGSG | | PLT | | ELK | |
| | LGDQASI | | | | LIY | | | | SGTDFTL | | | | | |
| | SCRSS | | | | | | | | KISRVEA | | | | | |
| | | | | | | | | | EDLGVYY | | | | | |
| | | | | | | | | | C | | | | | |
| H5S15- | DVVMTQT | 1061 | QSLLDSD | 1130 | LNWLLQR | 1193 | LVS | 1247 | KRDSGVP | 1301 | WQNTH-F | 1383 | FGGGTKL | 1427 |
| 17AL | PLTLSVT | | GKTY | | PGQSPKR | | | | DRFTGSG | | PQT | | EIK | |
| | VGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S15- | ENVLTQS | 1062 | SSV---- | 1133 | MHWYQQK | 1196 | DTS | 1233 | KLPSGVP | 1302 | FQGSG-F | 1384 | FGSGTKL | 1428 |
| 1AL | PAIMSAS | | --SY | | SSTSPKL | | | | GRFSGSG | | PLT | | EIK | |
| | PGEKVTM | | | | WIY | | | | SGNSYSL | | | | | |
| | TCSAG | | | | | | | | TISSMEA | | | | | |
| | | | | | | | | | EDVATYY | | | | | |
| | | | | | | | | | C | | | | | |
| H5S15- | DVVMTQT | 1059 | QSLLDSD | 1130 | LNWLLQR | 1193 | LVS | 1247 | KLDSGVP | 1298 | WQATH-F | 1385 | FGGGTNL | 1430 |
| 31AL | PLTLSVT | | GKTY | | PGQSPKR | | | | DRFTGSG | | PQT | | EIK | |
| | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S15- | DVLMTQT | 1060 | QSIVHSN | 1131 | LEWYLQK | 1194 | KVS | 1248 | NRFSGVP | 1299 | FQGSH-V | 1382 | FGAGTKL | 1429 |
| 3AL | PLSLPVS | | GNTY | | PGQSPKL | | | | DRFSGSG | | PLT | | ELK | |
| | LGDQASI | | | | LIY | | | | SGTDFTL | | | | | |
| | SCRSS | | | | | | | | KISRVEA | | | | | |
| | | | | | | | | | EDLGVYY | | | | | |
| | | | | | | | | | C | | | | | |
| H5S15- | DVLMTQT | 1063 | QSIVHSN | 1131 | LEWYLQK | 1194 | KVS | 1248 | NRFSGVP | 1299 | FQGSH-V | 1382 | FGAGTKL | 1429 |
| 3BL | PLSLTVS | | GNTY | | PGQSPKL | | | | DRFSGSG | | PLT | | ELK | |
| | LGHQASI | | | | LIY | | | | SGTDFTL | | | | | |
| | SCRSS | | | | | | | | KISRVEA | | | | | |
| | | | | | | | | | EDLGVYY | | | | | |
| | | | | | | | | | C | | | | | |
| H5S15- | DVVMTQT | 1059 | QSLLDSD | 1130 | LNWLLQR | 1193 | LVS | 1247 | KLDSGVP | 1303 | WQNTH-F | 1383 | FGGGTKL | 1427 |
| 6AL | PLTLSVT | | GKTY | | PGQSPKR | | | | DRFTGSG | | PQT | | EIK | |
| | IGQPASI | | | | LIY | | | | SGTEFTL | | | | | |
| | SCKSS | | | | | | | | KISRVEA | | | | | |
| | | | | | | | | | EDLGVYY | | | | | |
| | | | | | | | | | C | | | | | |
| H5S20- | DVVMTQT | 1059 | QSLLDSD | 1130 | LSWLLQR | 1197 | LVS | 1247 | KLDSGVP | 1298 | WQNTH-F | 1383 | FGGGTKL | 1427 |
| 19AL | PLTLSVT | | GKTY | | PGQSPKR | | | | DRFTGSG | | PQT | | EIK | |
| | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S20- | DVVMTQT | 1064 | QSLLDSD | 1130 | LSWLLQR | 1197 | LVS | 1247 | KLDSGVP | 1298 | WQNTH-F | 1383 | FGGGTKL | 1427 |
| 19BL | PLSLSVT | | GKTY | | PGQSPKR | | | | DRFTGSG | | PQT | | EIK | |
| | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S20- | DVLMTQT | 1060 | QSIVHSN | 1131 | LEWYLQK | 1194 | KVS | 1248 | NRVSGVP | 1304 | FQGSH-V | 1382 | FGAGTKL | 1429 |
| 21AL | PLSLPVS | | GNTY | | PGQSPKL | | | | DRFSGSG | | PLT | | ELK | |
| | LGDQASI | | | | LIY | | | | SGTDFTL | | | | | |
| | SCRSS | | | | | | | | KISRVEA | | | | | |
| | | | | | | | | | EDLGVYY | | | | | |
| | | | | | | | | | C | | | | | |
| H5S20- | DVVMTQT | 1059 | QSLLDSD | 1130 | LNWLLQR | 1193 | LVS | 1247 | KLDSGVP | 1298 | WQNTH-F | 1383 | LGGGTKL | 1437 |
| 24AL | PLTLSVT | | GKTY | | PGQSPKR | | | | DRFTGSG | | PQT | | EIK | |
| | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S20- | DVVMTQT | 1059 | QSLLDSD | 1130 | LNWLLQR | 1193 | LVS | 1247 | KLDSGVP | 1298 | WQGTH-F | 1381 | FGGGTKL | 1427 |
| 25AL | PLTLSVT | | GKTY | | PGQSPKR | | | | DRFTGSG | | PQT | | EIK | |
| | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S20- | DVVMTQT | 1059 | QSLLDSD | 1130 | LYWLLQR | 1198 | LVS | 1247 | KLDSGVP | 1305 | WQNTH-F | 1383 | FGGGTKL | 1427 |
| 26AL | PLTLSVT | | GKTY | | PGQSPKR | | | | DRFTGSG | | PQT | | EIK | |
| | IGQPASI | | | | LIY | | | | SGTYFTL | | | | | |
| | SCKSS | | | | | | | | KISRVEA | | | | | |
| | | | | | | | | | EDLGVYY | | | | | |
| | | | | | | | | | C | | | | | |
| H5S20- | DVVMTQT | 1059 | QSLLDSD | 1130 | LNWLLQR | 1193 | LVS | 1247 | KVDSGVP | 1306 | WQNTH-F | 1383 | FGGGTKL | 1427 |
| 31AL | PLTLSVT | | GKTY | | PGQSPKR | | | | DRFTGSG | | PQT | | EIK | |
| | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S20- | DVLMTQT | 1065 | QNIVHSN | 1134 | LEWYLĄK | 1194 | KVS | 1248 | NRFSGVP | 1299 | FQGSH-V | 1382 | FGAGTKL | 1429 |
| 39AL | PLSLPVS | | GNTY | | PGQSPKL | | | | DRFSGSG | | PLT | | ELK | |
| | LGDQVSI | | | | LIY | | | | SGTDFTL | | | | | |
| | SCRSS | | | | | | | | KISRVEA | | | | | |
| | | | | | | | | | EDLGVYY | | | | | |
| | | | | | | | | | C | | | | | |
| H5S20- | DVVMTQT | 1059 | QSLLDSD | 1130 | LNWLLQR | 1193 | LVS | 1247 | KLDSGVP | 1298 | WQATH-F | 1385 | FGGGTKL | 1427 |
| 3BL | PLTLSVT | | GKTY | | PGQSPKR | | | | DRFTGSG | | PQT | | EIK | |
| | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S20- | DVVMTQT | 1059 | QSLLYSN | 1135 | LNWLLQR | 1193 | LVS | 1247 | KLDSGVP | 1298 | VQGTHFP | 1386 | FGGGTKL | 1427 |
| 41AL | PLTLSVT | | GKTY | | PGQSPKR | | | | DRFTGSG | | MYT | | EIK | |
| | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S20- | DVVMTQT | 1059 | QSLLDSD | 1130 | LNWLLQR | 1193 | LVS | 1247 | KLDSGVP | 1298 | WQNTH-F | 1383 | FGGGTKL | 1427 |
| 7AL | PLTLSVT | | GKTY | | PGQSPKR | | | | DRFTGSG | | PQT | | EIK | |
| | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S20- | DVVMTQT | 1059 | QSLLDSD | 1130 | LNWLLQR | 1193 | LVS | 1247 | KLDSGVP | 1300 | WQNTH-F | 1383 | FGGGTKL | 1427 |
| 7BL | PLTLSVT | | GKTY | | PGQSPKR | | | | DRFTGSG | | PQT | | EIK | |
| | IGQPASI | | | | LIY | | | | SGTDFTL | | | | | |
| | SCKSS | | | | | | | | KISRVEA | | | | | |
| | | | | | | | | | EDLGIYY | | | | | |
| | | | | | | | | | C | | | | | |
| H5S20- | DVVMTQT | 1066 | QSLLDSD | 1130 | LNWLLQR | 1193 | LVS | 1247 | KLDSGVP | 1298 | WQNTH-F | 1383 | FGGGTKL | 1427 |
| 7CL | PLTLSIT | | GKTY | | PGQSPKR | | | | DRFTGSG | | PQT | | EIK | |
| | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S20- | DVVMTQT | 1059 | QSLLDSD | 1130 | LNWLLQR | 1193 | LVS | 1247 | KLDSGVP | 1298 | WQNTH-F | 1383 | FGGGTEL | 1438 |
| 7DL | PLTLSVT | | GKTY | | PGQSPKR | | | | DRFTGSG | | PQT | | EIK | |
| | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S20- | DVVMTQT | 1067 | QSLLDSD | 1130 | LNWLLQR | 1193 | LVS | 1247 | KLDSGVP | 1298 | WQNTH-F | 1383 | FGGGTKL | 1427 |
| 7EL | PLTLSVT | | GKTY | | PGQSPKR | | | | DRFTGSG | | PQT | | EIK | |
| | IGQTASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| H5S20- | DVLMTQT | 1068 | QSIVHSN | 1131 | LEWYLĄK | 1194 | KVS | 1248 | NRFSGVP | 1299 | FQGSH-V | 1382 | FGAGTKL | 1429 |
| 9AL | PLSLPVN | | GNTY | | PGQSPKL | | | | DRFSGSG | | PLT | | ELK | |
| | LGDQASI | | | | LIY | | | | SGTDFTL | | | | | |
| | SCRSS | | | | | | | | KISRVEA | | | | | |
| | | | | | | | | | EDLGVYY | | | | | |
| | | | | | | | | | C | | | | | |
| H5S20- | DVLMTQT | 1060 | QSIVHSN | 1131 | LEWYLĄK | 1194 | KVS | 1248 | NRFSGVP | 1299 | FQGSH-V | 1382 | FGTGTKL | 1439 |
| 9DL | PLSLPVS | | GNTY | | PGQSPKL | | | | DRFSGSG | | PLT | | ELK | |
| | LGDQASI | | | | LIY | | | | SGTDFTL | | | | | |
| | SCRSS | | | | | | | | KISRVEA | | | | | |
| | | | | | | | | | EDLGVYY | | | | | |
| | | | | | | | | | C | | | | | |
| H5S20- | DVLMTQT | 1060 | QSIVHSN | 1131 | LEWYLQK | 1199 | KVS | 1248 | NRFSGVP | 1299 | FQGSH-V | 1382 | FGTGTKL | 1439 |
| 9EL | PLSLPVS | | GNTY | | PGQSPNL | | | | DRFSGSG | | PLT | | ELK | |
| | LGDQASI | | | | LIY | | | | SGTDFTL | | | | | |
| | SCRSS | | | | | | | | KISRVEA | | | | | |
| | | | | | | | | | EDLGVYY | | | | | |
| | | | | | | | | | C | | | | | |
| ITI_ | DVVMTQT | 1059 | QSLLDSD | 1130 | LNWLLQR | 1193 | LVS | 1247 | KLDSGVP | 1307 | WQATH-F | 1385 | FGGGTKL | 1427 |
| 04 | PLTLSVT | | GKTY | | PGQSPKR | | | | DRFTGNG | | PQT | | EIK | |
| 7L | IGQPASI | | | | LIY | | | | SGTDFTL | | | | | |
| | SCKSS | | | | | | | | KISRVEA | | | | | |
| | | | | | | | | | EDLGVYY | | | | | |
| | | | | | | | | | C | | | | | |
| ITI_ | DVLMTQT | 1060 | QSIVHSN | 1131 | LEWYLQK | 1194 | KVS | 1248 | NRFSGVP | 1308 | FQGSH-V | 1382 | FGAGTKL | 1429 |
| 09 | PLSLPVS | | GNTY | | PGQSPKL | | | | DRFSGSG | | PLT | | ELK | |
| 1L | LGDQASI | | | | LIY | | | | SGTDFTL | | | | | |
| | SCRSS | | | | | | | | NISRVEA | | | | | |
| | | | | | | | | | EDLGVYY | | | | | |
| | | | | | | | | | C | | | | | |

Consensus Cluster #3

| | | | | | | | | | NLASGVP | | | | | |
|---|---|---|---|---|---|---|---|---|---|---|---|---|---|---|
| | | | | | | | | | [S/A/V] | | | | | |
| | | | | | | | | | RFSGSGS | | | | | |
| | QIVLTQS | | | | [M/L][H | | | | GT[F/S] | | | | | |
| | PAI[M/V | | | | /F/Y/Q] | | | | [Y/F][S | | HQW[S/N | | | |
| | /I]S[A/ | | | | W[Y/F]Q | | | | /Y]LT[I | | ][S/T/N | | | |
| | T]S[L/P | | | | Q[K/R][ | | | | /L]S[S/ | | /-][Y/F | | FG[G/A] | |
| | ]G[E/A] | | S[S/T]V | | S/P]G[S | | | | G][V/M] | | /S][T/L | | G[T/A][ | |
| | [E/K][I | | [-/D/S] | | /T]SPKL | | | | EAEDAA[ | | /A/P/Y] | | K/I/M/E | |
| | /V]TLTC | | [-/S]S[ | | [L/W][I | | [T/S/G] | | D/S]Y[Y | | [W/R/L/ | | ]LE[I/L | |
| | SA[S/R] | 1069 | Y/F] | 1136 | /L]Y | 1200 | [T/A]S | 1249 | /F]C | 1309 | P]T | 1387 | ][K/-] | 1440 |
| H5S14- | QIVLTQS | 1070 | SSV--SY | 1137 | MQWYQQK | 1201 | STS | 1241 | NLASGVP | 1310 | HQWS-SY | 1388 | FGGGTEL | 1441 |
| 3AL | PAIMSAS | | | | SGTSPKL | | | | SRFSGSG | | PT | | EI- | |
| | LGEEITL | | | | LIY | | | | SGTFYSL | | | | | |
| | TCSAS | | | | | | | | TISSVEA | | | | | |
| | | | | | | | | | EDAADYY | | | | | |
| | | | | | | | | | C | | | | | |
| H5S14- | QIVLTQS | 1071 | SSV--SY | 1137 | MHWYQQK | 1202 | STS | 1241 | NLASGVP | 1310 | HQWS-SY | 1389 | FGAGTKL | 1429 |
| 5AL | PAIISAS | | | | SGTSPKL | | | | SRFSGSG | | LT | | ELK | |
| | LGEEVTL | | | | LIY | | | | SGTFYSL | | | | | |
| | TCSAS | | | | | | | | TISSVEA | | | | | |
| | | | | | | | | | EDAADYY | | | | | |
| | | | | | | | | | C | | | | | |
| H5S15- | QIVLTQS | 1070 | SSV--SF | 1138 | MHWYQQK | 1202 | STS | 1241 | NLASGVP | 1310 | HQWSSYL | 1390 | FGGGAKL | 1431 |
| 8AL | PAIMSAS | | | | SGTSPKL | | | | SRFSGSG | | WT | | EIK | |
| | LGEEITL | | | | LIY | | | | SGTFYSL | | | | | |
| | TCSAS | | | | | | | | TISSVEA | | | | | |
| | | | | | | | | | EDAADYY | | | | | |
| | | | | | | | | | C | | | | | |
| H5S19- | QIVLTQS | 1072 | SSVDSSY | 1139 | LFWYQQK | 1203 | STS | 1241 | NLASGVP | 1311 | HQWSSYP | 1391 | FGGGTIL | 1442 |
| 10AL | PAIMSAS | | | | PGSSPKL | | | | ARFSGSG | | WT | | EIK | |
| | PGEKVTL | | | | WIY | | | | SGTSYSL | | | | | |
| | TCSAS | | | | | | | | TISSMEA | | | | | |
| | | | | | | | | | EDAASYF | | | | | |
| | | | | | | | | | C | | | | | |
| H5S19- | QIVLTQS | 1072 | SSVDSSY | 1139 | LFWYQQK | 1203 | STS | 1241 | NLASGVP | 1311 | HQWSSYP | 1391 | FGGGTIL | 1443 |
| 10BL | PAIMSAS | | | | PGSSPKL | | | | ARFSGSG | | WT | | EI1 | |
| | PGEKVTL | | | | WIY | | | | SGTSYSL | | | | | |
| | TCSAS | | | | | | | | TISSMEA | | | | | |
| | | | | | | | | | EDAASYF | | | | | |
| | | | | | | | | | C | | | | | |
| H5S19- | QIVLTQS | 1072 | SSVDSSY | 1139 | LFWYQQK | 1203 | STS | 1241 | NLASGVP | 1312 | HQWSNFA | 1392 | FGGGTIL | 1442 |
| 14AL | PAIMSAS | | | | PGSSPKL | | | | ARFSGSG | | WT | | EIK | |
| | PGEKVTL | | | | WIY | | | | SGTSYYL | | | | | |
| | TCSAS | | | | | | | | TLSSMEA | | | | | |
| | | | | | | | | | EDAASYF | | | | | |
| | | | | | | | | | C | | | | | |
| H5S19- | QIVLTQS | 1072 | SSVDSSY | 1139 | LYWFQQK | 1204 | GTS | 1243 | NLASGVP | 1311 | HQWNSYP | 1393 | FGGGTKL | 1427 |
| 16AL | PAIMSAS | | | | PGSSPKL | | | | ARFSGSG | | WT | | EIK | |
| | PGEKVTL | | | | WIY | | | | SGTSYSL | | | | | |
| | TCSAS | | | | | | | | TISSMEA | | | | | |
| | | | | | | | | | EDAASYF | | | | | |
| | | | | | | | | | C | | | | | |
| H5S19- | QIVLTQS | 1072 | SSVDSSY | 1139 | LYWYQQK | 1205 | GTS | 1243 | NLASGVP | 1311 | HQWNSYP | 1393 | FGGGTKL | 1427 |
| 16BL | PAIMSAS | | | | PGSSPKL | | | | ARFSGSG | | WT | | EIK | |
| | PGEKVTL | | | | WIY | | | | SGTSYSL | | | | | |
| | TCSAS | | | | | | | | TISSMEA | | | | | |
| | | | | | | | | | EDAASYF | | | | | |
| | | | | | | | | | C | | | | | |
| H5S19- | QIVLTQS | 1073 | SSVSSSY | 1140 | LYWYQQK | 1205 | STS | 1241 | NLASGVP | 1311 | HQWSTYP | 1394 | FGGGTKL | 1427 |
| 18AL | PAIMSTS | | | | PGSSPKL | | | | ARFSGSG | | WT | | EIK | |
| | PGEKVTL | | | | WIY | | | | SGTSYSL | | | | | |
| | TCSAS | | | | | | | | TISSMEA | | | | | |
| | | | | | | | | | EDAASYF | | | | | |
| | | | | | | | | | C | | | | | |
| H5S19- | QIVLTQS | 1072 | SSVSSSY | 1140 | LYWYQQK | 1205 | STS | 1241 | NLASGVP | 1311 | HQWSSYP | 1391 | FGGGTKL | 1427 |
| 19AL | PAIMSAS | | | | PGSSPKL | | | | ARFSGSG | | WT | | EIK | |
| | PGEKVTL | | | | WIY | | | | SGTSYSL | | | | | |
| | TCSAS | | | | | | | | TISSMEA | | | | | |
| | | | | | | | | | EDAASYF | | | | | |
| | | | | | | | | | C | | | | | |
| H5S19- | QIVLTQS | 1072 | SSVDSSY | 1139 | LFWYQQK | 1203 | STS | 1241 | NLASGVP | 1311 | HQWNSYP | 1393 | FGGGTKL | 1427 |
| 3AL | PAIMSAS | | | | PGSSPKL | | | | ARFSGSG | | WT | | EIK | |
| | PGEKVTL | | | | WIY | | | | SGTSYSL | | | | | |
| | TCSAS | | | | | | | | TISSMEA | | | | | |
| | | | | | | | | | EDAASYF | | | | | |
| | | | | | | | | | C | | | | | |
| H5S19- | QIVLTQS | 1072 | SSVDSSY | 1139 | LFWYQQK | 1203 | STS | 1241 | NLASGVP | 1313 | HQWSSYA | 1395 | FGGGTIL | 1442 |
| 4AL | PAIMSAS | | | | PGSSPKL | | | | ARFSGSG | | WT | | EIK | |
| | PGEKVTL | | | | WIY | | | | SGTSYYL | | | | | |
| | TCSAS | | | | | | | | TISSMEA | | | | | |
| | | | | | | | | | EDAASYF | | | | | |
| | | | | | | | | | C | | | | | |
| H5S19- | QIVLTQS | 1072 | SSVDSSY | 1139 | LFWYQQK | 1206 | STS | 1241 | NLASGVP | 1313 | HQWSNYA | 1396 | FGGGTIL | 1442 |
| 5AL | PAIMSAS | | | | PGSSPKL | | | | ARFSGSG | | WT | | EIK | |
| | PGEKVTL | | | | WLY | | | | SGTSYYL | | | | | |
| | TCSAS | | | | | | | | TISSMEA | | | | | |
| | | | | | | | | | EDAASYF | | | | | |
| | | | | | | | | | C | | | | | |
| H5S20- | QIVLTQS | 1070 | SSV--SY | 1137 | MHWYQQR | 1207 | STS | 1241 | NLASGVP | 1310 | HQWS-SY | 1397 | FGGGTKL | 1427 |
| 10AL | PAIMSAS | | | | SGTSPKL | | | | SRFSGSG | | RT | | EIK | |
| | LGEEITL | | | | LIY | | | | SGTFYSL | | | | | |
| | TCSAS | | | | | | | | TISSVEA | | | | | |
| | | | | | | | | | EDAADYY | | | | | |
| | | | | | | | | | C | | | | | |
| H5S20- | QIVLTQS | 1070 | SSV--SY | 1137 | MHWYQQK | 1202 | SAS | 1240 | NLASGVP | 1310 | HQWS-SY | 1397 | FGGGTKL | 1427 |
| 15AL | PAIMSAS | | | | SGTSPKL | | | | SRFSGSG | | RT | | EIK | |
| | LGEEITL | | | | LIY | | | | SGTFYSL | | | | | |
| | TCSAS | | | | | | | | TISSVEA | | | | | |
| | | | | | | | | | EDAADYY | | | | | |
| | | | | | | | | | C | | | | | |
| H5S20- | QIVLTQS | 1070 | SSV--SY | 1137 | MHWYQQK | 1208 | TTS | 1250 | NLASGVP | 1310 | HQWSSYT | 1398 | FGGGTKL | 1427 |
| 22AL | PAIMSAS | | | | SGSSPKL | | | | SRFSGSG | | WT | | EIK | |
| | LGEEITL | | | | LIY | | | | SGTFYSL | | | | | |
| | TCSAS | | | | | | | | TISSVEA | | | | | |
| | | | | | | | | | EDAADYY | | | | | |
| | | | | | | | | | C | | | | | |
| ITI_ | QIVLTQS | 1072 | SSVDSSY | 1139 | LFWYQQK | 1203 | STS | 1241 | NLASGVP | 1311 | HQWSSYP | 1391 | FGGGTKL | 1427 |
| 02 | PAIMSAS | | | | PGSSPKL | | | | ARFSGSG | | WT | | EIK | |
| 7L | PGEKVTL | | | | WIY | | | | SGTSYSL | | | | | |
| | TCSAS | | | | | | | | TISSMEA | | | | | |
| | | | | | | | | | EDAASYF | | | | | |
| | | | | | | | | | C | | | | | |
| ITI_ | QIVLTQS | 1072 | SSVDSSY | 1139 | LYWYQQK | 1205 | STS | 1241 | NLASGVP | 1311 | HQWNSYP | 1393 | FGGGTKL | 1427 |
| 02 | PAIMSAS | | | | PGSSPKL | | | | ARFSGSG | | WT | | EIK | |
| 8L | PGEKVTL | | | | WIY | | | | SGTSYSL | | | | | |
| | TCSAS | | | | | | | | TISSMEA | | | | | |
| | | | | | | | | | EDAASYF | | | | | |
| | | | | | | | | | C | | | | | |
| ITI_ | QIVLTQS | 1072 | SSVDSSY | 1139 | LFWYQQK | 1203 | STS | 1241 | NLASGVP | 1311 | HQWSSYA | 1395 | FGGGTIL | 1442 |
| 02 | PAIMSAS | | | | PGSSPKL | | | | ARFSGSG | | WT | | EIK | |
| 9L | PGEKVTL | | | | WIY | | | | SGTSYSL | | | | | |
| | TCSAS | | | | | | | | TISSMEA | | | | | |
| | | | | | | | | | EDAASYF | | | | | |
| | | | | | | | | | C | | | | | |
| ITI_ | QIVLTQS | 1072 | SSVDSSY | 1139 | LYWFQQK | 1204 | GTS | 1243 | NLASGVP | 1314 | HQWNSYP | 1393 | FGGGTKL | 1427 |
| 03 | PAIMSAS | | | | PGSSPKL | | | | VRFSGSG | | WT | | EIK | |
| 0L | PGEKVTL | | | | WIY | | | | SGTSYSL | | | | | |
| | TCSAS | | | | | | | | TISSMEA | | | | | |
| | | | | | | | | | EDAASYF | | | | | |
| | | | | | | | | | C | | | | | |
| ITI_ | QIVLTQS | 1072 | SSVDSSY | 1139 | LFWYQQK | 1203 | STS | 1241 | NLASGVP | 1311 | HQWSSYP | 1391 | FGGGTML | 1444 |
| 03 | PAIMSAS | | | | PGSSPKL | | | | ARFSGSG | | WT | | EIK | |
| 3L | PGEKVTL | | | | WIY | | | | SGTSYSL | | | | | |
| | TCSAS | | | | | | | | TISSMEA | | | | | |
| | | | | | | | | | EDAASYF | | | | | |
| | | | | | | | | | C | | | | | |
| ITI_ | QIVLTQS | 1072 | SSVDSSY | 1139 | LYWYQQK | 1205 | GTS | 1243 | NLASGVP | 1314 | HQWNSYP | 1393 | FGGGTKL | 1427 |
| 03 | PAIMSAS | | | | PGSSPKL | | | | VRFSGSG | | WT | | EIK | |
| 8L | PGEKVTL | | | | WIY | | | | SGTSYSL | | | | | |
| | TCSAS | | | | | | | | TISSMEA | | | | | |
| | | | | | | | | | EDAASYF | | | | | |
| | | | | | | | | | C | | | | | |
| ITI_ | QIVLTQS | 1074 | SSV--SY | 1137 | MFWYQQK | 1209 | TTS | 1250 | NLASGVP | 1315 | HQWSSYT | 1398 | FGGGTKL | 1427 |
| 12 | PAIMSAS | | | | SGTSPKL | | | | SRFSGSG | | WT | | EIK | |
| 7L | LGEEITL | | | | LIY | | | | SGTFFSL | | | | | |
| | TCSAR | | | | | | | | TISGVEA | | | | | |
| | | | | | | | | | EDAADYY | | | | | |
| | | | | | | | | | C | | | | | |
| ITI_ | QIVLTQS | 1075 | STV--SY | 1141 | MFWYQQK | 1209 | STS | 1241 | NLASGVP | 1316 | HQWSSYT | 1398 | FGGGTKL | 1427 |
| 12 | PAIVSAS | | | | SGTSPKL | | | | SRFSGSG | | WT | | EIK | |
| 8L | LGAEITL | | | | LIY | | | | SGTFYSL | | | | | |
| | TCSAR | | | | | | | | TISGVEA | | | | | |
| | | | | | | | | | EDAADYY | | | | | |
| | | | | | | | | | C | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #5

| | | | | | | | | | [N/T/R] | | | | | |
|---|---|---|---|---|---|---|---|---|---|---|---|---|---|---|
| | | | | | | | | | [L/R/G] | | | | | |
| | | | | | | | | | [A/D/E/ | | | | | |
| | [Q/D]I[ | | | | | | | | G/V][S/ | | | | | |
| | V/Q/L/K | | | | | | | | D]GVP[A | | | | | |
| | ][L/M][ | | | | | | | | /K/D/S] | | | | | |
| | T/S]QSP | | | | | | | | RF[S/T] | | | | | |
| | [A/S][I | | | | | | | | GS[G/R] | | | | | |
| | /S][M/L | | | | | | | | SG[T/S/ | | | | | |
| | ][S/A/P | | [S/Q][S | | [L/I][H | | | | A/Q][S/ | | | | | |
| | /G/Y][A | | /E/G/D] | | /S/A/G/ | | | | D][F/Y] | | | | | |
| | /V]S[L/ | | [V/I/L] | | N]W[Y/L | | | | [S/T]LT | | | | | |
| | V]G[E/D | | [S/-/L] | | /F]QQ[K | | | | IS[S/N] | | [H/L/Q/ | | | |
| | ][R/K/T | | [-/F/Y] | | /R]P[G/ | | | | [M/L/V] | | G]QY[H/ | | | |
| | /S][V/I | | [-/G/S] | | D][S/G/ | | | | [E/K][A | | A/Y/G/D | | | |
| | ][T/S/N | | [-/S][- | | Q/K][S/ | | | | /S/Y/F] | | ][-/R/S | | | |
| | ][M/L/I | | /N/D/I] | | T][P/I/ | | [N/D/A/ | | ED[A/F/ | | /T/Q/E] | | | |
| | ][T/S]C | | [-/Q][S | | F]K[L/R | | W/H/R][ | | L/M][A/ | | [R/S/Y/ | | FG[G/S] | |
| | [T/R/K/ | | /K/N][S | | /G/T][W | | T/A/S/G | | G][T/D/ | | F][S/Q/ | | GTKLE[I | |
| | H][A/S] | | /G/N][Y | | /L][I/V | | ][S/T/N | | V/I]Y[Y | | P][P/W/ | | /M][K/- | |
| | S | 1076 | /N] | 1142 | ]Y | 1210 | ] | 1251 | /F]C | 1317 | F/R/Y]T | 1399 | ] | 1445 |
| H5S14- | DIVMSQS | 1077 | QSLLYSS | 1143 | LAWYQQK | 1211 | WAS | 1245 | TRESGVP | 1318 | QQYYSYP | 1400 | FGGGTKL | 1427 |
| 21AL | PSSLAVS | | NQKNY | | PGQSPKL | | | | DRFTGSG | | WT | | EIK | |
| | VGEKVTM | | | | LIY | | | | SGTDFTL | | | | | |
| | SCKSS | | | | | | | | TISSVKA | | | | | |
| | | | | | | | | | EDLAVYY | | | | | |
| | | | | | | | | | C | | | | | |
| H5S14- | DIQMTQS | 1078 | QEI---- | 1144 | LSWLQQK | 1212 | AAS | 1239 | TLDSGVP | 1319 | LQYASYP | 1401 | FGSGTKL | 1428 |
| 22AL | PSSLSAS | | --SGY | | PDGTIKR | | | | KRFSGSR | | FT | | EIK | |
| | LGERVSL | | | | LIY | | | | SGSDYSL | | | | | |
| | TCRAS | | | | | | | | TISSLES | | | | | |
| | | | | | | | | | EDFADYY | | | | | |
| | | | | | | | | | C | | | | | |
| H5S14- | DIKMTQS | 1079 | QDI---- | 1145 | LSWFQQK | 1213 | RAN | 1252 | RLVDGVP | 1320 | LQYDEFP | 1402 | FGGGTKL | 1427 |
| 23AL | PSSMYAS | | --NSY | | PGKSPKT | | | | SRFSGSG | | WT | | EIK | |
| | LGERVTI | | | | LIY | | | | SGQDYSL | | | | | |
| | TCKAS | | | | | | | | TISSLEY | | | | | |
| | | | | | | | | | EDMGIYY | | | | | |
| | | | | | | | | | C | | | | | |
| H5S15- | QIVLTQS | 1080 | SSVS--- | 1108 | LHWYQQK | 1214 | DTS | 1233 | NLASGVP | 1268 | HQYHRSQ | 1403 | FGGGTKL | 1427 |
| 19AL | PAIMSAS | | --SSY | | PGSSPKL | | | | ARFSGSG | | WT | | EIK | |
| | LGERVTL | | | | WIY | | | | SGTSYSL | | | | | |
| | TCTAS | | | | | | | | TISSMEA | | | | | |
| | | | | | | | | | EDAATYY | | | | | |
| | | | | | | | | | C | | | | | |
| H5S15- | DIVMSQS | 1077 | QSLLYSS | 1143 | LAWYQQK | 1211 | WAS | 1245 | TRESGVP | 1318 | QQYYSYP | 1404 | FGGGTKL | 1427 |
| 21AL | PSSLAVS | | NQKNY | | PGQSPKL | | | | DRFTGSG | | YT | | EIK | |
| | VGEKVTM | | | | LIY | | | | SGTDFTL | | | | | |
| | SCKSS | | | | | | | | TISSVKA | | | | | |
| | | | | | | | | | EDLAVYY | | | | | |
| | | | | | | | | | C | | | | | |
| H5S15- | DIVMSQS | 1077 | QSLLYSS | 1143 | LAWYQQK | 1211 | WAS | 1245 | TRESGVP | 1318 | QQYYSYP | 1405 | FGGGTKL | 1427 |
| 26AL | PSSLAVS | | NQKNY | | PGQSPKL | | | | DRFTGSG | | RT | | EIK | |
| | VGEKVTM | | | | LIY | | | | SGTDFTL | | | | | |
| | SCKSS | | | | | | | | TISSVKA | | | | | |
| | | | | | | | | | EDLAVYY | | | | | |
| | | | | | | | | | C | | | | | |
| H5S19- | DIKMTQS | 1081 | QDI---- | 1145 | LNWFQQK | 1215 | RAN | 1252 | RLVDGVP | 1321 | LQYDEFP | 1406 | FGSGTKL | 1446 |
| 15AL | PSSMYAS | | --NSY | | PGKSPKT | | | | SRFSGSG | | FT | | EMK | |
| | LGESVTI | | | | LIY | | | | SGQDYSL | | | | | |
| | TCKAS | | | | | | | | TISSLEF | | | | | |
| | | | | | | | | | EDMGIYY | | | | | |
| | | | | | | | | | C | | | | | |
| H5S19- | DIKMTQS | 1081 | QDI---- | 1145 | LSWFQQK | 1213 | RAN | 1252 | RLVDGVP | 1321 | LQYDEFP | 1406 | FGSGTKL | 1446 |
| 9BL | PSSMYAS | | --NSY | | PGKSPKT | | | | SRFSGSG | | FT | | EMK | |
| | LGESVTI | | | | LIY | | | | SGQDYSL | | | | | |
| | TCKAS | | | | | | | | TISSLEF | | | | | |
| | | | | | | | | | EDMGIYY | | | | | |
| | | | | | | | | | C | | | | | |
| H5S20- | DIVMSQS | 1077 | QSLLYSS | 1143 | LAWYQQK | 1211 | WAS | 1245 | TRESGVP | 1318 | QQYYSYP | 1407 | FGSGTKL | 1428 |
| 12AL | PSSLAVS | | NQKNY | | PGQSPKL | | | | DRFTGSG | | FT | | EIK | |
| | VGEKVTM | | | | LIY | | | | SGTDFTL | | | | | |
| | SCKSS | | | | | | | | TISSVKA | | | | | |
| | | | | | | | | | EDLAVYY | | | | | |
| | | | | | | | | | C | | | | | |
| H5S20- | DIVMSQS | 1077 | QSLLYSS | 1143 | LAWYQQR | 1216 | WAS | 1245 | TRESGVP | 1318 | QQYYSYP | 1407 | FGSGTKL | 1428 |
| 12BL | PSSLAVS | | NQKNY | | PGQSPKL | | | | DRFTGSG | | FT | | EIK | |
| | VGEKVTM | | | | LIY | | | | SGTDFTL | | | | | |
| | SCKSS | | | | | | | | TISSVKA | | | | | |
| | | | | | | | | | EDLAVYY | | | | | |
| | | | | | | | | | C | | | | | |
| H5S20- | DIVMSQS | 1077 | QSLLYSS | 1146 | LAWYQQK | 1211 | WAS | 1245 | TRESGVP | 1318 | QQYYSYP | 1407 | FGSGTKL | 1428 |
| 1AL | PSSLAVS | | DQKNY | | PGQSPKL | | | | DRFTGSG | | FT | | EIK | |
| | VGEKVTM | | | | LIY | | | | SGTDFTL | | | | | |
| | SCKSS | | | | | | | | TISSVKA | | | | | |
| | | | | | | | | | EDLAVYY | | | | | |
| | | | | | | | | | C | | | | | |
| H5S20- | DIVMSQS | 1082 | QSLLYSS | 1146 | LAWYQQK | 1211 | WAS | 1245 | TRESGVP | 1318 | QQYYSYP | 1407 | FGSGTKL | 1428 |
| 1BL | PSSLGVS | | DQKNY | | PGQSPKL | | | | DRFTGSG | | FT | | EIK | |
| | VGEKVTM | | | | LIY | | | | SGTDFTL | | | | | |
| | SCKSS | | | | | | | | TISSVKA | | | | | |
| | | | | | | | | | EDLAVYY | | | | | |
| | | | | | | | | | C | | | | | |
| H5S20- | DIVMSQS | 1077 | QSLLYSS | 1146 | LAWYQQK | 1211 | WAS | 1245 | TGESGVP | 1322 | QQYYSYP | 1407 | FGSGTKL | 1428 |
| 27AL | PSSLAVS | | DQKNY | | PGQSPKL | | | | DRFTGSG | | FT | | EIK | |
| | VGEKVTM | | | | LIY | | | | SGTDFTL | | | | | |
| | SCKSS | | | | | | | | TISSVKA | | | | | |
| | | | | | | | | | EDLAVYY | | | | | |
| | | | | | | | | | C | | | | | |
| H5S20- | DIVMSQS | 1077 | QSLLYSS | 1146 | LAWYQQK | 1211 | WAS | 1245 | TRGSGVP | 1323 | QQYYSYP | 1407 | FGSGTKL | 1428 |
| 28AL | PSSLAVS | | DQKNY | | PGQSPKL | | | | DRFTGSG | | FT | | EIK | |
| | VGEKVTM | | | | LIY | | | | SGTDFTL | | | | | |
| | SCKSS | | | | | | | | TISSVKA | | | | | |
| | | | | | | | | | EDLAVYY | | | | | |
| | | | | | | | | | C | | | | | |
| H5S20- | DIVMSQS | 1083 | QSLLFSS | 1147 | LAWYQQK | 1211 | WAS | 1245 | TRESGVP | 1318 | QQYYSYP | 1407 | FGSGTKL | 1428 |
| 29AL | PSSLAVS | | NQKNY | | PGQSPKL | | | | DRFTGSG | | FT | | EIK | |
| | VGEKITM | | | | LIY | | | | SGTDFTL | | | | | |
| | SCKSS | | | | | | | | TISSVKA | | | | | |
| | | | | | | | | | EDLAVYY | | | | | |
| | | | | | | | | | C | | | | | |
| H5S20- | DIVMSQS | 1084 | QSLLYGS | 1148 | LAWYQQK | 1211 | WAS | 1245 | TRESGVP | 1318 | QQYYSYP | 1407 | FGSGTKL | 1428 |
| 30AL | PSSLPVS | | NQKNY | | PGQSPKL | | | | DRFTGSG | | FT | | EIK | |
| | VGEKVTM | | | | LIY | | | | SGTDFTL | | | | | |
| | TCKSS | | | | | | | | TISSVKA | | | | | |
| | | | | | | | | | EDLAVYY | | | | | |
| | | | | | | | | | C | | | | | |
| H5S20- | DIVMSQS | 1083 | QSLLFSS | 1147 | LAWYQQK | 1211 | WAS | 1245 | TRESGVP | 1318 | QQYYTYP | 1408 | FGSGTKL | 1446 |
| 33AL | PSSLAVS | | NQKNY | | PGQSPKL | | | | DRFTGSG | | FT | | EMK | |
| | VGEKITM | | | | LIY | | | | SGTDFTL | | | | | |
| | SCKSS | | | | | | | | TISSVKA | | | | | |
| | | | | | | | | | EDLAVYY | | | | | |
| | | | | | | | | | C | | | | | |
| H5S20- | DIVMSQS | 1083 | QSLLFGS | 1149 | LAWYQQK | 1211 | WAS | 1245 | TRESGVP | 1318 | QQYYTYP | 1408 | FGSGTKL | 1446 |
| 42AL | PSSLAVS | | NQKNY | | PGQSPKL | | | | DRFTGSG | | FT | | EMK | |
| | VGEKITM | | | | LIY | | | | SGTDFTL | | | | | |
| | SCKSS | | | | | | | | TISSVKA | | | | | |
| | | | | | | | | | EDLAVYY | | | | | |
| | | | | | | | | | C | | | | | |
| H5S20- | DIVMSQS | 1083 | QSLLFSS | 1150 | LAWYQQK | 1217 | WAS | 1245 | TRESGVP | 1318 | QQYYSYP | 1407 | FGSGTKL | 1428 |
| 4AL | PSSLAVS | | IQKNY | | PGQSPKL | | | | DRFTGSG | | FT | | EIK | |
| | VGEKITM | | | | LVY | | | | SGTDFTL | | | | | |
| | SCKSS | | | | | | | | TISSVKA | | | | | |
| | | | | | | | | | EDLAVYY | | | | | |
| | | | | | | | | | C | | | | | |
| H5S20- | DIVMSQS | 1077 | QSLLYSS | 1143 | LAWYQQK | 1211 | WSS | 1253 | TRESGVP | 1318 | QQYYSYP | 1407 | FGSGTKL | 1428 |
| 6AL | PSSLAVS | | NQKNY | | PGQSPKL | | | | DRFTGSG | | FT | | EIK | |
| | VGEKVTM | | | | LIY | | | | SGTDFTL | | | | | |
| | SCKSS | | | | | | | | TISSVKA | | | | | |
| | | | | | | | | | EDLAVYY | | | | | |
| | | | | | | | | | C | | | | | |
| ITI_ | QIVLTQS | 1085 | SSVS--- | 1108 | LHWYQQK | 1214 | NTS | 1254 | NLASGVP | 1324 | HQYH-RS | 1409 | FGGGTKL | 1427 |
| 12 | PAIMSAS | | --SSY | | PGSSPKL | | | | ARFSGSG | | PT | | EIK | |
| 2L | LGERVTM | | | | WIY | | | | SGTSFSL | | | | | |
| | TCTAS | | | | | | | | TISSMEA | | | | | |
| | | | | | | | | | EDAATYY | | | | | |
| | | | | | | | | | C | | | | | |
| ITI_ | DIKMTQS | 1081 | QDI---- | 1145 | LSWFQQK | 1213 | RAN | 1252 | RLVDGVP | 1325 | LQYDEFP | 1406 | FGSGTKL | 1446 |
| 173L | PSSMYAS | | --NSY | | PGKSPKT | | | | SRFSGSG | | FT | | EMK | |
| | LGESVTI | | | | LIY | | | | SGQDYSL | | | | | |
| | TCKAS | | | | | | | | TISSLEF | | | | | |
| | | | | | | | | | EDMGIYF | | | | | |
| | | | | | | | | | C | | | | | |
| ITI_ | DILMTQS | 1086 | QGI---- | 1151 | IGWLQQK | 1218 | HGT | 1255 | NLEDGVP | 1326 | GQYGQFP | 1410 | FGGGTKL | 1432 |
| 203L | PSSMSVS | | --SSN | | PGKSFKG | | | | SRFSGSG | | PT | | EI- | |
| | LGDTVNI | | | | LIY | | | | SGADYSL | | | | | |
| | TCHAS | | | | | | | | TISNLES | | | | | |
| | | | | | | | | | EDFADYY | | | | | |
| | | | | | | | | | C | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #1

| | | | | | | | | | NRFSGVP | | | | | |
|---|---|---|---|---|---|---|---|---|---|---|---|---|---|---|
| | | | | | | | | | DRF[S/I | | | | | |
| | | | | | | | | | ]G[S/G] | | SQS[T/I | | | |
| | DVVMTQT | | | | | | | | GSGTDFT | | ][-/H][ | | | |
| | PLSL[P/ | | | | LHWYLĄK | | | | L[K/R]I | | -/V][H/ | | | |
| | S]VSLGD | | QSLVH[S | | PGQSP[K | | | | SRVE[T/ | | P/V][V/ | | FG[S/G] | |
| | QAS[I/V | | /T]NGNT | | /R]L[L/ | | | | A]EDLGV | | P][P/W/ | | GTKLE[I | |
| | ]SCRSS | 1087 | Y | 1152 | M]IY | 1219 | KVS | 1248 | YFC | 1327 | F]T | 1411 | /V]K | 1447 |
| H5S14- | DVVMTQT | 1088 | QSLVHSN | 1153 | LHWYLĄK | 1220 | KVS | 1248 | NRFSGVP | 1328 | SQSIH-V | 1412 | FGSGTKL | 1428 |
| 10AL | PLSLPVS | | GNTY | | PGQSPKL | | | | DRFSGSG | | PFT | | EIK | |
| | LGDQASI | | | | LIY | | | | SGTDFTL | | | | | |
| | SCRSS | | | | | | | | KISRVEA | | | | | |
| | | | | | | | | | EDLGVYF | | | | | |
| | | | | | | | | | C | | | | | |
| H5S14- | DVVMTQT | 1088 | QSLVHSN | 1153 | LHWYLQK | 1220 | KVS | 1248 | NRFSGVP | 1328 | SQSTH-V | 1413 | FGSGTKL | 1428 |
| 7ALandH | PLSLPVS | | GNTY | | PGQSPKL | | | | DRFSGSG | | PFT | | EIK | |
| 5S15- | LGDQASI | | | | LIY | | | | SGTDFTL | | | | | |
| 23AL | SCRSS | | | | | | | | KISRVEA | | | | | |
| | | | | | | | | | EDLGVYF | | | | | |
| | | | | | | | | | C | | | | | |
| H5S15- | DVVMTQT | 1088 | QSLVHTN | 1154 | LHWYLQK | 1221 | KVS | 1248 | NRFSGVP | 1329 | SQST--H | 1414 | FGSGTKL | 1428 |
| 10AL | PLSLPVS | | GNTY | | PGQSPRL | | | | DRFSGGG | | VPT | | EIK | |
| | LGDQASI | | | | LIY | | | | SGTDFTL | | | | | |
| | SCRSS | | | | | | | | KISRVEA | | | | | |
| | | | | | | | | | EDLGVYF | | | | | |
| | | | | | | | | | C | | | | | |
| H5S15- | DVVMTQT | 1089 | QSLVHSN | 1153 | LHWYLQK | 1220 | KVS | 1248 | NRFSGVP | 1330 | SQST--H | 1414 | FGSGTKL | 1448 |
| 11AL | PLSLPVS | | GNTY | | PGQSPKL | | | | DRFIGSG | | VPT | | EVK | |
| | LGDQASV | | | | LIY | | | | SGTDFTL | | | | | |
| | SCRSS | | | | | | | | KISRVEA | | | | | |
| | | | | | | | | | EDLGVYF | | | | | |
| | | | | | | | | | C | | | | | |
| H5S15- | DVVMTQT | 1090 | QSLVHSN | 1153 | LHWYLQK | 1220 | KVS | 1248 | NRFSGVP | 1328 | SQST--H | 1414 | FGSGTKL | 1428 |
| 11BL | PLSLSVS | | GNTY | | PGQSPKL | | | | DRFSGSG | | VPT | | EIK | |
| | LGDQASI | | | | LIY | | | | SGTDFTL | | | | | |
| | SCRSS | | | | | | | | KISRVEA | | | | | |
| | | | | | | | | | EDLGVYF | | | | | |
| | | | | | | | | | C | | | | | |
| H5S15- | DVVMTQT | 1088 | QSLVHSN | 1153 | LHWYLQK | 1222 | KVS | 1248 | NRFSGVP | 1331 | SQST--H | 1414 | FGSGTKL | 1428 |
| 11CL | PLSLPVS | | GNTY | | PGQSPKL | | | | DRFSGSG | | VPT | | EIK | |
| | LGDQASI | | | | MIY | | | | SGTDFTL | | | | | |
| | SCRSS | | | | | | | | RISRVEA | | | | | |
| | | | | | | | | | EDLGVYF | | | | | |
| | | | | | | | | | C | | | | | |
| H5S15- | DVVMTQT | 1088 | QSLVHSN | 1153 | LHWYLQK | 1220 | KVS | 1248 | NRFSGVP | 1328 | SQST--H | 1414 | FGSGTKL | 1428 |
| 11DL | PLSLPVS | | GNTY | | PGQSPKL | | | | DRFSGSG | | VPT | | EIK | |
| | LGDQASI | | | | LIY | | | | SGTDFTL | | | | | |
| | SCRSS | | | | | | | | KISRVEA | | | | | |
| | | | | | | | | | EDLGVYF | | | | | |
| | | | | | | | | | C | | | | | |
| H5S15- | DVVMTQT | 1088 | QSLVHSN | 1153 | LHWYLQK | 1220 | KVS | 1248 | NRFSGVP | 1332 | SQST--H | 1414 | FGSGTKL | 1428 |
| 11EL | PLSLPVS | | GNTY | | PGQSPKL | | | | DRFSGSG | | VPT | | EIK | |
| | LGDQASI | | | | LIY | | | | SGTDFTL | | | | | |
| | SCRSS | | | | | | | | KISRVET | | | | | |
| | | | | | | | | | EDLGVYF | | | | | |
| | | | | | | | | | C | | | | | |
| H5S15- | DVVMTQT | 1088 | QSLVHSN | 1153 | LHWYLQK | 1220 | KVS | 1248 | NRFSGVP | 1328 | SQSTHVP | 1415 | FGGGTKL | 1427 |
| 22AL | PLSLPVS | | GNTY | | PGQSPKL | | | | DRFSGSG | | PWT | | EIK | |
| | LGDQASI | | | | LIY | | | | SGTDFTL | | | | | |
| | SCRSS | | | | | | | | KISRVEA | | | | | |
| | | | | | | | | | EDLGVYF | | | | | |
| | | | | | | | | | C | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #4

| | | | | | | | | | [T/N]LA | | | | | |
|---|---|---|---|---|---|---|---|---|---|---|---|---|---|---|
| | | | | | | | | | [E/D]GV | | | | | |
| | | | | | | | | | PSRFSGS | | | | | |
| | | | | | | | | | GSGT[Q/ | | | | | |
| | | | | | | | | | H][F/Y] | | QH[H/F] | | | |
| | DIQMTQS | | | | | | | | SLKIN[S | | [Y/W][- | | | |
| | PASLS[A | | [E/G]NI | | | | | | /N]LQ[P | | /G][G/T | | | |
| | /V]SVGE | | [Y/H][S | | L[A/T]W | | | | /S]E[D/ | | /S][T/P | | FG[G/S] | |
| | TVTITCR | | /N][Y/N | | YQQKQGK | | [N/A]A[ | | E]FGSYY | | /M][Y/R | | GTKLE[I | |
| | [A/P]S | 1091 | ] | 1155 | SPQLLVY | 1223 | K/T] | 1256 | C | 1333 | /F]T | 1416 | /V]K | 1449 |
| H5S14- | DIQMTQS | 1092 | ENIYSN | 1156 | LAWYQQK | 1224 | AAT | 1238 | NLADGVP | 1334 | QHFWGTP | 1417 | FGGGTKL | 1427 |
| 19AL | PASLSVS | | | | QGKSPQL | | | | SRFSGSG | | RT | | EIK | |
| | VGETVTI | | | | LVY | | | | SGTQYSL | | | | | |
| | TCRAS | | | | | | | | KINSLQS | | | | | |
| | | | | | | | | | EDFGSYY | | | | | |
| | | | | | | | | | C | | | | | |
| H5S14- | DIQMTQS | 1093 | ENIYSY | 1157 | LAWYQQK | 1224 | NAK | 1257 | TLAEGVP | 1335 | QHHYGTM | 1418 | FGGGTKL | 1427 |
| 9AL | PASLSAS | | | | QGKSPQL | | | | SRFSGSG | | YT | | EIK | |
| | VGETVTI | | | | LVY | | | | SGTQFSL | | | | | |
| | TCRAS | | | | | | | | KINSLQP | | | | | |
| | | | | | | | | | EDFGSYY | | | | | |
| | | | | | | | | | C | | | | | |
| H5S15- | DIQMTQS | 1093 | ENIYSY | 1157 | LAWYQQK | 1224 | NAK | 12577 | TLAEGVP | 1335 | QHHY-GT | 1419 | FGGGTKL | 1427 |
| 2AL | PASLSAS | | | | QGKSPQL | | | | SRFSGSG | | YT | | EIK | |
| | VGETVTI | | | | LVY | | | | SGTQFSL | | | | | |
| | TCRAS | | | | | | | | KINSLQP | | | | | |
| | | | | | | | | | EDFGSYY | | | | | |
| | | | | | | | | | C | | | | | |
| H5S15- | DIQMTQS | 1093 | GNIHNY | 1158 | LAWYQQK | 1224 | NAK | 1257 | TLADGVP | 1336 | QHFW-ST | 1420 | FGSGTKL | 1428 |
| 32AL | PASLSAS | | | | QGKSPQL | | | | SRFSGSG | | FT | | EIK | |
| | VGETVTI | | | | LVY | | | | SGTQYSL | | | | | |
| | TCRAS | | | | | | | | KINSLQP | | | | | |
| | | | | | | | | | EDFGSYY | | | | | |
| | | | | | | | | | C | | | | | |
| ITI_ | DIQMTQS | 1093 | GNIHNY | 1158 | LTWYQQK | 1225 | NAK | 1257 | TLADGVP | 1337 | QHFW-ST | 1420 | FGSGTKL | 1448 |
| 02 | PASLSAS | | | | QGKSPQL | | | | SRFSGSG | | FT | | EVK | |
| 3L | VGETVTI | | | | LVY | | | | SGTQYSL | | | | | |
| | TCRAS | | | | | | | | KINNLQP | | | | | |
| | | | | | | | | | EDFGSYY | | | | | |
| | | | | | | | | | C | | | | | |
| ITI_ | DIQMTQS | 1094 | ENIYSY | 1157 | LAWYQQK | 1224 | NAK | 1257 | TLAEGVP | 1338 | QHHYGTP | 1421 | FGGGTKL | 1427 |
| 24 | PASLSAS | | | | QGKSPQL | | | | SRFSGSG | | YT | | EIK | |
| 0L | VGETVTI | | | | LVY | | | | SGTHFSL | | | | | |
| | TCRPS | | | | | | | | KINSLQP | | | | | |
| | | | | | | | | | EEFGSYY | | | | | |
| | | | | | | | | | C | | | | | |
| | | Seq. | | Seq. | | Seq. | | Seq. | | Seq. | | Seq. | | Seq. |
| | FR1 | ID | CDR1 | ID | FR2 | ID | CDR2 | ID | FR3 | ID | CDR3 | ID | FR4 ID | ID |

Consensus Cluster #2

| | | | | | | | | | [N/T][L | | | | | |
|---|---|---|---|---|---|---|---|---|---|---|---|---|---|---|
| | | | | | | | | | /R][A/R | | | | | |
| | [D/E][I | | | | | | | | /F][S/P | | | | | |
| | /T/A][V | | | | | | | | ]GV[P/L | | | | | |
| | /T][M/V | | | | | | | | ][D/S]R | | | | | |
| | ]TQ[A/S | | | | | | | | FS[S/G] | | | | | |
| | /T][A/P | | | | | | | | SG[S/Y] | | | | | |
| | ][F/A/L | | | | | | | | GTDF[T/ | | | | | |
| | ]S[N/L] | | [K/T/Q] | | | | | | V][L/F] | | | | | |
| | [P/S/T] | | [S/D/T] | | | | | | [R/T/K] | | | | | |
| | V[T/A/S | | [L/I][L | | | | | | I[S/E][ | | | | | |
| | ][L/T]G | | /-/E][H | | [L/M][Y | | | | R/N][V/ | | | | | |
| | [T/E/D] | | /-/N][S | | /N]WY[L | | | | T][E/L] | | [A/L]Q[ | | | |
| | [S/K/Q] | | /-/T][N | | /Q]QKPG | | | | [A/S]ED | | N/S/V][ | | | |
| | [A/V][S | | /-][G/- | | [Q/E][S | | | | [V/L][G | | L/D/T][ | | | |
| | /T]I[S/ | | ][I/D/N | | /P]P[Q/ | | [Q/E/R] | | /A][V/ | | E/N/H][ | | | |
| | R]C[R/I | | ][T/D][ | | K]LLI[Y | | [M/G/V] | | D]Y[Y/F] | | L/M/V]P | | FGGGTKL | |
| | ][S/T]S | 1095 | Y/D] | 1159 | /S] | 1226 | [S/N] | 1258 | C | 1339 | YT | 1422 | EIK | 1427 |
| H5S14- | ETTVTQS | 1096 | TDI---- | 1160 | MNWYQQK | 1227 | EGN | 1259 | TLRPGVP | 1340 | LQSDNMP | 1423 | FGGGTKL | 1427 |
| 12AL | PASLSVA | | -DDD | | PGEPPKL | | | | SRFSSSG | | YT | | EIK | |
| | TGEKVTI | | | | LIS | | | | YGTDFVF | | | | | |
| | RCITS | | | | | | | | TIENTLS | | | | | |
| | | | | | | | | | EDVADYY | | | | | |
| | | | | | | | | | C | | | | | |
| H5S19- | DAVMTQT | 1097 | QTLENTN | 1161 | LNWYLQK | 1228 | RVS | 1260 | NRFSGVL | 1341 | LQVTHVP | 1424 | FGGGTKL | 1427 |
| 22AL | PLSLTVS | | GNTY | | PGQSPQL | | | | DRFSGSG | | YT | | EIK | |
| | LGDQASI | | | | LIY | | | | SGTDFTL | | | | | |
| | SCRSS | | | | | | | | KISRVEA | | | | | |
| | | | | | | | | | EDLGVYF | | | | | |
| | | | | | | | | | C | | | | | |
| H5S19- | DIVMTQA | 1098 | KSLLHSN | 1162 | LYWYLĄK | 1229 | QMS | 1261 | NLASGVP | 1342 | AQNLELP | 1425 | FGGGTKL | 1427 |
| 24AL | AFSNPVT | | GITY | | PGQSPQL | | | | DRESSSG | | YT | | EIK | |
| | LGTSASI | | | | LIY | | | | SGTDFTL | | | | | |
| | SCRSS | | | | | | | | RISRVEA | | | | | |
| | | | | | | | | | EDVGVYY | | | | | |
| | | | | | | | | | C | | | | | |

TABLE 3

| Seq. Name | Sequence | Seq. ID |
|---|---|---|
| H5S14-12AH | QVQLQQSGAELVRPGTSVKISCKASGNIFTNSWLGWIKQRPGHGLEWIGDVYPGGGYNKYNEKFKGKATLTIDTSASTAYMQ | 1 |
| | LSRLTSEDSGVYFCAGAMDYWGQGTSVTVSS | |
| H5S14-13AH | QVQLQQSGAELVRPGTSVKISCKASGYIFTNSWLGWIKQRPGHGLEWIGDVYPGGGYNKYNEKFKGKATLTIDTSASTAYMQ | 2 |
| | LSRLTSEDSAVYFCAGAMDYWGQGTSVTVSS | |
| H5S14-14AH | QVQLQQSGAELVRPGTSVKISCKASGYIFTNYWLGWIKQRPGHGLEWIGDVYPGGGYNKYNEKFKGKATLTVDTSASTAYM | 3 |
| | QLSRLTSEDSAVYFCAGAMDYWGQGTSVTVSS | |
| H5S14-19AH | QVQLQQSGAELVRPGTSVKISCKASGYTFTNSWLGWVKQRPGHGLEWIGDIYPGGGYNKYNEKFKGKATLTADTSSSTAYM | 4 |
| | QLSSLTSEDSAVYFCAGAMDSWGQGTSVTVSS | |
| H5S15-31AH | QVQLQQSGAELVRPGTSVKISCKASGYAFTNSWLGWVKQRPGHGLEWIGDIYPGGGYNKYNEKFKGKATLTADTSSSTAYM | 5 |
| | QLSSLTSEDSAVYFCAGALDYWGQGTSVTVSS | |
| H5S15-35AH | QVQLQQSGAELVRPGTSVKISCKASGYTFTNSWLGWVKQRPGHGLEWIGDIYPGGGYNKYNEKFKGKATLTADTSSSTAYM | 6 |
| | QLGSLTSEDSAVYFCAGAMDYWGQGTSVTVSS | |
| H5S20-23AH | QVQLQQSGAELVRPGTSVKISCKASGYTLINSWLGWVKQRPGHGLEWIGDIYPGGGYNKYNEKFKGKAILTADTSSSTAYMQ | 7 |
| | LSSLTSEDSAVYFCAGAMDYWGQGTSVTVSS | |
| H5S20-24AH | QVQLQQSGAELVRPGTSVKISCKASGYTFTNYWLGWVKQRPGHGLEWIGDIYPGGGYIIYNEKFKGKATLTVDSSATTAYIQL | 8 |
| | NSLTSEDSAVYFCAGAMDHWGQGTSVTVSS | |
| H5S20-28AH | QVQLQQSGAEVVRPGTSVQISCKASGFTLTNYWLGWVKQRPGHGLEWIGDIYPGGGYTNYNEKFKGKATLTADTSSNTAYM | 9 |
| | QLSGLTSEDSAVYFCAGAMDKWGQGTSVTVSS | |
| H5S20-28BH | QVQLQQSGAEVVRPGTSVKISCKASGFTLTNYWLGWVKQRPGHGLEWIGDIYPGGGYTNYNEKFKGKATLTADTSSNTAYM | 10 |
| | QLSGLTSEDSAVYFCAGAMDKWGQGTSVTVSS | |
| H5S20-30AH | QVQLQQSGTEVVRPGTSVKISCKASGFTLTNYWLGWVKQRPGHGLEWIGDIYPGGGYANYNEKFKGKATLTADTSSNTAYM | 11 |
| | QLSGLTSEDSAVYFCAGAMDKWGQGTSVTVSS | |
| H5S20-32AH | QVQLQQSGAELVRPGTSVKISCKASGYTFTNYWLGWVKQWPGHGLEWIGDIYPGGGYNIYNEKFKGKATLTVDTSSSTAYM | 12 |
| | QLSSLTSEDSAVYFCAGAMDYWGQGTSVTVSS | |
| H5S20-32BH | QVQLQQSGAELVRPGTSVKISCKASGYTFTNYWLGWVKQRPGHGLEWIGDIYPGGGYNIYNEKFKGKATLTVDTSSSTAYM | 13 |
| | QLSSLTSEDSAVYFCAGAMDYWGQGTSVTVSS | |
| H5S20-32CH | QVQLQQSGAELVRPGTSVKISCKASGYTFTNYWLGWVKQRPGHGLEWIGDIYPGGGYNIYNEKFKGKATLTIDTSSSTAYMQ | 14 |
| | LSSLTSEDSAVYFCAGAMDYWGQGTSVTVSS | |
| H5S20-33AH | QVQLQQSGAELVRPGTSVRISCKASGYTLTNSWLGWVKQRPGHGLEWIGDIYPGGGYTKYNENFKGKATLTADTSSSTAYM | 15 |
| | QLSRLTSEDSGVYFCVGAVAYWGQGTSVTVSS | |
| H5S20-35AH | QVQLQQSGAELVRPGTSVKISCKASGYTFTNYWLGWVKQRPGHGLEWIGDIYPGGGYNKYNEKFKGKATLTADTSSSTAYM | 16 |
| | QLSSLTSEDSAVYFCAGAMDYWGQGTSVTVSS | |
| H5S20-36AH | QVQLQQSGAELVRPGTSVKMSCKASGYTFTNSWLGWVKQRPGHGLEWIGDIYPGGGYIKYNEKFKGKATLTADTSSSTAYM | 17 |
| | QLSSLTSEDSAVYFCAGAMDYWGQGTSVTVSS | |
| H5S20-38AH | QVQLQQSGAELVRPGTSVRISCKASGYTLTNSWLGWVKQRPGHGLEWIGDIYPGGGYTKYNEKFKGKATLTADTSSSTAYM | 18 |
| | QLSSLTSEDSAVYFCAGAVAYWGQGTSVTVSS | |
| H5S20-3BH | QVQLQQSGAELVRPGTSVKISCKASGYTFTNSWLGWVKQRPGHGLEWIGDIYPGGGYNKYNEKFRGKATLTADTSSSTAYM | 19 |
| | QLSSLTSEDSAVYFCAGAMDYWGQGTSVTVSS | |
| H5S20-3CH | QVQLQQSGAELVRPGTSVKISCKASGYTFTNSWLGWVKQRPGHGLEWIGDIYPGGGYNKYNEKFKGKATLTADTSSSTAYM | 20 |
| | QLSSLTSEDSAVYFCAGAMDYWGQGTSVTVSS | |
| H5S20-41AH | QVQLQQSGAELVRPGTSVKISCKASGYRFTNYWLGWVKQRPGHGLEWIGDIYPGGGYNKYNEKFKGKATLTADTSSSTAYM | 21 |
| | QLSSLTSEDSAVYFCAGAMDYWGQGTSVTVSS | |
| H5S20-41BH | QVQLQQSGAELVRPGTSVKISCKASGYRFTNYWLGWIKQRPGHGLEWIGDIYPGGGYNKYNEKFKGKATLTADTSSSTAYM | 22 |
| | QLSSLTSEDSAVYFCAGAMDYWGQGTSVIVSS | |
| H5S20-43AH | QVQLQQSGAELVRPGTSVMISCKASGYTFTNYWLGWVKQRPGHGLEWIGDIYPGGGYIIYNEKFKGKATLTVDSSATTAYMQ | 23 |
| | LSSLTSEDSAVYFCAGAMDYWGQGTSVTVSS | |
| H5S20-48AH | QVQLQQSGAELVRPGTSVRISCKASGYTLTNSWLGWVKQRPGHGLEWIGDIYPGGGYNKYNEKFKGKATLTADTSSSTAYM | 24 |
| | QLSSLTSEDSAVYFCAGAVAYWGQGTSVTVSS | |
| H5S20-50AH | QVQLQQSGAELVRPGTSVKISCKASGYRFTNSWLGWVKQRPGHGLEWIGDIYPGGGYNKYNEKFKGKATLTADTSSNTAYM | 25 |
| | QLSSLTSEDSAVYFCAGALDYWGQGTSVTVSS | |
| H5S20-54AH | QVQLQQSGAEVVRPGTSVKISCKASGFTLTNYWLGWVKQRPGHGLEWIGDIYPGGGYTNYNEKFKGKATLTADTSSNTAYM | 26 |
| | QLSGLTSEDSAVYFCAGAMDNWGQGTSVTVSS | |
| H5S20-57AH | QVQLQQSGAELVRPGTSVKISCKASGYTFTNSWLGWVKQRPGHGLEWIGDIYPGGGYNKYNEKFKGKATLTADTSSSTAYM | 27 |
| | QLSSLTSEDSAVYFCIGAMDYWGQGTSVTVSS | |
| H5S20-60AH | QVQLQQSGAELVRPGTSVRISCKASGYTLTNSWLGWVKQRPGHGLEWIGDIYPGGGYTKYNENFKGKATLSADTSSSTAYM | 28 |
| | QLSSLTSGDSAVYFCAGAMAYWGQGTSVTVSS | |
| H5S20-62AH | QVQLQQSGAELVRPGTSVKISCKASGYSFTNYWLGWVKQRPGHGLEWIGDIYPGGGYNMYNEKFKGKATLTVDTSSSTAYM | 29 |
| | QLSSLTSEDSAVYFCAGAMDYWGQGTSVTVSS | |
| H5S20-7AH | QVQLQQSGAELVRPGTSVKISCKASGYRFTNSWLGWIKQRPGHGLEWIGDIYPGGGYNKYNEKFRGKATLTADTSSSTAYM | 30 |
| | QLSSLTSEDSAVYFCAGAMDSWGQGTSVTVSS | |
| H5S20-7BH | QVQLQQSGAELIRPGTSVKISCKASGYRFTNSWLGWIKLRPGHGLEWIGDIYPGGGYNKYNEKFRGKATLTADTSSSTAYMQ | 31 |
| | LSSLTSEDSAVYFCAGAMDSWGQGTSVTVSS | |
| ITI_047H | QVQLQQSGAELVRPGTSVKISCKASGYTFTNSWLGWVKQRPGHGLEWIGDIYPGGGYNKYNEKFKGKATLTADTSSSTAYM | 32 |
| | QLSSLTSEDSGVYFCAGAMDYWGQGTAVTVSS | |
| ITI_053H | QVQLQQSGAELVRPGTSVKISCKASGYAFTNSWLGWVRQRPGHGLEWIGDIYPGGGYNKYNEKFKGKATLTADTSSSTAYM | 33 |
| | QLSSLTSEDSAVYFCAGALDYWGQGTSVTVSS | |
| H5S14-11AH | EVKLVESGGGLVKPGGSLRLSCAASGFTFSSFAMSWIRQTPEKGLEWVASITTGGSSYSPDSLKGRFTISRDNVRNIVYLQMS | 34 |
| | SLRSEDTAMYACARGGGGNYFWFAYWGQGTLVTVSA | |
| H5S14-8AH | EVKLVESGGDLVKPGGSLKLSCAASGFTFSNYAMSWVRQTPEKRLEWVASISTGGTTSYYSDSVKGRFTISRDNARNILYLQ | 35 |
| | MSSLRSEDTAMYYCARGGGGNYFWFTYWGQGTLVTISA | |
| H5S15-10AH | DVQLVESGGGLVQPGGSRKLSCAASGFTFSSFGMHWIRQAPEKGLEWVASISSGSSTIYFADTVKGRFTISRDNPKNTLFLQ | 36 |
| | MTSLRSEDTAMYYCARGAYGNFAWFPYWGQGTLVTVSA | |
| H5S15-16AH | DVQLVESGGGLVQPGGSRKLSCAASGFTFSSFGMHWVRQAPEKGLEWVAHISSGSSTIYYADTVKGRFTISRDNPKNTLFLQ | 37 |
| | MTSLRSEDTAMYYCARGAYGNFAWFPYWGQGTLVTVSA | |
| H5S15-16BH | DVQLVESGGGLVQPGGSRKLSCAASGFTFSSFGMHWVRQAPEKGLEWVAHISSGSSTIYYADTVKGRFTISRDNPKNTLFLQ | 38 |
| | MTSLRSEDTAMYYCARGAYGNFAWFPYWGQGTLVTVST | |
| H5S15-20AH | DVQLVESGGGLVQPGGSRKLSCAASGFTFSSFGMHWVRQAPEKGLEWVASISSGSSTIYYADTVKGRFTISRDNPKNTLFLQ | 39 |
| | MTSLRSEDTAMYYCARGAYGNFAWFAFWGQGTLVTVSA | |
| H5S15-9AH | DVQLVESGGGLVQPGGSRKLSCAASGFTFSDFGMHWVRQAPEKGLEWVAHISSGSSTIYYADTVKGRFTISRDNPKNTLFLQ | 40 |
| | MTSLRSEDTAMYYCARGAYGNFAWFPYWGQGTLVTVSA | |
| H5S20-11AH | EVKLVESGGGLVKPGGSLKLSCAASGFTFSSYAMSWVRQTPETRLEWVASISSGGNTFYPDSVKGRFTISRDNVRDILYLQM | 41 |
| | SSLRSEDTAMYFCTRGGYGSSYVIWGQGTTVTVSS | |
| H5S20-11BH | EVKLVESGGGLVKPGGSLKLSCAASGFTFSSYAMSWVRQTPETRLEWVASISSGGNTFYPDSVKGRFTISRDNVRDILYLQM | 42 |
| | SSLRSEDTAMYFCTRGGYGSSYVIWGQGTTLTVSS | |
| H5S20-11CH | EVKLVESGGGLVKPGGSLKLSCAASGFTFSSYAMSWVRQTPETRLEWVASISSGGNTFYPDSVKGRFTISRDDVRDILYLQM | 43 |
| | SSLRSEDTAMYFCTRGGYGSSYVIWGQGTTLTVSS | |
| H5S20-14AH | EVKLVESGGGLVKPGGSLKLSCAASGFTFSSYAMSWVRQTPETRLEWVASISSGGNTYYPDSVKGRFTISRDNVRNILYLQM | 44 |
| | SSLRSEDTAMYFCTRGGYGSSYVIWGQGTTLTVSS | |
| H5S20-14BH | EVKLVESGGGLVKPGGSLKLSCAASGFTFSSYAMSWVRQTPETRLEWVASISSGGNTYYPDSVKGRFTISRDDVRNILYLQM | 45 |
| | SSLRSEDTAMYFCTRGGYGSSYVIWGQGTTLTVSS | |
| H5S20-14CH | EVKLVESGGGLVKPGGSLKLSCAASGFTFSSYAMSWVRQTPETRLEWVASISSGGNTYYPDSVKGRFTISRDNVRDILYLQM | 46 |
| | SSLKSEDTAMYFCTRGGYGSSYVIWGQGTTLTVSS | |
| H5S20-14DH | EVKLVESGGGLVMPGGSLKLSCAASGFTFSSYAMSWVRQTPETRLEWVASISSGGNTYYPDSVKGRFTISRDNVRNILYLQM | 47 |
| | SSLRSEDTAMYFCTRGGYGSSYVIWGQGTTLTVSS | |
| H5S20-1AH | EVKLVESGGGLVKPGGSLKLSCAASGFTFSSYAMSWVRQTPETRLEWVASISSGGDTFYRDSVKARFTISRDDVRDILYLQM | 48 |
| | SSLRSEDTAMYFCTRGGYGSSFVIWGQGTTLTVSS | |
| H5S20-29AH | EVKLVESGGGLVKPGGSLKLSCAASGFTFSSYAMSWVRQTPETRLEWVASISSGGNTFYPDSVKGRFTISRDDVRDILYLQM | 49 |
| | SSLRSEDTAMYFCTRGGYGSSFVIWGQGTTLTVSS | |
| H5S20-37AH | EVKLVESGGGLVKPGGSLKLSCAASGFTFSSYAMSWVRQTPETRLEWVASISSGGKTFYPDSVKGRFTISRDNVRDILYLQM | 50 |
| | SSLRSEDTAMYFCTRGGYGSSYVIWGQGTTLTVSS | |
| H5S20-39AH | EVKLVESGGGLVKPGGSLKLSCAASGFTFSSYAMSWVRQTPETRLEWVASISSGGNTYYPDSVKGRFTISRDNVRNILYLQM | 51 |
| | SSLRSEDTAMYFCTRGGYGSSHVIWGQGTTLTVSS | |
| H5S20-40AH | EVKLVESGGGLVKPGGSLKLSCAASGFTFSNYAMSWVRQTPETRLEWVASISSGGNTYYPDSVKGRFTISRDNVRNILYLQM | 52 |
| | SSLRSEDTAMYFCTRGGYGSSYVIWGQGTTLTVSS | |
| H5S20-4AH | EVKLVESGGGLMKPGGSLKLSCAASGFTFSSYAMSWVRQTPETRLEWVASISSGGSTYYPDSVKGRFTISRDNVRNILYLQM | 53 |
| | SSLRSEDTAMYFCTRGGYGSSYVIWGQGTTLTVSS | |
| H5S20-4BH | EVKLVESGGGLVKPGGSLKLSCAASGFTFSSYAMSWVRQTPETRLEWVASISSGGSTYYPDSVKGRFTISRDNVRNILYLQM | 54 |
| | SSLRSEDTAMYFCTRGGYGSSYVIWGQGTTLTVSS | |
| H5S20-61AH | EVKLVESGGGLVKPGGSLKLSCAASGFTFSSYAMSWVRQTPETRLEWVASISSGGSTYYPDSVKGRFTISRDNVRNILYLQM | 55 |
| | SSLRSEDTAMYFCTRGGYGSSHVIWGQGTTLTVSS | |
| H5S20-6AH | EVKLVESGGGLVKPGGSLKLSCAASGFTFSSFAMSWVRQTPEKRLEWVASISSGGNTYYPDNVKGRFTISRDNAGNILYLQM | 56 |
| | SSLRSEDTAMYYCARGGYGSSYVIWGQGTTLTVSS | |
| H5S19-12BH | QVQLKESGPGLVAPSRSLSITCTVSGFSLTTYGVHWVRQPPGKGLEWLGVIWAGGITNYNSALMSRLTISNDNSRSQVFLKM | 57 |
| | NSLQTDDTAMYYCARDWERDSSGPFPYWGQGTLVTVSA | |
| H5S19-12CH | QVQLKESGPGLVAPSRSLSITCTVSGFSLTTYGVHWVRQPPGKGLEWLGVIWAGGITNYNSALMSRLSISNDNSRSQVFLKM | 58 |
| | NSLQTDDTAMYYCARDWERDSSGPFPYWGQGTLVTVSA | |
| H5S19-14AH | QVQLKESGPGLVAPSQSLSITCTVSGFSLTSYGVHWVRQPPGKGLEWLGVIWAGGITNYNSALMSRLSISNDNSRSQVFLKM | 59 |
| | NSLQTDDTAMYYCARDWERDSSGPFAYWGQGTLVTVSA | |
| H5S19-14BH | QVQLKESGPGLVAPSQSLSITCTVSGFSLTSYGVHWVRQPPGKGLEWLGVIWAGGITNYNSALMSRLSINNDNSRSQVFLKM | 60 |
| | NSLQTDDTAMYYCARDWERDSSGPFAYWGQGTLVTVSA | |
| H5S19-17AH | QVQLKESGPGLVAPSQSLSITCTVSGFSLTTYGVHWVRQPPGKGLEWLGVIWAGGITNYNSALMSRLSISKDNSKSQVFLKM | 61 |
| | NSLQTGDTAMYYCARDWERDSSGPFAYWGQGTLVTVSA | |
| H5S19-18AH | QVQLKESGPGLVAPSQSLSITCTVSGFSLTIYGVHWRQPPGKGLEWLGVIWAGGIINYNSALMSRLSISKDNSKSQVFLKMN | 62 |
| | SLQSDDTAMYYCARDWERDSSGPFAYWGQGTLVTVSA | |
| H5S19-20AH | QVQLKESGPGLVAPSQSLSITCTVSGFSLTSYGVHWVRQPPGKGLEWLGVIWAGGITNYNSALMSRLSISKDNSKSQVFLKM | 63 |
| | NSLQTDDTAMYYCARDWERDSSGPFVYWGQGTLVTVSA | |
| H5S19-20BH | QVQLKESGPGLVAPSQSLSITCTVSGFSLTSYGVHWVRQPAGKGLEWLGVIWAGGITNYNSALMSRLSIIQDNSKSQVFLKM | 64 |
| | NSLQTDDTAMYYCARDWERDSSGPFVYWGQGTLVTVSA | |
| H5S19-20CH | QVQLKESGPVLVAPSQSLSITCTVSGFSLTSYGVHWVRQPPGKGLEWLGVIWAGGITNYNSALMSRLSISKDNSKSQVFLKM | 65 |
| | NSLQTDDTAMYYCARDWERDSSGPFVYWGQGTLVTVSA | |
| H5S19-21AH | QVQLKESGPGLVAPSQSLSITCTVSGFSLTSYGVHWVRQPPGKGLEWLGVIWAGGITNYNSALMSRLSISTDNSRSQVFLKM | 66 |
| | NSLQTDDTAMYYCARDWERDSSGPFPYWGQGTLVTVSA | |
| H5S19-22AH | QVQLKESGPGLVAPSQSLSITCTVSGFSLTNYGVHWLRQPPGKGLEWLGVIWAGGITNYNSALMSRLSISNDNSRSQVFLKM | 67 |
| | NSLQTDDTAMYYCARDWERDSSGPFAYWGQGTLVTVSA | |
| H5S19-26AH | QVQLKESGPGLVAPSQSLSITCTVSGFSLTTYGVYWVRQPPGKGLEWLGVIWAGGITNYNSALMSRLSISKDNSKSQVFLKM | 68 |
| | NSLQTGDTAMYYCARDWERDSSGPFAYWGQGTLVTVSA | |
| H5S19-27AH | QVQLKESGPGLVAPSQSLSITCTVSGFSLTSYGVHWVRQPPGKGLEWLGVIWAGGITSYNSALMSRLSISSDNSRSQVFLKM | 69 |
| | NSLQTDDTAMYYCARDWERDSSGPFPYWGQGTLVTVSA | |
| ITI_144H | QVQLKESGPGLVAPSQNLSITCTVSGFSLTTYGVHWVRQPPGKGLEWLGVIWAGGITNYNSALMSRLSISKDNFKSQVFLKM | 70 |
| | NSLQTDDTAIYYCARDWERDSSGPFPYWGQGTLVTVSA | |
| ITI_145H | QVQLKESGPGLVAPSQNLSITCTVSGFSLSTYGVHWVRQPPGKGLEWLGVIWAGGITNYNSALMSRLSISKDNFKSQVFLKM | 71 |
| | NSLQTDDTAIYYCARDWERDSSGPFPYWGQGTLVTVSA | |
| ITI_146H | QVQLKESGPGLVAPSQSLSITCTVSGFSLTTYGVHWVRQPPGKGLEWLGVIWAGGITNYNSALMSRLNISKDNSKSQVFLKM | 72 |
| | NSLQSDDTAMYYCARDWERDSSGPFAYWGQGTLVTVSA | |
| ITI_162H | QVQLKESGPGLVAPSQSLSITCTVSGFSLTSYGVHWVRQPPGKGLEWLGVIWAGGITNYNSALMSRLSINIDNSKSQVFLKM | 73 |
| | NSLQTDDTAMYYCARDWERDSSGPFPYWGQGTLVTVSA | |
| ITI_164H | GAAEGVRRPGLVAPSQSLSITCTVSGFSLTTYGVHWRQPPGKGLEWLGVIWAGGITNYNSALMSRLNINKDNSKSQVFLKM | 74 |
| | NSLQTGDTAMYYCARDWERDSSGPFAYWGQGTLVTVSA | |
| ITI_165H | QVQLKESGPGLVAPSQSLSITCTVSGFSLTSYGVHWVRQPPGKGLEWLGVIWAGGITNYNSALMSRLSITKDNSKSQVFLKM | 75 |
| | NSLQTDDTAMYYCARDWERDSSGPFAYWGQGTLVTVSA | |
| ITI_168H | QVQLKESGPGLVAPSQSLSITCTVSGFSLTTYGVHWVRQPPGKGLEWLGVIWAGGITNYNSALMSRLNINKDNSKSQVFLKM | 76 |
| | NSLQTGDTAMYYCARDWERDSSGPFAYWGQGTLVTVSA | |
| H5S14-15AH | QVQLKQSGPGLVQPSQSLSITCTVSGFSLTSYGVHWVRQSPGKGLEWLGVIWSGGSTDYNAAFICRLSISKDSSKSQVFFKM | 77 |
| | NSLQADDTAMYYCARNLGGSWVDYWGQGTSVTVSS | |
| H5S14-17AH | QVQLQQSGAELVRPGTSVKMSCKAAGYTFTNYWIGWVKQRPGHGLEWIGDIYPGGGYTNYNEKFKGKATLTADTSSSTAYM | 78 |
| | QLSSLTSEDSAIYYCARNGNSLDYWGQGTTLTVSS | |
| H5S14-25AH | QVQLQQSGAELVKPGASVKMSCKAFGYTFTTYPIEWMKQNHGKSLEWIGNFHPYNDDTKYNEKFKGKAKLTVEKSSSTVYL | 79 |
| | ELSRLTSDDSAVYYCARRLYGGAMDYWGQGTSVTVSS | |
| H5S14-6AH | EVKLVESGGGLVQPGGSLRLSCATSGFTFTDYYMSWVRQTPGKALEWLGFIRNQANAYTTEYSVSVKGRFTISRDNSQSILY | 80 |
| | LQMNTLRVEDSATYYCARVPDYWGQGTTLTVSS | |
| H5S15-12AH | EVQLQQSGAELVKPGASVKLSCTASGFNIKDTYMHWVKQRPEQGLEWIGRIDPANGNTKFDPKFQGKATITADTSSNTAYLQ | 81 |
| | LSSLTSEDTAVYYCASRGGSSFDYWGQGTTLTVSS | |
| H5S15-30AH | QVQLQQPGAELVKPGAPVKLSCKASGYTFTSYWMNWVKQRPGRGLEWIGRIDPSDSETHYNQKFKDKATLTVDKSSSTAYI | 82 |
| | QLSSLTSEDSAVYYCAREYYGNHFDYWGQGTTLTVSS | |
| H5S15-38AH | EVQLQQSGPELVKPGASVKMSCKASGYTFTSYVMHWVKQKPGQGLEWIGYINPYNDGTKYNEKFKGKATLTSDKSSSTAYM | 83 |
| | ELSSLTSEDSAVYYCARMYDYWGQGTTLTVSS | |
| H5S19-11AH | DVQLQESGPGLVNPSQSLSLTCTVTGYSITSDYAWNWIRQFPGNKLEWMGYISYSGNTGYNPSLKSRISITRDTSKNQFFLQL | 84 |
| | NSVTSEDTATYYCARWGLRIDYWGQGTTLTVSS | |
| H5S19-25AH | QVQLQQSGAELMKPGASVKISCKATGYTFSSYWIEWVKQRPGHGLEWIGEILPGSGSTNYNEKFKGKATFTADTSSNTAYM | 85 |
| | QLSSLTSEDSAVYYCARYPRWGKIDYWGQGTTLTVSS | |
| H5S19-6AH | EVQLVESGGGLVKPGGSLKLSCAASGFTFNIYTMSWVRQSPEMRLEWVAEISSGGSHTYYPDTVTGRFTISRDNAKNTLYLE | 86 |
| | MSSLRSEDTAIYYCARGGSLFDYWGQGTTLTVSS | |
| H5S20-15AH | EVQLQQSGAELVKPGASVKLSCTASGFNIKDTYMHWVKQRPEQGLEWIGRIDPANGNTKYDPKFQGKATITADTSSNTAYLQ | 87 |
| | LSSLTSEDTAVYYCATSGGSSYDYWGQGTTLTVSS | |
| H5S20-25AH | EVQLQQSGAELVKPGASVKLSCTASGFNIKDTYMHWVKQRPEQGLEWIGRIDPANGNTKYDPKFQGKATITADTSSNTAYLQ | 88 |
| | LSSLTSEDTAVYYCARSGGSSYDYWGQGTTLTVSS | |
| H5S20-34AH | EVQLQQSGAELVKPGASVKLSCTASGFNIKDTYMHWVKQRPEQGLEWIGRIDPANGNTKYDPKFQGKATITADTSSNTAYLQ | 89 |
| | LSSLTSEDTAVYYCASSGGSSFDYWGQGTTLTVSS | |
| H5S20-42AH | EVQLQQSGAELVKPGASVKLSCTASGFNIKDTYMHWVKQRPEQGLEWIGRIDPANGNTKYDPKFQGKATITADTSSNTAYLQ | 90 |
| | LSSLTSEDTAVYYCAGSGGSSYDYWGQGTTLTVSS | |
| H5S20-8AH | EVQLQQSGAELVKPGASVKLSCTASGFNIKDTYMHWVKQRPEQGLEWIGRIDPANGNTKYDPKFQGKATITADTSSNTAYLQ | 91 |
| | LSSLTSEDTAVYYCAISGGSSYDYWGQGTTLTVSS | |
| ITI_021H | DVQLQESGPGLVKPSQSLSLTCTVTGYSITSDYAWNWVRQFPGNKLEWVGYISYSGSTSYNPSLKSRISITRDTSKNQFFLQL | 92 |
| | NSVTTEDTATYYCARCYYGGRWDYWGQGTTLTVSS | |
| ITI_173H | DVQLQESGPGLVNPSQSLSLTCTVTGYSITSDYAWNWIRQFPGNKLEWMGYINYSGNTGYNPSLKSRISITRDTSKNQFFLQL | 93 |
| | NSVTSEDTATYYCARWGLRIDYWGQGTTLTVSS | |
| ITI_200H | EVQLQQSGAELVKPGASVKLSCTASGFNIKDTYIHWVKQRPEQGLEWIGRIDPANGNIKYDPKFQGKATIMADTSSNTAYLQL | 94 |
| | SSLTSEDTAVYYCAQGGGGAMDYWGQGTSVTVSS | |
| H5S15-11AH | EVQLQQSGADLVKPGASVKLSCTASGFNIKDTYMHWVKQRPEQGLEWIGRIAPANGRTKYDPKFQGKATITADTSSNTAYLQ | 95 |
| | LSSLTSEDTAVYYCANYYASSYDWFAYWGQGTLVTVSA | |
| H5S15-17AH | EVQLQQSGAELVKPGASVKLSCTASGFNIKDTYMHWVKQRPEQGLEWIGRIAPANGKTKFDPKFQGKATITADTSSNTAYLQ | 96 |
| | LSSLTSEDTAVYYCANYYGRSNDWFVYWGQGTLVTVSA | |
| H5S15-1AH | EVQLQQSGAELVKPGASVKLSCTASGFNIKDTYMHWVKQRPEQGLAWIGRIAPANGYTKYDPKFQGKATITTDTSSNTAYLH | 97 |
| | LSRLTSEDTAVYYCANYFGNTYDWFAFWGQGTLVTVSA | |
| H5S15-24AH | EVQLQQSGAELVKPGASVKLSCTASGFNIKDTYMHWVKQRPEQGLEWIGRIAPANGRTKYDPKFQGKATITADTSSNTAYLQ | 98 |
| | LSSLTSEDTAVYYCANYYASSYDWFVYWGQGTLVTVSA | |
| H5S15-36AH | EVQLQQSGAELVKPGASVKLSCTASGFNIKDTYMHWVKQRPEQGLEWIGRIDPANGNTKYDPKFQGKATITADTSSNTAYLQ | 99 |
| | LSSLTSEDTAVYYCARPYGNYGFAYWGQGTLVTVSA | |
| H5S15-37AH | QVQLQQPGAELVKPGAPVKLSCKASGYTFSTYWMNWVKQRPGRGLEWIGRIDPSDSETHYNQKFKDKATLTVDKSSSTAYI | 100 |
| | QLSSLTSEDSAVYYCAIYYSNPVFDYWGQGTTLTVSS | |
| H5S15-3AH | EVQLQQSGAELVKPGASVKLSCTASGFNIKDTYMHWVKQRPEQGLAWIGRIAPANGYTKYDPKFQGKATITTDTSSNTAYLQ | 101 |
| | LSSLTSEDTAVYYCVTYFGNTYDWFAYWGQGTLVTVSA | |
| H5S20-10AH | EVQLQQSGAELVKPGASVKLSCTASGFNIKDTYMHWVNQRPEQGLEWIGRIDPANGNTKYAPKFQGKATITADTSSNTVYLQ | 102 |
| | LSSLTSEDTAVYYCAGYGNSPWFAYWGQGTLVTVSA | |
| H5S20-10BH | EVQLQQSGAELVKPGASVKLSCTASGFNIKDTYMHWVNQRPEQGLEWIGRIDPANGNTKYAPKFQGKATITADTSSNTAYLQ | 103 |
| | LSSLTSEDTAVYYCAGYGNSPWFAYWGQGTLVTVSA | |
| H5S20-17AH | EVQLQQSGAELVKPGASVKLSCTASGFNIKDTYMHWVNQRPEQGLEWIGRIDPANGKTKYAPKFQDKATITADTSSNTAYLQ | 104 |
| | LSSLTSEDTAVYYCAGYGNSPWFAYWGQGTLVTVSA | |
| H5S20-49AH | EVQLQQSGAELVKPGASVKLSCTASGFNIKDTYVHWVKERPEQGLEWIGRIDPANDNTKYAPKFQVKATITADTSSNTAYLQL | 105 |
| | SSLTSEDNAVYYCAPYGNYPAWFAYWGQGTLVTVSA | |
| H5S20-52AH | EVQLQQSGAELVKPGASVKLSCTASGFNIKDTYIHWLNQRPEQGLEWIGRIDPANGKTKFAPKFQDKATITADTSSNTAYLQL | 106 |
| | SSLTSEDTAVYYCAGYGNSPWFAYWGQGTLVTVSA | |
| H5S20-58AH | EVQLQQSGAELVKPGASVMLSCTASGFYIKDTYMHWVNQRPEQGLEWIGRIDPANGKTKYAPKFQDKATITADSSSNTAYLQ | 107 |
| | LSSLTSEDTAVYYCAGYGNSPWFAYWGQGTLVTVSA | |
| H5S20-9AH | EVQLQQSGADLVKPGASVKLSCTASGFNIRDTYMHWVNQRPEQGLEWIGRIDPANGNTKYAPKFQGRATITADTSSNTAYLH | 108 |
| | LSSLTSEDTAVYYCAGYGNSPWFAYWGQGTLVTVSA | |
| ITI_091H | EVQLQQSGAELLKPGASVRLSCTASGFNFKDTYMHWVNQRPEQGLEWIGRIDPANGKTKYAPKFQGKATITADTSSNTAYLQ | 109 |
| | LSSLTSEDAAVFYCAAYGNSPWFAYWGQGTLVTVSA | |
| ITI_122H | QVQLQQSGAELVRPGTSVKVSCKASGYAFTNYLIEWVKQRPGQGLEWIGVINPGSGGTNYNEKFRGKATLTADKSSSTAYM | 110 |
| | QLSSLTSDDSAVYFCARRGHNYGPWFAYWGQGTLVTVSA | |
| H5S14-10AH | QVTLKESGPGILQPSQTLSLTCSFSGFSLSTSGMGVSWIRQPSGKGLEWLAHIYWDDDKRYNPSLKSRLTISKDTSRNQVFLK | 111 |
| | ITSVDTADTATYYCARSMYGNYNYAMDYWGQGTSVTVSS | |
| H5S14-16AH | EVQLVESGGGLVKPGGSLKLSCAASGFTFSDYYMYWVRQTPEKRLEWVATISDGGSYTYYPDSVKGRFTISRDNAKNNLYL | 112 |
| | QMSSLKSEDTAMYYCARDGNYYAMDYWGQGTSVTVSS | |
| H5S14-20AH | QVQLQQPGAELVRPGASVKLSCKASGYTFTNYWINWVKQRPGQGLEWIGNIYPSDSYTNYNQKFKDKATLTVDKSSSTAYM | 113 |
| | QLSSPTSEDSAVYYCTRGHYGNYDPYAMDYWGQGTSVTVSS | |
| H5S14-22AH | EVQLQQSGPELVKPGASVKMSCKASGYTFTSYVMHWVKQKPGQGLEWIGYINPYNDGTKYNEKFKGKATLTSDKSSSTAYM | 114 |
| | ELSSLTSEDSAVYYCARGIITTVIEPILYAMDYWGQGTSVTVSS | |
| H5S14-26AH | QVQLQQSGAELVRPGTSVKVSCKASGYAFTNYLIEWVKQRPGQGLEWIGVINPGSGGTKYNEKFKGKATLTADKSSSTAYM | 115 |
| | QLSSLTSDDSAVYFCARDYGSSYGYAMDYWGQGTSVTVSS | |
| H5S14-28AH | QVQLQQSGAELVRPGTSVKISCKASGYTFTNYWLSWVKQRPGHGLEWIGDIYPGGGYTNYNEKFKGKATLTADTSSSTAYM | 116 |
| | QLSSLTSEDSAVCFCARRVDYAMDYWGQGTSVTVSS | |
| H5S14-29AH | EVQLQQSGPELVKPGASVKMSCKASGYTFTSYVMHWVKQKPGQGLEWIGYINPYNDGTKYNEKFKGKATLTSDKSSSTAYM | 117 |
| | ELSSLTSEDSAVYYCARGIITTWVEPILYAMDYWGQGTSVTVSS | |
| H5S15-33AH | QVQLKQSGPGLVQPSQSLSITCTVSGFSLTTYGVHWVRQSPGKGLEWLGVIWSGGNTDYNPAFISRLSISKDNSKSQVFFKM | 118 |
| | NTLQASDTAIYYCARRGYNKGYAMDYWGQGTSFTVSS | |
| H5S15-40AH | EVQLVESGGGLVKPGGSLKLSCAASGFAFSSYDMSWVRQTPEKRLEWVAYISSGGGSTYYPDTVKGRFTISRDNAKNTLYL | 119 |
| | QMSSLKSEDTAMYYCARLLRYYAMDYWGQGTSVTVSS | |
| H5S19-9AH | QVQLKQSGPGLVQPSQSLSITCTVSGFSLTTYGVHWVRQSPGKGLEWLGVIWSGGSTDYNAAFISRLSISKDNSKSQVFFKM | 120 |
| | NSLQANDTAIYYCARRGYGSPYYYAMDYWGQGTSVTVSS | |
| H5S20-21AH | QVQLQQPGAELVKPGASVMMSCKASGYTFTNYWMHWVRQRPGQGLEWIGVIDPSDSFTNYNQSFRGKATLTVDTSSSTAY | 121 |
| | MRLSSLTSEDSAVYFCSRGERRGIYAMDYWGQGSSVTVSS | |
| ITI_130H | QVQLKQSGPGLVQPSQSLSITCTVSGFSLTTYGVHWVRQSPGKGLEWLGVIWSGGSTDYNAAFISRLSISKDNSKSQVFFKM | 122 |
| | NSLQANDTAIYYCARRGYGKGYAMDYWGQGTSVTVSS | |
| ITI_131H | QVQLKQSGPGLVQPSQSLSITCTVSGFSLTTYGVHWVRQSPGKGLEWLGVIWSGGSTDYNAPFISRLSISKDNSKSQVFFKM | 123 |
| | NSLQANDTAIYYCARRGYNKGYAMDYWGQGTSVTVSS | |
| ITI_180H | QVQLKQSGPGLVQPSQSLSITCTVSGFSLTTYGIHWVRQSPGKGLEWLGVIWSGGSTDYNAAFISRLSITKDKSKSQVFFKMN | 124 |
| | SLQANDTAIYYCARRGYGSPYYYAMDYWGQGTSVTVSS | |
| H5S15-15AH | QIQLVQSGPEVKKPGETVKISCKASGYTLTNYGMNWVKQAPGKGLKWVGWINTYTGEPTYADDFKGRFAFSLETSASTAYLQ | 125 |
| | INNLKNEDMATYFCARSFYGSEAYWGQGTLVTVSA | |
| H5S15-7AH | QIQLVQSGPELKKPGETVKISCKASGYTFTNYGMNWVKQAPGKGLKWVGWINTYTGEPTYAEDFKGRFAFSLETSASTAYLQ | 126 |
| | INNLKNEDMATYFCARSFYGSEAYWGQGTLVTVSA | |
| H5S15-7BH | QIQLVQSGPELKKPGETVKISCKASGYTFTNYGMNWVKQAPGKGLKWVGWINTYTGEPTYADDFKGRFAFSLETSASTAYLQ | 127 |
| | INNLKNEDMATYFCARSFYGSEAYWGQGTLVTVSA | |
| H5S19-13AH | QIQLVQSGPELKKPGETVKISCKASGYTFTNFGVNWVKQAPGKGLKWMGWINTYTGEPTYADDFKGRFAFSLETSASTAYLR | 128 |
| | INNLKNEDTATYFCARSYYANYAYWGQGTTLTVSS | |
| H5S19-3AH | QIQLVQSGPELKKPGETVKISCKASGYTFTNFGVNWVKQAPGKGLKWMGWINTYTGEPTYADDFKGRFAFSLETSASTAYLR | 129 |
| | INNLKNEDSATYFCARSFYKNYAFWGQGTILTVSS | |
| H5S19-3BH | QIQLVQSGPELKKPGETVKISCKASGYTFTNFGVNWVKQAPGKGLKWMGWINTYTGEPTYADDFKGRFAFSLETSASTAYLR | 130 |
| | INNLKNEDTATYFCARSFYKNYAFWGQGTILTVSS | |
| H5S19-3CH | QIQLVQSGPELKKPGETVKISCKASGYTFTNFGVNWVKQAPGKDLKWMGWINTYTGEPTYADDFKGRFAFSLETSASTAYLR | 131 |
| | INNLKNEDTATYFCARSFYKNYAFWGQGTILTVSS | |
| H5S19-8AH | QIQLVQSGPELKKPGETVKISCKASGYTFTNFGVNWVKQAPGKGLKWMGWINTYTGEPTYADDFKGRFAFSLETSASTAYLR | 132 |
| | INNLKNEDTASYFCARSYYANNAYWGQGTTLTVSS | |
| ITI_032H | QIQLVQSGPELKKPGETVKISCKASGYTFTNFGVNWVKQAPGKGLKWMGWINTYTGEPTYADDFKGRFAFSLETSASTAYLR | 133 |
| | INNLKNEDTATYFCARSFYKNYAFWGQSTILTVSS | |
| ITI_035H | QIQLVQSGPELKKPGETVKISCKASGYTFTNFGVNWVKQAPGKGLKWMGWINTYTGEPTYADDFKGRFAFSLETSASTAYLR | 134 |
| | INNLKNEDTATYFCARSYYGNYAYWGQGTTLTVSS | |
| ITI_082H | QIQLVQSGPELKKPGETVKISCKASGYTFTNYGMNWVKQAPGKGLKWMGWINTYTGEPTYADDFKGRFAFSLETSASTAYL | 135 |
| | QINNLKNEDTATYFCARSFTTATCYWGQGTTLTVSS | |
| ITI_083H | QIQLVQSGPELKKPGETVKISCKASGYTFTNYGMNWVKQAPGKGLKWMGWINTYTGEPTYDDDFKGRFAFSLETSASTAYL | 136 |
| | QINNLKNEDTATYFCARSFTTATCYWGQGTTLTVSS | |
| H5S14-18AH | EVQLVETGGGLVQPKGSLKLSCAASGFTFNTNAMNWVRQAPGKGLEWVARIRSKSNNYATYYADSVKDRFTISRDDSQSML | 137 |
| | YLQMNNLKTEDTAMYYCVREGGYGNYPYFDYWGQGTTLTVSS | |
| H5S14-23AH | LVKTGASVKISCKASGYSFTGYYMHWVKQSHGKSLEWIGYISCYNGATSYNQKFKGKATFTVDTSSSTAYMQFNSLTSEDSA | 138 |
| | VYYCARTYYYGSSYGAMDYWGQGTSVTVSS | |
| H5S14-30AH | EVQLVESGGDLVKPGGSLKLSCAASGFTFSSYGMSWVRQTPDKRLEWVATISSGGSYTYYPDSVKGRFTISRDNAKNTLYLQ | 139 |
| | MSSLKSEDTAMYYCARQGGHGNYGAMDYWGQGTSVTVSS | |
| H5S15-14AH | EVQLQQSGPELVKPGASVKMSCKASGYTFTSYVMHWVKQKPGQGLEWIGYINPYNDDTNCNEKFKGKATLTSDKSSSTAYM | 140 |
| | ELSRLTSEDSAVYYCAKARGYGSTFYYSMDYWGQGTSVTVSS | |
| H5S15-25AH | EVQLQQSGPELVKPGASVKMSCKASGYKFNSYVMHWVKQKPGQGPEWIGYINPYNDDTNCNEKFKGKATLTSDKSSSTAY | 141 |
| | MELSSLTSEDSAVYYCAKARGYGGNFYYSMDYWGQGTSVTVSS | |
| H5S15-5AH | EVQLQQSGPELVKPGASVKMSCKASGYTFTSYVMHWVKQKPGQGLEWIGYINPYNDDTNCNEKFKGKATLTSDKSSSTAYM | 142 |
| | DLSSLTSEDSAVYYCAKARGYGGSFYYSMDYWGQGTSVTVSS | |
| H5S20-44AH | EVKLVESGGGLVQPGGSLKLSCAASGFTFSSYTMSWVRQTPEKRLEWVAYISNGGGSTYYPDTVKGRFTISRDNAKNTLYLQ | 143 |
| | MSSLKSEDTAMYYCARHRGYGSSYNYAMDYWGQGTSVTVSS | |
| H5S20-46AH | EVKLVESGGGLVQPGGSLKLSCAASGFTFSSYTMSWVRQTPEKRLEWVAYISNGGGSTYYPDTVKGRFTISRDNAKNTLYLQ | 144 |
| | MSSLKSEDTAMYYCARHRGYGNYVYAMDYWGQGTSVTVSS | |
| H5S20-51AH | QVQLKQSGPGLVQPSQSLSITCTVSGFSLTSYGVHWVRQSPGKGLEWLGVIWRGGSTDYNAAFMSRLSITKDNSKSQVFFK | 145 |
| | MNSLQADDTAIYYCAKNRGYGEGYYAMDYWGQGTSVTVSS | |
| H5S20-53AH | EVQLVESGGGLVKPGGSLKLSCAASGFTFSSYAMSWVRQSPEKRLEWVAEISSGGSYTYYPDTVTGRFTISRDNAKNTLYLE | 146 |
| | MSSLRSEDTAMYYCAREGLRRDYYALDYWGQGTSVTVSS | |
| H5S20-55AH | EVKLVESGGGLVQPGGSLKLSCAASGFTFSSYTMSWVRQTPEKRLEWVADISNGGGSTYYPDTVKGRFTISRDNAKNTLYL | 147 |
| | QMSSLKSEDTAMYYCARHRGYGNYVYAMDYWGQGTSVTVSS | |
| H5S20-59AH | EVKLVESGGGLVKPGGSLKLSCAASGFTFSSYAMSWVRQTPEKRLEWVASISSGSSTYYPDSVKGRFTISRDNARNILYLQM | 148 |
| | SSLRSEDTAMYYCAKGRGYGNYLYAMDYWGQGTSVTVSS | |
| H5S15-19AH | QVQLKESGPGLVAPSQSLSITCTVSGFSLTGYGVNWVRQPPGKGLEWLGMIWGDGGTDYNSALKSRLSISKDNSKSQVFLK | 149 |
| | MNSLQTDDTARYYCARDNYHTVVNGDYWGQGTSVTVSS | |
| H5S15-32AH | QVQLKESGPGLVAPSQSLSITCTVSGFSLTGYGVNWVRQSPGKGLEWLGMIWGDGSTDYNSALKSRLSISKDNSKSQIFLKM | 150 |
| | NSVQTEDTARYYCARDSYRTMTNGDYWGQGTSVTVSS | |
| H5S19-16AH | QIQLVQSGPELKKPGETVKISCKASGYTFTNFGVNWVKQAPGKGLKWMGWINTYTGEPTYADDFKGRFAFSLETSASTAYLQ | 151 |
| | INNLKNEDTATYFCVRSYYGNSGYWGQGTTLTVSS | |
| H5S19-19AH | QIQLVQSGPELKKPGETVKISCKASGYTFTNYGMNWVKQAPGKGLKWMGWINTYTGEPTYADDFKGRFAFSLETSASTAYL | 152 |
| | QINNLKNEDTATYFCVRSYYGNSGYWGQGTTLTVSS | |
| H5S20-12AH | QIQLVQSGPELKKPGETVKISCKASGYTFTNFGMNWVRQAPGKGLKWMGWINTYTGEPTYADDFKGRFAFSLETSASTAYL | 153 |
| | QINNLKNDDMATYFCARSLYGNRDYWGQGTTLTVSS | |
| H5S20-18AH | QIQLVQSGPELKKPGETVKISCKASGYIFTNYGMNWVRQAPGKGLKWMGWINTYTGEPTYADDFKGRFAFSLETSASTAYLQ | 154 |
| | INNLKNEDMATYFCARSFYGNRDYWGQGTTLTVSS | |
| ITI_100H | QIQLVQSGPELKKPGETVKISCKASGYTLTNFGMNWMKQAPGKGLKWMGWINTYTGEPTYAGDFKGRFAFSLETSASTAYL | 155 |
| | QINNLKNEDTATYFCVRSYYGNSGYWGQGTTLTVSS | |
| ITI_101H | QIQLVQSGPELKKPGETVKISCKASGYTLTNFGMNWMKQAPGKDLKWMGWINTYTGEPTYAGDFKGRFAFSLETSASTAYL | 156 |
| | QINNLKNEDTATYFCVRSYYGNSGYWGQGTTLTVSS | |
| H5S15-13AH | QVQLKQSGPGLVQPSQSLSITCTVSGFSLTSYGVHWVRQSPGKGLEWLGVIWRGGSTDYNAAFMSRLSITKDNSKSQVFFK | 157 |
| | MNSLQADDTAIYYCAKTGFAYWGQGTLVTVSA | |
| H5S15-28AH | QVQLQQSGAELVKPGASVKMSCKAFGYTFTTYPIEWMKQNHGKSLEWIGNFHPYNDDTKYNEKFKGKAKLTVEKSSSTVYL | 158 |
| | ELSRLTSDDSAVYYCARGGFAYWGQGTLVTVSA | |
| H5S15-39AH | QIQLVQSGPELKKPGETVKISCKASGYTFTDYSMHWVKQAPGKGLKWMGWINTETGEPTYADDFKGRLAFSLETSASTAFLQ | 159 |
| | INNLKNEDTATYFCANWAGFAYWGQGTLVTVSA | |
| ITI_236H | EVQLQQSGPELVKPGASVKMSCKASGYTFTSYVMHWVKQKPGQGLEWIGYINPYNDGTKYNEKFKGKATLTSDKSSSTAYM | 160 |
| | ELSSLTSEDSAVYYCARERTGPFAYWGQGTLVTVSA | |
| ITI_238H | QVQLQQPGAELVMPGASVKMSCKASGYTFTDYWMHWVKQRPGQGLEWIGAIDTSDSYTSYNQKFKGKATLTVDESSSTAY | 161 |
| | MQLSSLTSEDSAVYYCARSARAAWFAYWGQGTLVTVSA | |
| ITI_240H | QIQLVQSGPELKKPGETVKISCKASGYTFTNYGMNWVKQAPGKGLKWMGWINTYTGEPTYADDFKGRFAFSLETSASTAYL | 162 |
| | QINNLKNDDTATYFCARELLRSAWFAYWGQGTLVTVSA | |
| H5S20-20AH | EVQLQQSGAELVRSGASVKLSCTASGFNIKDYYMHWVKQRPEQGLEWIGWIDPENGDTEYAPKFQGKATMTADTSSNAAYL | 163 |
| | QLSSLTSEDTAVYYCNSRTLGYWGQGTLVTVSA | |
| H5S20-20BH | EVQLQQSGAELVRSGASVKLSCTASGFNIKDYYMHWVKQRPEQGLEWIGWIDPENGDTEYAPKFQGKATMTADTSSNTAYL | 164 |
| | QLSSLTSEDTAVYYCNSRTLGYWGQGTLVTVSA | |
| H5S20-31AH | EVQLQQSGAELVRSGASVKLSCTTSGFNIKDYYMHWVKQRPEQGLEWIGWIDPENGDTEYAPKFQGKATMTADTSSNTAYL | 165 |
| | QLSSLTSEDTAVYYCNSRTLGYWGQGTLVTVSA | |
| H5S20-31BH | EVQLQQSGAELVRSGASVKLSCTTSGFNIKDYYMHWVKQRPEQGLEWIGWIDPENGDTEYAPKFQGKATMTADTCSNTAYL | 166 |
| | QLSSLTSEDTAVYYCNSRTLGYWGQGTLVTVSA | |
| H5S20-45AH | EVQLQQSGAELVRSGASVKLSCAASGFNIKDYYMHWVKQRPEQGLEWIGWIDPENGDTEFAPKFQGKATLTADTSSNTAYL | 167 |
| | QLSSLTSEDTAVYYCNTRTLGYWGQGTLVTVSA | |
| H5S15-18AH | QVQLQQSGAELVRPGTSVKMSCKAAGYTFTNSWIGWVKQRPGHGLEWIGDIYPGGGYTNYYEKFKVKATLTADTSSSTAYM | 168 |
| | QLSSLTSEDSAIYYCASSGAYWGQGTTLTVSS | |
| H5S15-26AH | QVQLQQSGAELVRPGTSVKMSCKAAGYTFTNSWMGWVKQRPGHGLEWIADIYPGGGYSNYNEKFKGKATLTADTSSSTAY | 169 |
| | MQLSSLTSEDSAIYYCASSGAYWGQGTTLTVSS | |
| H5S15-8AH | QVQLQQSGAELVRPGTSVKMSCKAAGYTFTDSWIGWVKQRPGHGLEWIGDIYPGGGYTNYNEKFKGKATLTADTSSSTAYM | 170 |
| | QLSRLTSEDSAIYYCASSGAYWGQGTTLTVSS | |
| H5S19-4AH | QVQLKESGPGLVAPSQSLSITCTVSGFSLTGYGVNWVRQPPGKGLEWLGMIWGDGNTDYNSALKSRLSISKDNSKSQVFLK | 171 |
| | MNSLQTDDTARYYCARSYGSYWGQGTLVTVSA | |
| H5S20-56AH | EVKLVESGGGLVKPGGSLKLSCAASGFTFSSYGMSWVRQTPEKRLEWVATISGGGSYTFYPDSVKGRFTISRDNAKNNLYL | 172 |
| | QVSSLRSEDTALYYCIYDGSYWGQGTLVTVSA | |
| H5S14-24AH | EVQLQQSGPELVKPGTSVKISCKTSGYTITEYTMHWVKQSHGKSLEWIGGINPNNGGTINNQKFKDKATLTVDMSSSTAYME | 173 |
| | LRSLTSEDSAVYYCAGSVVDRYWYFDVWGAGTTVTVSS | |
| H5S14-4AH | EVQLQQSGPELVKPGASVKISCKTSGYTFTEYTMHWVKQSHGKSLEWIGGINPYNGGTINNQKFKGKATLTVDMSSSTAYME | 174 |
| | LRSLTSEDSAVYYCAGSVVDRYWYFDVWGAGTTVTVSS | |
| H5S14-7AH | EVQLQQSGPELVKPGASVKISCKTSGYTFTEYTMHWVKQSHGKSLEWIGGINPNNGGTINNQKFKGKATLTVDMSSSTAYME | 175 |
| | LRSLTSEDSAVYYCAGSVVDRYWYFDVWGAGTTVTVSS | |
| ITI_040H | EVQLQQSGPELVKPGTSVKISCKTSGYTFTEYTMHWVKQSHGKSLEWIGGINPNNGGTINNQKFKGKATLTVDMSSSTAYME | 176 |
| | LRSLTSEDSAVYYCAGSVVDRYWYFDVWGAGTTVTVSS | |
| H5S14-2AH | QVQLQQPGAELVKPGTSVKMSCKASGYTFTSYWMHWVKQRPGQGLEWIGDIYPGSDSTNYNEKFKSKATLTVDTSSSTAY | 177 |
| | MQLSSLTSEDSAVYYCARSGYYGSYLDYWGQGTTLTVSS | |
| H5S15-27AH | EVQLQQSGAELVKPGASVKLSCTASGFNIKDTYMHWVKQRPEQGLEWIGRIDPANGNTKYDPKFQGKATITADTSSNTAYLQ | 178 |
| | LSSLTSEDTAVYYCARSRRYFDVWGAGTTVTVSS | |
| H5S20-22AH | EVQLQQSGAELVKPGASVKLSCTASGFNIKDTYMHWVKQRPEQGLEWIGRIDPANGNTKYAPKFQGKATITADTSSNTAYLQ | 179 |
| | LSSLTSEDTAVYYCARSFGNYFDYWGQGTTLTVSS | |
| ITI_237H | LQQSGAGLVKPGASVKLSCKASGYTFTEYIIYWVKQRSGQGLEWIGWFYPGSGSIRYNEKFKDKATLTADKSSTTVYMDLSR | 180 |
| | LTSEDSAVYFCARHEDGYLDYWGQGTTLTVSS | |
| H5S15-21AH | QIQLVQSGPDLKKPGETVKISCKASGYTFTNYGMNWVKQAPGKDLKWMGWINTYTGEPTYADDFKGRFAFSLETSASTAYL | 181 |
| | QINNLKNDDTATYFCARSINYDSDEKWGQGTSVTVSS | |
| H5S20-26AH | QIQLVQSGPELKKPGETVKISCRASGYTFTNYGMNWVKQAPGKGLKWMGWINTYTGEPTYADDFKGRFAFSLETSASSAYL | 182 |
| | QINNLKNEDMATYFCARSLYYGDNYEAYWGQGTLVTVSA | |
| ITI_127H | QIQLVQSGPEVKKHGETVKISCKASGYIFTNYGINWVKQAPGKGLKWMGWINTYTREPTYADDFKGRFAFSLETSASSAYLQI | 183 |
| | SNLTTEDMATYFCARSLYYVNNYEAYWGQGTLVTVSA | |
| ITI_128H | QIQLVQSGPELKKHGETVRISCKASGYIFTDYGINWVKQAPGKGLKWMGWINTYTGKPTYADDFKGRFAFSLETSASTAYLQI | 184 |
| | NNLKTEDMATYFCARSLYYGNNYEACWGQGTLVTVS | |
| H5S15-6AH | QVQLQQSGAELVRPGTSVKMSCKAAGYTFTNSWIGWVKQRPGHGLEWIGDIYPGGAYTKYNEKFKGKATLTADTSSSTAYM | 185 |
| | QLSSLTSEDSAIYYCASGRDYWGQGTTLTVSS | |
| ITI_045H | QVQLKQSGPGLVQPSQNLSITCTVSGFSLTSYGVHWIRQSPGKGLEWLGVIWSGGGTDYNAAFISRLSISKDNSKSQVFFKM | 186 |
| | NSLQADDTAIYYCASLYYWGQGTLVTVSA | |
| ITI_046H | QVQLKQSGPGLVQPSQNLSITCTVSGFSLTSYGVHWIRQSPGKGLEWLGVIWSGGGTDYNAAFISRLTISKDNSKSQVFFKM | 187 |
| | NSLQADDTAIYYCASLYYWGQGTLVTVSA | |
| H5S15-29AH | EVQLQQSGAELVRPGALVKLSCKASGFNIKDDYMHWVKQRPEQGLEWIGWIDPENGNTIYDPKFQGKASITADTSSNTAYLQ | 188 |
| | LSSLTSEDTAVYYCARGYSSSPYWGQGTLVTVSA | |
| H5S15-2AH | EVQLQQSGAELVKPGASVKLSCTASGFNIKDTYMHWVKQRPEQGLEWIGRIDPANDNTKYDPKFQGKATITADTSSNTAYLQ | 189 |
| | LSSLTSEDTAVYYCARVYYAMDYWGQGTSVTVSS | |
| H5S19-24AH | QVQLQQSGGELLRPGTSVKVSCKASGYAFTNYLIEWVKQRPGQGLEWIGVINPGSGGIYYKEKFKDKAILTADKSSSTAYMQL | 190 |
| | SSLTSDDSAVYFCARGDAMDFWGQGTSVTVSS | |
| H5S14-1AH | DVKLVESGGGLVKPGGSLKLSCAASVFTFSRYTMSWVRQTPEKRLEWVATISSGGSYTYYPDSVKGRFTISRDNAKNTLYLQ | 191 |
| | MSSLKSEDTAIYYCTRGGDGLFDYWGQGTALTVSS | |
| H5S19-5AH | DVKLVESGGGLVKPGGSLKLSCAASGFTFSSYTMSWVRQTPEKRLEWVATISSGGSYTYYPDSVKGRFTISRDNAKNTLYLQ | 192 |
| | MSSLKSEDTAMYYCTRGDDYGFDYWGQGTTLTVSS | |
| ITI_203H | EVQLVESGGDLVKPGGSLKLSCAASGFTFSSYGMSWVRQTPDKRLEWVATISSGGSSTYYPDSVKGRFTISRDNAKNTLYLQ | 193 |
| | MNSLKSEDTAMYYCTRHELGNRSRFPYWGQGTLVTVSA | |
| H5S14-21AH | QVQLQQPGSELVRPGASVKLSCKASGYTFTSYWMHWVKQRPGQGLEWIGNIYPGSGSTNYDEKFKSKATLTVDTSSSTAY | 194 |
| | MQLSSLTSEDSAVYYCTIYDGYYWGQGTLVTVSA | |
| H5S14-27AH | EVKLDETGGGLVQPGRPMKLSCVASGFTFSDYWMNWVRQSPEKGLEWVAQIRNKPYNYETYYSDSVKGRFTISRDDSKSS | 195 |
| | VYLQMNNLRAEDMGIYYCTWGNYWGQGTTLTVSS | |
| H5S14-5AH | DVQLQESGPGLVKPSQSLSLTCTVTGYSITSDFAWNWIRQFPGNKLEWMGYIVYSGSTSYNPSLKSRISITRDTSKNQFFLQL | 196 |
| | NSVTAEDTATYFCTRGMDYWGQGTSVTVSS | |
| H5S19-1AH | DVQLQESGPGLVKPSQSLSLTCTVTGYSITSDYAWNWIRQFPGNKLEWMGYIAYSGGTSYSPSLKSRISITRDTSKNQFFLQL | 197 |
| | NSVTTEDTATYYCVYFKYGGAFAYWGQGTLVTVSA | |
| ITI_023H | EVQLQQSGAELMSPGASVNLSCTASGFNIKDTYIHWVKQRPEQGLEWIGKIDPANGNTKYDPKFQDKATITTDASSNTAYLQL | 198 |
| | SSLTSEDTAVYYCTKSLLWSLGGFAYWGQGTLVTVSA | |
| H5S14-3AH | QIQLVQSGPELKKPGETVKISCKASGYTFTNYGVNWVKQAPGKDLKWMGWINTYTGEPTYADDFKGRFAFSLETSASTAYLQ | 199 |
| | INNLKNEDMATYFCTSRSWVLWGQGTLVTVSA | |
| H5S20-27AH | EVQLQQSGTVLARPGASVKMSCKASGYSFTSYWMHWLKQRPGQGLEWIGAIYPGNSDTVFNQKFKGKAKLTAVTSATTAY | 200 |
| | MELSSLTNEDSAVYYCTKEPRTIEGAWFTYWGQGTLVTVSA | |
| H5S15-23AH | EVQLQQSGTVLARPGASVKMSCKASGYTFTSFWMHWVKQRPGQGLEWIGAISPGNSETTYNQKFTGKAKLTAVTSTSTAYM | 201 |
| | ELSSLTNEDSAVYYCTKIYYDYDDGYWGQGTTLTVSS | |
| H5S14-15AL | SIVMTQTPKFLLVSAGDRVTITCKASQSVSNDVAWYQQKPGQSPKLLIYYESNRYTGVPDRFTGSGYGTDFTFTISTVQAEDL | 874 |
| | AVYFCQQDYSSPWTFGGGTKLEIK | |
| H5S14-16AL | SIVMTQTPKFLLVSAGDRVTITCKASQSVSNDVAWYQQKPGQSPKLLIYYASNRYTGVPDRFTGSGYGTDFTFTISTVQAEDL | 875 |
| | AVYFCQQDYSSPWTFGGGTKLEIK | |
| H5S14-18AL | QIVLTQSPAIMSASPGEKVTMTCSASSSVSYMHWYQQKSGTSPKRWIYDTSKLASGVPARFSGSGSGTSYSLTISSMEAEDA | 876 |
| | ATYYCQQWSSNPFTFGSGTKLEIK | |
| H5S14-1AL | DVQITQSPSYLAASPGETITINCRASKSISKYLAWYQEKPGKTNQLLIYSGSTLQSGIPSRFSGSGSGTDFTLTISSLEPEDFAM | 877 |
| | YYCQQHNEYPWTFGGGTKLEIK | |
| H5S14-20AL | NIVLTQSPASLAVSLGQRATISCRASESVDSYGNSFMHWYQQKPGQPPKLLIYLASNLESGVPARFSGSGSRTDFTLTIDPVE | 878 |
| | ADDAATYYCQQNNEDPYTFGGGTKLEIK | |
| H5S14-24AL | SIVMTQTPKFLLVSAGDRVTITCKASQSVSNDVAWYQQKPGQSPKLLIYYASNRYTGVPDRFTGSGYGTDFTFTISTVQAEDL | 879 |
| | AVYFCQQDYSSPLTFGAGTKLELK | |
| H5S14-25AL | DIQMTQSSSYLSVSLGGRVTITCKASDHINNWLAWYQQKPGNAPRLLISGATSLETGVPPRFSGSGSGKDYTLSITSLQTEDV | 880 |
| | ATYYCQQYWSTPYTFGGGTKLEIK | |
| H5S14-4AL | QIVLTQSPAIMSASPGEKVTISCSASSSVSYMYWYQQKPGSSPKPWIYRTSNLASGVPARFSGSGSGTSYSLTISSMEAEDAA | 881 |
| | TYYCQQYHSYPLTFGAGTKLELK | |
| H5S14-4BL | QIVLTQSPAIMSASPGEKVTISCSASSSVSYMYWYQQKPGSSPKPWIYRTSNLASGVPARFSGSGSGSSYSLTISSMEAEDAA | 882 |
| | TYYCQQYHSYPLTFGAGTKLELK | |
| H5S14-6AL | DIQMTQSPASQSASLGESVTITCLASQTIGTWLAWYQQKPGKSPQLLIYAATSLADGVPSRFSGSGSGTKFSFKISSLQAEDF | 883 |
| | VSYYCQQLHSTPYTFGGGTKLEIK | |
| H5S15-13AL | DIVLTQSPASLAVSLGQRATISCKASQSVDYDGESYMNWYQQKPGQPPKLLIYAASNLDSGIPARFSGSGSGTDFTLNIHPVE | 884 |
| | EEDAATYYCQQSNEDPLTFGAGTKLELK | |
| H5S15-14AL | DIVMTQSQKFMSTSVGDRVSVTCKASQNVGTNVAWYQQKPGQSPKALIYSASYRYSGVPDRFTGSGSGTDFTLTISNVQSE | 885 |
| | DLAEYFCQQYNSYPYTFGGGTNLEIK | |
| H5S15-14BL | DIVMTQSQKFMSTSVGDRVSVTCKASQNVGTNVAWYQQRPGQSPKSLIYSASYRYSGVPDRFTGSGSGTDFTLTISNVQSE | 886 |
| | DLADYFCQQYNSYPYTFGGGAKLEIK | |
| H5S15-14CL | DIVMTQSQKFMSTSVGDRVSVTCKASQNVGTNVAWYQQKPGQSPKALIYSASYRYSGVPDRFTGSGSGTDFTLTISNVQSE | 887 |
| | DLAEYFCQQYNSYPYTFGGGTKLEI | |
| H5S15-15AL | QIVLTQSPAIMSASPGEKVTMTCSASSSVSYMHWYQQKSGTSPKRWIYDTSKLASGVPARFSGSGSGTSYSLTISSMEAEDA | 888 |
| | ATYYCQQWSSNPLTFGAGTKLELK | |
| H5S15-18AL | ENVLTQSPAIMSASPGEKVTMTCRASSSVSSSYLHWYQQKSGASPKLWIYSTSNLASGVPARFSGSGSGTSYSLTISSVEAE | 889 |
| | DAATYYCQQYSGYPLTFGGGTKLEIK | |
| H5S15-24AL | DIVLTQSPASLAVSLGQRATISCKASQSVDYDGNSYMNWYQQKPRQPPKLLIYAASNLESGIPARFRGSGSGTDFTLNIHPVE | 890 |
| | EEDAATYYCQQSNEDPFTFGSGTKLEIK | |
| H5S15-28AL | DIQMTQTTSSLSVSLGDRVTISCRASQDISNYLNWYQQKPDGTVKLLIYYTSRLHSGVPSRFSGSGSGSDYSLTISNLEQEDIA | 891 |
| | TYFCQQGNSLPWTFGGGTKLEIK | |
| H5S15-29AL | EIVLTQSPALMTASPGEKVTITCSVSSTISSRNLHWYQQKSEASPKPWIYGTSNLASGVPVRFSGSGSGTSYSLTISSMEAEDA | 892 |
| | ATYYCQQWNSYPLTFGSGTKLEIK | |
| H5S15-4AL | EIVLTQSPALMAASPGEKVTITCSVSSGIRSSNLHWYQQKSETSPKPWIYGTSNLASGVPIRFSGSGSGTSYSLTISSMEAEDA | 893 |
| | ATYYCQQWSSYPLTFGSGTKLEIK | |
| H5S19-11AL | EIVLTQSPALMAASPGEKVTITCSVSSSISSSNLHWYQQKSETSPKPWIYGTSNLASGVPVRFSGSGSGTSYSLTISSMEAEDA | 894 |
| | ATYYCQQWSSYPLTFGAGTKLELK | |
| H5S19-17AL | DIQMTQTTSSLSASLGDRVTISCSASQGISNYLNWYQQKPDGTVKLLIYYTSSLHSGVPSRFSGSGSGTDYSLTISNLEPEDIA | 895 |
| | TYYCQQYSKLPWTFGGGTKLEIK | |
| H5S19-17BL | DIQMTQTTSSLSASLGDRVTISCSASQGISNYLNWYQQKPDGTVKLLIYYTSSLHSGVPSRFRGSGSGTDYSLTISNLEPEDIA | 896 |
| | TYYCQQYSKLPWTFGGGTKLEIK | |
| H5S19-17CL | DIQMTQTTSSLSASLGDRVTISCSASQGISNYLNWYQQKPDGTVKLLIYYTSSLHSGVPSRFSGSGSGTDYSLTISNLEPEDIA | 897 |
| | TYFCQQYSKLPWTFGGGTKLEIK | |
| H5S19-17DL | DIQMTQTTSSLSASLGDRVTISCSASQGISNYLNWYQQKPDGTVKLLIYYTSSLHSGVPSRFSGSGSGTDYSLSISNLEPEDIA | 898 |
| | TYYCQQYSKLPWTFGGGTKLEIK | |
| H5S19-17EL | DIQMTQTTSSLSASLGDRVTISCSASQGISNYLNWYQQKPDGTVKLLIYYTSSLHSGVPSRFSGSGSGTDYSLTFSNLEPEDIA | 899 |
| | TYYCQQYSKLPWTFGGGTKLEIK | |
| H5S19-17FL | DIQMTQTTSSLSASLGDRVTISCSASQGISNYLNWYQQKPDGTVKLLIYYTSSLHSGVPSRFSGSGSGTDYSLTISNLEPEDIA | 900 |
| | TYYCQQYSKLPWTFGGGTKLEI | |
| H5S19-1AL | NIVLTQSPASLAVSLGQRATISCRASESVDSYGNSLMHWYQQKPGQPPKLLIYLASNLESGVPARFSGSGSRTDFTLTIDPVE | 901 |
| | ADDAATYYCQQNNEDPPTFGGGTKLEI | |
| H5S19-20AL | DIQMTQTTSSLSASLGDRVTISCTASQGISNYLNWYQQKPDGTVKLLIYYTSTLHSGVPSRFSGSGSGTDYSLTISNLEPEDIAT | 902 |
| | YYCQQYFKLPWTFGGGTKLEIK | |
| H5S19-23AL | DIQMTQTTSSLSASLGDRVTISCSASQGINNYLNWYQQKPDGTVKLLIYYTSSLHSGVPSRFSGSGSGTDYSLTISNLEPEDIA | 903 |
| | TYYCQQYSKLPWTFGGGTKLEIK | |
| H5S19-26AL | DIQMTQTTSSLSASLGDRVTISCTASQGINNYLNWYQQKPDGTVKLLIYYTSTLHSGVPSRFSGSGSGTDYSLTISNLEPEDIAT | 904 |
| | YYCQQYFKLPWTFGGGTKLEIK | |
| H5S19-28AL | DIQMTQTTSSLSASLGDRVTISCTASQGISNYLNWYQQKPDGTVKLLIYYTSTLHSGVPSRFSGSGSGTDYSLTISNLEPEDIAT | 905 |
| | YYCQQYSKLPWTFGGGTKLAIK | |
| H5S19-6AL | QIVLTQSPAIMSASPGEKVTMTCSASSSVSYMYWYQQKPGSSPRLLIYDTSNLASGVPVRFSGSGSGTSYSLTISRMEAEDAA | 906 |
| | TYYCQQWSSYPFTFGSGTKLEIK | |
| H5S19-8AL | DVQITQSPSYLAASPGETITINCRTSKNISKYLAWYQEKPGKTNKLLIYSGSTLQSGIPSRFSGSGSGTDFTLTISSLEPEDFVM | 907 |
| | YHCQQHNEYPWTFGGGTKLEIK | |
| H5S20-13AL | DIVLTQSPASLAVSLGQRATISCKASQSVDYDGNSYMNWYQQKPGQPPKLLIYAASNLESGIPARFSGSGSGTDFTLNIHPVE | 908 |
| | EEDAATYYCQQSNEDPWTFGGGTKLEIK | |
| H5S20-16AL | QIVLTQSPALMSASPGEKVTMTCSASSSVSFIYWYQQKPRSSPKPWISLTSNLASGVPARFSGSGSGTSYSLTISSMEAEDAA | 909 |
| | TYYCQQWSSNPLTFGAGTKLEL | |
| H5S20-16BL | QIVLTQSPALMSASPGEKVTMTCSASSSVSFIYWYQQKPRSSPKPWIYLTSNLASGVPARFSGSGSGTSYSLTISSMEAEDAA | 910 |
| | TYYCQQWSSNPLTFGAGTKLEL | |
| H5S20-17AL | DIVMTQSPATLSVTPGDRVSLSCRASRTISDYLHWYQQKSHESPRLLIKYASQSISGIPSRFSGSGSGSDFTLSISSVEPEDVG | 911 |
| | MYYCQNGHSFPLTFGAGTKLELK | |
| H5S20-18AL | NIVLTQSPASLAVSLGQRATISCRASESVDSYGNSFMHWYQQKPGQPPKLLIYLASNLESGVPARFSGSGSRTDFTLTIDPVE | 912 |
| | ADDAATYYCQQNYADPWTFGGGTKLEIK | |
| H5S20-20AL | DIVLTQSPASLAVSLGQRATISCKASQSVDYDGDSFMNWYQQKPGQPPKLLIYAASNLESGIPARFSGSGSGTDFTLNIHPVE | 913 |
| | EEDAATYYCQQSNEDPWTFGGGTKLEIK | |
| H5S20-23AL | DIVMTQSPSSLTVPAGEKVTMSCKSSQSLLNSENQKNYLTWYQQKPGQPPKLLIYWASTRESGVPDRFTGSGSGTDFTLTIS | 914 |
| | SVQAEDLAVYYCQSDYSYPLTFGAGTKLELK | |
| H5S20-32AL | DIVMTQSQKFMSTSVGDRVSVTCKASQNVGTNVAWYQQKPGQSPKALIYSASYRYSGVPDRFTGSGSGTDFTLTISNVQSE | 915 |
| | DLAEYFCQQYNSYPFTFGSGTKLEIK | |
| H5S20-34AL | QIVLTQSPALMSASPGEKVTMTCTASSSVSYMYWYQQKPRSSPKPWIYLTSNLASGVPARFSGSGSGTSYSLTISSMEAEDA | 916 |
| | ATYYCQQWSSNPLTFGAGTKLELR | |
| H5S20-36AL | DIVMTQSQKFMSTSVGDRVSVTCKASQNVGTNVAWYQQKPGQSPKALIYSASYRYSGVPDRFTGSGSGTDFTLTISNVQSE | 917 |
| and H5S20- | DLAEYFCQQYNSYPLTFGGGTKLEIK | |
| 43AL | | |
| H5S20-40AL | DIVMTQSQKFMSTSVGDRVSVTCKASQNVGTNVAWYQQKPGQSPKALIYSASYRYSGVPDRFTGSGSGTDFTLTISNVQSE | 918 |
| | DLAEYFCQQYNSYPLTFGAGTKLELK | |
| H5S20-8AL | DIVLTQSPASLAVSLGQRATISCKASQSVDYDGDSYMNWYQQKPGQPPKLLIYAASNLESGIPARFSGSGSGTDFTLNIHPVE | 919 |
| | EEDAATYYCQQSNEDPWTFGGGTKLEIK | |
| H5S20-8BL | DIVLTQSPASLAVSLGQRATISCKASQSVDYDGDSYMNWFQQKPGQPPKLLIYAASNLESGIPARFSGSGSGTDFTLNIHPVE | 920 |
| | EEDAATYYCQQSNEDPWTFGGGTKLEIK | |
| ITI_021L | NIVLTQSPASLAVSLGQRATISCRASESVDSYGNSFMHWYQQKPGQPPKLLIYLASNLESGVPARFSGSGSRTDFTLTIDPVE | 921 |
| | ADDAATYYCQQNNEDPLTFGGGTKLEIK | |
| ITI_040L | QIVLTQSPAIMSASPGEKVTISCSASSSISYMYWYQQKPGSSPKPWIYRTSNLASGVPARFSGSGSGTSYSLTISSMEAEDAA | 922 |
| | TYYCQQYHSYPLTFGAGTKLELK | |
| ITI_045L | EIVLTQSPTTMAASPGEKITITCSASSSINSNYLHWYQQKPGFSPKLLIYRTSNLASGVPPRFSGSGSGTSYSLTIGTMEAEDVA | 923 |
| | TYYCQQGSTIPYTFGGGAKLEIK | |
| ITI_082L | ENVLTQSPAIMSASLGEKVTMSCRASSSVNYMYWYQQKSDASPKLWIYYTSNLAPGVPARFSGSGSGNSYSLTISSMEGED | 924 |
| | AATYYCQQFTSSTWTFGGGTKLEIK | |
| ITI_083L | ENVLTQSPAIMSASLGEKVTMSCRASSSVNYMFWYQQKSDASPKLWIYSTSNLTPGVPARFSGSGSGNSYSLTISSMEAEDA | 925 |
| | ATYYCQQFTSSTWTFGGGTKLEIK | |
| ITI_131L | EIVLTQSPALMAASPGEKVTITCSVSSSISSSNLHWYQQKSETSPKPWIYGTSNLASGVPVRFSGSGSGTSYSLTISSMEAEDA | 926 |
| | ATYYCQQWSSYPLTFGSGTKLEIK | |
| ITI_144L | DIQMTQTPSSLSASLGDRVTISCSASQDINNYLNWYQQKPDGTVKLLIYYTSSLHSGAPSRFSGSGSGTDYSLTISNLDPEDIA | 927 |
| | TYYCQQYSKLPWTFGGGTKLEIK | |
| ITI_145L | DIQMTQTPSSLSASLGDRVTISCSASQDINNYLNWYQQKPDGTVKLLIFYTSSLHSGAPSRFSGSGSGTDYSLTISNLDPEDIA | 928 |
| | TYYCQQYSKLPWTFGGGTKLEIK | |
| ITI_146L | DIQMTQTTSSLSASLGDRVTISCSASQGIRNYLNWYQQKPDGTVKLLIYYTSSLHSGVPSRFSGSGSGTDYSLTISNLEPEDIA | 929 |
| | TYYCQQYSKLPWTFGGGTKLEIK | |
| ITI_162L | DIQMTQTTSSLSASLGDRVTISCTASQGISNYLNWYQQKPDGTVKLLIYYTSTLHSGVPSRFSGSGSGTDYSLTINNLEPEDIAT | 930 |
| | YYCQQYSKLPWTFGGGTKLEIK | |
| ITI_166L | DIQMTQTTSSLSASLGDRVTISCSASQDISNYLNWYQQKPDGTVKLLIYYTSSLHSGVPSRFSGSGSGTDYSLTISNLEPEDIAT | 931 |
| | YYCQQYSKLPWTFGGGTKLEIK | |
| ITI_169L | DIQMTQTTSSLSASLGDRVTISCSASQGITKYLNWYQQKPDGTVKLLIYYTSSLHSGVPSRFSGSGSGTDYSLTISNLEPEDIAT | 932 |
| | YYCQQYSKLPWTFGGGTKLEIK | |
| ITI_200L | DIVLTQSPASLAVSLGQRATISCKASQSVDYDGESYINWYQQRPGQPAKLLIFAASNLESGIPARFSGSGSGTDFTLNIHPVEE | 933 |
| | EDAASYYCQHCYEDPWTFGGGTKLEIK | |
| ITI_236L | DIQMTQTTSSLSASLGDRVTISCRASQDISNYLNWYQQKPDGTVKLLIYYTSRLHSGVPSRFSGSGSGTDYSLTISNLEQEDIA | 934 |
| | TYFCQQGNTLPYTFGGGTKLEIK | |
| ITI_237L | DIQMTQTTSSLSASLGDRVTISCSASQGISNYLNWYQQKPDGTVKLLIYYTSSLHSGVPSRFSGSGSGTDYSLTISNLEPEDIA | 935 |
| | TYYCQQYSKLPRTFGGGTKLEIK | |
| H5S14-11AL | DVVMTQTPLTLSVTIGQPASISCKSSQSLLDSDGKTYLNWLLQRPGQSPKRLIYLVSKLDSGVPDRFTGSGSGTDFTLKISRVE | 936 |
| | AEDLGVYYCWQLTHFPQTFGGGTKLEIK | |
| H5S14-13AL | DVLMTQTPLSLPVSLGDQASISCRSSQSIVHSNGNTYLEWYLQKPGQSPKLLIYKVSNRFSGVPDRFSGSGSGTDFTLKISRV | 937 |
| and H5S19-7AL | EAEDLGVYYCFQGSHVPWTFGGGTKLEIK | |
| H5S14-17AL | DWVMTQTPLTLSVTIGQPASISCKSSQSLLDSDGKTYLNWLLQRPGQSPKRLIYLVSKLDSGVPDRFTGSGSGTDFTLKISRVE | 938 |
| | AEDLGVYYCWQVTHFPQTFGGGTKLEIK | |
| H5S14-2AL | DVVMTQTPLTLSVTIGQPASISCKSSQSLLDSDGKTYLNWLLQRPGQSPKRLIYLVSKLDSGVPDRFTGSGSGTDFTLKISRVE | 939 |
| | AEDLGVYYCWQGTHFPHAFGGGTKLEIK | |
| H5S15-12CL | DVVMTQTPLTLSVTIGQPASISCKSSQSLLDSDGKTYLNWLLQRPGQSPKRLIYLVSKLDSGVPDRFTGSGSGTDFTLKISRVE | 940 |
| | AEDLGIYYCWQGTHFPQTFGGGTKLEIK | |
| H5S15-16AL | DVLMTQTPLSLPVSLGDQASISCRSSQSIVHSNGITYLEWYLQKPGQSPELLIYKVSNRFSGVPDRFSGSGSGTDFTLKISRVE | 941 |
| | AEDLGVYYCFQGSHVPLTFGAGTKLELK | |
| H5S15-17AL | DVVMTQTPLTLSVTVGQPASISCKSSQSLLDSDGKTYLNWLLQRPGQSPKRLIYLVSKRDSGVPDRFTGSGSGTDFTLKISRV | 942 |
| | EAEDLGVYYCWQNTHFPQTFGGGTKLEIK | |
| H5S15-1AL | ENVLTQSPAIMSASPGEKVTMTCSAGSSVSYMHWYQQKSSTSPKLWIYDTSKLPSGVPGRFSGSGSGNSYSLTISSMEAED | 943 |
| | VATYYCFQGSGFPLTFGSGTKLEIK | |
| H5S15-31AL | DWVMTQTPLTLSVTIGQPASISCKSSQSLLDSDGKTYLNWLLQRPGQSPKRLIYLVSKLDSGVPDRFTGSGSGTDFTLKISRVE | 944 |
| | AEDLGVYYCWQATHFPQTFGGGTNLEIK | |
| H5S15-3AL | DVLMTQTPLSLPVSLGDQASISCRSSQSIVHSNGNTYLEWYLQKPGQSPKLLIYKVSNRFSGVPDRFSGSGSGTDFTLKISRV | 945 |
| | EAEDLGVYYCFQGSHVPLTFGAGTKLELK | |
| H5S15-3BL | DVLMTQTPLSLTVSLGHQASISCRSSQSIVHSNGNTYLEWYLQKPGQSPKLLIYKVSNRFSGVPDRFSGSGSGTDFTLKISRV | 946 |
| | EAEDLGVYYCFQGSHVPLTFGAGTKLELK | |
| H5S15-6AL | DVVMTQTPLTLSVTIGQPASISCKSSQSLLDSDGKTYLNWLLQRPGQSPKRLIYLVSKLDSGVPDRFTGSGSGTEFTLKISRVE | 947 |
| | AEDLGVYYCWQNTHFPQTFGGGTKLEIK | |
| H5S20-19AL | DVVMTQTPLTLSVTIGQPASISCKSSQSLLDSDGKTYLSWLLQRPGQSPKRLIYLVSKLDSGVPDRFTGSGSGTDFTLKISRVE | 948 |
| | AEDLGVYYCWQNTHFPQTFGGGTKLEIK | |
| H5S20-19BL | DWVMTQTPLSLSVTIGQPASISCKSSQSLLDSDGKTYLSWLLQRPGQSPKRLIYLVSKLDSGVPDRFTGSGSGTDFTLKISRVE | 949 |
| | AEDLGVYYCWQNTHFPQTFGGGTKLEIK | |
| H5S20-21AL | DVLMTQTPLSLPVSLGDQASISCRSSQSIVHSNGNTYLEWYLQKPGQSPKLLIYKVSNRVSGVPDRFSGSGSGTDFTLKISRV | 950 |
| | EAEDLGVYYCFQGSHVPLTFGAGTKLELK | |
| H5S20-24AL | DWVMTQTPLTLSVTIGQPASISCKSSQSLLDSDGKTYLNWLLQRPGQSPKRLIYLVSKLDSGVPDRFTGSGSGTDFTLKISRVE | 951 |
| | AEDLGVYYCWQNTHFPQTLGGGTKLEIK | |
| H5S20-25AL | DVVMTQTPLTLSVTIGQPASISCKSSQSLLDSDGKTYLNWLLQRPGQSPKRLIYLVSKLDSGVPDRFTGSGSGTDFTLKISRVE | 952 |
| | AEDLGVYYCWQGTHFPQTFGGGTKLEIK | |
| H5S20-26AL | DWVMTQTPLTLSVTIGQPASISCKSSQSLLDSDGKTYLYWLLQRPGQSPKRLIYLVSKLDSGVPDRFTGSGSGTYFTLKISRVE | 953 |
| | AEDLGVYYCWQNTHFPQTFGGGTKLEIK | |
| H5S20-31AL | DVVMTQTPLTLSVTIGQPASISCKSSQSLLDSDGKTYLNWLLQRPGQSPKRLIYLVSKVDSGVPDRFTGSGSGTDFTLKISRVE | 954 |
| | AEDLGVYYCWQNTHFPQTFGGGTKLEIK | |
| H5S20-39AL | DVLMTQTPLSLPVSLGDQVSISCRSSQNIVHSNGNTYLEWYLQKPGQSPKLLIYKVSNRFSGVPDRFSGSGSGTDFTLKISRV | 955 |
| | EAEDLGVYYCFQGSHVPLTFGAGTKLELK | |
| H5S20-3BL | DWVMTQTPLTLSVTIGQPASISCKSSQSLLDSDGKTYLNWLLQRPGQSPKRLIYLVSKLDSGVPDRFTGSGSGTDFTLKISRVE | 956 |
| | AEDLGVYYCWQATHFPQTFGGGTKLEIK | |
| H5S20-41AL | DVVMTQTPLTLSVTIGQPASISCKSSQSLLYSNGKTYLNWLLQRPGQSPKRLIYLVSKLDSGVPDRFTGSGSGTDFTLKISRVE | 957 |
| | AEDLGVYYCVQGTHFPMYTFGGGTKLEIK | |
| H5S20-7AL | DWVMTQTPLTLSVTIGQPASISCKSSQSLLDSDGKTYLNWLLQRPGQSPKRLIYLVSKLDSGVPDRFTGSGSGTDFTLKISRVE | 958 |
| | AEDLGVYYCWQNTHFPQTFGGGTKLEIK | |
| H5S20-7BL | DWVMTQTPLTLSVTIGQPASISCKSSQSLLDSDGKTYLNWLLQRPGQSPKRLIYLVSKLDSGVPDRFTGSGSGTDFTLKISRVE | 959 |
| | AEDLGIYYCWQNTHFPQTFGGGTKLEIK | |
| H5S20-7CL | DWVMTQTPLTLSITIGQPASISCKSSQSLLDSDGKTYLNWLLQRPGQSPKRLIYLVSKLDSGVPDRFTGSGSGTDFTLKISRVE | 960 |
| | AEDLGVYYCWQNTHFPQTFGGGTKLEIK | |
| H5S20-7DL | DWVMTQTPLTLSVTIGQPASISCKSSQSLLDSDGKTYLNWLLQRPGQSPKRLIYLVSKLDSGVPDRFTGSGSGTDFTLKISRVE | 961 |
| | AEDLGVYYCWQNTHFPQTFGGGTELEIK | |
| H5S20-7EL | DVVMTQTPLTLSVTIGQTASISCKSSQSLLDSDGKTYLNWLLQRPGQSPKRLIYLVSKLDSGVPDRFTGSGSGTDFTLKISRVE | 962 |
| | AEDLGVYYCWQNTHFPQTFGGGTKLEIK | |
| H5S20-9AL | DVLMTQTPLSLPVNLGDQASISCRSSQSIVHSNGNTYLEWYLQKPGQSPKLLIYKVSNRFSGVPDRFSGSGSGTDFTLKISRV | 963 |
| | EAEDLGVYYCFQGSHVPLTFGAGTKLELK | |
| H5S20-9DL | DVLMTQTPLSLPVSLGDQASISCRSSQSIVHSNGNTYLEWYLQKPGQSPKLLIYKVSNRFSGVPDRFSGSGSGTDFTLKISRV | 964 |
| | EAEDLGVYYCFQGSHVPLTFGTGTKLELK | |
| H5S20-9EL | DVLMTQTPLSLPVSLGDQASISCRSSQSIVHSNGNTYLEWYLQKPGQSPNLLIYKVSNRFSGVPDRFSGSGSGTDFTLKISRV | 965 |
| | EAEDLGVYYCFQGSHVPLTFGTGTKLELK | |
| ITI_047L | DVVMTQTPLTLSVTIGQPASISCKSSQSLLDSDGKTYLNWLLQRPGQSPKRLIYLVSKLDSGVPDRFTGNGSGTDFTLKISRVE | 966 |
| | AEDLGVYYCWQATHFPQTFGGGTKLEIK | |
| ITI_091L | DVLMTQTPLSLPVSLGDQASISCRSSQSIVHSNGNTYLEWYLQKPGQSPKLLIYKVSNRFSGVPDRFSGSGSGTDFTLNISRV | 967 |
| | EAEDLGVYYCFQGSHVPLTFGAGTKLELK | |
| H5S14-3AL | QIVLTQSPAIMSASLGEEITLTCSASSSVSYMQWYQQKSGTSPKLLIYSTSNLASGVPSRFSGSGSGTFYSLTISSVEAEDAAD | 968 |
| | YYCHQWSSYPTFGGGTELEI | |
| H5S14-5AL | QIVLTQSPAIISASLGEEVTLTCSASSSVSYMHWYQQKSGTSPKLLIYSTSNLASGVPSRFSGSGSGTFYSLTISSVEAEDAAD | 969 |
| | YYCHQWSSYLTFGAGTKLELK | |
| H5S15-8AL | QIVLTQSPAIMSASLGEEITLTCSASSSVSFMHWYQQKSGTSPKLLIYSTSNLASGVPSRFSGSGSGTFYSLTISSVEAEDAAD | 970 |
| | YYCHQWSSYLWTFGGGAKLEIK | |
| H5S19-10AL | QIVLTQSPAIMSASPGEKVTLTCSASSSVDSSYLFWYQQKPGSSPKLWIYSTSNLASGVPARFSGSGSGTSYSLTISSMEAED | 971 |
| | AASYFCHQWSSYPWTFGGGTILEIK | |
| H5S19-10BL | QIVLTQSPAIMSASPGEKVTLTCSASSSVDSSYLFWYQQKPGSSPKLWIYSTSNLASGVPARFSGSGSGTSYSLTISSMEAED | 972 |
| | AASYFCHQWSSYPWTFGGGTILEI | |
| H5S19-14AL | QIVLTQSPAIMSASPGEKVTLTCSASSSVDSSYLFWYQQKPGSSPKLWIYSTSNLASGVPARFSGSGSGTSYYLTLSSMEAED | 973 |
| | AASYFCHQWSNFAWTFGGGTILEIK | |
| H5S19-16AL | QIVLTQSPAIMSASPGEKVTLTCSASSSVDSSYLYWFQQKPGSSPKLWIYGTSNLASGVPARFSGSGSGTSYSLTISSMEAED | 974 |
| | AASYFCHQWNSYPWTFGGGTKLEIK | |
| H5S19-16BL | QIVLTQSPAIMSASPGEKVTLTCSASSSVDSSYLYWYQQKPGSSPKLWIYGTSNLASGVPARFSGSGSGTSYSLTISSMEAED | 975 |
| | AASYFCHQWNSYPWTFGGGTKLEIK | |
| H5S19-18AL | QIVLTQSPAIMSTSPGEKVTLTCSASSSVSSSYLYWYQQKPGSSPKLWIYSTSNLASGVPARFSGSGSGTSYSLTISSMEAED | 976 |
| | AASYFCHQWSTYPWTFGGGTKLEIK | |
| H5S19-19AL | QIVLTQSPAIMSASPGEKVTLTCSASSSVSSSYLYWYQQKPGSSPKLWIYSTSNLASGVPARFSGSGSGTSYSLTISSMEAED | 977 |
| | AASYFCHQWSSYPWTFGGGTKLEIK | |
| H5S19-3AL | QIVLTQSPAIMSASPGEKVTLTCSASSSVDSSYLFWYQQKPGSSPKLWIYSTSNLASGVPARFSGSGSGTSYSLTISSMEAED | 978 |
| | AASYFCHQWNSYPWTFGGGTKLEIK | |
| H5S19-4AL | QIVLTQSPAIMSASPGEKVTLTCSASSSVDSSYLFWYQQKPGSSPKLWIYSTSNLASGVPARFSGSGSGTSYYLTISSMEAED | 979 |
| | AASYFCHQWSSYAWTFGGGTILEIK | |
| H5S19-5AL | QIVLTQSPAIMSASPGEKVTLTCSASSSVDSSYLFWYQQKPGSSPKLWLYSTSNLASGVPARFSGSGSGTSYYLTISSMEAED | 980 |
| | AASYFCHQWSNYAWTFGGGTILEIK | |
| H5S20-10AL | QIVLTQSPAIMSASLGEEITLTCSASSSVSYMHWYQQRSGTSPKLLIYSTSNLASGVPSRFSGSGSGTFYSLTISSVEAEDAAD | 981 |
| | YYCHQWSSYRTFGGGTKLEIK | |
| H5S20-15AL | QIVLTQSPAIMSASLGEEITLTCSASSSVSYMHWYQQKSGTSPKLLIYSASNLASGVPSRFSGSGSGTFYSLTISSVEAEDAAD | 982 |
| | YYCHQWSSYRTFGGGTKLEIK | |
| H5S20-22AL | QIVLTQSPAIMSASLGEEITLTCSASSSVSYMHWYQQKSGSSPKLLIYTTSNLASGVPSRFSGSGSGTFYSLTISSVEAEDAAD | 983 |
| | YYCHQWSSYTWTFGGGTKLEIK | |
| ITI_027L | QIVLTQSPAIMSASPGEKVTLTCSASSSVDSSYLFWYQQKPGSSPKLWIYSTSNLASGVPARFSGSGSGTSYSLTISSMEAED | 984 |
| | AASYFCHQWSSYPWTFGGGTKLEIK | |
| ITI_028L | QIVLTQSPAIMSASPGEKVTLTCSASSSVDSSYLYWYQQKPGSSPKLWIYSTSNLASGVPARFSGSGSGTSYSLTISSMEAED | 985 |
| | AASYFCHQWNSYPWTFGGGTKLEIK | |
| ITI_029L | QIVLTQSPAIMSASPGEKVTLTCSASSSVDSSYLFWYQQKPGSSPKLWIYSTSNLASGVPARFSGSGSGTSYSLTISSMEAED | 986 |
| | AASYFCHQWSSYAWTFGGGTILEIK | |
| ITI_030L | QIVLTQSPAIMSASPGEKVTLTCSASSSVDSSYLYWFQQKPGSSPKLWIYGTSNLASGVPVRFSGSGSGTSYSLTISSMEAED | 987 |
| | AASYFCHQWNSYPWTFGGGTKLEIK | |
| ITI_033L | QIVLTQSPAIMSASPGEKVTLTCSASSSVDSSYLFWYQQKPGSSPKLWIYSTSNLASGVPARFSGSGSGTSYSLTISSMEAED | 988 |
| | AASYFCHQWSSYPWTFGGGTMLEIK | |
| ITI_038L | QIVLTQSPAIMSASPGEKVTLTCSASSSVDSSYLYWYQQKPGSSPKLWIYGTSNLASGVPVRFSGSGSGTSYSLTISSMEAED | 989 |
| | AASYFCHQWNSYPWTFGGGTKLEIK | |
| ITI_127L | QIVLTQSPAIMSASLGEEITLTCSARSSVSYMFWYQQKSGTSPKLLIYTTSNLASGVPSRFSGSGSGTFFSLTISGVEAEDAAD | 990 |
| | YYCHQWSSYTWTFGGGTKLEIK | |
| ITI_128L | QIVLTQSPAIVSASLGAEITLTCSARSTVSYMFWYQQKSGTSPKLLIYSTSNLASGVPSRFSGSGSGTFYSLTISGVEAEDAAD | 991 |
| | YYCHQWSSYTWTFGGGTKLEIK | |
| H5S14-21AL | DIVMSQSPSSLAVSVGEKVTMSCKSSQSLLYSSNQKNYLAWYQQKPGQSPKLLIYWASTRESGVPDRFTGSGSGTDFTLTIS | 992 |
| | SVKAEDLAVYYCQQYYSYPWTFGGGTKLEIK | |
| H5S14-22AL | DIQMTQSPSSLSASLGERVSLTCRASQEISGYLSWLQQKPDGTIKRLIYAASTLDSGVPKRFSGSRSGSDYSLTISSLESEDFA | 993 |
| | DYYCLQYASYPFTFGSGTKLEIK | |
| H5S14-23AL | DIKMTQSPSSMYASLGERVTITCKASQDINSYLSWFQQKPGKSPKTLIYRANRLVDGVPSRFSGSGSGQDYSLTISSLEYEDM | 994 |
| | GIYYCLQYDEFPWTFGGGTKLEIK | |
| H5S15-19AL | QIVLTQSPAIMSASLGERVTLTCTASSSVSSSYLHWYQQKPGSSPKLWIYDTSNLASGVPARFSGSGSGTSYSLTISSMEAED | 995 |
| | AATYYCHQYHRSQWTFGGGTKLEIK | |
| H5S15-21AL | DIVMSQSPSSLAVSVGEKVTMSCKSSQSLLYSSNQKNYLAWYQQKPGQSPKLLIYWASTRESGVPDRFTGSGSGTDFTLTIS | 996 |
| | SVKAEDLAVYYCQQYYSYPYTFGGGTKLEIK | |
| H5S15-26AL | DIVMSQSPSSLAVSVGEKVTMSCKSSQSLLYSSNQKNYLAWYQQKPGQSPKLLIYWASTRESGVPDRFTGSGSGTDFTLTIS | 997 |
| | SVKAEDLAVYYCQQYYSYPRTFGGGTKLEIK | |
| H5S19-15AL | DIKMTQSPSSMYASLGESVTITCKASQDINSYLNWFQQKPGKSPKTLIYRANRLVDGVPSRFSGSGSGQDYSLTISSLEFEDM | 998 |
| | GIYYCLQYDEFPFTFGSGTKLEMK | |
| H5S19-9BL | DIKMTQSPSSMYASLGESVTITCKASQDINSYLSWFQQKPGKSPKTLIYRANRLVDGVPSRFSGSGSGQDYSLTISSLEFEDM | 999 |
| | GIYYCLQYDEFPFTFGSGTKLEMK | |
| H5S20-12AL | DIVMSQSPSSLAVSVGEKVTMSCKSSQSLLYSSNQKNYLAWYQQKPGQSPKLLIYWASTRESGVPDRFTGSGSGTDFTLTIS | 1000 |
| | SVKAEDLAVYYCQQYYSYPFTFGSGTKLEIK | |
| H5S20-12BL | DIVMSQSPSSLAVSVGEKVTMSCKSSQSLLYSSNQKNYLAWYQQRPGQSPKLLIYWASTRESGVPDRFTGSGSGTDFTLTIS | 1001 |
| | SVKAEDLAVYYCQQYYSYPFTFGSGTKLEIK | |
| H5S20-1AL | DIVMSQSPSSLAVSVGEKVTMSCKSSQSLLYSSDQKNYLAWYQQKPGQSPKLLIYWASTRESGVPDRFTGSGSGTDFTLTIS | 1002 |
| | SVKAEDLAVYYCQQYYSYPFTFGSGTKLEIK | |
| H5S20-1BL | DIVMSQSPSSLGVSVGEKVTMSCKSSQSLLYSSDQKNYLAWYQQKPGQSPKLLIYWASTRESGVPDRFTGSGSGTDFTLTIS | 1003 |
| | SVKAEDLAVYYCQQYYSYPFTFGSGTKLEIK | |
| H5S20-27AL | DIVMSQSPSSLAVSVGEKVTMSCKSSQSLLYSSDQKNYLAWYQQKPGQSPKLLIYWASTGESGVPDRFTGSGSGTDFTLTIS | 1004 |
| | SVKAEDLAVYYCQQYYSYPFTFGSGTKLEIK | |
| H5S20-28AL | DIVMSQSPSSLAVSVGEKVTMSCKSSQSLLYSSDQKNYLAWYQQKPGQSPKLLIYWASTRGSGVPDRFTGSGSGTDFTLTIS | 1005 |
| | SVKAEDLAVYYCQQYYSYPFTFGSGTKLEIK | |
| H5S20-29AL | DIVMSQSPSSLAVSVGEKITMSCKSSQSLLFSSNQKNYLAWYQQKPGQSPKLLIYWASTRESGVPDRFTGSGSGTDFTLTISS | 1006 |
| | VKAEDLAVYYCQQYYSYPFTFGSGTKLEIK | |
| H5S20-30AL | DIVMSQSPSSLPVSVGEKVTMTCKSSQSLLYGSNQKNYLAWYQQKPGQSPKLLIYWASTRESGVPDRFTGSGSGTDFTLTIS | 1007 |
| | SVKAEDLAVYYCQQYYSYPFTFGSGTKLEIK | |
| H5S20-33AL | DIVMSQSPSSLAVSVGEKITMSCKSSQSLLFSSNQKNYLAWYQQKPGQSPKLLIYWASTRESGVPDRFTGSGSGTDFTLTISS | 1008 |
| | VKAEDLAVYYCQQYYTYPFTFGSGTKLEMK | |
| H5S20-42AL | DIVMSQSPSSLAVSVGEKITMSCKSSQSLLFGSNQKNYLAWYQQKPGQSPKLLIYWASTRESGVPDRFTGSGSGTDFTLTISS | 1009 |
| | VKAEDLAVYYCQQYYTYPFTFGSGTKLEMK | |
| H5S20-4AL | DIVMSQSPSSLAVSVGEKITMSCKSSQSLLFSSIQKNYLAWYQQKPGQSPKLLVYWASTRESGVPDRFTGSGSGTDFTLTISS | 1010 |
| | VKAEDLAVYYCQQYYSYPFTFGSGTKLEIK | |
| H5S20-6AL | DIVMSQSPSSLAVSVGEKVTMSCKSSQSLLYSSNQKNYLAWYQQKPGQSPKLLIYWSSTRESGVPDRFTGSGSGTDFTLTIS | 1011 |
| | SVKAEDLAVYYCQQYYSYPFTFGSGTKLEIK | |
| ITI_122L | QIVLTQSPAIMSASLGERVTMTCTASSSVSSSYLHWYQQKPGSSPKLWIYNTSNLASGVPARFSGSGSGTSFSLTISSMEAED | 1012 |
| | AATYYCHQYHRSPTFGGGTKLEIK | |
| ITI_173L | DIKMTQSPSSMYASLGESVTITCKASQDINSYLSWFQQKPGKSPKTLIYRANRLVDGVPSRFSGSGSGQDYSLTISSLEFEDM | 1013 |
| | GIYFCLQYDEFPFTFGSGTKLEMK | |
| ITI_203L | DILMTQSPSSMSVSLGDTVNITCHASQGISSNIGWLQQKPGKSFKGLIYHGTNLEDGVPSRFSGSGSGADYSLTISNLESEDF | 1014 |
| | ADYYCGQYGQFPPTFGGGTKLEI | |
| H5S14-10AL | DVVMTQTPLSLPVSLGDQASISCRSSQSLVHSNGNTYLHWYLQKPGQSPKLLIYKVSNRFSGVPDRFSGSGSGTDFTLKISRV | 1015 |
| | EAEDLGVYFCSQSIHVPFTFGSGTKLEIK | |
| H5S14-7AL and | DWVMTQTPLSLPVSLGDQASISCRSSQSLVHSNGNTYLHWYLQKPGQSPKLLIYKVSNRFSGVPDRFSGSGSGTDFTLKISRV | 1016 |
| H5S15-23AL | EAEDLGVYFCSQSTHVPFTFGSGTKLEIK | |
| H5S15-10AL | DVVMTQTPLSLPVSLGDQASISCRSSQSLVHTNGNTYLHWYLQKPGQSPRLLIYKVSNRFSGVPDRFSGGGSGTDFTLKISR | 1017 |
| | VEAEDLGVYFCSQSTHVPTFGSGTKLEIK | |
| H5S15-11AL | DWVMTQTPLSLPVSLGDQASVSCRSSQSLVHSNGNTYLHWYLQKPGQSPKLLIYKVSNRFSGVPDRFIGSGSGTDFTLKISRV | 1018 |
| | EAEDLGVYFCSQSTHVPTFGSGTKLEVK | |
| H5S15-11BL | DWVMTQTPLSLSVSLGDQASISCRSSQSLVHSNGNTYLHWYLQKPGQSPKLLIYKVSNRFSGVPDRFSGSGSGTDFTLKISRV | 1019 |
| | EAEDLGVYFCSQSTHVPTFGSGTKLEIK | |
| H5S15-11CL | DWVMTQTPLSLPVSLGDQASISCRSSQSLVHSNGNTYLHWYLQKPGQSPKLMIYKVSNRFSGVPDRFSGSGSGTDFTLRISR | 1020 |
| | VEAEDLGVYFCSQSTHVPTFGSGTKLEIK | |
| H5S15-11DL | DVVMTQTPLSLPVSLGDQASISCRSSQSLVHSNGNTYLHWYLQKPGQSPKLLIYKVSNRFSGVPDRFSGSGSGTDFTLKISRV | 1021 |
| | EAEDLGVYFCSQSTHVPTFGSGTKLEIK | |
| H5S15-11EL | DVVMTQTPLSLPVSLGDQASISCRSSQSLVHSNGNTYLHWYLQKPGQSPKLLIYKVSNRFSGVPDRFSGSGSGTDFTLKISRV | 1022 |
| | ETEDLGVYFCSQSTHVPTFGSGTKLEIK | |
| H5S15-22AL | DWVMTQTPLSLPVSLGDQASISCRSSQSLVHSNGNTYLHWYLQKPGQSPKLLIYKVSNRFSGVPDRFSGSGSGTDFTLKISRV | 1023 |
| | EAEDLGVYFCSQSTHVPPWTFGGGTKLEIK | |
| H5S14-19AL | DIQMTQSPASLSVSVGETVTITCRASENIYSNLAWYQQKQGKSPQLLVYAATNLADGVPSRFSGSGSGTQYSLKINSLQSEDF | 1024 |
| | GSYYCQHFWGTPRTFGGGTKLEIK | |
| H5S14-9AL | DIQMTQSPASLSASVGETVTITCRASENIYSYLAWYQQKQGKSPQLLVYNAKTLAEGVPSRFSGSGSGTQFSLKINSLQPEDF | 1025 |
| | GSYYCQHHYGTMYTFGGGTKLEIK | |
| H5S15-2AL | DIQMTQSPASLSASVGETVTITCRASENIYSYLAWYQQKQGKSPQLLVYNAKTLAEGVPSRFSGSGSGTQFSLKINSLQPEDF | 1026 |
| | GSYYCQHHYGTYTFGGGTKLEIK | |
| H5S15-32AL | DIQMTQSPASLSASVGETVTITCRASGNIHNYLAWYQQKQGKSPQLLVYNAKTLADGVPSRFSGSGSGTQYSLKINSLQPEDF | 1027 |
| | GSYYCQHFWSTFTFGSGTKLEIK | |
| ITI_023L | DIQMTQSPASLSASVGETVTITCRASGNIHNYLTWYQQKQGKSPQLLVYNAKTLADGVPSRFSGSGSGTQYSLKINNLQPEDF | 1028 |
| | GSYYCQHFWSTFTFGSGTKLEVK | |
| ITI_240L | DIQMTQSPASLSASVGETVTITCRPSENIYSYLAWYQQKQGKSPQLLVYNAKTLAEGVPSRFSGSGSGTHFSLKINSLQPEEF | 1029 |
| | GSYYCQHHYGTPYTFGGGTKLEIK | |
| H5S14-12AL | ETTVTQSPASLSVATGEKVTIRCITSTDIDDDMNWYQQKPGEPPKLLISEGNTLRPGVPSRFSSSGYGTDFVFTIENTLSEDVA | 1030 |
| | DYYCLQSDNMPYTFGGGTKLEIK | |
| H5S19-22AL | DAVMTQTPLSLTVSLGDQASISCRSSQTLENTNGNTYLNWYLQKPGQSPQLLIYRVSNRFSGVLDRFSGSGSGTDFTLKISRV | 1031 |
| | EAEDLGVYFCLQVTHVPYTFGGGTKLEIK | |
| H5S19-24AL | DIVMTQAAFSNPVTLGTSASISCRSSKSLLHSNGITYLYWYLQKPGQSPQLLIYQMSNLASGVPDRFSSSGSGTDFTLRISRVE | 1032 |
| | AEDVGVYYCAQNLELPYTFGGGTKLEIK | |

TABLE 4

| Antibody | FR1 | CDR1 | FR2 | CDR2 | FR3 | CDR3 | FR4 |
|---|---|---|---|---|---|---|---|
| Chain | Seq. IDs | Seq. IDs | Seq. IDs | Seq. IDs | Seq. IDs | Seq. IDs | Seq. IDs |
| Heavy | 202-284 | 285-376 | 377-463 | 464-560 | 561-708 | 709-846 | 847-873 |
| Light | 1033-1098 | 1099-1162 | 1163-1229 | 1230-1261 | 1262-1342 | 1343-1425 | 1426-1449 |

The anti-HVEM antibodies were raised against amino acids 59-240 (i.e., the extracellular domain) of the human HVEM protein.

Thus, the invention provides the disclosed antibodies comprising an amino acid sequence of any one of SEQ ID NOS: referred to Tables 2-3. In particular, the present invention encompasses antibodies that immunospecifically bind to a HVEM polypeptide, a polypeptide fragment or variant, or an epitope of HVEM expressed on human monocytes as determined by immunoassays known in the art for assaying specific antibody-antigen binding. The sequences described in the each of Tables 2-3 can be used to construct the antibodies as described herein.

Variants of the anti-HVEM antibodies described herein are also contemplated. These antibody variants have at least 60%, at least 70%, at least 75%, at least 80%, at least 85%, at least 90%, at least 91%, at least 92%, at least 93%, at least 94%, at least 95%, at least 96%, at least 97%, at least 98%, or at least 99% amino acid sequence identity to any of the amino acid sequences identified in Tables 2 and/or 3. These variant antibodies must retain the ability to bind to HVEM. In preferred embodiments, the variants comprise the CDRs described in Table 2.

Polynucleotides encoding any anti-HVEM antibodies described herein (including the variants described in the previous paragraph) are preferred embodiments of the invention, along with polynucleotides at least about 60%, at least about 70%, at least about 75%, at least about 80%, at least 85%, at least 90%, at least 91%, at least 92%, at least 93%, at least 94%, at least 95%, at least 96%, at least 97%, at least 98%, or at least 99% amino acid sequence identity to a polynucleotide encoding an anti-HVEM antibody as described herein (including variants).

In particular embodiments, anti-HVEM antibodies comprise a heavy chain comprising VH CDR1, VH CDR2, and VH CDR3 comprising, respectively: SEQ ID Nos 285, 464, and 709 (consensus cluster 11); SEQ ID Nos 298, 470, and 720 (consensus cluster 20); SEQ ID Nos 304, 478, and 729 (consensus cluster 5); SEQ ID Nos 310, 481, and 733 (consensus cluster 23); SEQ ID Nos 321, 495, and 751 (consensus cluster 21); SEQ ID Nos 328, 504, and 753 (consensus cluster 10); SEQ ID Nos 336, 513, and 776 (consensus cluster 8); SEQ ID Nos 340, 514, and 783 (consensus cluser 15); SEQ ID Nos 347, 522, and 795 (consensus cluster 19); SEQ ID Nos 351, 525, and 801 (consensus cluster 14); SEQ ID Nos 355, 530, and 808 (consensus cluster 6); SEQ ID Nos 356, 531, and 811 (consensus cluster 12); SEQ ID Nos 358, 535, and 815 (consensus cluster 4); SEQ ID Nos 361, 538, and 816 (consensus cluster 9); SEQ ID Nos 364, 541, and 821 (consensus cluster 17); SEQ ID Nos 366, 544, and 826 (consensus cluster 7); SEQ ID Nos 367, 547, and 829 (consensus cluster 13); SEQ ID Nos 369, 550, and 833 (consensus cluster 18); SEQ ID Nos 371, 553, and 837 (consensus cluster 22); SEQ ID Nos 374, 557, and 841 (consensus cluster 16); SEQ ID Nos 338, 513, and 844 (consensus cluster 1); SEQ ID Nos 375, 559, and 845 (consensus cluster 2); or SEQ ID Nos 376, 560, and 846 (consensus cluster 3). In particular embodiments, anti-HVEM antibodies comprise a light chain comprising VL CDR1, VL CDR2, and VL CDR3 comprising, respectively: SEQ ID Nos 1099, 1230, and 1343 (consensus cluster 6); SEQ ID Nos 1129, 1246, and 1376 (consensus cluster 7); SEQ ID Nos 1136, 1249, and 1387 (consensus cluster 3); SEQ ID Nos 1142, 1251, and 1399 (consensus cluster 5); SEQ ID Nos 1152, 1248, and 1411 (consensus cluster 1); SEQ ID Nos 1155, 1256, and 1416 (consensus cluster 4); and SEQ ID Nos 1159, 1258, and 1422 (consensus cluster 2).

In further embodiments, anti-HVEM antibodies comprise both a heavy hain comprising VH CDR1, VH CDR2, and VH CDR3 comprising, respectively: SEQ ID Nos 285, 464, and 709 (consensus cluster 11); SEQ ID Nos 298, 470, and 720 (consensus cluster 20); SEQ ID Nos 304, 478, and 729 (consensus cluster 5); SEQ ID Nos 310, 481, and 733 (consensus cluster 23); SEQ ID Nos 321, 495, and 751 (consensus cluster 21); SEQ ID Nos 328, 504, and 753 (consensus cluster 10); SEQ ID Nos 336, 513, and 776 (consensus cluster 8); SEQ ID Nos 340, 514, and 783 (consensus cluser 15); SEQ ID Nos 347, 522, and 795 (consensus cluster 19); SEQ ID Nos 351, 525, and 801 (consensus cluster 14); SEQ ID Nos 355, 530, and 808 (consensus cluster 6); SEQ ID Nos 356, 531, and 811 (consensus cluster 12); SEQ ID Nos 358, 535, and 815 (consensus cluster 4); SEQ ID Nos 361, 538, and 816 (consensus cluster 9); SEQ ID Nos 364, 541, and 821 (consensus cluster 17); SEQ ID Nos 366, 544, and 826 (consensus cluster 7); SEQ ID Nos 367, 547, and 829 (consensus cluster 13); SEQ ID Nos 369, 550, and 833 (consensus cluster 18); SEQ ID Nos 371, 553, and 837 (consensus cluster 22); SEQ ID Nos 374, 557, and 841 (consensus cluster 16); SEQ ID Nos 338, 513, and 844 (consensus cluster 1); SEQ ID Nos 375, 559, and 845 (consensus cluster 2); or SEQ ID Nos 376, 560, and 846 (consensus cluster 3), and further comprise a light chain comprising VL CDR1, VL CDR2, and VL CDR3 comprising, respectively: SEQ ID Nos 1099, 1230, and 1343 (consensus cluster 6); SEQ ID Nos 1129, 1246, and 1376 (consensus cluster 7); SEQ ID Nos 1136, 1249, and 1387 (consensus cluster 3); SEQ ID Nos 1142, 1251, and 1399 (consensus cluster 5); SEQ ID Nos 1152, 1248, and 1411 (consensus cluster 1); SEQ ID Nos 1155, 1256, and 1416 (consensus cluster 4); and SEQ ID Nos 1159, 1258, and 1422 (consensus cluster 2). In some embodiments, the antibody further comprises at least the VH FR2 and VH FR3 corresponding to the consensus cluster of the VH CDRs listed above. And in some embodiments, the antibody further comprises the VH FR1, VH, FR2, VH FR3, and FH FR4 corresponding to the consensus cluster of the VH CDRs listed above (i.e., SEQ ID Nos 202, 377, 561, and 847 in the case of consensus cluster 11). In some embodiments, the antibody further comprises at least the VL FR2 and VL FR3 corresponding to the consensus cluster of the VL CDRs listed above. And in some embodiments, the antibody further comprises the VL FR1, VL, FR2, VL FR3, and FL FR4 corresponding to the consensus cluster of the VL CDRs listed above (i.e., SEQ ID Nos 1033, 1163, 1262, and 1426 in the case of consensus cluster 6).

In some embodiments, the anti-HVEM antibody comprises VH CDR1, VH CDR2, and VH CDR3 of an antibody listed in Table 1 herein. In some embodiments, the anti-HVEM antibody comprises VL CDR1, VLCDR2, and VL CDR3 of an antibody listed in Table 1 herein. In some embodiments, the anti-HVEM antibody comprises VH CDR1, VH CDR2, and VH CDR3 and the VL CDR1, VL CDR2, and VL CDR3 of an antibody listed in Table 1 herein.

In some embodiments, the anti-HVEM antibody comprises the VH CDR1, VH CDR2, and VH CDR3 and the VL CDR1, VH CDR2, and VH CDR3 of any one of antibodies Ab_001 (H5S14-1A1A) (i.e., SEQ ID Nos. 370, 551, 834, 1102, 1234, and 1346, respectively), Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087. In some embodiments, the anti-HVEM antibody comprises the VH CDR1, VH CDR2, and VH CDR3 and the VL CDR1, VH CDR2, and VH CDR3 of any one of antibodies Ab_001 (H5S14-1A1A) (i.e., SEQ ID Nos. 370, 551, 834, 1102, 1234, and 1346, respectively), Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087, and further comprises a VH region with an amino acid sequence that is at least 90%, at least 95%, at least 97%, or at least 99% identical to that of the VH of the corresponding Ab_001, Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087 antibody, and/or further comprises a VL region with an amino acid sequence that is at least 90%, at least 95%, at least 97%, or at least 99% identical to that of the VL of the corresponding Ab_001, Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087 antibody. (For example, in the case of Ab_001, the antibody comprises CDRs comprising SEQ ID Nos. 370, 551, 834, 1102, 1234, and 1346, respectively, and a VH comprising an amino acid sequence at least 80%, at least 90%, at least 95%, at least 97%, at least 98%, or at least 99% identical to that of SEQ ID No. 191 (H5S14-1AH of Ab_001), and or comprises a VL comprising an amino acid sequence at least 80%, at least 90%, at least 95%, at least 97%, at least 98%, or at least 99% identical to that of SEQ ID No. 877 (H5S14-1AL of Ab_001). In some embodiments, the anti-HVEM antibody comprises the VH CDR1, VH CDR2, and VH CDR3 and the VL CDR1, VH CDR2, and VH CDR3 of any one of antibodies Ab_001 (H5S14-1A1A) (i.e., SEQ ID Nos. 370, 551, 834, 1102, 1234, and 1346, respectively), Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087, and further comprises a VH and a VL region, each with an amino acid sequence that is at least 80%, at least 90%, at least 95%, at least 97%, at least 98%, or at least 99% identical to that of the VH and/or the VL of the corresponding Ab_001, Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087 antibody. In some embodiments, the antibody comprises both the VH and the VL region of the Ab_001, Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087 antibody. In some embodiments above, the antibody binds to HVEM with a K_{D} of 100 nM or less, 50 nM or less, or 10 nM or less (i.e. 1E-07 or less, 5E-08 or less, or 1E-08 or less) (e.g., as determined in a bio-layer interferometry (BLI) assay such as Biacore® or OctetRed®). In some embodiments, above, the antibody also binds to cynomolgus monkey HVEM. In some embodiments above, the antibody blocks binding of human BTLA to human HVEM and/or blocks binding of human LIGHT to human HVEM.

In some embodiments, the anti-HVEM antibody blocks binding of human BTLA to human HVEM with an IC50 of 10 nM or less (e.g. in a competitive binding assay as described in the Examples herein). In some such cases, the anti-HVEM antibody comprises the VH CDR1, VH CDR2, VH CDR3, VL CDR1, VL CDR2, and VL CDR3 of Ab_001, Ab_008, Ab_009, Ab_025, Ab_026, Ab_027, Ab_028, Ab_029, Ab_034, Ab_035, Ab_036, Ab_043, Ab_050, Ab_051, Ab_058, Ab_063, Ab_159, Ab_064, Ab_065, Ab_066, Ab_072, Ab_073, Ab_074, Ab_078, Ab_080, Ab_083, Ab_153, or Ab_087. In some embodiments, the anti-HVEM antibody comprises the VH CDR1, VH CDR2, and VH CDR3 and the VL CDR1, VH CDR2, and VH CDR3 of any one of antibodies Ab_001, Ab_008, Ab_009, Ab_025, Ab_026, Ab_027, Ab_028, Ab_029, Ab_034, Ab_035, Ab_036, Ab_043, Ab_050, Ab_051, Ab_058, Ab_063, Ab_159, Ab_064, Ab_065, Ab_066, Ab_072, Ab_073, Ab_074, Ab_078, Ab_080, Ab_083, Ab_153, or Ab_087, and further comprises a VH region with an amino acid sequence that is at least 90%, at least 95%, at least 97%, or at least 99% identical to that of the VH of the corresponding Ab_001, Ab_008, Ab_009, Ab_025, Ab_026, Ab_027, Ab_028, Ab_029, Ab_034, Ab_035, Ab_036, Ab_043, Ab_050, Ab_051, Ab_058, Ab_063, Ab_159, Ab_064, Ab_065, Ab_066, Ab_072, Ab_073, Ab_074, Ab_078, Ab_080, Ab_083, Ab_153, or Ab_087 antibody, and/or further comprises a VL region with an amino acid sequence that is at least 90%, at least 95%, at least 97%, or at least 99% identical to that of the VL of the corresponding Ab_001, Ab_008, Ab_009, Ab_025, Ab_026, Ab_027, Ab_028, Ab_029, Ab_034, Ab_035, Ab_036, Ab_043, Ab_050, Ab_051, Ab_058, Ab_063, Ab_159, Ab_064, Ab_065, Ab_066, Ab_072, Ab_073, Ab_074, Ab_078, Ab_080, Ab_083, Ab_153, or Ab_087 antibody. In some embodiments, the anti-HVEM antibody comprises the VH CDR1, VH CDR2, and VH CDR3 and the VL CDR1, VH CDR2, and VH CDR3 of any one of antibodies Ab_001, Ab_008, Ab_009, Ab_025, Ab_026, Ab_027, Ab_028, Ab_029, Ab_034, Ab_035, Ab_036, Ab_043, Ab_050, Ab_051, Ab_058, Ab_063, Ab_159, Ab_064, Ab_065, Ab_066, Ab_072, Ab_073, Ab_074, Ab_078, Ab_080, Ab_083, Ab_153, or Ab_087, and further comprises a VH and a VL region, each with an amino acid sequence that is at least 90%, at least 95%, at least 97%, or at least 99% identical to that of the VH of the corresponding Ab_001, Ab_008, Ab_009, Ab_025, Ab_026, Ab_027, Ab_028, Ab_029, Ab_034, Ab_035, Ab_036, Ab_043, Ab_050, Ab_051, Ab_058, Ab_063, Ab_159, Ab_064, Ab_065, Ab_066, Ab_072, Ab_073, Ab_074, Ab_078, Ab_080, Ab_083, Ab_153, or Ab_087 antibody. In some embodiments, the antibody comprises both the VH and the VL region of the Ab_001, Ab_008, Ab_009, Ab_025, Ab_026, Ab_027, Ab_028, Ab_029, Ab_034, Ab_035, Ab_036, Ab_043, Ab_050, Ab_051, Ab_058, Ab_063, Ab_159, Ab_064, Ab_065, Ab_066, Ab_072, Ab_073, Ab_074, Ab_078, Ab_080, Ab_083, Ab_153, or Ab_087 antibody. In some embodiments, the anti-HVEM antibody blocks binding of human BTLA to human HVEM with an IC50 of 3 nM or less (e.g. in a competitive binding assay as described in the Examples herein), or of 2 nM or less.

In some embodiments, the anti-HVEM antibody blocks binding of human LIGHT to human HVEM with an IC50 of 30 nM or less (e.g. in a competitive binding assay as described in the Examples herein). In some such cases, the anti-HVEM antibody comprises the VH CDR1, VH CDR2, VH CDR3, VL CDR1, VL CDR2, and VL CDR3 of Ab_006, Ab_011, Ab_012, Ab_013, Ab_030, Ab_031, Ab_036, Ab_043, Ab_045, Ab_046, Ab_051, Ab_063, Ab_159, Ab_064, Ab_065, Ab_066, Ab_067, Ab_068, Ab_069, Ab_155, Ab_070, Ab_071, Ab_149, or Ab_078. In some embodiments, the anti-HVEM antibody comprises the VH CDR1, VH CDR2, and VH CDR3 and the VL CDR1, VH CDR2, and VH CDR3 of any one of antibodies Ab_006, Ab_011, Ab_012, Ab_013, Ab_030, Ab_031, Ab_036, Ab_043, Ab_045, Ab_046, Ab_051, Ab_063, Ab_159, Ab_064, Ab_065, Ab_066, Ab_067, Ab_068, Ab_069, Ab_155, Ab_070, Ab_071, Ab_149, or Ab_078, and further comprises a VH region with an amino acid sequence that is at least 90%, at least 95%, at least 97%, or at least 99% identical to that of the VH of the corresponding Ab_006, Ab_011, Ab_012, Ab_013, Ab_030, Ab_031, Ab_036, Ab_043, Ab_045, Ab_046, Ab_051, Ab_063, Ab_159, Ab_064, Ab_065, Ab_066, Ab_067, Ab_068, Ab_069, Ab_155, Ab_070, Ab_071, Ab_149, or Ab_078 antibody, and/or further comprises a VL region with an amino acid sequence that is at least 90%, at least 95%, at least 97%, or at least 99% identical to that of the VL of the corresponding Ab_006, Ab_011, Ab_012, Ab_013, Ab_030, Ab_031, Ab_036, Ab_043, Ab_045, Ab_046, Ab_051, Ab_063, Ab_159, Ab_064, Ab_065, Ab_066, Ab_067, Ab_068, Ab_069, Ab_155, Ab_070, Ab_071, Ab_149, or Ab_078 antibody. In some embodiments, the anti-HVEM antibody comprises the VH CDR1, VH CDR2, and VH CDR3 and the VL CDR1, VH CDR2, and VH CDR3 of any one of antibodies Ab_006, Ab_011, Ab_012, Ab_013, Ab_030, Ab_031, Ab_036, Ab_043, Ab_045, Ab_046, Ab_051, Ab_063, Ab_159, Ab_064, Ab_065, Ab_066, Ab_067, Ab_068, Ab_069, Ab_155, Ab_070, Ab_071, Ab_149, or Ab_078, and further comprises a VH and a VL region, each with an amino acid sequence that is at least 90%, at least 95%, at least 97%, or at least 99% identical to that of the VH of the corresponding Ab_006, Ab_011, Ab_012, Ab_013, Ab_030, Ab_031, Ab_036, Ab_043, Ab_045, Ab_046, Ab_051, Ab_063, Ab_159, Ab_064, Ab_065, Ab_066, Ab_067, Ab_068, Ab_069, Ab_155, Ab_070, Ab_071, Ab_149, or Ab_078 antibody. In some embodiments, the antibody comprises both the VH and the VL region of the Ab_006, Ab_011, Ab_012, Ab_013, Ab_030, Ab_031, Ab_036, Ab_043, Ab_045, Ab_046, Ab_051, Ab_063, Ab_159, Ab_064, Ab_065, Ab_066, Ab_067, Ab_068, Ab_069, Ab_155, Ab_070, Ab_071, Ab_149, or Ab_078 antibody.

In some embodiments, the anti-HVEM antibody blocks binding of human LIGHT to human HVEM with an IC50 of 20 nM or less (e.g. in a competitive binding assay as described in the Examples herein), or of 10 nM or less.

In some embodiments, the antibody blocks binding of human BTLA to human HVEM with an IC50 of 10 nM or less, and also blocks binding of human LIGHT to human HVEM with an IC50 of 100 nM or less. In some such cases, the anti-HVEM antibody comprises the VH CDR1, VH CDR2, VH CDR3, VL CDR1, VL CDR2, and VL CDR3 of Ab_036, Ab_051, Ab_063, Ab_159, Ab_064, Ab_065, Ab_066, Ab_078, or Ab_080. In some embodiments, the anti-HVEM antibody comprises the VH CDR1, VH CDR2, and VH CDR3 and the VL CDR1, VH CDR2, and VH CDR3 of any one of antibodies Ab_036, Ab_051, Ab_063, Ab_159, Ab_064, Ab_065, Ab_066, Ab_078, or Ab_080, and further comprises a VH region with an amino acid sequence that is at least 90%, at least 95%, at least 97%, or at least 99% identical to that of the VH of the corresponding Ab_036, Ab_051, Ab_063, Ab_159, Ab_064, Ab_065, Ab_066, Ab_078, or Ab_080 antibody, and/or further comprises a VL region with an amino acid sequence that is at least 90%, at least 95%, at least 97%, or at least 99% identical to that of the VL of the corresponding Ab_036, Ab_051, Ab_063, Ab_159, Ab_064, Ab_065, Ab_066, Ab_078, or Ab_080 antibody. In some embodiments, the anti-HVEM antibody comprises the VH CDR1, VH CDR2, and VH CDR3 and the VL CDR1, VH CDR2, and VH CDR3 of any one of antibodies Ab_036, Ab_051, Ab_063, Ab_159, Ab_064, Ab_065, Ab_066, Ab_078, or Ab_080, and further comprises a VH and a VL region, each with an amino acid sequence that is at least 90%, at least 95%, at least 97%, or at least 99% identical to that of the VH of the corresponding Ab_036, Ab_051, Ab_063, Ab_159, Ab_064, Ab_065, Ab_066, Ab_078, or Ab_080 antibody. In some embodiments, the antibody comprises both the VH and the VL region of the Ab_036, Ab_051, Ab_063, Ab_159, Ab_064, Ab_065, Ab_066, Ab_078, or Ab_080 antibody.

In some embodiments, the antibody blocks binding of human BTLA to human HVEM with an IC50 of 10 nM or less, and also blocks binding of human LIGHT to human HVEM with a higher IC50 as compared to the IC50 for the BTLA competitive binding experiment. In some such cases, the anti-HVEM antibody comprises the VH CDR1, VH CDR2, VH CDR3, VL CDR1, VL CDR2, and VL CDR3 of Ab_001, Ab_043, Ab_050, Ab_051, Ab_066, Ab_072, Ab_078, or Ab_080. In some embodiments, the anti-HVEM antibody comprises the VH CDR1, VH CDR2, and VH CDR3 and the VL CDR1, VH CDR2, and VH CDR3 of any one of antibodies Ab_001, Ab_043, Ab_050, Ab_051, Ab_066, Ab_072, Ab_078, or Ab_080, and further comprises a VH region with an amino acid sequence that is at least 90%, at least 95%, at least 97%, or at least 99% identical to that of the VH of the corresponding Ab_001, Ab_043, Ab_050, Ab_051, Ab_066, Ab_072, Ab_078, or Ab_080 antibody, and/or further comprises a VL region with an amino acid sequence that is at least 90%, at least 95%, at least 97%, or at least 99% identical to that of the VL of the corresponding Ab_001, Ab_043, Ab_050, Ab_051, Ab_066, Ab_072, Ab_078, or Ab_080 antibody. In some embodiments, the anti-HVEM antibody comprises the VH CDR1, VH CDR2, and VH CDR3 and the VL CDR1, VH CDR2, and VH CDR3 of any one of antibodies Ab_001, Ab_043, Ab_050, Ab_051, Ab_066, Ab_072, Ab_078, or Ab_080, and further comprises a VH and a VL region, each with an amino acid sequence that is at least 90%, at least 95%, at least 97%, or at least 99% identical to that of the VH of the corresponding Ab_001, Ab_043, Ab_050, Ab_051, Ab_066, Ab_072, Ab_078, or Ab_080 antibody. In some embodiments, the antibody comprises both the VH and the VL region of the Ab_001, Ab_043, Ab_050, Ab_051, Ab_066, Ab_072, Ab_078, or Ab_080 antibody.

In some embodiments, the antibody binds to cynomolgus monkey HVEM as well as to human HVEM (e.g. via an ELISA assay as described herein or via a BLI assay as described herein). In some such cases, the anti-HVEM antibody comprises the VH CDR1, VH CDR2, VH CDR3, VL CDR1, VL CDR2, and VL CDR3 of Ab_002, Ab_003, Ab_006, Ab_008, Ab_009, Ab_011, Ab_012, Ab_013, Ab_025, Ab_028, Ab_030, Ab_031, Ab_032, Ab_33, Ab_039, Ab_045, Ab_046, Ab_052, Ab_053, Ab_054, Ab_055, Ab_060, Ab_061, Ab_062, Ab_063, Ab_065, Ab_067, Ab_068, Ab_069, Ab_070, Ab_071, Ab_075, Ab_076, or Ab_080. In some embodiments, the anti-HVEM antibody comprises the VH CDR1, VH CDR2, and VH CDR3 and the VL CDR1, VH CDR2, and VH CDR3 of any one of antibodies Ab_002, Ab_003, Ab_006, Ab_008, Ab_009, Ab_011, Ab_012, Ab_013, Ab_025, Ab_028, Ab_030, Ab_031, Ab_032, Ab_33, Ab_039, Ab_045, Ab_046, Ab_052, Ab_053, Ab_054, Ab_055, Ab_060, Ab_061, Ab_062, Ab_063, Ab_065, Ab_067, Ab_068, Ab_069, Ab_070, Ab_071, Ab_075, Ab_076, or Ab_080, and further comprises a VH region with an amino acid sequence that is at least 90%, at least 95%, at least 97%, or at least 99% identical to that of the VH of the corresponding Ab_002, Ab_003, Ab_006, Ab_008, Ab_009, Ab_011, Ab_012, Ab_013, Ab_025, Ab_028, Ab_030, Ab_031, Ab_032, Ab_33, Ab_039, Ab_045, Ab_046, Ab_052, Ab_053, Ab_054, Ab_055, Ab_060, Ab_061, Ab_062, Ab_063, Ab_065, Ab_067, Ab_068, Ab_069, Ab_070, Ab_071, Ab_075, Ab_076, or Ab_080 antibody, and/or further comprises a VL region with an amino acid sequence that is at least 90%, at least 95%, at least 97%, or at least 99% identical to that of the VL of the corresponding Ab_002, Ab_003, Ab_006, Ab_008, Ab_009, Ab_011, Ab_012, Ab_013, Ab_025, Ab_028, Ab_030, Ab_031, Ab_032, Ab_33, Ab_039, Ab_045, Ab_046, Ab_052, Ab_053, Ab_054, Ab_055, Ab_060, Ab_061, Ab_062, Ab_063, Ab_065, Ab_067, Ab_068, Ab_069, Ab_070, Ab_071, Ab_075, Ab_076, or Ab_080 antibody. In some embodiments, the anti-HVEM antibody comprises the VH CDR1, VH CDR2, and VH CDR3 and the VL CDR1, VH CDR2, and VH CDR3 of any one of antibodies Ab_002, Ab_003, Ab_006, Ab_008, Ab_009, Ab_011, Ab_012, Ab_013, Ab_025, Ab_028, Ab_030, Ab_031, Ab_032, Ab_33, Ab_039, Ab_045, Ab_046, Ab_052, Ab_053, Ab_054, Ab_055, Ab_060, Ab_061, Ab_062, Ab_063, Ab_065, Ab_067, Ab_068, Ab_069, Ab_070, Ab_071, Ab_075, Ab_076, or Ab_080, and further comprises a VH and a VL region, each with an amino acid sequence that is at least 90%, at least 95%, at least 97%, or at least 99% identical to that of the VH of the corresponding Ab_002, Ab_003, Ab_006, Ab_008, Ab_009, Ab_011, Ab_012, Ab_013, Ab_025, Ab_028, Ab_030, Ab_031, Ab_032, Ab_33, Ab_039, Ab_045, Ab_046, Ab_052, Ab_053, Ab_054, Ab_055, Ab_060, Ab_061, Ab_062, Ab_063, Ab_065, Ab_067, Ab_068, Ab_069, Ab_070, Ab_071, Ab_075, Ab_076, or Ab_080 antibody. In some embodiments, the antibody comprises both the VH and the VL region of the Ab_002, Ab_003, Ab_006, Ab_008, Ab_009, Ab_011, Ab_012, Ab_013, Ab_025, Ab_028, Ab_030, Ab_031, Ab_032, Ab_33, Ab_039, Ab_045, Ab_046, Ab_052, Ab_053, Ab_054, Ab_055, Ab_060, Ab_061, Ab_062, Ab_063, Ab_065, Ab_067, Ab_068, Ab_069, Ab_070, Ab_071, Ab_075, Ab_076, or Ab_080 antibody.

In some embodiments, the antibody binds to cynomolgus monkey HVEM as well as to human HVEM (e.g. via an ELISA assay as described herein or via a BLI assay as described herein) and also blocks binding of human BTLA to human HVEM with an IC50 of 10 nM or less. In some such cases, the anti-HVEM antibody comprises the VH CDR1, VH CDR2, VH CDR3, VL CDR1, VL CDR2, and VL CDR3 of Ab_002, Ab_003, Ab_008, Ab_009, Ab_028, Ab_063, Ab_065, or Ab_080. In some embodiments, the anti-HVEM antibody comprises the VH CDR1, VH CDR2, and VH CDR3 and the VL CDR1, VH CDR2, and VH CDR3 of any one of antibodies Ab_002, Ab_003, Ab_008, Ab_009, Ab_028, Ab_063, Ab_065, or Ab_080, and further comprises a VH region with an amino acid sequence that is at least 90%, at least 95%, at least 97%, or at least 99% identical to that of the VH of the corresponding Ab_002, Ab_003, Ab_008, Ab_009, Ab_028, Ab_063, Ab_065, or Ab_080 antibody, and/or further comprises a VL region with an amino acid sequence that is at least 90%, at least 95%, at least 97%, or at least 99% identical to that of the VL of the corresponding Ab_002, Ab_003, Ab_008, Ab_009, Ab_028, Ab_063, Ab_065, or Ab_080 antibody. In some embodiments, the anti-HVEM antibody comprises the VH CDR1, VH CDR2, and VH CDR3 and the VL CDR1, VH CDR2, and VH CDR3 of any one of antibodies Ab_002, Ab_003, Ab_008, Ab_009, Ab_028, Ab_063, Ab_065, or Ab_080, and further comprises a VH and a VL region, each with an amino acid sequence that is at least 90%, at least 95%, at least 97%, or at least 99% identical to that of the VH of the corresponding Ab_002, Ab_003, Ab_008, Ab_009, Ab_028, Ab_063, Ab_065, or Ab_080 antibody. In some embodiments, the antibody comprises both the VH and the VL region of the Ab_002, Ab_003, Ab_008, Ab_009, Ab_028, Ab_063, Ab_065, or Ab_080 antibody. In some such embodiments, the antibody also detectably blocks the binding of human LIGHT to human HVEM in a competition assay as described herein.

In some embodiments, the antibody binds to cynomolgus monkey HVEM as well as to human HVEM (e.g. via an ELISA assay as described herein or via a BLI assay as described herein) and also blocks binding of human LIGHT to human HVEM with an IC50 of 30 nM or less. In some such cases, the anti-HVEM antibody comprises the VH CDR1, VH CDR2, VH CDR3, VL CDR1, VL CDR2, and VL CDR3 of Ab_006, Ab_008, Ab_009, Ab_011, Ab_012, Ab_023, Ab_028, Ab_030, Ab_031, Ab_045, Ab_046, Ab_052, Ab_053, Ab_054, Ab_063, Ab_065, Ab_067, Ab_068, Ab_069, Ab_070, Ab_071, or Ab_080. In some embodiments, the anti-HVEM antibody comprises the VH CDR1, VH CDR2, and VH CDR3 and the VL CDR1, VH CDR2, and VH CDR3 of any one of antibodies Ab_002, Ab_003, Ab_008, Ab_009, Ab_028, Ab_063, Ab_065, or Ab_080, and further comprises a VH region with an amino acid sequence that is at least 90%, at least 95%, at least 97%, or at least 99% identical to that of the VH of the corresponding Ab_006, Ab_008, Ab_009, Ab_011, Ab_012, Ab_023, Ab_028, Ab_030, Ab_031, Ab_045, Ab_046, Ab_052, Ab_053, Ab_054, Ab_063, Ab_065, Ab_067, Ab_068, Ab_069, Ab_070, Ab_071, or Ab_080 antibody, and/or further comprises a VL region with an amino acid sequence that is at least 90%, at least 95%, at least 97%, or at least 99% identical to that of the VL of the corresponding Ab_006, Ab_008, Ab_009, Ab_011, Ab_012, Ab_023, Ab_028, Ab_030, Ab_031, Ab_045, Ab_046, Ab_052, Ab_053, Ab_054, Ab_063, Ab_065, Ab_067, Ab_068, Ab_069, Ab_070, Ab_071, or Ab_080 antibody. In some embodiments, the anti-HVEM antibody comprises the VH CDR1, VH CDR2, and VH CDR3 and the VL CDR1, VH CDR2, and VH CDR3 of any one of antibodies Ab_006, Ab_008, Ab_009, Ab_011, Ab_012, Ab_023, Ab_028, Ab_030, Ab_031, Ab_045, Ab_046, Ab_052, Ab_053, Ab_054, Ab_063, Ab_065, Ab_067, Ab_068, Ab_069, Ab_070, Ab_071, or Ab_080, and further comprises a VH and a VL region, each with an amino acid sequence that is at least 90%, at least 95%, at least 97%, or at least 99% identical to that of the VH of the corresponding Ab_006, Ab_008, Ab_009, Ab_011, Ab_012, Ab_023, Ab_028, Ab_030, Ab_031, Ab_045, Ab_046, Ab_052, Ab_053, Ab_054, Ab_063, Ab_065, Ab_067, Ab_068, Ab_069, Ab_070, Ab_071, or Ab_080 antibody. In some embodiments, the antibody comprises both the VH and the VL region of the Ab_006, Ab_008, Ab_009, Ab_011, Ab_012, Ab_023, Ab_028, Ab_030, Ab_031, Ab_045, Ab_046, Ab_052, Ab_053, Ab_054, Ab_063, Ab_065, Ab_067, Ab_068, Ab_069, Ab_070, Ab_071, or Ab_080 antibody.

### Anti-HVEM Antibody Expression

Procedures for constructing the anti-HVEM antibodies as described herein are well known in the art (see e.g., Williams, et al., J. Cell Biol. 111: 955, 1990). For example, the polynucleotides encoding the antibodies described in Tables 1-3 can be assembled with appropriate control and signal sequences using routine procedures of recombinant DNA methodology. See, e.g., as described in U.S. Pat. No. 4,593,002, and Langford, et al., Molec. Cell. Biol. 6: 3191, 1986.

Such polynucleotide sequence encoding the antibodies described herein can be synthesized chemically or isolated by one of several approaches. The polynucleotide sequence to be synthesized can be designed with the appropriate codons for the desired amino acid sequence. In general, one will select preferred codons for the intended host in which the sequence will be used for expression. The complete sequence may be assembled from overlapping oligonucleotides prepared by standard methods and assembled into a complete coding sequence. See, e.g., Edge, Nature 292: 756, 1981; Nambair, et al. Science 223: 1299, 1984; Jay, et al., J.

Biol. Chem. 259: 6311, 1984.

In one aspect, polynucleotides encoding an an-HVEM antibody described herein are isolated individually using the polymerase chain reaction and/or are chemically synthesized (M. A. Innis, et al., In PCR Protocols: A Guide to Methods and Applications, Academic Press, 1990). Preferably, isolated fragments are bordered by compatible restriction endonuclease sites which allow for easy cloning into an expression construct. This technique is well known to those of skill in the art. Sequences may be fused directly to each other (e.g., with no intervening sequences), or inserted into one another (e.g., where domain sequences are discontinuous), or may be separated by intervening sequences (e.g., such as linker sequences).

The basic strategies for preparing oligonucleotide primers, probes and DNA libraries, as well as their screening by nucleic acid hybridization, are well known to those of ordinary skill in the art. See, e.g., Sambrook, et al., 1989, supra; Perbal, 1984, supra. The construction of an appropriate genomic DNA or cDNA library is within the skill of the art. See, e.g., Perbal, 1984, supra. Alternatively, suitable DNA libraries or publicly available clones are available from suppliers of biological research materials, such as Clonetech and Stratagene, as well as from public depositories such as the American Type Culture Collection.

Selection may be accomplished by expressing sequences from an expression library of DNA and detecting the expressed anti-HVEM antibodies. Such selection procedures are well known to those of ordinary skill in the art (see, e.g., Sambrook, et al., 1989, supra). The anti-HVEM antibody sequence can preferably be cloned into a vector comprising an origin of replication for maintaining the sequence in a host cell.

In preferred embodiments, polynucleotides encoding an an-HVEM antibody described herein further comprises a polynucleotide sequence for insertion into a target cell and an expression control sequence operably linked thereto to control expression of the polynucleotide sequence (e.g., transcription and/or translation) in the cell. Examples include plasmids, phages, autonomously replicating sequences (ARS), centromeres, and other sequences which are able to replicate or be replicated in vitro or in a host cell (e.g., such as a bacterial, yeast, or insect cell) and/or target cell (e.g., such as a mammalian cell, preferably an antigen presenting cell) and/or to convey the polynucleotides encoding an an-HVEM antibody described herein to a desired location within the target cell.

Recombinant expression vectors may be derived from micro-organisms which readily infect animals, including horses, cows, pigs, llamas, giraffes, dogs, cats or chickens. Preferred vectors include those which have already been used as live vaccines, such as vaccinia. These recombinants can be directly inoculated into a host, conferring immunity not only to the microbial vector, but also to express the anti-HVEM antibodies described herein. Preferred vectors contemplated herein as live recombinant vaccines include RNA viruses, adenovirus, herpesviruses, poliovirus, and vaccinia and other pox viruses, as taught in Flexner, Adv. Pharmacol. 21: 51, 1990, for example.

Expression control sequences include, but are not limited to, promoter sequences to bind RNA polymerase, enhancer sequences or negative regulatory elements to bind to transcriptional activators and repressors, respectively, and/or translation initiation sequences for ribosome binding. For example, a bacterial expression vector can include a promoter such as the lac promoter and for transcription initiation, the Shine-Dalgarno sequence and the start codon AUG (Sambrook, et al., 1989, supra). Similarly, a eukaryotic expression vector preferably includes a heterologous, homologous, or chimeric promoter for RNA polymerase II, a downstream polyadenylation signal, the start codon AUG, and a termination codon for detachment of a ribosome.

Expression control sequences may be obtained from naturally occurring genes or may be designed. Designed expression control sequences include, but are not limited to, mutated and/or chimeric expression control sequences or synthetic or cloned consensus sequences. Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, Calif.) and Promega Biotech (Madison, Wis.).

In order to optimize expression and/or transcription, it may be necessary to remove, add or alter 5′ and/or 3′ untranslated portions of the vectors to eliminate extra, or alternative translation initiation codons or other sequences that may interfere with, or reduce, expression, either at the level of transcription or translation. Alternatively, consensus ribosome binding sites can be inserted immediately 5′ of the start codon to enhance expression. A wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma, adenovirus, herpes virus and other sequences known to control the expression of genes of mammalian cells, and various combinations thereof.

In one aspect, an anti-HVEM antibody expressing construct comprises an origin of replication for replicating the vector. Preferably, the origin functions in at least one type of host cell which can be used to generate sufficient numbers of copies of the sequence for use in delivery to a target cell. Suitable origins therefore include, but are not limited to, those which function in bacterial cells (e.g., such as Escherichia sp., Salmonella sp., Proteus sp., Clostridium sp., Klebsiella sp., Bacillus sp., Streptomyces sp., and Pseudomonas sp.), yeast (e.g., such as Saccharamyces sp. or Pichia sp.), insect cells, and mammalian cells. In one preferred aspect, an origin of replication is provided which functions in the target cell into which the vehicle is introduced (e.g., a mammalian cell, such as a human cell). In another aspect, at least two origins of replication are provided, one that functions in a host cell and one that functions in a target cell.

The constructs comprising the polynucleotides encoding the anti-HVEM antibody as described herein may alternatively, or additionally, comprise sequences to facilitate integration of at least a portion of the polynucleotide into a target cell chromosome. For example, the construct may comprise regions of homology to target cell chromosomal DNA. In one aspect, the construct comprises two or more recombination sites which flank a nucleic acid sequence encoding the polynucleotide encoding the anti-HVEM antibody described herein.

The vector may additionally comprise a detectable and/or selectable marker to verify that the vector has been successfully introduced in a target cell and/or can be expressed by the target cell. These markers can encode an activity, such as, but not limited to, production of RNA, peptide, or protein, or can provide a binding site for RNA, peptides, proteins, inorganic and organic compounds or compositions and the like.

Examples of detectable/selectable markers genes include, but are not limited to: polynucleotide segments that encode products which provide resistance against otherwise toxic compounds (e.g., antibiotics); polynucleotide segments that encode products which are otherwise lacking in the recipient cell (e.g., tRNA genes, auxotrophic markers); polynucleotide segments that encode products which suppress the activity of a gene product; polynucleotide segments that encode products which can be readily identified (e.g., phenotypic markers such as beta-galactosidase, a fluorescent protein (GFP, CFP, YFG, BFP, RFP, EGFP, EYFP, EBFP, dsRed, mutated, modified, or enhanced forms thereof, and the like), and cell surface proteins); polynucleotide segments that bind products which are otherwise detrimental to cell survival and/or function; polynucleotide segments that otherwise inhibit the activity of other nucleic acid segments (e.g., antisense oligonucleotides); polynucleotide segments that bind products that modify a substrate (e.g., restriction endonucleases); polynucleotide segments that can be used to isolate or identify a desired molecule (e.g., segments encoding specific protein binding sites); primer sequences; polynucleotide segments, which when absent, directly or indirectly confer resistance or sensitivity to particular compounds; and/or polynucleotide segments that encode products which are toxic in recipient cells.

The marker gene can be used as a marker for conformation of successful gene transfer and/or to isolate cells expressing transferred genes and/or to recover transferred genes from a cell.

In another preferred embodiment, a polynucleotide encoding an anti-HVEM antibody can be delivered to cells such as by microinjection of DNA into the nucleus of a cell (Capechi, et al., 1980, Cell 22: 479-488); transfection with CaPO₄ (Chen and Okayama, 1987, Mol. Cell Biol. 7: 2745 2752), electroporation (Chu, et al., 1987, Nucleic Acid Res. 15: 1311-1326); lipofection/liposome fusion (Feigner, et al., 1987, Proc. Natl. Acad. Sci. USA 84: 7413-7417) and particle bombardment (Yang, et al., 1990, Proc. Natl. Acad. Sci. USA 87: 9568-9572).

The anti-HVEM antibody constructs according to the invention can be expressed in a variety of host cells, including, but not limited to: prokaryotic cells (e.g., E. coli, Staphylococcus sp., Bacillus sp.); yeast cells (e.g., Saccharomyces sp.); insect cells; nematode cells; plant cells; amphibian cells (e.g., Xenopus); avian cells; and mammalian cells (e.g., human cells, mouse cells, mammalian cell lines, primary cultured mammalian cells, such as from dissected tissues).

In one aspect, anti-HVEM antibody constructs are expressed in host cells in vitro, e.g., in culture. In another aspect, anti-HVEM antibody constructs are expressed in a transgenic organism (e.g., a transgenic mouse, rat, rabbit, pig, primate, etc.) that comprises somatic and/or germline cells comprising nucleic acids encoding the anti-HVEM antibody constructs. Methods for constructing transgenic animals are well known in the art and are routine. The anti-HVEM antibody constructs also can be introduced into cells in vitro, and the cells (e.g., such as stem cells, hematopoietic cells, lymphocytes, and the like) can be introduced into the host organism. The cells may be heterologous or autologous with respect to the host organism. For example, cells can be obtained from the host organism, anti-HVEM antibody constructs introduced into the cells in vitro, and then reintroduced into the host (non-human vertebrate).

Additionally, the anti-HVEM antibodies disclosed herein can be affinity matured using techniques well known in the art, such as display technology, such as for example, phage display, yeast display or ribosome display. In one example, single chain anti-HVEM antibody molecules (“scFvs”) displayed on the surface of phage particles are screened to identify those scFvs that immunospecifically bind to a HVEM antigen. The present invention encompasses both scFvs and portions thereof that are identified to immunospecifically bind to a HVEM antigen. Such scFvs can routinely be “converted” to immunoglobulin molecules by inserting, for example, the nucleotide sequences encoding the VH and/or VL domains of the scFv into an expression vector containing the constant domain sequences and engineered to direct the expression of the immunoglobulin molecule.

Recombinant expression of the raised antibodies (including scFvs and other molecules comprising, or alternatively consisting of, antibody fragments or variants thereof (e.g., a heavy or light chain of an antibody of the invention or a portion thereof or a single chain antibody of the invention)), requires construction of an expression vector(s) containing a polynucleotide that encodes the anti-HVEM antibody comprising the sequences disclosed in Tables 2-3. Once a polynucleotide encoding such an antibody molecule (e.g., a whole antibody, a heavy or light chain of an antibody, or variant or portion thereof (preferably, but not necessarily, containing the heavy or light chain variable domain)), of the invention has been obtained, the vector(s) for the production of the antibody molecule may be produced by recombinant DNA technology using techniques well known in the art. Thus, methods for preparing an anti-HVEM antibody described herein can occur simply by expressing a polynucleotide encoding the anti-HVEM antibody described in Tables 1-3 using techniques well known in the art. Methods which are well known to those skilled in the art can be used to construct expression vectors containing the antibody coding sequences and appropriate transcriptional and translational control signals. These methods include, for example, in vitro recombinant DNA techniques, synthetic techniques, and in vivo genetic recombination and are described herein. The invention, thus, provides replicable vectors comprising a nucleotide sequence encoding the anti-HVEM antibody obtained and isolated as described herein (e.g., a whole antibody, a heavy or light chain of an antibody, a heavy or light chain variable domain of an antibody, or a portion thereof, or a heavy or light chain CDR, a single chain Fv, or fragments or variants thereof), operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy chain, the entire light chain, or both the entire heavy and light chains.

The expression vector(s) can be transferred to a host cell by conventional techniques and the transfected cells are then cultured by conventional techniques to produce the anti-HVEM antibody. Thus, the invention includes host cells containing polynucleotide(s) encoding the anti-HVEM antibody (e.g., whole antibody, a heavy or light chain thereof, or portion thereof, or a single chain antibody of the invention, or a fragment or variant thereof), operably linked to a heterologous promoter. In preferred embodiments, for the expression of entire antibody molecules, vectors encoding both the heavy and light chains may be co-expressed in the host cell for expression of the entire immunoglobulin molecule, as detailed below.

A variety of host-expression vector systems may be utilized to express anti-HVEM antibody. Such host-expression systems represent vehicles by which the coding sequences of interest may be produced and subsequently purified, but also represent cells which may, when transformed or transfected, with the appropriate nucleotide coding sequences, express the anti-HVEM antibody. These include, but are not limited to, microorganisms such as bacteria (e.g., E. coli, B. subtilis) transformed with recombinant bacteriophage DNA, plasmid DNA or cosmid DNA expression vectors containing sequences; yeast (e.g., Saccharomyces, Pichia) transformed with recombinant yeast expression vectors containing coding sequences; insect cell systems infected with recombinant virus expression vectors (e.g., baculovirus) containing coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, are used for the expression of the anti-HVEM antibody. For example, mammalian cells such as Chinese hamster ovary cells (CHO), in conjunction with a vector such as the major intermediate early gene promoter element from human cytomegalovirus is an effective expression system (Foecking et al., Gene 45:101 (1986); Cockett et al., Bio/Technology 8:2 (1990)).

In bacterial systems, a number of expression vectors may be advantageously selected depending upon the intended use. For example, when a large quantity of a protein is to be produced, vectors which direct the expression of high levels of protein products that are readily purified may be desirable. Such vectors include, but are not limited to, the E. coli expression vector pUR278 (Ruther et al., EMBO 1. 2:1791 (1983)), in which the coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or Factor Xa protease cleavage sites so that the cloned target gene product can be released from the GST moiety.

In an insect system, Autographa californica nuclear polyhedrosis virus (AcNPV) may be used as a vector to express an anti-HVEM antibody. The virus grows in Spodoptera frugiperda cells. Coding sequences may be cloned individually into non-essential regions (for example, the polyhedrin gene) of the virus and placed under control of an AcNPV promoter (for example, the polyhedrin promoter).

In mammalian host cells, a number of viral-based expression systems may be utilized express an anti-HVEM antibody. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination.

Insertion in a non-essential region of the viral genome (e.g., region E1 or E3) will result in a recombinant virus that is viable and capable of expressing the anti-HVEM antibody or the encoded polypeptides of the LAMP Construct in infected hosts (e.g., see Logan & Shenk, Proc. Natl. Acad. Sci. USA 8 1:355-359 (1984)).

Specific initiation signals may also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed, to this end, eukaryotic host cells which possess the cellular machinery for proper processing of the primary transcript, glycosylation, and phosphorylation of the gene product may be used. Such mammalian host cells include, but are not limited to, CHO, VERY, BHK, Hela, COS, NSO, MDCK, 293, 3T3, W138, and in particular, breast cancer cell lines such as, for example, BT483, Hs578T, HTB2, BT20 and T47D, and normal mammary gland cell line such as, for example, CRL7030 and HsS78Bst.

For long-term, high-yield production of recombinant proteins, stable expression is preferred. For example, cell lines which stably express the anti-HVEM antibody may be engineered. Rather than using expression vectors which contain viral origins of replication, host cells can be transformed with a polynucleotide controlled by appropriate expression control elements (e.g., promoter, enhancer, sequences, transcription terminators, polyadenylation sites, etc.), and a selectable marker. Following the introduction of the foreign polynucleotide, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the anti-HVEM antibody.

A number of selection systems may be used, including but not limited to, the herpes simplex virus thymidine kinase (Wigler et al., Cell 11:223 (1977)), hypoxanthineguanine phosphoribosyltransferase (Szybalska & Szybalski, Proc. Natl. Acad. Sci. USA 48:202 (1992)), and adenine phosphoribosyltransferase (Lowy et al., Cell 22:8 17 (1980)) genes can be employed in tk-, hgprt- or aprt- cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Goldspiel et al., Clinical Pharmacy, 12: 488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62: 191-217 (1993); TIB TECH 11(5):155-2 15 (May; 1993)); and hygro, which confers resistance to hygromycin (Santerre et al., Gene 30:147 (1984)). Methods commonly known in the art of recombinant DNA technology may be routinely applied to select the desired recombinant clone, and such methods are described, for example; in Ausubel et al. (eds.), Current Protocols in Molecular Biology, John Wiley & Sons, N Y (1993); Kriegler, Gene Transfer and Expression, A Laboratory Manual, Stockton Press, N Y (1990); and in Chapters 12 and 13, Dracopoli et al. (eds), Current Protocols in Human Genetics, John Wiley & Sons, N Y (1994); Colberre-Garapin et al., J. Mol. Biol. 150:1 (1981).

The expression levels of an anti-HVEM antibody can be increased by vector amplification (for a review, see Bebbington and Hentschel, The Use Of Vectors Based On Gene Amplification For The Expression Of Cloned Genes In Mammalian Cells In DNA Cloning, Vol. 3. (Academic Press, New York, 1987)). When a marker in the vector system expressing an anti-HVEM antibody is amplifiable, an increase in the level of inhibitor present in the host cell culture will increase the number of copies of the marker gene. Since the amplified region is associated with the coding sequence, production of the anti-HVEM antibody express will also increase (Crouse et al., Mol. Cell. Biol. 3:257 (1983)).

Other elements that can be included in vector sequences include heterologous signal peptides (secretion signals), membrane anchoring sequences, introns, alternative splice sites, translation start and stop signals, inteins, biotinylation sites and other sites promoting post-translational modifications, purification tags, sequences encoding fusions to other proteins or peptides, separate coding regions separated by internal ribosome reentry sites, sequences encoding “marker” proteins that, for example, confer selectability (e.g., antibiotic resistance) or sortability (e.g., fluorescence), modified nucleotides, and other known polynucleotide cis-acting features not limited to these examples.

The host cell may be co-transfected with two expression vectors of the invention, for example, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain anti-HVEM polypeptides. In such situations, the light chain is preferably placed before the heavy chain to avoid an excess of toxic free heavy chain (Proudfoot, Nature 322:52 (1986); Kohler, Proc. Natl. Acad. Sci. USA 77:2 197 (1980)). The coding sequences for the heavy and light chains may comprise cDNA or genomic DNA or synthetic DNA sequences.

Once an anti-HVEM antibody has been produced by recombinant expression, it may be purified by any method known in the art for purification of a protein, for example, by chromatography (e.g., ion exchange, affinity (particularly by Protein A affinity and immunoaffinity), and sizing column chromatography), centrifugation, differential solubility, or by any other standard technique for the purification of proteins. Further, an anti-HVEM antibody may be fused to heterologous polypeptide sequences described herein or otherwise known in the art to facilitate purification.

In one example, the anti-HVEM antibody may be fused with the constant domain of immunoglobulins (IgA, IgE, IgG, IgM), or portions thereof (CH1, CH2, CH3, or any combination thereof and portions thereof), or albumin (including but not limited to recombinant human albumin or fragments or variants thereof (see, e.g., U.S. Pat. No. 5,876,969, issued Mar. 2, 1999, EP Patent 0 413 622, and U.S. Pat. No. 5,766,883, issued Jun. 16, 1998), resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fe fragments (see, e.g., PCT Publications WO 96/22024 and WO 99/04813). IgG Fusion proteins that have a disulfide-linked dimeric structure due to the IgG portion disulfide bonds have also been found to be more efficient in binding and neutralizing other molecules than monomeric polypeptides or fragments thereof alone. See, e.g., Fountoulakis et al., J. Biochem., 270:3958-3964 (1995). Nucleic acids encoding the anti-HVEM antibody described herein can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix-binding domain for the fusion protein. Extracts from cells infected with the recombinant vaccinia virus are loaded onto Ni2+nitriloacetic acid-agarose column and histidine-tagged proteins can be selectively eluted with imidazole-containing buffers.

### Tumor Therapy Treated by Anti-HVEM Antibodies

Tumor therapy, as referred to herein, includes using the anti-HVEM antibody described herein which reduce the rate of tumor growth, that is slow down, but may not necessarily eliminate all tumor growth. Reduction in the rate of tumor growth can be, for example, a reduction in at least 10%, 20%, 30%, 40%, 50%, 75%, 100%, 150%, 200% or more of the rate of growth of a tumor. For example, the rate of growth can be measured over 1, 2, 3, 4, 5, 6 or 7 days, or for longer periods of one or more weeks. In some embodiments, the invention may result in the arrest of tumor growth, or the reduction in tumor size or the elimination of a tumor.

The anti-HVEM antibodies as described herein may be used to treat a subject suffering from a tumor alone, or in combination with a second therapy, such as one directed to a tumor antigen as described below.

A subject suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human. Thus, in some embodiments, the subject is a human. In other embodiments, non-human mammals, especially mammals that are conventionally used as models for demonstrating therapeutic efficacy in humans (e.g. murine, primate, porcine, canine, or rabbit animals) may be employed.

In some embodiments, the subject may have minimal residual disease (MRD) after an initial cancer treatment. A subject with cancer may display at least one identifiable sign, symptom, or laboratory finding that is sufficient to make a diagnosis of cancer in accordance with clinical standards known in the art. Examples of such clinical standards can be found in textbooks of medicine such as Harrison's Principles of Internal Medicine, 15th Ed., Fauci A S et al., eds., McGraw-Hill, New York, 2001. In some instances, a diagnosis of a cancer in a subject may include identification of a particular cell type (e.g. a cancer cell) in a sample of a body fluid or tissue obtained from the subject.

In some embodiments, the cancer cells may express one or more antigens that are not expressed by normal somatic cells in the subject (i.e. tumor antigens). Tumor antigens are known in the art and may elicit immune responses in the subject. In particular, tumor antigens may elicit T-cell-mediated immune responses against cancer cells in the subject i.e. the tumor antigens may be recognized by CD8+ T-cells in the subject.

Tumor antigens expressed by cancer cells in a cancerous tumor may include, for example, cancer-testis (CT) antigens encoded by cancer-germ line genes, such as MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-I, GAGE-2, GAGE-3, GAGE-4, GAGE-5, GAGE-6, GAGE-7, GAGE-8, BAGE-I, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-I, SSX-I, SSX-2(HOM-MEL-40), SSX-3, SSX-4, SSX-5, SCP-I and XAGE and immunogenic fragments thereof (Simpson et al., Nature Rev (2005) 5, 615-625, Gure et al., Clin Cancer Res (2005) 11, 8055-8062; Velazquez et al., Cancer Immun (2007) 7, 11; Andrade et al., Cancer Immun (2008) 8, 2; Tinguely et al., Cancer Science (2008); Napoletano et al., Am J of Obstet Gyn (2008) 198, 99 e91-97).

Other tumor antigens that may be expressed include, for example, overexpressed or mutated proteins and differentiation antigens particularly melanocyte differentiation antigens such as p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR.alpha. fusion protein, PTPRK, K-ras, N-ras, Triosephosphate isomeras, GnTV, Herv-K-mel, NA-88, SP17, and TRP2-Int2, (MART-1), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, pi85erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, and TPS and tyrosinase related proteins such as TRP-1, TRP-2, and mesothelin.

Other tumor antigens that may be expressed include out-of-frame peptide-MHC complexes generated by the non-AUG translation initiation mechanisms employed by “stressed” cancer cells (Malarkannan et al. Immunity 1999). Other prefer examples of tumor antigens that may be expressed are well-known in the art (see for example WO00/20581; Cancer Vaccines and Immunotherapy (2000) Eds Stern, Beverley and Carroll, Cambridge University Press, Cambridge) The sequences of these tumor antigens are readily available from public databases but are also found in WO 1992/020356 A1, WO 1994/005304 A1, WO 1994/023031 A1, WO 1995/020974 A1, WO 1995/023874 A1 & WO 1996/026214 A1.

### Formulations

The anti-HVEM antibody as described herein may be administered together with other anti-cancer therapies, such as conventional chemotherapeutic agents, radiation therapy or cancer immunotherapy. For example, the anti-HVEM antibody is administered together with an anti-cancer compound. The anti-HVEM antibody and the anti-cancer compound may be separate compounds or molecules or they may be covalently or non-covalently linked in a single compound, molecule, particle or complex.

An anti-cancer compound may be any anti-cancer drug or medicament which has activity against cancer cells. Suitable anti-cancer compounds for use in combination with the anti-HVEM antibody as disclosed herein may include aspirin, sulindac, curcumin, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); thylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics, such as actimomycin D, daunomycin (rubidomycin), doxorubicin, mitoxantrone, idarubicin, bleomycins, plicamycin (mithramycin), mitomycinC, and actinomycin; enzymes such as L-asparaginase, cytokines such as interferon (IFN)-gamma, tumor necrosis factor (TNF)-alpha, TNF-beta and GM-CSF, anti-angiogenic factors, such as angiostatin and endostatin, inhibitors of FGF or VEGF such as soluble forms of receptors for angiogenic factors, including soluble VGFNEGF receptors, platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Glivac) and erlotinib (an EGF receptor inhibitor) now marketed as Tarveca; and anti-virals such as oseltamivir phosphate, Amphotericin B, and palivizumab.

While it is possible for anti-HVEM antibody and anti-cancer compounds to be administered alone, it is preferable (when possible) to present the compounds in the same or separate pharmaceutical compositions (e.g. formulations).

A pharmaceutical composition may comprise, in addition to the anti-HVEM antibody and/or an anti-cancer compound, one or more pharmaceutically acceptable carriers, adjuvants, excipients, diluents, fillers, buffers, stabilizers, preservatives, lubricants, or other materials well known to those skilled in the art. Suitable materials will be sterile and pyrogen-free, with a suitable isotonicity and stability. Examples include sterile saline (e.g. 0.9% NaCl), water, dextrose, glycerol, ethanol or the like or combinations thereof. Such materials should be non-toxic and should not interfere with the efficacy of the active compound. The precise nature of the carrier or other material will depend on the route of administration, which may be by bolus, infusion, injection or any other suitable route, as discussed below. Suitable materials will be sterile and pyrogen free, with a suitable isotonicity and stability. Examples include sterile saline (e.g. 0.9% NaCl), water, dextrose, glycerol, ethanol or the like or combinations thereof. The composition may further contain auxiliary substances such as wetting agents, emulsifying agents, pH buffering agents or the like.

Suitable carriers, excipients, etc. can be found in standard pharmaceutical texts, for example, Remington's Pharmaceutical Sciences, 18th edition, Mack Publishing Company, Easton, Pa., 1990.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

In some embodiments, one or both of the anti-HVEM antibody and anti-cancer compound may be provided in a lyophilized form for reconstitution prior to administration. For example, lyophilized reagents may be re-constituted in sterile water and mixed with saline prior to administration to a subject

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols. Optionally, other therapeutic or prophylactic agents may be included in a pharmaceutical composition or formulation.

Increasing immune response to tumors as described herein may be useful in immunotherapy for the treatment of cancer. Treatment may be any treatment and therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment as a prophylactic measure (i.e. prophylaxis) is also included. For example, a subject susceptible to or at risk of the occurrence or re-occurrence of cancer may be treated as described herein. Such treatment may prevent or delay the occurrence or re-occurrence of cancer in the subject.

In particular, treatment may include inhibiting cancer growth, including complete cancer remission, and/or inhibiting cancer metastasis. Cancer growth generally refers to any one of a number of indices that indicate change within the cancer to a more developed form. Thus, indices for measuring an inhibition of cancer growth include a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of cytolytic T-lymphocytes, and a decrease in levels of tumor-specific antigens. Increasing immune response to tumors in a subject may improve the capacity of the subject to resist cancer growth, in particular growth of a cancer already present the subject and/or decrease the propensity for cancer growth in the subject.

The anti-HVEM antibody may be administered as described herein in therapeutically-effective amounts. The term “therapeutically-effective amount” as used herein, pertains to that amount of an active compound, or a combination, material, composition or dosage form comprising an active compound, which is effective for producing some desired therapeutic effect, commensurate with a reasonable benefit/risk ratio. It will be appreciated that appropriate dosages of the active compounds can vary from patient to patient. Determining the optimal dosage will generally involve the balancing of the level of therapeutic benefit against any risk or deleterious side effects of the administration. The selected dosage level will depend on a variety of factors including, but not limited to, the route of administration, the time of administration, the rate of excretion of the active compound, other drugs, compounds, and/or materials used in combination, and the age, sex, weight, condition, general health, and prior medical history of the patient. The amount of active compounds and route of administration will ultimately be at the discretion of the physician, although generally the dosage will be to achieve concentrations of the active compound at a site of therapy without causing substantial harmful or deleterious side-effects.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

For example, an anti-HVEM antibody as described herein, such as such as, for example, a bispecific anti-HVEM antibody, a scFV antibody, or CAR T-cells may be administered by continuous intravenous infusion in an amount sufficient to maintain the serum concentration at a level that inhibits tumor growth. Other anti-HVEM targeted agents described herein can also be used in this same manner.

Administration in vivo can be effected in one dose, continuously or intermittently (e.g., in divided doses at appropriate intervals). Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the physician.

Administration of anti-cancer compounds and the anti-HVEM antibody may be simultaneous, separate or sequential. By “simultaneous” administration, it is meant that the anti-cancer compounds and the anti-HVEM antibody are administered to the subject in a single dose by the same route of administration. By “separate” administration, it is meant that the anti-cancer compounds and the anti-HVEM antibody are administered to the subject by two different routes of administration which occur at the same time. This may occur for example where one agent is administered by infusion or parenterally and the other is given orally during the course of the infusion or parenteral administration. By “sequential” it is meant that the anti-cancer compounds and the anti-HVEM antibody are administered at different points in time, provided that the activity of the first administered agent is present and ongoing in the subject at the time the second agent is administered. For example, the anti-cancer compounds may be administered first, such that an immune response against a tumor antigen is generated, followed by administration of the anti-HVEM antibody, such that the immune response at the site of the tumor is enhanced, or vice versa. Preferably, a sequential dose will occur such that the second of the two agents is administered within 48 hours, preferably within 24 hours, such as within 12, 6, 4, 2 or 1 hour(s) of the first agent.

Multiple doses of the anti-HVEM antibody may be administered, for example 2, 3, 4, 5 or more than 5 doses may be administered after administration of the anti-cancer compounds. The administration of the anti-HVEM antibody may continue for sustained periods of time after administration of the anti-cancer compounds. For example, treatment with the anti-HVEM antibody may be continued for at least 1 week, at least 2 weeks, at least 3 weeks, at least 1 month or at least 2 months. Treatment with the anti-HVEM antibody may be continued for as long as is necessary to achieve complete tumor rejection.

Multiple doses of the anti-cancer compounds may be administered, for example 2, 3, 4, 5 or more than 5 doses may be administered after administration of the HVEM-targeted immune response agent. The administration of the anti-cancer compounds may continue for sustained periods of time after administration of the anti-HVEM antibody. For example, treatment with the anti-cancer compounds may be continued for at least 1 week, at least 2 weeks, at least 3 weeks, at least 1 month or at least 2 months. Treatment with the anti-cancer compounds may be continued for as long as is necessary to achieve complete tumor rejection.

The active compounds or pharmaceutical compositions comprising the active compounds may be administered to a subject by any convenient route of administration, whether systemically/peripherally or at the site of desired action, including but not limited to, oral (e.g. by ingestion); and parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly. Usually administration will be by the intravenous route, although other routes such as intraperitoneal, subcutaneous, transdermal, oral, nasal, intramuscular or other convenient routes are not excluded.

The pharmaceutical compositions comprising the active compounds may be formulated in suitable dosage unit formulations appropriate for the intended route of administration.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Preferred formulations for anti-HVEM antibody delivery include formulations suitable for parenteral administration (e.g. by injection, including cutaneous, subcutaneous, intramuscular, intravenous and intradermal), and include aqueous and non-aqueous isotonic, pyrogen-free, sterile injection solutions which may contain anti-oxidants, buffers, preservatives, stabilizers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

Compositions comprising anti-cancer compounds and/or anti-HVEM antibody may be prepared in the form of a concentrate for subsequent dilution, or may be in the form of divided doses ready for administration. Alternatively, the reagents may be provided separately within a kit, for mixing prior to administration to a human or animal subject.

The anti-HVEM antibody may be administered alone or in combination with other treatments, either simultaneously or sequentially dependent upon the individual circumstances. For example, anti-HVEM antibodies as described herein may be administered in combination with one or more additional active compounds.

In some embodiments, the treatment of a subject using an anti-HVEM antibody as described herein may further comprise administering one or more additional immunotherapeutic agents to the subject. An immunotherapeutic agent may facilitate or enhance the targeting of cancer cells by the immune system, in particular T-cells, through the recognition of antigens expressed by the cancer cells. Suitable agents include cancer vaccine preparations designed to induce T lymphocytes (T-cells) recognizing a localized region of an antigen or epitope specific to the tumor cell.

A cancer vaccine is an agent, a cell-based agent, molecule, or immunogen which stimulates or elicits an endogenous immune response in a subject or subject against one or more tumor antigens. Suitable cancer vaccines are known in the art and may be produced by any convenient technique.

The use of tumor antigens to generate immune responses is well-established in the art (see for example; Kakimi K, et al. Int J Cancer. 2011 Feb. 3; Kawada J, Int J Cancer. 2011 Mar. 16; Gnjatic S, et al. Clin Cancer Res. 2009 Mar. 15; 15(6):2130-9; Yuan J, et al. Proc Natl Acad Sci USA. 2008 Dec. 23; 105(51):20410-5; Sharma P, et al. J Immunother. 2008 November-December; 31(9):849-57; Wada H, et al. Int J Cancer. 2008 Nov. 15; 123(10):2362-9; Diefenbach C S, et al. Clin Cancer Res. 2008 May 1; 14(9):2740-8; Bender A, et al. Cancer Immun. 2007 Oct. 19; 7:16; Odunsi K, et al. Proc Natl Acad Sci USA. 2007 Jul. 31; 104(31):12837-42; Valmori D, et al. Proc Natl Acad Sci USA. 2007 May 22; 104(21):8947-52; Uenaka A, et al. Cancer Immun. 2007 Apr. 19; 7:9; Kawabata R, et al. Int J Cancer. 2007 May 15; 120(10):2178-84; Jsger E, et al. Proc Natl Acad Sci USA. 2006 Sep. 26; 103(39):14453-8; Davis ID Proc Natl Acad Sci USA. 2005 Jul. 5; 102(27):9734; Chen Q, Proc Natl Acad Sci USA. 2004 Jun. 22; 101(25):9363-8; Jsger E, Proc Natl Acad Sci USA. 2000 Oct. 24; 97(22):12198-203; Carrasco J, et al. J Immunol. 2008 Mar. 1; 180(5):3585-93; van Baren N, et al. J Clin Oncol. 2005 Dec. 10; 23(35):9008-21; Kruit W H, et al. Int J Cancer. 2005 Nov. 20; 117(4):596-604; Marchand M, et al. Eur J Cancer. 2003 January; 39(1):70-7; Marchand M et al. Int J Cancer. 1999 Jan. 18; 80(2):219-30; Atanackovic D, et al. Proc Natl Acad Sci USA. 2008 Feb. 5; 105(5):1650-5).

Cancer cells from the subject may be analyzed to identify a tumor antigen expressed by the cancer cells. For example, a method as described herein may comprise the step of identifying a tumor antigen which is displayed by one or more cancer cells in a sample obtained from the subject. A cancer vaccine comprising one or more epitopes of the identified tumor antigen may then be administered to the subject whose cancer cells express the antigen. The vaccine may induce or increase an immune response, preferably a T-cell mediated immune response, in the subject against the cancer cells expressing the identified tumor antigen.

The cancer vaccine may be administered before, at the same time, or after the anti-HVEM antibody is administered to the subject as described here.

Adoptive T-cell therapy involves the administration to a subject of tumor-specific T-cells to a subject. Preferably, the T-cells were previously isolated from the subject and expanded ex vivo. Suitable adoptive T-cell therapies are well known in the art (J. Clin Invest. 2007 June 1; 117(6): 1466-1476.) For example, adoptive T-cell therapy using CAR T-cells (chimeric antigen receptor) would be greatly improved if used in combination with an anti-HVEM antibody. CAR T-cells must migrate into a tumor to get in proximity to the cancer cells within the tumor in order to mediate their killing activity.

In some embodiments, the treatment of an individual using an anti-HVEM antibody may further comprise administering one or more tumor therapies to treat the cancerous tumor. Such therapies include, for example, tumor medicaments, radiation and surgical procedures.

A tumor medicament is an agent which is administered to a subject for the purpose of treating a cancer. Suitable medicaments for the treatment of tumors are well known in the art.

Suitable medicaments for use in combination with an anti-HVEM antibody as disclosed herein may include aspirin, sulindac, curcumin, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); thylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics, such as actimomycin D, daunomycin (rubidomycin), doxorubicin, mitoxantrone, idarubicin, bleomycins, plicamycin (mithramycin), mitomycinC, and actinomycin; enzymes such as L-asparaginase, cytokines such as interferon (IFN)-gamma, tumour necrosis factor (TNF)-alpha, TNF-beta and GM-CSF, anti-angiogenic factors, such as angiostatin and endostatin, inhibitors of FGF or VEGF such as soluble forms of receptors for angiogenic factors, including soluble VGFNEGF receptors, platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Glivac) and erlotinib (an EGF receptor inhibitor) now marketed as Tarveca; and anti-virals such as oseltamivir phosphate, Amphotericin B, and palivizumab.

Additionally, other T-cell checkpoint antagonists, like anti-Lag-3, anti-PD-1, anti-PD-L1, or inhibitors of IDO1/ID02 (indoleamine 2,3-dioxygenase) could also be used in combination with the present invention. These latter enzymes catabolize tryptophan in the tumor microenvironment, which impairs T-cell function. By using an anti-HVEM antibody, such as for example, a bispecific anti-HVEM antibody, or a CAR T-cells, in combination with a T-cell checkpoint antagonist may synergistically increase cancer cell killing within a tumor.

Various embodiments are disclosed above for an anti-HVEM antibody. Aspects and embodiments of the invention relating to an anti-HVEM antibody and optionally one or more other agents disclosed above include disclosure of the administration of the compounds or agents separately (sequentially or simultaneously) or in combination (co-formulated or mixed). For each aspect or embodiment, the specification further discloses a composition comprising the anti-HVEM antibody and optionally one or more other agents co-formulated or in admixture with each other and further discloses a kit or unit dose containing the anti-HVEM antibody. Optionally, such compositions, kits or doses further comprise one or more carriers in admixture with the agent or co-packaged for formulation prior to administration to an individual.

Various embodiments are also disclosed above for combinations of a check-point inhibitor, such as a PD-1 signaling inhibitor, and an anti-HVEM antibody. Aspects and embodiments of the invention relating to combinations of a PD-1 signaling inhibitor and anti-HVEM antibody and optionally one or more other agents disclosed above include disclosure of the administration of the compounds or agents separately (sequentially or simultaneously) or in combination (co-formulated or mixed). For each aspect or embodiment, the specification further discloses a composition comprising the PD-1 signaling inhibitor and anti-HVEM antibody and optionally one or more other agents co-formulated or in admixture with each other and further discloses a kit or unit dose containing the PD-1 signaling inhibitor and anti-HVEM antibody packaged together, but not in admixture. Optionally, such compositions, kits or doses further comprise one or more carriers in admixture with one or both agents or co-packaged for formulation prior to administration to a subject.

Various further aspects and embodiments of the present invention will be apparent to those skilled in the art in view of the present disclosure.

### EXAMPLES

The invention will now be further illustrated with reference to the following examples. It will be appreciated that what follows is by way of example only and that modifications to detail may be made while still falling within the scope of the invention.

### Example 1—Generation of Anti-HVEM Antibodies

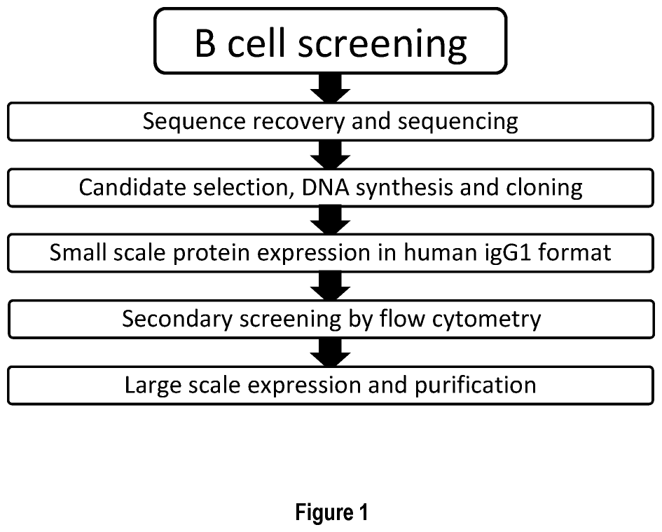

The workflow shown in FIG. 1 illustrates the binding confirmation process after a repertoire of B cells have been screened for B cells of interest (e.g., B cells that may secret the antibodies of interest). The B cell screening can be performed with droplet-based microfluidic technology, such as for example, as described in G6rard et al., “High-throughput single-cell activity-based screening and sequencing of antibodies using droplet microfluidics,” Nature Biotechnology, volume 38, pages 715-721(2020) (herein incorporated by reference in its entirety).

To illustrate, human or immunized animal enriched B cells, and optionally further ex vivo activated, in cell culture medium are introduced into a microfluidic chip where they are encapsulated into microdroplets following a Poisson statistics distribution, such that no more than 5% of the droplet contains two cells. These droplets are <40 pL volume. Cells are co-encapsulated with bio-assay reagents including streptravidin-coated magnetic colloid beads and fluorescently-labeled antigen of interest, and optionally a fluorescently labelled detection reagent used to identify antibody secreting cells.

The encapsulated B cells in the droplets can be screened and sorted for B cells that produce secreted IgG antibodies, detected optionally with the detection reagent, that specifically bind to the fluorescently-labeled antigen of interest. The droplets of interest are deflected from main channel to sorting channel by surface acoustic wave mediated process. The B cells in these droplets of interest are then collected and subjected to single-cell reverse transcription with primers for VH and VL, as detailed, e.g., in Gérard et al. The cDNAs generated from each cell carry a different barcode, allowing cognate VH and VL pairs to be identified after next generation sequencing (NGS) to obtain the cDNA sequences.

To illustrate, the cDNA sequences can be analyzed using an IMGT V-gene database such as for example, the database described in G6rard et al. An exemplary sequence analysis may include: 1) after immune characterization of consensus reads by VDJFasta, reads containing frameshifts, stop-codons or lacking identifiable CDRs were filtered out. VH-VL pairing was carried out by identifying the most abundant VH and VL consensus sequence (by number of reads that contributed to that consensus) in each barcode cluster; 2) the paired VH and VL sequences must be larger than any other VH or VL present in the cluster by at least 1 read; 3) to minimize VH-VL mispairing, antibody sequences were only considered for further analysis if both the paired VH/VL consensus sequences comprised at least 25, 30, 40, 50, 60 or more reads; 4) low-level mispairing (wrong assignment of light chain with heavy chain) was removed by clustering all heavy chains with the same V-J gene combination and a CDR3 amino acid sequence within a hamming distance of 2 and using the paired light chain associated with the largest number of independent barcodes.

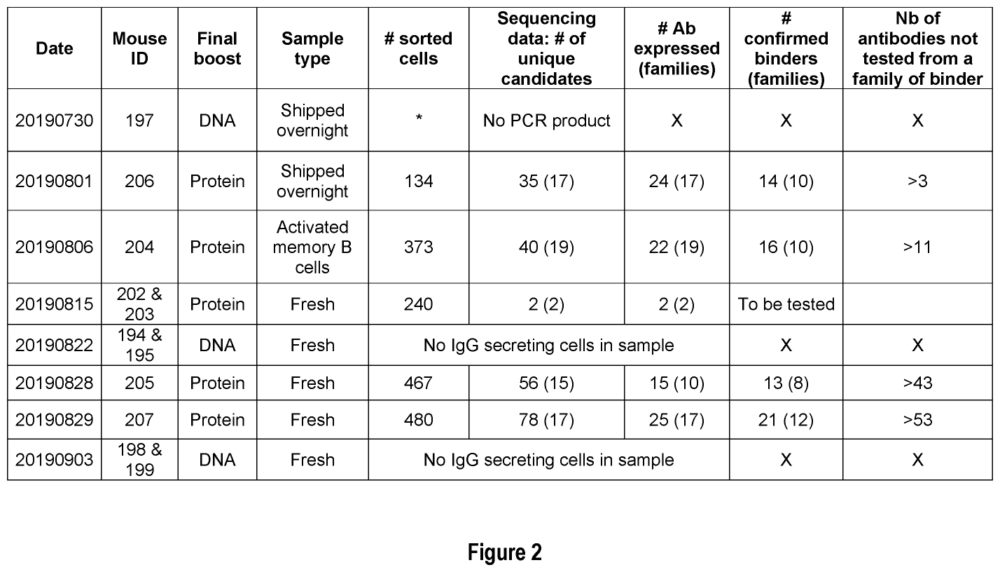

FIG. 2 summarizes the screening results with samples from 11 immunized mice. The results indicate that the mice that received a final protein boost produced more antibodies of interest (e.g., mice IDs. 206, 204, 205 and 207). “Fresh” refers to fresh plasma cells from the mice, as compared to “shipped overnight” (i.e., overnight shipped spleen) and memory activated B cells.

### Example 2—Expression of the Anti-HVEM Antibody

The anti-HVEM antibodies as described herein can be constructed using standard molecular biology techniques well known to the skilled artisan. For example, plasmids comprising a polynucleotide encoding an anti-HVEM antibody can be designed to express a polypeptide comprising the amino acid sequences disclosed in Tables 2-3.

It will be appreciated that Fab and F(ab′)2 and other fragments of the anti-HVEM antibodies may be used according to the methods disclosed herein. Such fragments are typically produced by proteolytic cleavage, using enzymes such as papain (to produce Fab fragments) or pepsin (to produce F(ab′)2 fragments). Alternatively, secreted protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

### Example 3: Membrane HVEM Expression Using Retrovirus

Flow Cytometry (FACS) analysis of a cell line expressing the HVEM receptor in its natural conformation is used to measure the serum titer and/or antibody binding. To create such a cell line, retroviral vectors can be used to stably integrate target HVEM gene into the host cell chromosome using standard techniques. By stably integrating the target gene into the host genome, the host cell will permanently and stably express the HVEM receptor without selection pressure and and the cell can be banked.

In this example, an internal ribosome entry site-enhanced green fluorescent protein (IRES-EGFP) sequence is cloned into a retroviral PMV vector. EGFP can be expressed with the target protein together and used as indicator for verifying the transfection effect or target protein expression level. EGFP can be used as indicator for verifying the transfection using Fluorescence microscope or FACS (EGFP use the same channel with FITC or 488 channel).

The HVEM sequence is cloned into the multiple cloning site of the retroviral vector pMV. This vector is then transformed into packaging cell, such as Plat-E cells, although many packing cell lines are publicly available with a chemical method, such as Lipofectamine LTR and Plus agent. The retrovirus encoding HVEM is created and secreted into the cell culture medium. The supernatant will be collected and directly be applied for transfection without super centrifugation or other concentrate processing.

Plates coated with Retronectin Protein solution are used as we have found that this protein can fix the virus to the plate surface without over-night supercentrifugation, thereby dramatically increasing the transfection efficiency. The supernatant containing retrovirus is added into the plate which is captured by the Retronectin and fixing the retrovirus to the plate surface.

A mouse pro-B, IL-3 dependent cell-line that grows in suspension (BaF3 cells) are added to the plate without any additional treatment for transfection. BaF3 also will be captured by the Retronectin protein, dramatically increasing the contact frequency of BaF3 cell and retrovirus leading to an increase in successful transfection. By performing a limited-dilution results in obtaining the top-3 single BaF3 cell clones with high EGFP/HVEM protein expression level and allows for the ability to banik a single clone.

### Example 4a: Measurement of Binding Affinity Via ELISA

For ELISA measurements, human HVEM recombinant protein (Sino Biological, 10334-H03H, 1 ug/ml, 100 ul/well) was coated to ELISA plate (Thermo Scientific, 469949, 4C overnight). HVEM antibody clone's concentration was diluted to 125 ng/ml and 100 ul was added to the ELISA plate after blocking with 3% BSA (200 ul/well, RT, 2 Hr) for 1 Hr at RT. Plate was washed with PBST; diluted HRP-anti-mouse IgG (Southern BioTech, 1030-05, 1:6000) with PBS containing 5% FBS was added 100 ul per well for 1 Hr at RT. TMB substrate (KPL, 52-00-00) 100 ul per well after washing and incubate at room temperature for 15 minutes; then stop the development by adding 100 ul stop solution (KPL, 50-85-06). Plate was read at 450 nm.

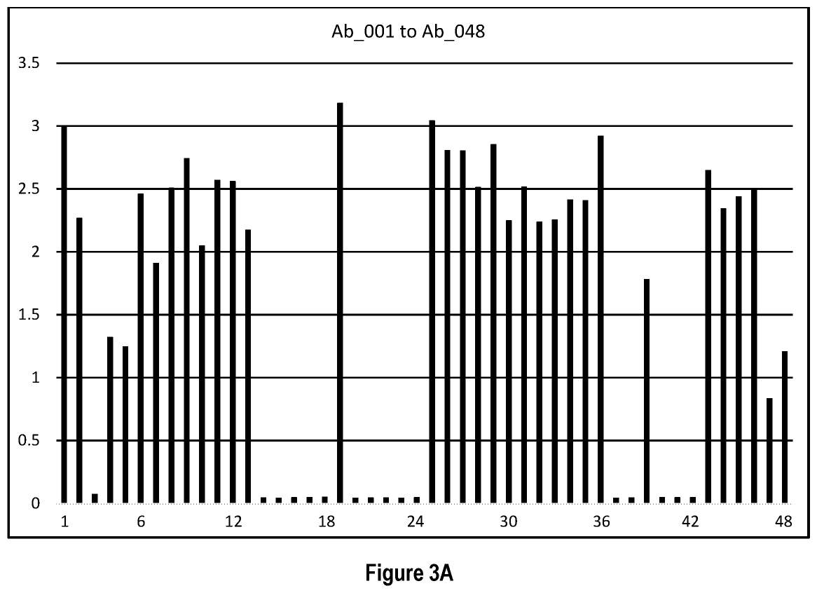

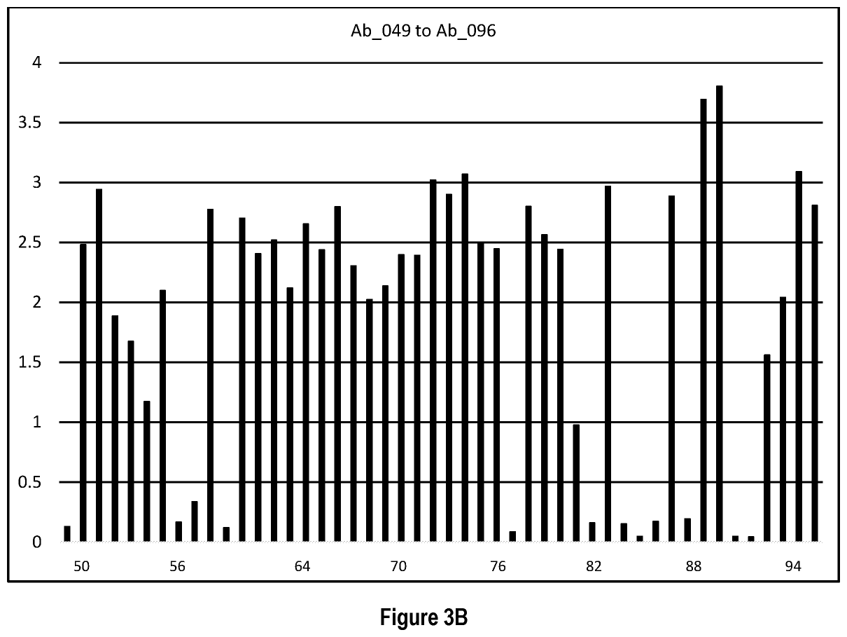

Data were obtained for antibodies Ab_001 to Ab_096 (see Table 1 for description of the antibodies) using a 96-well plate, with intensity of absorbance at 450 nm correlating with affinity of an antibody to the human HVEM. Bar graphs showing the intensities are provided in FIGS. 3a and 3b, with higher intensity indicating stronger binding to HVEM in the assay. As indicated in FIGS. 3a and 3b, intensities at 450 nm ranged from 0 to 4, with antibodies Ab_001, Ab_019, Ab_025, Ab_072, Ab_074, Ab_083, Ab_089, Ab_090, and Ab_095 showing intensities between 3.0 and 4.0, indicating relatively strong binding by ELISA; antibodies Ab_006, Ab_008, Ab_009, Ab_011, Ab_012, Ab_26, Ab_027, Ab_028, Ab_029, Ab_031, Ab_036, Ab_043, Ab_046, Ab_050, Ab_051, Ab_058, Ab_060, Ab_062, Ab_064, Ab_066, Ab_073, Ab_075, Ab_077, Ab_078, Ab_079, Ab_087, and Ab_096 showing intensities between 2.5 and 3.0, antibodies Ab_002, Ab_004, Ab_005, Ab_007, Ab_010, Ab_013, Ab_030, Ab_032, Ab_033, Ab_034, Ab_035, Ab_039, Ab_044, Ab_045, Ab_048, Ab_052, Ab_053, Ab_054, Ab_055, Ab_061, Ab_063, Ab_065, Ab_067, Ab_068, Ab_069, Ab_070, Ab_071, Ab_076, Ab_080, Ab_093, and Ab_094 showing intensities between 1.0 and 2.5, and antibodies Ab_003, Ab_014, Ab_015, Ab_016, Ab_017, Ab_018, Ab_020, Ab_021, Ab_022, Ab_023, Ab_024, Ab_037, Ab_038, Ab_040, Ab_041, Ab_042, Ab_049, Ab_056, Ab_057, Ab_059, Ab_077, Ab_082, Ab_084, Ab_085, Ab_086, Ab_088, Ab_091, and Ab_092 showing intensities between 0.01 and 0.5, indicating weak to no binding.

ELISA was also used to assess comparative binding of antibodies to human, cynomolgus monkey, and murine HVEM. Results are shown in Table 5 below (with higher numbers indicating stronger binding).

TABLE 5

| Clone | Human | Cyno | Mouse |
|---|---|---|---|
| Ab_1 | 3.299 | 0.072 | 0.057 |
| Ab_2 | 2.801 | 1.981 | 0.043 |
| Ab_3 | 2.397 | 2.285 | 0.051 |
| Ab_4 | 2.421 | 0.062 | 0.044 |
| Ab_5 | 2.442 | 0.063 | 0.048 |
| Ab_6 | 3.138 | 2.688 | 0.043 |
| Ab_7 | 2.557 | 0.739 | 0.042 |
| Ab_8 | 3.397 | 2.937 | 0.077 |
| Ab_9 | 3.113 | 1.878 | 0.047 |
| Ab_10 | 2.679 | 0.068 | 0.044 |
| Ab_11 | 3.161 | 2.97 | 0.049 |
| Ab_12 | 3.081 | 2.893 | 0.044 |
| Ab_13 | 3.003 | 2.74 | 0.048 |
| Ab_14 | 0.05 | 0.064 | 0.049 |
| Ab_15 | 0.045 | 0.058 | 0.042 |
| Ab_16 | 0.047 | 0.061 | 0.042 |
| Ab_17 | 0.051 | 0.06 | 0.044 |
| Ab_18 | 0.05 | 0.059 | 0.043 |
| Ab_20 | 0.049 | 0.064 | 0.043 |
| Ab_21 | 0.046 | 0.061 | 0.042 |
| Ab_22 | 0.157 | 0.165 | 0.183 |
| Ab_23 | 0.045 | 0.057 | 0.042 |
| Ab_24 | 0.048 | 0.063 | 0.044 |
| Ab_25 | 3.01 | 3.157 | 0.045 |
| Ab_26 | 3.103 | 0.088 | 0.043 |
| Ab_27 | 3.126 | 0.064 | 0.043 |
| Ab_28 | 3.16 | 2.187 | 0.05 |
| Ab_29 | 3.328 | 0.068 | 0.045 |
| Ab_30 | 3.082 | 2.978 | 0.046 |
| Ab_31 | 3.084 | 3.06 | 0.042 |
| Ab_32 | 3.04 | 2.87 | 0.045 |
| Ab_33 | 3.097 | 3.015 | 0.043 |
| Ab_34 | 3.195 | 0.466 | 0.045 |
| Ab_35 | 3.198 | 0.616 | 0.045 |
| Ab_36 | 3.257 | 0.079 | 0.046 |
| Ab_37 | 0.059 | 0.06 | 0.043 |
| Ab_38 | 0.049 | 0.06 | 0.043 |
| Ab_39 | 2.867 | 3.03 | 0.042 |
| Ab_40 | 0.069 | 0.098 | 0.05 |
| Ab_41 | 0.059 | 0.076 | 0.045 |
| Ab_42 | 0.049 | 0.062 | 0.046 |
| Ab_43 | 3.22 | 0.058 | 0.043 |
| Ab_44 | 2.869 | 0.384 | 0.706 |
| Ab_45 | 3.079 | 2.897 | 0.042 |
| Ab_46 | 2.937 | 3.034 | 0.042 |
| Ab_47 | 1.19 | 0.743 | 0.051 |
| Ab_48 | 2.495 | 0.826 | 0.044 |
| Ab_49 | 1.074 | 0.065 | 0.044 |
| Ab_50 | 3.016 | 0.094 | 0.047 |
| Ab_51 | 3.179 | 0.563 | 0.043 |
| Ab_52 | 2.881 | 2.324 | 0.042 |
| Ab_53 | 2.893 | 2.099 | 0.042 |
| Ab_54 | 2.496 | 2.232 | 0.042 |
| Ab_55 | 2.946 | 2.212 | 0.042 |
| Ab_56 | 0.452 | 0.06 | 0.043 |
| Ab_57 | 1.066 | 1.059 | 0.045 |
| Ab_58 | 3.164 | 0.064 | 0.043 |
| Ab_59 | 0.462 | 0.405 | 0.043 |
| Ab_60 | 3.087 | 3.034 | 0.044 |
| Ab_61 | 2.984 | 3.026 | 0.043 |
| Ab_62 | 2.949 | 2.984 | 0.045 |
| Ab_63 | 3.103 | 3.133 | 0.043 |
| Ab_64 | 3.072 | 0.987 | 0.044 |
| Ab_65 | 3.242 | 1.525 | 0.044 |
| Ab_66 | 3.282 | 0.883 | 0.044 |
| Ab_67 | 3.052 | 3.042 | 0.047 |
| Ab_68 | 3.031 | 3.038 | 0.044 |
| Ab_69 | 3.132 | 3.039 | 0.054 |
| Ab_70 | 3.227 | 3.101 | 0.062 |
| Ab_71 | 3.077 | 2.983 | 0.047 |
| Ab_72 | 3.361 | 0.062 | 0.045 |
| Ab_73 | 3.24 | 0.062 | 0.045 |
| Ab_74 | 3.26 | 0.06 | 0.044 |
| Ab_75 | 3.043 | 3.044 | 0.048 |
| Ab_76 | 2.892 | 2.96 | 0.044 |
| Ab_77 | 0.147 | 0.079 | 0.043 |
| Ab_78 | 3.077 | 0.063 | 0.046 |
| Ab_79 | 2.966 | 0.062 | 0.054 |
| Ab_80 | 3.072 | 2.259 | 0.048 |
| Ab_81 | 1.589 | 0.076 | 0.048 |
| Ab_82 | 0.844 | 0.06 | 0.044 |
| Ab_83 | 3.193 | 0.06 | 0.043 |
| Ab_84 | 0.171 | 0.061 | 0.043 |
| Ab_85 | 0.049 | 0.061 | 0.046 |
| Ab_86 | 0.575 | 0.065 | 0.044 |
| Ab_87 | 3.218 | 0.069 | 0.045 |

Binding of antibodies to human HVEM may also be assessed by flow cytometry and by bio-layer interferometry (BLI).

### Example 4b: Measurement of Binding Affinity by Bio-Layer Interferometry (BLI) by an OctetRed96® Assay

Binding of antibodies to HVEM may also be determined by bio-layer interferometry (BLI) on an OctetRed96@ system (Sartorius). (See http://www.fortebio.com/bli_technology.html for general description of a BLI assay.) For this experiment, murine anti-human HVEM antibodies were captured from culture supernatant using anti-mouse IgG Fc capture and immobilized to dip and read biosensors. Sensors were then dipped into a solution of 200 nM His-tagged human HVEM in phosphate-buffered saline (PBS). Probes were dipped into PBS assay buffer and the dissociation rate (k_{off}) was measured. The association rate (kₒₙ) and affinity (K_{D}) were determined by curve fitting analysis.

Binding data for exemplary antibodies are provided above in Table 1.

### Example 5—Competitive Assays with HVEM Ligands BTLA and LIGHT

The competitive activity of HVEM antibody to BTLA or LIGHT was evaluated with ELISA-based competitive assay. Briefly Human HEVM recombinant protein (Sino Biological, 10334-H02H, 4 ug/ml, 100 ul/well) was coated to ELISA plate (Thermo Scientific, 469949, 4C overnight). A pre-mixture of HVEM antibody clone with seral dilution and 400 nM BTLA-His (R&D systems, 9235-BT-050) or LIGHT-His (SinoBiological, 10386-H07H) recombinant protein was made and added to the ELISA plate after blocking with 3% BSA (200 ul/well, RT 2 Hr) for 1 Hr at RT. The serial dilutions of HVEM antibody clone involve 7 different concentrations, with a 3-fold dilution performed start from 100 nM for BTLA or 325 nM for LIGHT competitive assay. The concentration was the final concentration. Plate was washed with PBST; diluted HRP-anti-His (Biolegend, 652504, 1:1000) with PBS containing 5% FBS was added 100 ul per well for 1 Hr at RT. TMB substrate (KPL, 52-00-00) 100 ul per well after washing and incubate at room temperature for 15 minutes; then stop the development by adding 100 ul stop solution (KPL, 50-85-06). Plate was read at 450 nm. The IC50 was calculated using GraphPad Prism software (GraphPad Software, Inc. San Diego, CA, USA).

As shown in Table 1, binding to HVEM to inhibit HVEM's ligands, LIGHT and BTLA, from binding to HVEM was confirmed for a number of the disclosed antibodies.

Variations, modifications, and other implementations of what is described herein will occur to those of ordinary skill in the art without departing from the spirit and scope of the invention and the claims. All of the patents, patent applications, international applications, and references identified are expressly incorporated herein by reference in their entireties.

What is claimed is:

1. An isolated antibody that binds to HVEM, comprising:
(a) a heavy chain comprising VH CDR1, VH CDR2, and VH CDR3 comprising, respectively: SEQ ID Nos 285, 464, and 709 (consensus cluster 11); SEQ ID Nos 298, 470, and 720 (consensus cluster 20); SEQ ID Nos 304, 478, and 729 (consensus cluster 5); SEQ ID Nos 310, 481, and 733 (consensus cluster 23); SEQ ID Nos 321, 495, and 751 (consensus cluster 21); SEQ ID Nos 328, 504, and 753 (consensus cluster 10); SEQ ID Nos 336, 513, and 776 (consensus cluster 8); SEQ ID Nos 340, 514, and 783 (consensus cluser 15); SEQ ID Nos 347, 522, and 795 (consensus cluster 19); SEQ ID Nos 351, 525, and 801 (consensus cluster 14); SEQ ID Nos 355, 530, and 808 (consensus cluster 6); SEQ ID Nos 356, 531, and 811 (consensus cluster 12); SEQ ID Nos 358, 535, and 815 (consensus cluster 4); SEQ ID Nos 361, 538, and 816 (consensus cluster 9); SEQ ID Nos 364, 541, and 821 (consensus cluster 17); SEQ ID Nos 366, 544, and 826 (consensus cluster 7); SEQ ID Nos 367, 547, and 829 (consensus cluster 13); SEQ ID Nos 369, 550, and 833 (consensus cluster 18); SEQ ID Nos 371, 553, and 837 (consensus cluster 22); SEQ ID Nos 374, 557, and 841 (consensus cluster 16); SEQ ID Nos 338, 513, and 844 (consensus cluster 1); SEQ ID Nos 375, 559, and 845 (consensus cluster 2); or SEQ ID Nos 376, 560, and 846 (consensus cluster 3); and
(b) a light chain comprising VL CDR1, VL CDR2, and VL CDR3 comprising, respectively: SEQ ID Nos 1099, 1230, and 1343 (consensus cluster 6); SEQ ID Nos 1129, 1246, and 1376 (consensus cluster 7); SEQ ID Nos 1136, 1249, and 1387 (consensus cluster 3); SEQ ID Nos 1142, 1251, and 1399 (consensus cluster 5); SEQ ID Nos 1152, 1248, and 1411 (consensus cluster 1); SEQ ID Nos 1155, 1256, and 1416 (consensus cluster 4); and SEQ ID Nos 1159, 1258, and 1422 (consensus cluster 2).

2. The antibody of claim 1, wherein the heavy chain further comprises an FR1, FR2, FR3, and FR4 corresponding to the consensus cluster of the VH CDR1, VH CDR2, and VH CDR3, and/or wherein the light chain further comprises an FR1, FR2, FR3, and FR4 corresponding to the consensus cluster of the VL CDR1, VL CDR2, and VL CDR3.

3. An isolated antibody that binds to HVEM, comprising a heavy chain comprising VH CDR1, VH CDR2, and VH CDR3 and the VL CDR1, VL CDR2, and VL CDR3 of any one of Ab_001, Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087.

4. The antibody of claim 3, wherein the heavy chain comprises a heavy chain variable region (VH) with an amino acid sequence that is at least 90%, at least 95%, or at least 97% identical to that of the VH of Ab_001, Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087, and/or wherein the light chain comprises a light chain variable region (VL) with an amino acid sequence that is at least 90%, at least 95%, or at least 97% identical to that of the VL of Ab_001, Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087.

5. The antibody of claim 3, wherein the heavy chain comprises a VH with an amino acid sequence comprising the amino acid sequence of the VH of Ab_001, Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087, and/or wherein the light chain comprises a VL with an amino acid sequence comprising the amino acid sequence of the VL of Ab_001, Ab_006, Ab_008, Ab_009, Ab_010, Ab_011, Ab_012, Ab_013, Ab_025, Ab_026, Ab_027, Ab_028, Ab_029, Ab_030, Ab_031, Ab_034, Ab_035, Ab_036, Ab_043, Ab_044, Ab_045, Ab_046, Ab_050, Ab_051, Ab_058, Ab_063, Ab_159, Ab_064, Ab_065, Ab_066, Ab_067, Ab_068, Ab_069, Ab_155, Ab_070, Ab_071, Ab_149, Ab_072, Ab_073, Ab_074, Ab_078, Ab_079, Ab_080, Ab_083, Ab_153, or Ab_087.

6. An isolated antibody that binds to HVEM, comprising:
(a) an antibody selected from any one of the antibodies listed by either AntibodyID or Ab_Num_Id as described in Table 1;
(b) an antibody comprising a heavy chain comprising an amino acid sequence selected from any one of the amino acid sequences of SEQ ID NO:1-201;
(c) an antibody comprising a light chain comprising an amino acid sequence selected from any one of the amino acid sequences of SEQ ID NO:874-1032;
(d) an antibody comprising a heavy chain comprising an amino acid sequence selected from any one of the amino acid sequences of SEQ ID NO:1-201 and a light chain comprising an amino acid sequence selected from any one of the amino acid sequences of SEQ ID NO:874-1032;
(e) an amino acid sequence having at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% sequence identity to any one of (a)-(d);
(f) the amino acid sequence of (e), wherein CDRH1, CDRH2 and CDRH3 of SEQ ID NO:1-201 is maintained;
(g) the amino acid sequence of (e), wherein CDRL1, CDRL2 and CDRL3 of SEQ ID NO:874-1032 is maintained;
(h) the amino acid sequence of (e), wherein the CDRH1, CDRH2, and CDRH3 of of SEQ ID NO:1-201 and CDRL1, CDRL2 and CDRL3 of SEQ ID NO:874-1032 is maintained;
(i) an antibody comprising a CDRH1, a CDRH2, and a CDRH3 selected from an amino acid sequence of any one of SEQ ID NO:1-201;
(j) an antibody comprising a CDRL1, a CDRL2, and a CDRL3 selected from an amino acid sequence of any one of SEQ ID NO:874-1032;
(k) an antibody comprising a CDRH1, a CDRH2, and a CDRH3 selected from an amino acid sequence of any one of SEQ ID NO:1-201 and a CDRL1, a CDRL2, and a CDRL3 selected from an amino acid sequence of any one of SEQ ID NO:874-1032;
(l) an antibody comprising a CDRH1, a CDRH2, and a CDRH3 selected from an amino acid sequence of any one of SEQ ID NO:1-201 and a CDRL1, a CDRL2, and a CDRL3 selected from an amino acid sequence of any one of SEQ ID NO:874-1032, wherein said selection of CDRH1, CDRH2, CDRH3, CDRL1, CDRL2, and CDRL3 are selected from the same AntibodyId as described in Table 1;
(m) an antibody comprising at least one of SEQ ID NO: 202-873 and/or at least one of SEQ ID NO:1033-1449;
(n) a single-chain variable fragment (“scFV”) comprising any one of (a)-(m); or
(o) a heavy chain and/or a light chain variable domain comprising any one of (a)-(m).

7. The antibody of any one of claims 1-6, wherein (a) the heavy chain comprises:
(1) a human IgM constant domain; (2) a human IgGI constant domain; (3) a human IgG2 constant domain; (4) a human IgG3 constant domain; (5) a human IgG4 constant domain; or (6) a human IgA constant domain;
(b) the light chain comprises a human Ig kappa constant domain or a human Ig lambda constant domain; or
(c) the heavy chain comprises: (1) a human IgM constant domain; (2) a human IgGI constant domain; (3) a human IgG2 constant domain; (4) a human IgG3 constant domain; (5) a human IgG4 constant domain; or (6) a human IgA constant domain; and
the light chain comprises a human Ig kappa constant domain or a human Ig lambda constant domain.

8. The antibody of any one of claims 1-7, wherein the antibody comprises a full length heavy chain constant region and/or a full length light chain constant region.

9. The antibody of any one of claims 1-7, wherein the antibody is a Fab fragment, a Fab′ fragment, a F(ab′)2 fragment, a Fv fragment, a disulfide linked F fragment, or a scFv fragment.

10. The antibody of any one of claims 1-9, wherein the antibody:
(a) blocks the binding of human BTLA to human HVEM with an IC50 of 10 nM or less, 3 nM or less, or 2 nM or less;
(b) blocks the binding of human LIGHT to human HVEM with an IC50 of 30 nM or less, 20 nM or less, or 10 nM or less;
(c) blocks the binding of human BTLA to human HVEM with an IC50 of 10 nM or less, 3 nM or less, or 2 nM or less, and also blocks the binding of human LIGHT to human HVEM; or
(d) blocks the binding of human LIGHT to human HVEM with an IC50 of 30 nM or less, 20 nM or less, or 10 nM or less, and also blocks the binding of human BTLA to human HVEM.

11. The antibody of any one of claims 1-10, wherein the antibody binds to human HVEM with a K_{D} of 50 nM or less, or 10 nM or less.

12. The antibody of any one of claims 1-11, wherein the antibody binds to cynomolgus monkey HVEM with a K_{D} of 50 nM or less, or 10 nM or less.

13. The antibody of any one of claims 1-12, wherein the antibody is bispecific or multispecific.

14. The antibody of claim 13, wherein the antibody is a bispecific antibody selected from: a bispecific T-cell engager (BiTE) antibody, a dual-affinity retargeting molecule (DART), a CrossMAb antibody, a DutaMab™ antibody, a DuoBody antibody; a Triomab, a TandAb, a bispecific NanoBody, Tandem scFv, a diabody, a single chain diabody, a HSA body, a (scFv)2 HSA Antibody, an scFv-IgG antibody, a Dock and Lock bispecific antibody, a DVD-IgG antibody, a TBTI DVD-IgG, an IgG-fynomer, a Tetravalent bispecific tandem IgG antibody, a dual-targeting domain antibody, a chemically linked bispecific (Fab′)2 molecule, a crosslinked mAb, a Dual-action Fab IgG (DAF-IgG), an orthoFab-IgG, a bispecific CovX-Body, a bispecific hexavalent trimerbody, 2 scFv linked to diphtheria toxin, and an ART-Ig.

15. The antibody of either claim 13 or 14, wherein the antibody is a bispecific antibody comprising (a) an anti-CXCL12 antibody; (b) an anti-CXCR4 antibody; (c) an anti-CD47 antibody; (d) a checkpoint inhibitor antibody, preferably an anti-PD-1 antibody, an anti-PD-L1 antibody, an anti-CTLA-4 antibody, an anti-TIM-3 antibody, and/or an anti-LAG3 antibody, (e) an anti-T-cell co-receptor antibody (e.g., an anti-4-1 BB (CD137) antibody or an anti-ICOS (CD278) antibody); or (f) an anti-neoantigen antibody.

16. The antibody of claim 15, wherein the antibody is an anti-neoantigen antibody, wherein the neoantigen is selected from: MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-1, LAGE-1, SSX-I, SSX-2(HOM-MEL-40), SSX-3, SSX-4, SSX-5, SCP-1 and XAGE, melanocyte differentiation antigens, p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-1), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding proteincyclophilin C-associated protein), TAAL6, TAG72, TLP, TPS, tyrosinase related proteins, TRP-1, TRP-2, or mesothelin.

17. The antibody of any one of claims 1-16, wherein the antibody further comprises:
(a) a detectable label, such as a radiolabel, an enzyme, a fluorescent label, a luminescent label, or a bioluminescent label; or
(b) a conjugated therapeutic or cytotoxic agent.

18. The antibody of claim 17, wherein:
(a) the detectable label is selected from 1251, 1311, In, 90Y, 99Tc, 177Lu, 166Ho, or 153Sm, or a biotinylated molecule; or
(b) the conjugated therapeutic or cytotoxic agent is selected from (a) an anti-metabolite; (b) an alkylating agent; (c) an antibiotic; (d) a growth factor; (e) a cytokine;
(f) an anti-angiogenic agent; (g) an anti-mitotic agent; (h) an anthracycline; (i) toxin; and/or (j) an apoptotic agent.

19. An isolated antibody that competes with an antibody according to any one of claims 1-18 for binding to HVEM.

20. A kit comprising the isolated antibody of any one of claims 1-18.

21. A pharmaceutical composition comprising the isolated antibody according to any one of claims 1-19, and further comprising a pharmaceutical acceptable carrier and/or excipient.

22. An isolated nucleic acid encoding the antibody of any one of claims 1-19, or encoding the heavy chain or light chain of the antibody.

23. A set of isolated nucleic acids encoding the antibody of any one of claims 1-19.

24. A vector comprising the nucleic acid or the set of nucleic acids of claim 22 of 23.

25. An isolated host cell comprising the nucleic acid of claim 22, the set of nucleic acids of claim 23, or the vector of claim 24, or an isolated host cell engineered to express the antibody of any one of claims 1-19.

26. Use of the antibody of any one of claims 1-19, wherein said use is selected from:
(a) a method of detecting aberrant expression of the HVEM protein in a sample in vitro or in a subject;
(b) a method for diagnosing a disease or disorder associated with aberrant HVEM protein expression or activity;
(c) a method of inhibiting HVEM activity in a sample in vitro or in a subject;
(d) a method of increasing HVEM activity in a sample in vitro or in a subject;
(e) a method of inhibiting HVEM binding to BTLA and/or LIGHT in a sample in vitro or in a subject; and/or
(f) a method of treating a disease or disorder in a subject associated with aberrant HVEM expression or activity.

27. Use of the antibody of any one of claims 1-19 in preparation of a medicament for diagnosis or treatment of a disease or disorder in a human subject.

28. The use of claim 27, wherein:
(a) the disease or disorder is HIV infection;
(b) the disease or disorder is cancer, such as an adenocarcinoma, sarcoma, skin cancer, melanoma, bladder cancer, brain cancer, breast cancer, uterus cancer, ovarian cancer, prostate cancer, lung cancer, colorectal cancer, cervical cancer, liver cancer, head and neck cancer, esophageal cancer, pancreas cancer, pancreatic ductal adenocarcinoma (PDA), renal cancer, stomach cancer, multiple myeloma or cerebral cancer;
(c) the use further comprises co-administering other anti-cancer therapies, such as a chemotherapeutic agent, radiation therapy, a cancer therapy, an immunotherapy, or a cancer vaccine, a cytokine, a toxin, a pro-apoptotic protein or a chemotherapeutic agent.

29. The use of claim 27 or 28, wherein the use comprises co-administering a cancer vaccine, and wherein the cancer vaccine recognizes one or more tumor antigens expressed on cancer cells, preferably, wherein the tumor antigen is selected from: MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-I, GAGE-2, GAGE-3, GAGE-4, GAGE-5, GAGE-6, GAGE-7, GAGE-8, BAGE-I, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-I, SSX-I, SSX-2(HOM-MEL-40), SSX-3, SSX-4, SSX-5, SCP-I and XAGE, melanocyte differentiation antigens, p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-1), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding proteincyclophilin C-associated protein), TAAL6, TAG72, TLP, TPS, tyrosinase related proteins, TRP-1, TRP-2, or mesothelin.

30. The use of claim 28, wherein use comprises co-administering another anti-cancer therapy selected from: aspirin, sulindac, curcumin, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); thylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics, such as actimomycin D, daunomycin (rubidomycin), doxorubicin, mitoxantrone, idarubicin, bleomycins, plicamycin (mithramycin), mitomycinC, and actinomycin; enzymes such as L-asparaginase, cytokines such as interferon (IFN)-gamma, tumor necrosis factor (TNF)-alpha, TNF-beta and GM-CSF, anti-angiogenic factors, such as angiostatin and endostatin, inhibitors of FGF or VEGF such as soluble forms of receptors for angiogenic factors, including soluble VGF/VEGF receptors, platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; non-steroidal antiandrogens such as flutamide; kinase inhibitors, histone deacetylase inhibitors, methylation inhibitors, proteasome inhibitors, monoclonal antibodies, oxidants, anti-oxidants, telomerase inhibitors, BH3 mimetics, ubiquitin ligase inhibitors, stat inhibitors and receptor tyrosin kinase inhibitors such as imatinib mesylate (marketed as Gleevac or Glivac) and erlotinib (an EGF receptor inhibitor) now marketed as Tarveca; and anti-virals such as oseltamivir phosphate, Amphotericin B, and palivizumab.

31. The use of any one of claims 28-30, wherein the anti-HVEM antibody is co-administered with a molecule selected from: (a) an anti-CXCL12 antibody; (b) an anti-CXCR4 antibody; (c) an anti-CD47 antibody; (d) a checkpoint inhibitor antibody, preferably an anti-PD-1 antibody, an anti-PD-L1 antibody, an anti-CTLA-4 antibody, an anti-TIM-3 antibody, and/or an anti-LAG3 antibody, (e) an anti-T-cell co-receptor antibody (e.g., an anti-4-1 BB (CD137) antibody or an anti-ICOS (CD278) antibody); or (f) an anti-neoantigen antibody.

32. The use of claim 31, wherein the anti-HVEM antibody is co-administered with an anti-neoantigen antibody, and the neoantigen is selected from: MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-I, GAGE-2, GAGE-3, GAGE-4, GAGE-5, GAGE-6, GAGE-7, GAGE-8, BAGE-I, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-1, SSX-I, SSX-2(HOM-MEL-40), SSX-3, SSX-4, SSX-5, SCP-1 and XAGE, melanocyte differentiation antigens, p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAA0205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-1), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding proteincyclophilin C-associated protein), TAAL6, TAG72, TLP, TPS, tyrosinase related proteins, TRP-1, TRP-2, or mesothelin.

33. The use of any one of claims 28-32, wherein the co-administration occurs simultaneously, separately, or sequentially with the anti-HVEM antibody.

34. A method of detecting HVEM in vitro in a sample, comprising contacting the sample with the antibody of any one of claims 1-19.
